(19) United States
(12) Patent Application Publication
(10) Pub. No.: US 2024/0092867 A1
(43) Pub. Date: Mar. 21, 2024

(54) METHODS AND COMPOSITIONS FOR THE TREATMENT OF WOUNDS

(71) Applicants: The University of Chicago, Chicago, IL (US); Imperial College Innovations Limited, London (GB)

(72) Inventors: Jeffrey A. HUBBELL, Chicago, IL (US); Anna M. RANDI, London (GB); Jun ISHIHARA, London (GB); Ako ISHIHARA, London (GB); Priscilla BRIQUEZ, Chicago, IL (US); Richard STARKE, London (GB)

(73) Assignees: The University of Chicago, Chicago, IL (US); Imperial College Innovations Limited, London (GB)

(21) Appl. No.: 18/352,101

(22) Filed: Jul. 13, 2023

Related U.S. Application Data
(63) Continuation of application No. 15/733,085, filed on May 13, 2020, now Pat. No. 11,732,029.
(60) Provisional application No. 62/758,845, filed on Nov. 12, 2018.
(60) Provisional application No. 62/585,101, filed on Nov. 13, 2017.

(51) Int. Cl. C07K 14/78; A61K 9/00; A61K 9/70; A61K 38/00; A61K 38/18; A61K 38/39; A61K 47/64; A61K 47/69; A61L 27/22; A61P 17/02; C07K 14/475; C07K 14/705; C07K 14/81; C07K 16/18

(52) U.S. Cl. CPC C07K 14/78; A61K 9/0024; A61K 9/7007; A61K 38/00; A61K 38/18; A61K 38/39; A61K 47/6435; A61K 47/6903; A61L 27/22; A61P 17/02; C07K 14/475; C07K 14/70546; C07K 14/8121; C07K 16/18; A61L 2300/412; C07K 2317/55; C07K 2319/70

(57) ABSTRACT

The methods and compositions described herein address the need in the art by providing peptides and polypeptides comprising a growth factor binding domain. In some embodiments, the peptides have an amino acid sequence that is at least 80% identical to one of SEQ ID NOS:1-7, 13-15, 49-50, or 66-70, or a fragment thereof; wherein the peptide is less than 300 amino acids in length.

### CROSS-REFERENCE TO RELATED APPLICATIONS

This application is a continuation of U.S. patent application Ser. No. 15/733,085, filed May 13, 2020, which is a national phase application under 37 U.S.C. § 371 of International Application No. PCT/US2018/060760, filed Nov. 13, 2018, which claims the benefit of priority to U.S. Provisional Patent Application No. 62/758,845, filed Nov. 12, 2018, and U.S. Provisional Patent Application No. 62/585,101, filed Nov. 13, 2017. The entire contents of each of the above-referenced disclosures are specifically incorporated herein by reference without disclaimer.

### STATEMENT OF GOVERNMENT SUPPORT

The invention was made with government support under DK108215 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Jul. 12, 2023, is named “ARCDP0644USC1” and is 117 kilobytes in size.

### BACKGROUND

### Field of the Invention

The invention generally relates to the field of medicine. More particularly, it concerns compositions and methods involving peptides providing for the delivery and/or in vivo recruitment of growth factors.

### Background

GFs are considered as crucial molecules in regenerative medicine, including the treatment of chronic diabetic ulcers as well as the treatment of non-regenerating bone defect (chronic non-union fractures, critical bone defects). However, GFs have had only modest effects in the clinic to date (Fonder, M. A. et al. Journal of the American Academy of Dermatology 58, 185-206, (2008) and Falanga, V. Lancet (London, England) 366, 1736-1743, (2005)). For example, recombinant human VEGF-A has not been approved for clinical use by the U.S. Food and Drug Administration (FDA) due to a negative result in phase II clinical trials (Whittam, A. J. et al. Advances in wound care 5, 79-88 (2016)). PDGF-BB (Regranex in the clinic) has shown clinical efficacy, but safety issues such as cancer risk have been flagged, potentially due to high dosing (Marti-Carvajal, A. J. et al. The Cochrane database of systematic reviews, Cd008548, (2015) and Papanas, D. & Maltezos, E. Drug safety 33, 455-461 (2010)). As another example, the bone morphogenetic protein-2 (BMP-2) was delivered through collagen sponges in InFUSE® Bone Graft (Medtronic) at supraphysiological doses, and led to serious side effects as ectopic bone growth, increased cancer risk and nerve injuries. Therefore, engineering GF delivery approaches for regenerative medicine, including for wound healing and bone repair, to enhance efficacy and reduce GF doses and side effects is crucial. Due to the challenges of delivering growth factors, there is a need in the art for more advanced growth factor delivery and/or in vivo treatments.

### SUMMARY OF INVENTION

The methods and compositions described herein address the need in the art by providing peptides and polypeptides comprising a growth factor binding domain that are useful in tissue regeneration, wound healing, and the treatment of certain disorders. In some embodiments, the peptides have an amino acid sequence that is at least 80% identical to one of SEQ ID NOS:1-7, 13-15, 49-50, or 66-70, or a fragment thereof; wherein the peptide is less than 300 amino acids in length.

In some embodiments, the peptides have an amino acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to one of SEQ ID NOS:1-7, 13-15, 49-50, or 66-70, or a fragment thereof.

In some embodiments, the peptide is less than 300, 275, 250, 225, 200, 190, 180, 170, 160, 150, 140, 130, 120, 110, 100, 90, 80, 70, 60, 50, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, or 8 amino acids in length (or any derivable range therein).

In some embodiments, the peptide is attached to a transglutaminase-reactive peptide. In some embodiments, the transglutaminase-reactive peptide is attached to the amino or carboxy end of the growth factor binding domain peptide. In some embodiments, the transglutaminase-reactive peptide is from the α2-plasmin inhibitor. In some embodiments, the transglutaminase-reactive peptide comprises an amino acid sequence that is at least 80% identical to SEQ ID NO:12 or a fragment thereof. In some embodiments, the transglutaminase-reactive peptide comprises an amino acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to SEQ ID NO:12 or a fragment thereof.

In some embodiments, the peptide comprises an amino acid sequence that is at least 80% identical to SEQ ID NO:8, 16-13, or a fragment thereof. In some embodiments, the peptide comprises an amino acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to SEQ ID NO:8, 16-13, or a fragment thereof.

In some embodiments, the peptide comprises an amino acid sequence that is at least 80% identical to SEQ ID NO:49 or 50, or a fragment thereof. In some embodiments, the peptide comprises an amino acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to SEQ ID NO:49 or 50. In some embodiments, the peptide comprises a positively charged residue at position 14 of SEQ ID NO:49 or 50. In some embodiments, the positively charged residue comprises lysine, arginine, or histidine. In some embodiments, the peptide is unsubstituted at position 14 of SEQ ID NO:49 or 50. In some embodiments, the positively charged residues are unsubstituted or substituted with another positively charged residue. In some embodiments, the arginine residues are unsubstituted.

In some embodiments, the peptide is linked to one or more additional peptides, wherein each additional peptide has an amino acid sequence that is at least 80% identical to one of SEQ ID NOS:1-7, 13-15, 49-50, or 66-70, or a fragment thereof. In some embodiments, at least 2, 3, 4, 5, 6, or 7 peptides are linked together, wherein each linked peptide has an amino acid sequence that is at least 80% identical to one of SEQ ID NOS:1-7, 13-15, 49-50, or 66-70, or a fragment thereof. In some embodiments, the peptides are separated by one or more linkers. In some embodiments, the linker comprises SEQ ID NO:60, wherein x=1, 2, 3, 4, 5, or 6 or comprises SEQ ID NO:61. In some embodiments, the linker(s) comprises a flexible linker. In some embodiments, the flexible linker comprises glycine and serine amino acid residues.

In some embodiments, the peptide is attached to a collagen binding peptide. In some embodiments, the collagen binding peptide comprises the A3 domain of von Willebrand Factor (vWF A3) or fragment thereof, or a peptide with at least 80% identity to vWF A3 or fragment thereof. In some embodiments, the collagen binding peptide comprises a peptide having an amino acid sequence of SEQ ID NO:47 or a fragment thereof, or a peptide with at least 80% identity to SEQ ID NO:47 or fragment thereof. In some embodiments, the collagen binding peptide comprises a peptide with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:47 or fragment thereof. In some embodiments, the collagen binding peptide comprises a decorin polypeptide or fragment thereof, or a peptide with at least 80% identity to a decorin polypeptide or fragment thereof. In some embodiments, the collagen binding peptide comprises a peptide having an amino acid sequence of SEQ ID NO:48 or a fragment thereof, or a peptide with at least 80% identity to SEQ ID NO:48 or fragment thereof. In some embodiments, the collagen binding peptide comprises a peptide with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to SEQ ID NO:48 or fragment thereof.

In some embodiments, the collagen binding peptide comprises one or more complementarity determining regions (CDRs) from an anti-collagen antibody. In some embodiments, the collagen binding peptide comprises a CDR1, CDR2, and/or CDR3 from a light chain variable region of an anti-collagen antibody. In some embodiments, the collagen binding peptide comprises a CDR1, CDR2, and CDR3 from a light chain variable region of an anti-collagen antibody. In some embodiments, the collagen binding peptide comprises a CDR1, CDR2, and/or CDR3 from a heavy chain variable region of an anti-collagen antibody. In some embodiments, the collagen binding peptide comprises a CDR1, CDR2, and CDR3 from a heavy chain variable region of an anti-collagen antibody. In some embodiments, the collagen binding peptide comprises a heavy or light chain variable region from an anti-collagen antibody. In some embodiments, the collagen binding peptide comprises a collagen-binding fragment from an anti-collagen antibody or a collagen-binding fragment derived from an anti-collagen antibody. In some embodiments, the collagen binding peptide comprises an anti-collagen antibody, or a Fab, scFv, nanobody, minibody, or unibody from an anti-collagen antibody or derived from an anti-collagen antibody. In some embodiments, the collagen binding peptide is humanized or chimeric. In some embodiments, the collagen binding peptide comprises human constant regions or a human framework. In some embodiments, the collagen binding peptide is chemically conjugated to the peptide. In some embodiments, there is a linker between the collagen binding peptide and the peptide comprising a growth factor binding domain. In some embodiments, the linker comprises SEQ ID NO:60, wherein x=1, 2, 3, 4, 5, or 6 or comprises SEQ ID NO:61. In some embodiments, the linker(s) comprises a flexible linker. In some embodiments, the flexible linker comprises glycine and serine amino acid residues. In some embodiments, the peptide is attached to the carboxy terminus of the collagen binding peptide. In some embodiments, the peptide is attached to the amino terminus of the collagen binding peptide.

In some embodiments, the collagen-binding domain is derived from variable regions of an anti-collagen antibody. In some embodiments, the collagen-binding domain comprises one or both of a heavy chain variable region and a light chain variable region of a collagen-binding antibody. Examples include single-chain variable fragments (scFv), antigen-binding fragments (Fab), and third-generation (3G) molecules such as nanobodies, minibodies, and unibodies.

In some embodiments, the peptide is chemically synthesized. In some embodiments, the peptide comprises a methionine as the amino-terminal amino acid. In some embodiments, the methionine is immediately adjacent to the first amino acid of one of SEQ ID NOS:1-7, 13-15, 49-50, or 66-70. In some embodiments, the amino terminal methionine is immediately adjacent to one of the peptide embodiments of the disclosure.

In some embodiments, the peptide is attached to a cell adhesion moiety. In some embodiments, the cell adhesion moiety comprises a ligand for a glycoprotein or a cell surface receptor. In some embodiments, the cell adhesion moiety comprises an integrin-binding peptide.

In some embodiments, the peptide is attached to a tag. In some embodiments, the tag comprises a purification tag, a signaling sequence, a post-translational modifier, or a targeting moiety. In some embodiments, the peptide is attached to a tag described herein. In some embodiments, the peptide is conjugated to a functional moiety. In some embodiments, the functional moiety comprises an antibody, an enzyme, a fluorescent compound, an imaging agent, or a therapeutic agent. In some embodiments, the functional moiety comprises a gadolinium chelation moiety. In some embodiments, the peptide is attached to a functional moiety described herein. In some embodiments, the tag and/or functional moiety is at the carboxy or amino terminus of the peptide.

In some embodiments, the peptide comprises two or more growth factor binding domains, wherein each growth factor binding domain has an amino acid sequence that is at least 80% identical to one of SEQ ID NOS:1-7, 13-15, 49-50, or 66-70. In some embodiments, the peptide comprises two or more growth factor binding domains, wherein each growth factor binding domain has an amino acid sequence that is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to one of SEQ ID NOS:1-7, 13-15, 49-50, or 66-70.

In some embodiments, the peptide comprises one or more substitutions relative to SEQ ID NOS:1-7, 13-15, 49-50, or 66-70. For example, the peptide may comprise at least, at most, or exactly 1, 2, 3, 4, 5, 6, 7, 8, 9, 10, 11, 12, 13, 14, or 15 (or any derivable range therein) substitutions at position(s) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and/or 200. In some embodiments, the one or more substitutions are conservative substitutions. In further embodiments, the one or more substitutions are non-conservative. In other embodiments, the one or more substitutions are a mix of conservative and non-conservative substitutions.

Further aspects of the disclosure relate to a molecular complex comprising any of the peptide embodiments described herein and one or more growth factors or cytokines are bound to the peptide. In some embodiments, the growth factors are bound by non-covalent interactions with the peptide. In some embodiments, the growth factors comprise one or more of VEGF, PlGF, PDGF, FGF, and BMP. In some embodiments, the growth factor comprises one or more of VEGF-A 165, PlGF2, PDGF-BB, PDGF-CC, FGF-2, and BMP-2. In some embodiments, the molecular complex comprises one or more growth factors or cytokines described herein. In some embodiments, the growth factor is linked to an ECM-binding domain. In some embodiments, the ECM-binding domain is from PlGF or from PlGF2. In some embodiments, the ECM-binding domain is linked to the peptide through a peptide bond. Further examples of ECM binding domains are described in WO2014006082A1.

Further aspects of the disclosure relate to a composition comprising any of the peptide or molecular complex embodiments described herein. In some embodiments, the composition further comprises one or more growth factors. In some embodiments, the growth factors comprise one or more of VEGF, PlGF, PDGF, FGF and BMP. In some embodiments, the growth factor comprises one or more of VEGF-A 165, PlGF2, PDGF-BB, FGF-2 and BMP-2. In some embodiments, the composition comprises one or more growth factors or cytokines described herein.

Further aspects of the disclosure relate to a biomaterial scaffold comprising any of the peptide or molecular complex embodiments described herein. In some embodiments, the scaffold comprises fibrin. In some embodiments, the peptide is covalently linked to the fibrin. In some embodiments, the covalent linkage is through the α2 plasmin inhibitor peptide (α₂PI₁₋₈). In some embodiments, the scaffold comprises one or more of collagen, heparin, ceramic, a synthetic polymer, proteoglycans alginate-based substrates, chitosan, hyaluronic acid and/or methylcellulose substrates. In some embodiments, the biomaterial comprises less than 50 mg of exogenous growth factors. The term exogenous refers to materials, such as growth factors, that are added outside the body and do not include any of those materials that may be present in the body and associate with the scaffold or peptide in vivo. The exogenous components may be polypeptides and proteins that have been recombinantly or chemically produced.

In some embodiments, the dose of a growth factor is adminstered according to a dosage amount and schedule described herein.

In some embodiments, with respect to PDGF or specifically PDGF-BB or PDGF-CC, the dosage may be at most, at least, or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 (or any derivable range therein) μg/kg body weight. In some embodiments, with respect to PDGF or specifically PDGF-BB, the dosage may be at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500 mg, 1 μg, or ng/dose (or any derivable range therein). In some embodiments, with respect to PDGF or specifically PDGF-BB, the dosage may be at most, at least, or exactly 1, 2, 3, 4, 5, 6, 7, 8, 9, 10, 11, 12, 13, 14, 15, 16, 17, 18, 19, or 20 μg/cm2 wound or tissue area (or any derivable range therein). The administration may be repeated daily or every 2, 3, 4, 5 6, or 7 days (or any derivable range therein for at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or 1 2, 3, 4, 5, 6, 7, 8, 9, or 10 weeks (or any derivable range therein). In some embodiments, the dose refers to a total prescribed dose that is to be administered over a period of time.

In some embodiments, with respect to VEGF or specifically VEGF-A or VEGF-A 165, the dosage may be at most, at least, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.75, 2, 2.25, 2.5, 2.75, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 54, 55, 56, 58, 60, 62, 64, 66, 68, 70, 72, 75, or 100 mg, 1 μg, or ng/dose (or any derivable range therein). In some embodiments, with respect to VEGF or specifically VEGF-A or VEGF-A 165, the dosage may be at most, at least, or exactly 100, 125, 150, 175, 200, 225, 250, 275, 300, 325, 350, 375, 400, 425, 450, 475, 500, 525, 550, 575, 600, 625, 650, 670, 675, 700, 725, 750, 775, or 800 μg/cm2 wound or tissue area (or any derivable range therein). The administration may be repeated daily or every 2, 3, 4, 5 6, or 7 days (or any derivable range therein for at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or 1 2, 3, 4, 5, 6, 7, 8, 9, or 10 weeks (or any derivable range therein). In some embodiments, the dose refers to a total prescribed dose that is to be administered over a period of time.

In some embodiments, with respect to FGF or specifically FGF-2, the dosage may be at most, at least, or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 (or any derivable range therein) μg/kg body weight. In some embodiments, with respect to FGF or specifically FGF-2, the dosage may be at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500 mg, 1 μg, or ng/dose (or any derivable range therein). In some embodiments, with respect to FGF or specifically FGF-2, the dosage may be at most, at least, or exactly 1, 2, 3, 4, 5, 6, 7, 8, 9, 10, 11, 12, 13, 14, 15, 16, 17, 18, 19, or 20 μg/cm2 wound or tissue area (or any derivable range therein). The administration may be repeated daily or every 2, 3, 4, 5 6, or 7 days (or any derivable range therein for at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or 1 2, 3, 4, 5, 6, 7, 8, 9, or 10 weeks (or any derivable range therein). In some embodiments, the dose refers to a total prescribed dose that is to be administered over a period of time.

In some embodiments, with respect to PlGF or specifically PlGF2, the dosage may be at most, at least, or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 (or any derivable range therein) μg/kg body weight. In some embodiments, with respect to PlGF or specifically PlGF2, the dosage may be at least, at most, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500 mg, 1 μg, or ng/dose (or any derivable range therein). In some embodiments, with respect to PlGF or specifically PlGF2, the dosage may be at most, at least, or exactly 1, 2, 3, 4, 5, 6, 7, 8, 9, 10, 11, 12, 13, 14, 15, 16, 17, 18, 19, or 20 μg/cm2 wound or tissue area (or any derivable range therein). The administration may be repeated daily or every 2, 3, 4, 5 6, or 7 days (or any derivable range therein for at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or 1 2, 3, 4, 5, 6, 7, 8, 9, or 10 weeks (or any derivable range therein). In some embodiments, the dose refers to a total prescribed dose that is to be administered over a period of time.

In some embodiments, with respect to BMP or specifically BMP-2, the dosage may be at most, at least, or exactly 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5 (or any derivable range therein) μg/kg body weight. In some embodiments, with respect to BMP or specifically BMP-2, the dosage may be at least, at most, or exactly 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 or 500 mg, μg, or ng/dose (or any derivable range therein). In some embodiments, with respect to BMP or specifically BMP-2, the dosage may be at most, at least, or exactly 1, 2, 3, 4, 5, 6, 7, 8, 9, 10, 11, 12, 13, 14, 15, 16, 17, 18, 19, or 20 μg/cm2 wound or tissue area (or any derivable range therein). The administration may be repeated daily or every 2, 3, 4, 5 6, or 7 days (or any derivable range therein for at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or 1 2, 3, 4, 5, 6, 7, 8, 9, or 10 weeks (or any derivable range therein). In some embodiments, the dose refers to a total prescribed dose that is to be administered over a period of time.

In some embodiments, externally added VEGF-A165 is in an amount of less than 20 μg, less than 10 μg, less than 1 μg, less than 500 ng, less than 400 ng, less than 300 ng, less than 200 ng, less than 100 ng, or less than 1 ng. In some embodiments, externally added PDGF-BB is in an amount of less than 10 μg, less than 1 μg, less than 500 ng, less than 400 ng, less than 300 ng, less than 200 ng, less than 100 ng, or less than 1 ng.

In some embodiments, the biomaterial scaffold or implant is one that retains at least 80% of exogenously added growth factors for at least 3 days. In some embodiments, the biomaterial scaffold or implant is one that retains at least 50, 60, 70, 75, 80, 85, 90, 91, 92, 93, 94, 95, 96, 97, 98, 99, or 100% (or any derivable range therein) of exogenously added growth factors for at least 1, 2, 3, 4, 5, 6, 7, 8, 9, or 10 days (or any derivable range therein).

Further aspects of the disclosure relate to an implant comprising any one of the peptide, molecular complex, composition, or biomaterial embodiments described herein. In some embodiment, the implant comprises a medical device, a stent, or a vascular graft.

Further aspects relate to a method for regenerating tissue in a subject, the method comprising administering a peptide, molecular complex, composition, biomaterial scaffold, or implant embodiment of the disclosure to the subject.

Further aspects relate to a method for facilitating wound or tissue healing in a subject, the method comprising administering a peptide, molecular complex, composition, biomaterial scaffold, or implant embodiment of the disclosure to the subject.

Yet further aspects relate to a method for treating angiodysplasia and/or von mucosal/cutaneous bleeding in a subject, the method comprising administering a biomaterial scaffold, composition, or implant of the disclosure to the subject. Yet further aspects relate to a method for treating von Willebrand disease (VWD) in a subject, the method comprising administering a biomaterial scaffold, composition, or implant of the disclosure to the subject. In some embodiments, von Willebrand disease comprises acquired von Willebrand disease (AVWD). In some embodiments, von Willebrand disease comprises congenital von Willebrand disease (AVWD). In some embodiments, VWD comprises type 1 VWD. In some embodiments, VWD comprises type 2 VWD. In some embodiments, VWD comprises type 3 VWD. In some embodiments, VWD comprises type 2A VWD. In some embodiments, VWD comprises type 2B VWD. In some embodiments, the method is for treating GI bleeding associated with angiodysplasia. In some embodiments, the subject is one that has reduced high molecular weight multimers (HMWM) of the vWF protein.

Yet further aspects of the disclosure relate to the treatment of diabetic ulcers in a subject, the method comprising administering a biomaterial scaffold, composition, or implant of the disclosure to the subject.

In some embodiments, the peptide, molecular complex, composition, biomaterial scaffold, or implant is administered locally to a specific tissue or wound. In some embodiments, the subject has or has been diagnosed with a deficiency in wound healing. In some embodiments, the subject has diabetes. In some embodiments, the wound comprises a diabetic ulcer. In some embodiments, the tissue comprises bone. In some embodiments, the tissue is one disclosed herein. In some embodiments, the biomaterial scaffold or implant is administered locally to bone or a location adjacent thereto. In some embodiments, the percentage of wound closure after seven days of administration is at least 60%. In some embodiments, the percentage of wound closure after 1, 2, 3, 4, 5, 6, 7, 8, 9, 10, 11, 12, 13, 14, 15, 16, 17, 18, 19, or 20 days (or any derivable range therein) of administration is at least 40, 45, 50, 55, 60, 65, 70, 75, 80, 85, 90, 95, 96, 97, 98, or 99%, or any derivable range therein. In some embodiments, the amount of granulation of the tissue after seven days of administration is at least 1 mm². In some embodiments, the amount of granulation of the tissue after 1, 2, 3, 4, 5, 6, 7, 8, 9, 10, 11, 12, 13, 14, 15, 16, 17, 18, 19, or 20 days (or any derivable range therein) of administration is at least 0.5, 0.6, 0.7, 0.8, 0.9, 1.0, 1.1, 1.2, 1.3, 1.4, 1.5, 1.6, 1.8, 1.9, or 2 mm², or any derivable range therein.

In some embodiments, the subject has and/or has been diagnosed with von Willebrand disease (VWD). In some embodiments, VWD comprises type 1 VWD. In some embodiments, VWD comprises severe type 1 VWD. In some embodiments, VWD comprises type 2 VWD. In some embodiments, VWD comprises type 3 VWD. In some embodiments, VWD comprises type 2A VWD. In some embodiments, VWD comprises type 2B VWD. In some embodiments, the subject has and/or has been diagnosed with acquired von Willebrand disease (AVWD). In some embodiments, the subject has and/or has been diagnosed with congenital von Willebrand disease. In some embodiments, the subject is deficient for the vWF protein. In some embodiments, the subject has been determined to be deficient for the vWF protein. In some embodiments, the subject has and/or has been determined to have a mutant vWF protein. In some embodiments, the subject has been identified with having blood vessel abnormalities. In some embodiments, the subject has and/or has been determined to have a mutation in the A1 domain of vWF. In some embodiments, the subject has a mutant vWF with increased affinity for GPIbα. In some embodiments, the subject has been shown to have one or more of spontaneous platelet aggregation, loss of active high molecular weight vWF multimers, thrombocytopenia and/or bleeding. In some embodiments, the subject has been determined to have mutations in exon 28 of the vWF gene. In some embodiments, the subject has been determined to have a R1341 substitution or deletion in the vWF protein, or a mutation in the vWF gene which results in a R1341 substitution or deletion in the vWF protein. In some embodiments, the subject is determined to have a R1341 substitution, wherein the arginine is substituted with Leu, Pro, Gln, Trp, or Ser. In some embodiments, the subject has been diagnosed with angiodysplasia. In some embodiments, the subject has been determined to have GI bleeding. In some embodiments, the subject is one that has reduced high molecular weight multimers (HMWM) of the vWF protein.

In some embodiments, the patient has been previously treated for a condition or indication described herein. In some embodiments, the subject was resistant to the previous treatment. In some embodiments, the patient has been diagnosed with and/or is susceptible to a condition or indication described herein. In some embodiments, the method further comprises administration of an additional therapy, such as, for example, additional therapies described herein.

The terms “protein”, “polypeptide” and “peptide” are used interchangeably herein when referring to a gene product or synthetic amino acid polymer.

The terms “subject,” “mammal,” and “patient” are used interchangeably. In some embodiments, the subject being treated is a mammal. In some embodiments, the subject is a human. In some embodiments, the subject is a mouse, rat, rabbit, dog, donkey, sheep, goat, pig, or a laboratory test animal such as fruit fly, zebrafish, etc.

It is contemplated that the methods and compositions include exclusion of any of the embodiments described herein.

The terms “a” and “an” are defined as one or more unless this disclosure explicitly requires otherwise.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements, but is not limited to possessing only those one or more elements. Likewise, an element of a method or system of the present invention that “comprises,” “has,” “includes” or “contains” one or more features possesses those one or more features, but is not limited to possessing only those one or more features.

The feature or features of one embodiment may be applied to other embodiments, even though not described or illustrated, unless expressly prohibited by this disclosure or the nature of the embodiments.

Any method or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb. A composition “consisting essentially of” the recited elements excludes any further active ingredients but does not exclude pharmaceutical excipients, buffers, structural components, etc.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Multiple isoforms of laminin bind promiscuously to GFs and chemokines with high affinities. (A) Binding of multiple isoforms of full-length laminin (−111, −211, −332, −411, −421, −511, and −521) to GFs and CXCL chemokines were measured by ELISA. A450 nm represents absorbance at 450 nm. BSA-coated wells served as negative controls (n=4, mean±SEM). Signals greater than 0.1 (grey box) are considered to be significant. (B) Affinities (K_{D} values are shown) of full-length laminin against VEGF-A165, PlGF-2 and PDGF-BB were measured by SPR. A SPR chip was functionalized with laminin-521 (˜2000 RU), and each GF was flowed over the chip at indicated concentrations. Curves represent the specific responses (in RU) to laminin obtained. Experimental curves were fitted with Langmuir binding kinetics. Binding kinetics values [dissociation constants (K_{D}) and rate constants (Kₒₙ and K_{off})] determined from the fitted curves are shown.

FIGS. 2A-C. Excess heparin inhibits GF-laminin binding. Inhibition of GF-binding to laminin (−111, −211, −221, −411, −421, −511, and −521) by excess heparin. ELISA plates were coated with 10 μg/mL laminin and further incubated with a 1 μg/mL (A) VEGF-A165, (B) PlGF-2, or (C) FGF-2 solution in the absence or presence of excess (10 μM) heparin. Bound GFs were detected using a specific antibody for each GF (n=4, mean±SEM). Statistical analyses were done using the Mann-Whitney U test by comparing the signals with and without heparin. *p<0.05, **p<0.01.

FIGS. 3A-D. GFs bind to recombinant LG domain protein derived from laminin α3, α4 and α5 chains. Affinity of GFs against recombinant laminin LG domains. ELISA plates were coated with 1 μg/mL (A) α3₂₉₂₈₋₃₁₅₀, (B) α4₈₂₆₋₁₈₁₆, or (C) α5₃₀₂₆₋₃₄₈₂ and further incubated with 1 μg/mL of VEGF-A165, VEGF-A121, PlGF-2, PlGF-1, PDGF-BB, or FGF-2 solution. Bound GFs were detected using a specific antibody for each GF (n=4, mean±SEM). Statistical analyses were done using the Mann-Whitney U test by comparing the signals obtained from the laminin domain- and the BSA-coated wells. *p<0.05, **p<0.01. (D) Affinities (K_{D} values are shown) of laminin α3₂₉₂₈₋₃₁₅₀ against VEGF-A165 and PDGF-BB were measured by SPR. A SPR chip was functionalized with the laminin α3₂₉₂₈₋₃₁₅₀ recombinant protein (˜1000 RU), and each GF was flowed over the chip at indicated concentrations. Curves represent the specific responses (in RU) to laminin. Experimental curves were fitted with Langmuir binding kinetics. Binding kinetics values [dissociation constants (K_{D}) and rate constants (Kₒₙ and K_{off})] determined from the fitted curves are shown.

FIGS. 4A-F. GFs bind to chemically synthesized laminin HBD peptides derived from the LG domain of laminin α3, α4, and α5 chains. (A) The location of laminin-derived peptides in the LG domain of laminin α3, α4, and α5 chains. (B-F) Affinity of heparin and GFs against chemically synthesized peptides derived from the LG domain of laminin α3, α4, and α5 chains. ELISA plates were coated with 10 μg/mL laminin peptide and further incubated with (B) biotinylated heparin, (C) VEGF-A165 and VEGF-A121, (D) PlGF-2 and PlGF-1, (E) PDGF-BB, or (F) FGF-2. Concentrations were 1 μg/mL for GFs and 10 μg/mL for heparin. Bound heparin was detected with streptavidin, and bound GFs with a specific antibody for each GF (n=4, mean±SEM). Statistical analyses were done using the Mann-Whitney U test by comparing the signals obtained from the laminin peptide- and the BSA-coated wells. *p<0.05, **p<0.01.

FIGS. 5A-D. Chemically synthesized peptides derived from the LG domain of laminin α3, α4 and α5 chains bind to syndecans. Affinity of syndecans to chemically synthesized peptides derived from the laminin α3, α4 and α5 LG domains. ELISA plates were coated with 10 μg/mL laminin peptide and further incubated with 1 μg/mL of (A) syndecan-1, (B) syndecan-2, (C) syndecan-3, or (D) syndecan-4. Bound syndecans were detected using an antibody against histidine-tag on the recombinant syndecans (n=8, mean±SEM). Statistical analyses were done using the Mann-Whitney U test by comparing the signals obtained from the laminin peptide- and the BSA-coated wells. *p<0.05, **p<0.01.

FIGS. 6A-D. Laminin HBD peptides promote fibroblast and endothelial cell adhesion in vitro. (A, B) 3000 cells/well human lung fibroblasts were cultured (A) without or (B) with 5 mM EDTA in FGM-2 culture media containing 1% FBS. (C, D) 3000 cells/well HUVEC were cultured (C) without or (D) with 5 mM EDTA in EBM-2 culture media containing 100 ng/ml VEGF-A165 and 1% FBS. Cells were plated on 1 μg/mL laminin peptide pre-coated non-tissue culture treated plates and incubated for 30 min at 37° C. After plate washes, cell numbers were quantified using a CyQUANT assay (n=10, mean±SEM). The signals obtained from BSA-coated wells are normalized to 1, and relative fold increases of cell numbers were calculated. Statistical analyses were done using ANOVA with Tukey's test. Kruskal-Wallis test followed by Dunn's multiple comparison was used in (B, C). *p<0.05, **p<0.01.

FIGS. 7A-C. GF retention in fibrin matrices is enhanced by incorporating laminin HBD peptide. (A,B) GF retention in fibrin matrix. α₂PI₁₋₈-α3₃₀₄₃₋₃₀₆₇ or α₂PI₁₋₈-α5₃₄₁₇₋₃₄₃₆ peptide-functionalized fibrin matrices were made in the presence of VEGF-A165 or PDGF-BB, and incubated in 8 volumes of physiological buffer for 5 days. The buffer was changed each day, and released GFs were quantified daily. Graphs show the cumulative release of (A) VEGF-A165 or (B) PDGF-BB over 5 days (n=4; mean±SEM). All data points for laminin HBD peptides were statistically significant compared to controls without laminin HBD peptide (p<0.01, Mann-Whitney U test). (C) Fibrin matrices containing VEGF-A165 (200 ng/wound) with or without α₂PI₁₋₈-α3₃₀₄₃₋₃₀₆₇ peptide were placed on the full-thickness back-skin wounds in db/db diabetic mice. After 3 and 6 days, retention of VEGF-A165 after 3 and 6 days in the fibrin matrix and the tissue surrounding the wound (2 mm beyond the wound margin) were quantified. n≥4 per time point, mean±SEM. Student's t-test; **p<0.01.

FIGS. 8A-G. Delivering GFs within laminin HBD peptide-functionalized fibrin matrices enhances skin wound healing in db/db diabetic mice. Full-thickness back-skin wounds were treated with combined VEGF-A165 (100 ng/wound) and PDGF-BB (50 ng/wound). Four groups were tested: fibrin only, fibrin functionalized with α₂PI₁₋₈-α3₃₀₄₃₋₃₀₆₇ peptide, fibrin containing admixed GFs, and fibrin functionalized with α₂PI₁₋₈-α3₃₀₄₃₋₃₀₆₇ peptide containing GFs. After 4, 7, and 10 days, (A-B) wound closure and (C) granulation tissue area were evaluated by histology (means±SEM, day 4: n=6, day 7: fibrin only and α₂PI₁₋₈-α3₃₀₄₃₋₃₀₆₇ peptide+GFs, n=10; other treatment groups, n=11, day 10: α2PI₁₋₈-α3₃₀₄₃₋₃₀₆₇ peptide, n=8, α₂PI₁₋₈-α3₃₀₄₃₋₃₀₆₇ peptide+GFs, n=9, other treatment groups, n=7). (B) The proportions of the mice were categorized by the degree of healing after day 7 of wound treatment. (D) Wound histology (hematoxylin and eosin staining) at day 7. Red arrows indicate tips of the epithelium tongue. The granulation tissue (pink-violet) is characterized by a large number of granulocytes with nuclei that stain in dark-violet or black. Muscle under the wounds is stained in red. Fat tissue appears as transparent bubbles. Scale bar=800 μm. (E-G) 5 days after the wound treatment, (E) proliferation of CD31⁺CD45⁻ endothelial cells is assessed by Ki67⁺ marker, and (F) the frequency of Ly6G⁺CD11b⁺ neutrophils within CD45⁺ cells and (G) the frequency of Ly6C⁺CD11b⁺ monocytes within CD45⁺ cells were determined using flow cytometry (means±SEM). *P<0.05, **P<0.01, ANOVA with Tukey's test.

FIG. 9. Scrambling the sequence of laminin HBD peptide decreases the GF binding capacity. Affinity of GFs against chemically synthesized peptides that are scrambled (Scr) the sequence of α3₃₀₄₃₋₃₀₆₂. ELISA plates were coated with 10 μg/mL laminin peptide and further incubated with VEGF-A165, PlGF-2, PDGF-BB, or FGF-2. Concentrations were 1 μg/mL for GFs. Bound GF was detected with a specific antibody for each GF (n=4, mean±SEM). Statistical analyses were done using one-way ANOVA. *p<0.05, **p<0.01. Sequence of the peptides are described in Table 2.

FIG. 10. Laminin HBD peptide did not enhance the migration of endothelial cells in vitro. 4×10⁴ HUVEC cells were added to the transwell upper parts. Solutions containing 30 ng/mL of VEGF-A165 preincubated with or without 0.1 μM of α3₃₀₄₃₋₃₀₆₇ peptide were added to the bottom side of the transwell. The signals of the cells that passed through a migration transwell after 6 hr of incubation were measured. (means±SEM, n=4). Statistical analyses were done using one-way ANOVA. **P<0.01

FIG. 11. Photos of the wounds. Full-thickness back-skin wounds were treated with combined VEGF-A165 (100 ng/wound) and PDGF-BB (50 ng/wound). Four groups were tested: fibrin only, fibrin functionalized with α₂PI₁₋₈-α3₃₀₄₃₋₃₀₆₇ peptide, fibrin containing admixed GFs, and fibrin functionalized with α₂PI₁₋₈-α3₃₀₄₃₋₃₀₆₇ peptide containing GFs. Representative pictures of wounds after 0 and 7 days are presented.

FIG. 12. Bipartite «bridge» proteins composed of a growth factor-binding domain linked to a collagen I-binding domain.

FIGS. 13A-F. Conjugation of a collagen-binding domain FabCol to a growth factor-binding domain FgHBD.

FIGS. 14A-F. Engineering recombinant fusion protein linking a collagen-binding domain FabCol to LamLG4 or FgHBD growth factor-binding domains to sequester rhBMP-2 into collagen biomaterials.

FIGS. 15A-E. vWF-deficient mouse shows impaired wound healing through poor angiogenesis. Full-thickness back-skin wounds were made in wild-type (WT) and vWF-deficient mice. After 5 d, (A) wound closure and (B) granulation tissue area were evaluated by histomorphometry. (means±SEM). Proliferation of (C) CD31+CD45− endothelial cells and (D) SMA+CD45− SMCs assessed by Ki67+ marker determined using flow cytometry (means±SEM). (E) The amounts of VEGF-A in the wounds were quantified by ELISA. *p<0.05, **p<0.01, ANOVA with Tukey's test.

FIGS. 16A-D. Human plasma-derived vWF binds promiscuously to GFs with high affinity. vWF binding to (A) GFs and (B) chemokines were measured by ELISA. A450 nm represents absorbance at 450 nm. Signals from VEGF-A121 served as a baseline, and bovine serum albumin (BSA) served as a negative control (n=4, mean±SD). Affinity (KD values are shown) of vWF against (C) VEGF-A165 and (D) PDGF-BB was measured by SPR. SPR chips were functionalized with vWF (2000 RU), and VEGF-A165 or PDGF-BB was flowed over the chips at indicated concentrations. Curves represent the specific responses (in resonance units (RU)) to vWF obtained. Experimental curves were fitted with (C) 1:1 Langmuir fit model and (D) heterogeneous ligand-parallel reactions binding. Binding kinetics values [dissociation constants (KD) and rate constants (kon and koff)] determined from the fitted curves are shown.

FIGS. 17A-B. vWF binds to VEGF-A in human serum. (A) ELISA plates were coated with 10 μg/mL anti-human vWF monoclonal antibody and further incubated with human serum. Bound VEGF-A was detected using a specific antibody for VEGF-A (n=3, mean±SD). (B) Human serum was subjected to immunoprecipitation with anti-human vWF monoclonal antibody or anti-human VEGF-A monoclonal antibody. Western blotting was performed with collected proteins using anti-human VEGF-A antibody. Representative image of 3 human serum. Statistical analyses were done using Student's t-test. **p<0.01.

FIGS. 18A-C. The HBD within the A1 domain of vWF mediates GF binding. (A) The location of the A1 domain and HBD within vWF. (B-C) Affinity of VEGF-A, PlGF, PDGF-BB, FGF-2, or CXCL-12 against (B) recombinant vWF A1 domain protein or (C) vWF A1 HBD peptide. ELISA plates were coated with 10 μg/mL recombinant vWF A1 domain protein or 10 μg/mL vWF A1 HBD peptide and further incubated with a 1 μg/mL VEGF-A, PlGF, PDGF-BB, FGF-2, or CXCL-12 solution. Bound GFs were detected using a specific antibody for each GF (n=4, mean±SD). Statistical analyses were done using ANOVA with Tukey's test or Student's t-test. *p<0.05, **p<0.01.

FIGS. 19A-C. R1341 mutations observed in vWD type 2B patients impaired vWF-GF binding. (A) Binding of VEGF-A165, PDGF-BB, and FGF-2 to vWF A1 HBDs with R1341 substitutions. (n=4, mean±SD). (B) Binding of VEGF-A165, PDGF-BB, and FGF-2 to recombinant human (rh)vWF with R1341Q substitution. (n=4, mean±SD). (C) Binding of VEGF-A165, PDGF-BB, and FGF-2 to vWF in healthy donor or type 2B vWD patient serum (n=3, mean±SD). Statistical comparisons were carried out using (A) ANOVA with Tukey's test compared with BSA control and (B-C) Student's t-test **p<0.01.

FIGS. 20A-F. Delivering GFs within vWF HBD-functionalized fibrin matrices enhance skin wound healing in diabetic mice. (A-B) GF retention in fibrin matrix. Graph showing the cumulative release of (A) VEGF-A165 or (B) PDGF-BB over 5 d (n=4; mean±SEM). Full-thickness back-skin wounds were treated with combined 100 ng of VEGF-A165 and 50 ng of PDGF-BB. Four groups were tested: fibrin only, fibrin functionalized with □₂PI₁₋₈-vWF HBD only, fibrin containing GFs only, and fibrin functionalized with □₂PI₁₋₈-vWF HBD containing GFs. (C) After 7 d, wound closure and (D) granulation tissue area were evaluated by histomorphometry. (means±SEM, n=11-13 per treatment group). (E-F) 5 d after the wound treatment, (E) the frequency of CD31⁺CD45⁻ endothelial cells within total alive cells and (F) proliferation of SMA⁺CD45⁻ SMC assessed by Ki67⁺ marker were determined using flow cytometry (means±SEM). *p<0.05, **p<0.01, ANOVA with Tukey's test.

FIG. 21. No binding was observed between VEGF-A121 and vWF. Affinity of VEGF-A121 for vWF, estimated by SPR. SPR chips were functionalized with plasma derived vWF, and VEGF-A121 was flowed over the chips at various concentrations (50-800 nM). Curves represent the responses (in RU) to vWF obtained.

FIGS. 22A-C. Excess heparin inhibits GF binding to vWF. Inhibition of GF binding to vWF by excess heparin. ELISA plates were coated with 10 μg/mL vWF and further incubated with a 1 μg/mL (A) VEGF-A165, (B) PlGF-2, or (C) FGF-2 solution containing 10 μM heparin. Bound GFs were detected using a specific antibody for each GF (n=4, mean±SD).

FIGS. 23A-D. vWF A1 HBD binds to VEGF-A145 and VEGF-A165. (A) Diagram of exon sequence of VEGF-A showing inclusion (+) or exclusion (−) of heparin binding domain exons for the different VEGF-A isoforms. (B-D) Binding of (B) VEGF-A165, (C) VEGF-A145, or (D) VEGF-A121 to vWF domains. ELISA plates were coated with 50 nM vWF domains and further incubated with recombinant human VEGF-A121, VEGF-A145 or VEGF-A165 (1 μg/mL, each). Bound VEGF-A was detected using a specific antibody for VEGF-A (n=4, mean±SD). Statistical comparisons were done using ANOVA with Tukey's test compared with BSA control. **p<0.01.

FIGS. 24A-B. The vWF A1 HBD retains GFs when incorporated into synthetic matrices. Retention of GFs in PEG-based synthetic matrix functionalized with C-terminus Cys added vWF HBD peptide using a Michael addition reaction. The graph shows the cumulative release of (A) FGF-2 or (B) CXCL-12γ over 5 d. (n=3; mean±SEM). All data points for vWF HBD were statistically significant compared to controls without vWF HBD (p<0.01, Student's t-test)

FIGS. 25A-D. Fibroblast attachment and proliferation on the vWF HBD peptide coated plate in vitro. Cell adhesion assays. 3000 cells/well human lung fibroblasts were cultured (A) without or (B) with 5 mM EDTA in FGM-2 culture media. Cells were plated on 1 μg/mL vWF HBD pre-coated non-tissue culture treated plates and incubated for 30 min at 37° C. After plate washes, cell numbers were quantified using a CyQUANT assay (n=4, mean±SD). (C) 1000 cells/well human lung fibroblasts or (D) 1000 cells/well human umbilical vein endothelial cells (HUVEC) were cultured on 1 μg/mL vWF HBD pre-coated 96-well tissue culture plates. Cell numbers were quantified after 72 hrs using a CyQUANT assay (n=4, mean±SD). The signals obtained from non-coated wells are normalized to 1, and relative fold increase of cell numbers were calculated. Statistical comparisons were carried out by Student's t-test. *p<0.05, N.S.=not significant.

### DETAILED DESCRIPTION

Lamin and von Willebrand (vWF) peptides that bind certain growth factors are useful in would healing and tissue repair.

Laminins have been reported as crucial molecules for adhesion of various cell types, both in vitro and in vivo, thus serving as a cell scaffold protein. The inventors found that multiple isoforms of laminin promiscuously bind several growth factors (GFs) from the VEGF/PDGF, FGF, BMP, and NT families, in addition to HB-EGF and CXCL12γ, through their heparin binding domains (HBDs). By engineering a fibrin matrix displaying the laminin peptide, the inventors have demonstrated that the laminin peptide linked to fibrin matrix promotes wound closure when applied to skin wounds in the db/db mouse, as a model of delayed wound healing, when applied with VEGF-A165 and PDGF-BB. In addition to showing a GF-modulating function for laminin, an important tissue repair protein, the examples also show that both GF- and cell-binding character promotes tissue repair when incorporated within fibrin matrix, which may be clinically useful. In addition, the inventors have demonstrated that the laminin HBD peptide can be fused or conjugated to collagen-binding domain to allow retention of GFs into collagen-based biomaterials. The inventors showed this art focusing on the sequestration of BMP-2 into collagen hydrogels and sponges for application in bone regeneration.

von Willebrand factor is a large plasma glycoprotein synthesized by endothelial cells and megakaryocytes. It is best known for its role in hemostasis, where it mediates platelet adhesion to the subendothelium at sites of endothelial damage and acts as a carrier to coagulation factor VIII. In patients with von Willebrand disease (vWD), the most common inherited bleeding disorder caused by defects in or deficiency of vWF, blood vessel abnormalities have been identified. In a subset of patients, vascular malformations in the gastrointestinal tract (i.e. angiodysplasia) can cause severe, intractable bleeding. vWF is comprised of a number of subunits, made up of conserved modular domains in the order D1-D2-D′-D3-A1-A2-A3-D4-B1-B2-B3-C1-C2. Mature vWF is formed after proteolysis of the vWF propeptide, i.e. the D1 and D2 domains. The A1 domain contains the binding site for platelet glycoprotein glycoprotein Ibα (GPIbα and also binds heparin and types I and III collagen. This disclosure describes the use of vWF as a growth factor reservoir for the enhancement of angiogenesis and wound healing.

### I. GROWTH FACTOR BINDING PEPTIDES AND POLYPEPTIDES

Embodiments of the disclosure relate to laminin peptides and von Willebrand factor peptides that bind to growth factors.

Laminins are major basement membrane extracellular matrix (ECM) proteins for which at least 16 isoforms exist. Five α (LAMA1-5), three β (LAMB1-3), and three γ (LAMC1-3) chains have been identified. Laminin's structure is a heterotrimer comprising an α, a β, and a γ chain that assemble into a cross shape.

A common hallmark of the laminin α chain structure is the presence of five laminin-type G domain (LG) modules arranged at the C-terminus in a tandem array. LG modules consist of 180-200 amino acids, and all the laminin α chains contain five LG domains (LG1-5). The laminin LG modules bind to heparin sulfate, perlecan and fibulin-1, as well as cellular receptors including α6β1, α7β1 and α6β4 integrins and syndecan. The laminin α3, α4, and α5 chains are processed in vivo in tissue through cleavage by proteases such as plasmin and elastase at the linker between the LG3 and LG4 domains.

### A. Exemplary Growth Factor Binding Peptides and Polypeptides

In some embodiments, the growth factor binding domain comprises a heparin binding domain (HBD). In some embodiments, the growth factor binding domain is from a laminin polypeptide. In some embodiments, the growth factor binding domain is from a vWF polypeptide. In some embodiments, the growth factor binding domain is not a heparin binding domain and/or does not bind to heparin. In some embodiments, the growth factor binding domain comprises a peptide from LAMA1. In some embodiments, the growth factor binding domain comprises a peptide from LAMA2. In some embodiments, the growth factor binding domain comprises a peptide from LAMA3. In some embodiments, the growth factor binding domain comprises a peptide from LAMA4. In some embodiments, the growth factor binding domain comprises a peptide from LAMA5. In some embodiments, the growth factor binding domain comprises a peptide from LAMB1. In some embodiments, the growth factor binding domain comprises a peptide from LAMB2. In some embodiments, the growth factor binding domain comprises a peptide from LAMB3. In some embodiments, the growth factor binding domain comprises a peptide from LAMB4. In some embodiments, the growth factor binding domain comprises a peptide from LAMC1. In some embodiments, the growth factor binding domain comprises a peptide from LAMC2. In some embodiments, the growth factor binding domain comprises a peptide from LAMC3. Exemplary laminin polypeptides are shown below:

| Human | |
|---|---|
| Laminin | Sequence |
| Laminin | MRGGVLLVLLLCVAAQCRQRGLFPAILNLASNAHISTNATCGEKGPE |
| subunit | MFCKLVEHVPGRPVRNPQCRICDGNSANPRERHPISHAIDGTNNWWQ |
| alpha-1 | SPSIQNGREYHWVTITLDLRQVFQVAYVIIKAANAPRPGNWILERSLDG |
| precursor | TTFSPWQYYAVSDSECLSRYNITPRRGPPTYRADDEVICTSYYSRLVPL |
| (LAMA1); | EHGEIHTSLINGRPSADDLSPKLLEFTSARYIRLRLQRIRTLNADLMTLS |
| SEQ ID | HREPKELDPIVTRRYYYSIKDISVGGMCICYGHASSCPWDETTKKLQC |
| NO: 25 | QCEHNTCGESCNRCCPGYHQQPWRPGTVSSGNTCEACNCHNKAKDC |
| | YYDESVAKQKKSLNTAGQFRGGGVCINCLQNTMGINCETCIDGYYRP |
| | HKVSPYEDEPCRPCNCDPVGSLSSVCIKDDLHSDLHNGKQPGQCPCKE |
| | GYTGEKCDRCQLGYKDYPTCVSCGCNPVGSASDEPCTGPCVCKENVE |
| | GKACDRCKPGFYNLKEKNPRGCSECFCFGVSDVCSSLSWPVGQVNSM |
| | SGWLVTDLISPRKIPSQQDALGGRHQVSINNTAVMQRLAPKYYWAAP |
| | EAYLGNKLTAFGGFLKYTVSYDIPVETVDSNLMSHADVIIKGNGLTLS |
| | TQAEGLSLQPYEEYLNVVRLVPENFQDFHSKRQ1DRDQLMTVLANVT |
| | HLLIRANYNSAKMALYRLESVSLDIASSNAIDLVVAADVEHCECPQGY |
| | TGTSCESCLSGYYRVDGILFGGICQPCECHGHAAECNVHGVCIACAHN |
| | TTGVHCEQCLPGFYGEPSRGTPGDCQPCACPLTIASNNFSPTCHLNDGD |
| | EVVCDWCAPGYSGAWCERCADGYYGNPTVPGESCVPCDCSGNVDPS |
| | EAGHCDSVTGECLKCLGNTDGAHCERCADGFYGDAVTAKNCRACEC |
| | HVKGSHSAVCHLETGLCDCKPNVTGQQCDQCLHGYYGLDSGHGCRP |
| | CNCSVAGSVSDGCTDEGQCHCVPGVAGKRCDRCAHGFYAYQDGSCT |
| | PCDCPHTQNTCDPETGECVCPPHTQGVKCEECEDGHWGYDAEVGCQ |
| | ACNCSLVGSTHHRCDVVTGHCQCKSKFGGRACDQCSLGYRDFPDCVP |
| | CDCDLRGTSGDACNLEQGLCGCVEETGACPCKENVFGPQCNECREGT |
| | FALRADNPLGCSPCFCSGLSHLCSELEDYVRTPVTLGSDQPLLRVVSQS |
| | NLRGTTEGVYYQAPDFLLDAATVRQHIRAEPFYWRLPQQFQGDQLMA |
| | YGGKLKYSVAFYSLDGVGTSNFEPQVLIKGGRIRKQVIYMDAPAPENG |
| | VRQEQEVAMRENFWKYFNSVSEKPVTREDFMSVLSDIEYILIKASYGQ |
| | GLQQSRISDISMEVGRKAEKLHPEEEVASLLENCVCPPGTVGFSCQDC |
| | APGYHRGKLPAGSDRGPRPLVAPCVPCSCNNHSDTCDPNTGKCLNCG |
| | DNTAGDHCDVCTSGYYGKVTGSASDCALCACPHSPPASFSPTCVLEG |
| | DHDFRCDACLLGYEGKHCERCSSSYYGNPQTPGGSCQKCDCNPHGSV |
| | HGDCDRTSGQCVCRLGASGLRCDECEPRHILMETDCVSCDDECVGVL |
| | LNDLDEIGDAVLSLNLTGIIPVPYGILSNLENTTKYLQESLLKENMQKD |
| | LGKIKLEGVAEETDNLQKKLTRMLASTQKVNRATERIFKESQDLAIAIE |
| | RLQMSITEIMEKTTLNQTLDEDFLLPNSTLQNMQQNGTSLLEIMQIRDF |
| | TQLHQNATLELKAAEDLLSQIQENYQKPLEELEVLKEAASHVLSKHNN |
| | ELKAAEALVREAEAKMQESNHLLLMVNANLREFSDKKLHVQEEQNL |
| | TSELIVQGRGLIDAAAAQTDAVQDALEHLEDHQDKLLLWSAKIRHHID |
| | DLVMHMSQRNAVDLVYRAEDHAAEFQRLADVLYSGLENIRNVSLNA |
| | TSAAYVHYNIQSLIEESEELARDAHRTVTETSLLSESLVSNGKAAVQRS |
| | SRFLKEGNNLSRKLPGIALELSELRNKTNRFQENAVEITRQTNESLLILR |
| | AIPKGIRDKGAKTKELATSASQSAVSTLRDVAGLSQELLNTSASLSRVN |
| | TTLRETHQLLQDSTMATLLAGRKVKDVEIQANLLFDRLKPLKMLEEN |
| | LSRNLSEIKLLISQARKQAASIKVAVSADRDCIRAYQPQISSTNYNTLTL |
| | NVKTQEPDNLLFYLGSSTASDFLAVEMRRGRVAFLWDLGSGSTRLEFP |
| | DEPIDDNRWHSIHVAREGNIGSLSVKEMSSNQKSPTKTSKSPGTANVLD |
| | VNNSTLMFVGGLGGQIKKSPAVKVTHFKGCLGEAFLNGKSIGLWNYIE |
| | REGKCRGCFGSSQNEDPSEHFDGSGYSVVEKSLPATVTQIIMLENTESP |
| | NGLLLYLGSYGTKDFLSIELFRGRVKVMTDLGSGPITLLTDRRYNNGT |
| | WYKIAFQRNRKQGVLAVIDAYNTSNKETKQGETPGASSDLNRLDKDPI |
| | YVGGLPRSRVVRRGVTTKSFVGCIKNLEISRSTFDLLRNSYGVRKGCLL |
| | EPIRSVSFLKGGYIELPPKSLSPESEWLVTFATTNSSGIILAALGGDVEKR |
| | GDREEAHVPFFSVMLIGGNIEVHVNPGDGTGLRKALLHAPTGTCSDGQ |
| | AHSISLVRNRRIITVQLDENNPVEMKLGTLVESRTINVSNLYVGGIPEGE |
| | GTSLLTMRRSEHGCIKNLIENLELLDENSAVGHEQVDLDTCWLSERPK |
| | LAPDAEDSKLLPEPRAFPEQCVVDAALEYVPGAHQEGLTQNSHEILPEN |
| | QSAVRKKLSVELSIRTFASSGLIYYMAHQNQADYAVLQLHGGRLHFM |
| | FDLGKGRTKVSHPALLSDGKWHTVKTDYVKRKGFITVDGRESPMVTV |
| | VGDGTMLDVEGLFYLGGLPSQYQARKIGNITHSIPACIGDVTVNSKQL |
| | DKDSPVSAFTVNRCYAVAQEGTYFDGSGYAALVKEGYKVQSDVNITL |
| | EFRTSSQNGVLLGISTAKVDAIGLELVDGKVLFHVNNGAGRITAAYEP |
| | KTATVLCDGKWHTLQANKSKHRITLIVDGNAVGAESPHTQSTSVDTN |
| | NPIYVGGYPAGVKQKCLRSQTSFRGCLRKLALIKSPQVQSEDESRAFEL |
| | HGVFLHSCPGTES |
| laminin | MPGAAGVLLLLLLSGGLGGVQAQRPQQQRQSQAHQQRGLFPAVLNL |
| subunit | ASNALITTNATCGEKGPEMYCKLVEHVPGQPVRNPQCRICNQNSSNPN |
| alpha-2 | QRHPITNAIDGKNTWWQSPSIKNGIEYHYVTITLDLQQVFQIAYVIVKA |
| isoform a | ANSPRPGNWILERSLDDVEYKPWQYHAVTDTECLTLYNIYPRTGPPSY |
| precursor | AKDDEVICTSFYSKIHPLENGEIHISLINGRPSADDPSPELLEFTSARYIRL |
| (LAMA2- | RFQRIRTLNADLMMFAHKDPREIDPIVTRRYYYSVKDISVGGMCICYG |
| isoform a); | HARACPLDPATNKSRCECEHNTCGDSCDQCCPGFHQKPWRAGTFLTK |
| SEQ ID | TECEACNCHGKAEECYYDENVARRNLSLNIRGKYIGGGVCINCTQNT |
| NO: 26 | AGINCETCTDGFFRPKGVSPNYPRPCQPCHCDPIGSLNEVCVKDEKHA |
| | RRGLAPGSCHCKTGFGGVSCDRCARGYTGYPDCKACNCSGLGSKNED |
| | PCFGPCICKENVEGGDCSRCKSGFFNLQEDNWKGCDECFCSGVSNRCQ |
| | SSYWTYGKIQDMSGWYLTDLPGRIRVAPQQDDLDSPQQISISNAEARQ |
| | ALPHSYYWSAPAPYLGNKLPAVGGQLTFTISYDLEEEEEDTERVLQLM |
| | IILEGNDLSISTAQDEVYLHPSEEHTNVLLLKEESFTIHGTHFPVRRKEF |
| | MTVLANLKRVLLQITYSFGMDAIFRLSSVNLESAVSYPTDGSIAAAVE |
| | VCQCPPGYTGSSCESCWPRHRRVNGTIFGGICEPCQCFGHAESCDDVT |
| | GECLNCKDHTGGPYCDKCLPGFYGEPTKGTSEDCQPCACPLNIPSNNF |
| | SPTCHLDRSLGLICDGCPVGYTGPRCERCAEGYFGQPSVPGGSCQPCQ |
| | CNDNLDFSIPGSCDSLSGSCLICKPGTTGRYCELCADGYFGDAVDAKN |
| | CQPCRCNAGGSFSEVCHSQTGQCECRANVQGQRCDKCKAGTFGLQSA |
| | RGCVPCNCNSFGSKSFDCEESGQCWCQPGVTGKKCDRCAHGYFNFQE |
| | GGCTACECSHLGNNCDPKTGRCICPPNTIGEKCSKCAPNTWGHSITTG |
| | CKACNCSTVGSLDFQCNVNTGQCNCHPKFSGAKCTECSRGHWNYPRC |
| | NLCDCFLPGTDATTCDSETKKCSCSDQTGQCTCKVNVEGIHCDRCRPG |
| | KFGLDAKNPLGCSSCYCFGTTTQCSEAKGLIRTWVTLKAEQTILPLVD |
| | EALQHTTTKGIVFQHPEIVAHMDLMREDLHLEPFYWKLPEQFEGKKL |
| | MAYGGKLKYAIYFEAREETGFSTYNPQVIIRGGTPTHARIIVRHMAAPL |
| | IGQLTRHEIEMTEKEWKYYGDDPRVHRTVTREDFLDILYDIHYILIKAT |
| | YGNFMRQSRISEISMEVAEQGRGTTMTPPADLIEKCDCPLGYSGLSCEA |
| | CLPGFYRLRSQPGGRTPGPTLGTCVPCQCNGHSSLCDPETSICQNCQHH |
| | TAGDFCERCALGYYGIVKGLPNDCQQCACPLISSSNNFSPSCVAEGLD |
| | DYRCTACPRGYEGQYCERCAPGYTGSPGNPGGSCQECECDPYGSLPVP |
| | CDPVTGFCTCRPGATGRKCDGCKHWHAREGWECVFCGDECTGLLLG |
| | DLARLEQMVMSINLTGPLPAPYKMLYGLENIVITQELKHLLSPQRAPER |
| | LIQLAEGNLNTLVTEMNELLTRATKVTADGEQTGQDAERTNTRAKSL |
| | GEFIKELARDAEAVNEKAIKLNETLGTRDEAFERNLEGLQKEIDQMIKE |
| | LRRKNLETQKEIAEDELVAAEALLKKVKKLFGESRGENEEMEKDLRE |
| | KLADYKNKVDDAWDLLREATDKIREANRLFAVNQKNMTALEKKKEA |
| | VESGKRQIENTLKEGNDILDEANRLADEINSIIDYVEDIQTKLPPMSEEL |
| | NDKIDDLSQEIKDRKLAEKVSQAESHAAQLNDSSAVLDGILDEAKNISF |
| | NATAAFKAYSNIKDYIDEAEKVAKEAKDLAHEATKLATGPRGLLKED |
| | AKGCLQKSFRILNEAKKLANDVKENEDHLNGLKTRIENADARNGDLL |
| | RTLNDTLGKLSAIPNDTAAKLQAVKDKARQANDTAKDVLAQITELHQ |
| | NLDGLKKNYNKLADSVAKTNAVVKDPSKNKIIADADATVKNLEQEA |
| | DRLIDKLKPIKELEDNLKKNISEIKELINQARKQANSIKVSVSSGGDCIR |
| | TYKPEIKKGSYNNIVVNVKTAVADNLLFYLGSAKFIDFLAIEMRKGKV |
| | SFLWDVGSGVGRVEYPDLTIDDSYWYRIVASRTGRNGTISVRALDGPK |
| | ASIVPSTHHSTSPPGYTILDVDANAMLFVGGLTGKLKKADAVRVITFT |
| | GCMGETYFDNKPIGLWNFREKEGDCKGCTVSPQVEDSEGTIQFDGEG |
| | YALVSRPIRWYPNISTVMFKFRTFSSSALLMYLATRDLRDFMSVELTD |
| | GHIKVSYDLGSGMASVVSNQNHNDGKWKSFTLSRIQKQANISIVDIDT |
| | NQEENIATSSSGNNFGLDLKADDKIYFGGLPTLRNLSMKARPEVNLKK |
| | YSGCLKDIEISRTPYNILSSPDYVGVTKGCSLENVYTVSFPKPGFVELSP |
| | VPIDVGTEINLSFSTKNESGIILLGSGGTPAPPRRKRRQTGQAYYVILLN |
| | RGRLEVHLSTGARTMRKIVIRPEPNLFHDGREHSVHVERTRGIFTVQV |
| | DENRRYMQNLTVEQPIEVKKLFVGGAPPEFQPSPLRNIPPFEGCIWNLV |
| | INSVPMDFARPVSFKNADIGRCAHQKLREDEDGAAPAEIVIQPEPVPTP |
| | AFPTPTPVLTHGPCAAESEPALLIGSKQFGLSRNSHIAIAFDDTKVKNRL |
| | TIELEVRTEAESGLLFYMARINHADFATVQLRNGLPYFSYDLGSGDTH |
| | TMIPTKINDGQWHKIKIMRSKQEGILYVDGASNRTISPKKADILDVVG |
| | MLYVGGLPINYTTRRIGPVTYSIDGCVRNLHMAEAPADLEQPTSSFHV |
| | GTCFANAQRGTYFDGTGFAKAVGGFKVGLDLLVEFEFRTTTTTGVLL |
| | GISSQKMDGMGIEMIDEKLMFHVDNGAGRFTAVYDAGVPGHLCDGQ |
| | WHKVTANKIKHRIELTVDGNQVEAQSPNPASTSADTNDPVFVGGFPD |
| | DLKQFGLTTSIPFRGCIRSLKLTKGTGKPLEVNFAKALELRGVQPVSCP |
| | AN |
| laminin | MPGAAGVLLLLLLSGGLGGVQAQRPQQQRQSQAHQQRGLFPAVLNL |
| subunit | ASNALITTNATCGEKGPEMYCKLVEHVPGQPVRNPQCRICNQNSSNPN |
| alpha-2 | QRHPITNAIDGKNTWWQSPSIKNGIEYHYVTITLDLQQVFQIAYVIVKA |
| isoform b | ANSPRPGNWILERSLDDVEYKPWQYHAVTDTECLTLYNIYPRTGPPSY |
| precursor | AKDDEVICTSFYSKIHPLENGEIHISLINGRPSADDPSPELLEFTSARYIRL |
| (LAMA2- | RFQRIRTLNADLMMFAHKDPREIDPIVTRRYYYSVKDISVGGMCICYG |
| isoform b); | HARACPLDPATNKSRCECEHNTCGDSCDQCCPGFHQKPWRAGTFLTK |
| SEQ ID | TECEACNCHGKAEECYYDENVARRNLSLNIRGKYIGGGVCINCTQNT |
| NO: 27 | AGINCETCTDGFFRPKGVSPNYPRPCQPCHCDPIGSLNEVCVKDEKHA |
| | RRGLAPGSCHCKTGFGGVSCDRCARGYTGYPDCKACNCSGLGSKNED |
| | PCFGPCICKENVEGGDCSRCKSGFFNLQEDNWKGCDECFCSGVSNRCQ |
| | SSYWTYGKIQDMSGWYLTDLPGRIRVAPQQDDLDSPQQISISNAEARQ |
| | ALPHSYYWSAPAPYLGNKLPAVGGQLTFTISYDLEEEEEDTERVLQLM |
| | IILEGNDLSISTAQDEVYLHPSEEHTNVLLLKEESFTIHGTHFPVRRKEF |
| | MTVLANLKRVLLQITYSFGMDAIFRLSSVNLESAVSYPTDGSIAAAVE |
| | VCQCPPGYTGSSCESCWPRHRRVNGTIFGGICEPCQCFGHAESCDDVT |
| | GECLNCKDHTGGPYCDKCLPGFYGEPTKGTSEDCQPCACPLNIPSNNF |
| | SPTCHLDRSLGLICDGCPVGYTGPRCERCAEGYFGQPSVPGGSCQPCQ |
| | CNDNLDFSIPGSCDSLSGSCLICKPGTTGRYCELCADGYFGDAVDAKN |
| | CQPCRCNAGGSFSEVCHSQTGQCECRANVQGQRCDKCKAGTFGLQSA |
| | RGCVPCNCNSFGSKSFDCEESGQCWCQPGVTGKKCDRCAHGYFNFQE |
| | GGCTACECSHLGNNCDPKTGRCICPPNTIGEKCSKCAPNTWGHSITTG |
| | CKACNCSTVGSLDFQCNVNTGQCNCHPKFSGAKCTECSRGHWNYPRC |
| | NLCDCFLPGTDATTCDSETKKCSCSDQTGQCTCKVNVEGIHCDRCRPG |
| | KFGLDAKNPLGCSSCYCFGTTTQCSEAKGLIRTWVTLKAEQTILPLVD |
| | EALQHTTTKGIVFQHPEIVAHMDLMREDLHLEPFYWKLPEQFEGKKL |
| | MAYGGKLKYAIYFEAREETGFSTYNPQVIIRGGTPTHARIIVRHMAAPL |
| | IGQLTRHEIEMTEKEWKYYGDDPRVHRTVTREDFLDILYDIHYILIKAT |
| | YGNFMRQSRISEISMEVAEQGRGTTMTPPADLIEKCDCPLGYSGLSCEA |
| | CLPGFYRLRSQPGGRTPGPTLGTCVPCQCNGHSSLCDPETSICQNCQHH |
| | TAGDFCERCALGYYGIVKGLPNDCQQCACPLISSSNNFSPSCVAEGLD |
| | DYRCTACPRGYEGQYCERCAPGYTGSPGNPGGSCQECECDPYGSLPVP |
| | CDPVTGFCTCRPGATGRKCDGCKHWHAREGWECVFCGDECTGLLLG |
| | DLARLEQMVMSINLTGPLPAPYKMLYGLENMTQELKHLLSPQRAPER |
| | LIQLAEGNLNTLVTEMNELLTRATKVTADGEQTGQDAERTNTRAKSL |
| | GEFIKELARDAEAVNEKAIKLNETLGTRDEAFERNLEGLQKEIDQMIKE |
| | LRRKNLETQKEIAEDELVAAEALLKKVKKLFGESRGENEEMEKDLRE |
| | KLADYKNKVDDAWDLLREATDKIREANRLFAVNQKNMTALEKKKEA |
| | VESGKRQIENTLKEGNDILDEANRLADEINSIIDYVEDIQTKLPPMSEEL |
| | NDKIDDLSQEIKDRKLAEKVSQAESHAAQLNDSSAVLDGILDEAKNISF |
| | NATAAFKAYSNIKDYIDEAEKVAKEAKDLAHEATKLATGPRGLLKED |
| | AKGCLQKSFRILNEAKKLANDVKENEDHLNGLKTRIENADARNGDLL |
| | RTLNDTLGKLSAIPNDTAAKLQAVKDKARQANDTAKDVLAQITELHQ |
| | NLDGLKKNYNKLADSVAKTNAVVKDPSKNKIIADADATVKNLEQEA |
| | DRLIDKLKPIKELEDNLKKNISEIKELINQARKQANSIKVSVSSGGDCIR |
| | TYKPEIKKGSYNNIVVNVKTAVADNLLFYLGSAKFIDFLAIEMRKGKV |
| | SFLWDVGSGVGRVEYPDLTIDDSYWYRIVASRTGRNGTISVRALDGPK |
| | ASIVPSTHHSTSPPGYTILDVDANAMLFVGGLTGKLKKADAVRVITFT |
| | GCMGETYFDNKPIGLWNFREKEGDCKGCTVSPQVIEDSEGTIQFDGEG |
| | YALVSRPIRWYPNISTVMEKERTFSSSALLMYLATRDLRDFMSVELTD |
| | GHIKVSYDLGSGMASVVSNQNHNDGKWKSFTLSRIQKQANISIVDIDT |
| | NQEENIATSSSGNNFGLDLKADDKIYFGGLPTLRNLRPEVNLKKYSGC |
| | LKDIEISRTPYNILSSPDYVGVTKGCSLENVYTVSFPKPGEVELSPVPID |
| | VGTEINLSFSTKNESGIILLGSGGTPAPPRRKRRQTGQAYYVILLNRGRL |
| | EVHLSTGARTMRKIVIRPEPNLFHDGREHSVHVERTRGIFTVQVDENR |
| | RYMQNLTVEQPIEVKKLFVGGAPPEFQPSPLRNIPPFEGCIWNLVINSVP |
| | MDFARPVSFKNADIGRCAHQKLREDEDGAAPAEIVIQPEPVPTPAFPTP |
| | TPVLTHGPCAAESEPALLIGSKQFGLSRNSHIAIAFDDTKVKNRLTIELE |
| | VRTEAESGLLFYMARINHADFATVQLRNGLPYFSYDLGSGDTHTMIPT |
| | KINDGQWHKIKIMRSKQEGILYVDGASNRTISPKKADILDVVGMLYVG |
| | GLPINYTTRRIGPVTYSIDGCVRNLHMAEAPADLEQPTSSFHVGTCFAN |
| | AQRGTYFDGTGFAKAVGGEKVGLDLLVEFEERTTTTTGVLLGISSQKM |
| | DGMGIEMIDEKLMFHVDNGAGRFTAVYDAGVPGHLCDGQWHKVTA |
| | NKIKHRIELTVDGNQVEAQSPNPASTSADTNDPVFVGGFPDDLKQFGL |
| | TTSIPERGCIRSLKLTKGTGKPLEVNFAKALELRGVQPVSCPAN |
| laminin | MAAAARPRGRALGPVLPPTPLLLLVLRVLPACGATARDPGAAAGLSL |
| subunit | HPTYFNLAEAARIWATATCGERGPGEGRPQPELYCKLVGGPTAPGSGH |
| alpha-3 | TIQGQFCDYCNSEDPRKAHPVTNAIDGSERWWQSPPLSSGTQYNRVNL |
| isoform 1 | TLDLGQLFHVAYILIKFANSPRPDLWVLERSVDFGSTYSPWQYFAHSK |
| precursor | VDCLKEFGREANMAVTRDDDVLCVTEYSRIVPLENGEVVVSLINGRPG |
| (LAMA3- | AKNFTFSHTLREFTKATNIRLRFLRTNTLLGHLISKAQRDPTVTRRYYY |
| isoform 1); | SIKDISIGGQCVCNGHAEVCNINNPEKLFRCECQHHTCGETCDRCCTGY |
| SEQ ID | NQRRWRPAAWEQSHECEACNCHGHASNCYYDPDVERQQASLNTQGI |
| NO: 28 | YAGGGVCINCQHNTAGVNCEQCAKGYYRPYGVPVDAPDGCIPCSCDP |
| | EHADGCEQGSGRCHCKPNFHGDNCEKCAIGYYNFPFCLRIPIFPVSTPS |
| | SEDPVAGDIKGCDCNLEGVLPEICDAHGRCLCRPGVEGPRCDTCRSGF |
| | YSFPICQACWCSALGSYQMPCSSVTGQCECRPGVTGQRCDRCLSGAY |
| | DFPHCQGSSSACDPAGTINSNLGYCQCKLHVEGPTCSRCKLLYWNLD |
| | KENPSGCSECKCHKAGTVSGTGECRQGDGDCHCKSHVGGDSCDTCED |
| | GYFALEKSNYFGCQGCQCDIGGALSSMCSGPSGVCQCREHVVGKVCQ |
| | RPENNYYFPDLHHMKYEIEDGSTPNGRDLRFGFDPLAFPEFSWRGYAQ |
| | MTSVQNDVRITLNVGKSSGSLFRVILRYVNPGTEAVSGHITIYPSWGAA |
| | QSKEIIFLPSKEPAFVTVPGNGFADPFSITPGIWVACIKAEGVLLDYLVL |
| | LPRDYYEASVLQLPVTEPCAYAGPPQENCLLYQHLPVTRFPCTLACEA |
| | RHFLLDGEPRPVAVRQPTPAHPVMVDLSGREVELHLRLRIPQVGHYVV |
| | VVEYSTEAAQLFVVDVNVKSSGSVLAGQVNIYSCNYSVLCRSAVIDH |
| | MSRIAMYELLADADIQLKGHMARFLLHQVCIIPIEEFSAEYVRPQVHCI |
| | ASYGRFVNQSATCVSLAHETPPTALILDVLSGRPFPHLPQQSSPSVDVL |
| | PGVTLKAPQNQVTLRGRVPHLGRYVFVIHFYQAAHPTFPAQVSVDGG |
| | WPRAGSFHASFCPHVLGCRDQVIAEGQIEFDISEPEVAATVKVPEGKSL |
| | VLVRVLVVPAENYDYQILHKKSMDKSLEFITNCGKNSFYLDPQTASRF |
| | CKNSARSLVAFYHKGALPCECHPTGATGPHCSPEGGQCPCQPNVIGRQ |
| | CTRCATGHYGFPRCKPCSCGRRLCEEMTGQCRCPPRTVRPQCEVCETH |
| | SFSFHPMAGCEGCNCSRRGTIEAAMPECDRDSGQCRCKPRITGRQCDR |
| | CASGFYRFPECVPCNCNRDGTEPGVCDPGTGACLCKENVEGTECNVC |
| | REGSFHLDPANLKGCTSCFCFGVNNQCHSSHKRRTKFVDMLGWHLET |
| | ADRVDIPVSFNPGSNSMVADLQELPATIHSASWVAPTSYLGDKVSSYG |
| | GYLTYQAKSFGLPGDMVLLEKKPDVQLTGQHMSIIYEETNTPRPDRLH |
| | HGRVHVVEGNFRHASSRAPVSREELMTVLSRLADVRIQGLYFTETQRL |
| | TLSEVGLEEASDTGSGRIALAVEICACPPAYAGDSCQGCSPGYYRDHK |
| | GLYTGRCVPCNCNGHSNQCQDGSGICVNCQHNTAGEHCERCQEGYY |
| | GNAVHGSCRACPCPHTNSFATGCVVNGGDVRCSCKAGYTGTQCERC |
| | APGYFGNPQKFGGSCQPCSCNSNGQLGSCHPLTGDCINQEPKDSSPAE |
| | ECDDCDSCVMTLLNDLATMGEQLRLVKSQLQGLSASAGLLEQMRHM |
| | ETQAKDLRNQLLNYRSAISNHGSKIEGLERELTDLNQEFETLQEKAQV |
| | NSRKAQTLNNNVNRATQSAKELDVKIKNVIRNVHILLKQISGTDGEGN |
| | NVPSGDFSREWAEAQRMMRELRNRNFGKHLREAEADKRESQLLLNRI |
| | RTWQKTHQGENNGLANSIRDSLNEYEAKLSDLRARLQEAAAQAKQA |
| | NGLNQENERALGAIQRQVKEINSLQSDFTKYLTTADSSLLQTNIALQL |
| | MEKSQKEYEKLAASLNEARQELSDKVRELSRSAGKTSLVEEAEKHAR |
| | SLQELAKQLEEIKRNASGDELVRCAVDAATAYENILNAIKAAEDAANR |
| | AASASESALQTVIKEDLPRKAKTLSSNSDKLLNEAKMTQKKLKQEVSP |
| | ALNNLQQTLNIVTVQKEVIDTNLTTLRDGLHGIQRGDIDAMISSAKSM |
| | VRKANDITDEVLDGLNPIQTDVERIKDTYGRTQNEDFKKALTDADNSV |
| | NKLTNKLPDLWRKIESINQQLLPLGNISDNMDRIRELIQQARDAASKVA |
| | VPMRFNGKSGVEVRLPNDLEDLKGYTSLSLFLQRPNSRENGGTENMF |
| | VMYLGNKDASRDYIGMAVVDGQLTCVYNLGDREAELQVDQILTKSE |
| | TKEAVMDRVKFQRIYQFARLNYTKGATSSKPETPGVYDMDGRNSNTL |
| | LNLDPENVVFYVGGYPPDFKLPSRLSFPPYKGCIELDDLNENVLSLYNF |
| | KKTFNLNTTEVEPCRRRKEESDKNYFEGTGYARVPTQPHAPIPTFGQTI |
| | QTTVDRGLLFFAENGDRFISLNIEDGKLMVRYKLNSELPKERGVGDAI |
| | NNGRDHSIQIKIGKLQKRMWINVDVQNTIIDGEVFDFSTYYLGGIPIAIR |
| | ERFNISTPAFRGCMKNLKKTSGVVRLNDTVGVTKKCSEDWKLVRSAS |
| | FSRGGQLSFTDLGLPPTDHLQASFGFQTFQPSGILLDHQTWTRNLQVTL |
| | EDGYIELSTSDSGGPIFKSPQTYMDGLLHYVSVISDNSGLRLLIDDQLLR |
| | NSKRLKHISSSRQSLRLGGSNFEGCISNVFVQRLSLSPEVLDLTSNSLKR |
| | DVSLGGCSLNKPPFLMLLKGSTRFNKTKTFRINQLLQDTPVASPRSVK |
| | VWQDACSPLPKTQANHGALQFGDIPTSHLLFKLPQELLKPRSQFAVDM |
| | QTTSSRGLVFHTGTKNSFMALYLSKGRLVFALGTDGKKLRIKSKEKCN |
| | DGKWHTVVFGHDGEKGRLVVDGLRAREGSLPGNSTISIRAPVYLGSPP |
| | SGKPKSLPTNSFVGCLKNFQLDSKPLYTPSSSFGVSSCLGGPLEKGIYFS |
| | EEGGHVVLAHSVLLGPEFKLVFSIRPRSLTGILIHIGSQPGKHLCVYLEA |
| | GKVTASMDSGAGGTSTSVTPKQSLCDGQWHSVAVTIKQHILHLELDT |
| | DSSYTAGQIPFPPASTQEPLHLGGAPANLTTLRIPVWKSFFGCLRNIHV |
| | NHIPVPVTEALEVQGPVSLNGCPDQ |
| laminin | MPPAVRRSACSMGWLWIFGAALGQCLGYSSQQQRVPFLQPPGQSQLQ |
| subunit | ASYVEFRPSQGCSPGYYRDHKGLYTGRCVPCNCNGHSNQCQDGSGIC |
| alpha-3 | VNCQHNTAGEHCERCQEGYYGNAVHGSCRACPCPHTNSFATGCVVN |
| isoform 2 | GGDVRCSCKAGYTGTQCERCAPGYFGNPQKFGGSCQPCSCNSNGQLG |
| precursor | SCHPLTGDCINQEPKDSSPAEECDDCDSCVMTLLNDLATMGEQLRLVK |
| (LAMA3- | SQLQGLSASAGLLEQMRHMETQAKDLRNQLLNYRSAISNHGSKIEGLE |
| isoform 2); | RELTDLNQEFETLQEKAQVNSRKAQTLNNNVNRATQSAKELDVKIKN |
| SEQ ID | VIRNVHILLKQISGTDGEGNNVPSGDFSREWAEAQRMMRELRNRNFG |
| NO: 29 | KHLREAEADKRESQLLLNRIRTWQKTHQGENNGLANSIRDSLNEYEA |
| | KLSDLRARLQEAAAQAKQANGLNQENERALGAIQRQVKEINSLQSDF |
| | TKYLTTADSSLLQTNIALQLMEKSQKEYEKLAASLNEARQELSDKVRE |
| | LSRSAGKTSLVEEAEKHARSLQELAKQLEEIKRNASGDELVRCAVDAA |
| | TAYENILNAIKAAEDAANRAASASESALQTVIKEDLPRKAKTLSSNSD |
| | KLLNEAKMTQKKLKQEVSPALNNLQQTLNIVTVQKEVIDTNLTTLRD |
| | GLHGIQRGDIDANTISSAKSMVRKANDITDEVLDGLNPIQTDVERIKDTY |
| | GRTQNEDFKKALTDADNSVNKLTNKLPDLWRKIESINQQLLPLGNISD |
| | NMDRIRELIQQARDAASKVAVPMRFNGKSGVEVRLPNDLEDLKGYTS |
| | LSLFLQRPNSRENGGTENMFVMYLGNKDASRDYIGMAVVDGQLTCV |
| | YNLGDREAELQVDQILTKSETKEAVMDRVKFQRIYQFARLNYTKGAT |
| | SSKPETPGVYDMDGRNSNTLLNLDPENVVFYVGGYPPDFKLPSRLSFP |
| | PYKGCIELDDLNENVLSLYNFKKTFNLNTTEVEPCRRRKEESDKNYFE |
| | GTGYARVPTQPHAPIPTFGQTIQTTVDRGLLFFAENGDRFISLNIEDGKL |
| | MVRYKLNSELPKERGVGDAINNGRDHSIQIKIGKLQKRMWINVDVQN |
| | TIIDGEVFDFSTYYLGGIPIAIRERFNISTPAFRGCMKNLKKTSGVVRLN |
| | DTVGVTKKCSEDWKLVRSASFSRGGQLSFTDLGLPPTDHLQASFGFQT |
| | FQPSGILLDHQTWTRNLQVTLEDGYIELSTSDSGGPIFKSPQTYMDGLL |
| | HYVSVISDNSGLRLLIDDQLLRNSKRLKHISSSRQSLRLGGSNFEGCISN |
| | VFVQRLSLSPEVLDLTSNSLKRDVSLGGCSLNKPPFLMLLKGSTRFNKT |
| | KTFRINQLLQDTPVASPRSVKVWQDACSPLPKTQANHGALQFGDIPTS |
| | HLLFKLPQELLKPRSQFAVDMQTTSSRGLVFHTGTKNSFMALYLSKGR |
| | LVFALGTDGKKLRIKSKEKCNDGKWHTVVFGHDGEKGRLVVDGLRA |
| | REGSLPGNSTISIRAPVYLGSPPSGKPKSLPTNSFVGCLKNFQLDSKPLY |
| | TPSSSFGVSSCLGGPLEKGIYFSEEGGHVVLAHSVLLGPEFKLVFSIRPR |
| | SLTGILIHIGSQPGKHLCVYLEAGKVTASMDSGAGGTSTSVTPKQSLCD |
| | GQWHSVAVTIKQHILHLELDTDSSYTAGQIPFPPASTQEPLHLGGAPAN |
| | LTTLRIPVWKSFFGCLRNIHVNHIPVPVTEALEVQGPVSLNGCPDQ |
| laminin | MAAAARPRGRALGPVLPPTPLLLLVLRVLPACGATARDPGAAAGLSL |
| subunit | HPTYFNLAEAARIWATATCGERGPGEGRPQPELYCKLVGGPTAPGSGH |
| alpha-3 | TIQGQFCDYCNSEDPRKAHPVTNAIDGSERWWQSPPLSSGTQYNRVNL |
| isoform | TLDLGQLFHVAYILIKFANSPRPDLWVLERSVDFGSTYSPWQYFAHSK |
| 3precursor | VDCLKEFGREANMAVTRDDDVLCVTEYSRIVPLENGEVVVSLINGRPG |
| (LAMA3- | AKNFTFSHTLREFTKATNIRLRFLRTNTLLGHLISKAQRDPTVTRRYYY |
| isoform 3); | SIKDISIGGQCVCNGHAEVCNINNPEKLFRCECQHHTCGETCDRCCTGY |
| SEQ ID | NQRRWRPAAWEQSHECEACNCHGHASNCYYDPDVERQQASLNTQGI |
| NO: 30 | YAGGGVCINCQHNTAGVNCEQCAKGYYRPYGVPVDAPDGCIPCSCDP |
| | EHADGCEQGSGRCHCKPNFHGDNCEKCAIGYYNFPFCLRIPIFPVSTPS |
| | SEDPVAGDIKGCDCNLEGVLPEICDAHGRCLCRPGVEGPRCDTCRSGF |
| | YSFPICQACWCSALGSYQMPCSSVTGQCECRPGVTGQRCDRCLSGAY |
| | DFPHCQGSSSACDPAGTINSNLGYCQCKLHVEGPTCSRCKLLYWNLD |
| | KENPSGCSECKCHKAGTVSGTGECRQGDGDCHCKSHVGGDSCDTCED |
| | GYFALEKSNYFGCQGCQCDIGGALSSMCSGPSGVCQCREHVVGKVCQ |
| | RPENNYYFPDLHHMKYEIEDGSTPNGRDLRFGFDPLAFPEFSWRGYAQ |
| | MTSVQNDVRITLNVGKSSGSLFRVILRYVNPGTEAVSGHITIYPSWGAA |
| | QSKEIIFLPSKEPAFVTVPGNGFADPFSITPGIWVACIKAEGVLLDYLVL |
| | LPRDYYEASVLQLPVTEPCAYAGPPQENCLLYQHLPVTRFPCTLACEA |
| | RHFLLDGEPRPVAVRQPTPAHPVMVDLSGREVELHLRLRIPQVGHYVV |
| | VVEYSTEAAQLFVVDVNVKSSGSVLAGQVNIYSCNYSVLCRSAVIDH |
| | MSRIAMYELLADADIQLKGHMARFLLHQVCIIPIEEFSAEYVRPQVHCI |
| | ASYGRFVNQSATCVSLAHETPPTALILDVLSGRPFPHLPQQSSPSVDVL |
| | PGVTLKAPQNQVTLRGRVPHLGRYVFVIHFYQAAHPTFPAQVSVDGG |
| | WPRAGSFHASFCPHVLGCRDQVIAEGQIEFDISEPEVAATVKVPEGKSL |
| | VLVRVLVVPAENYDYQILHKKSMDKSLEFITNCGKNSFYLDPQTASRF |
| | CKNSARSLVAFYHKGALPCECHPTGATGPHCSPEGGQCPCQPNVIGRQ |
| | CTRCATGHYGFPRCKPCSCGRRLCEEMTGQCRCPPRTVRPQCEVCETH |
| | SFSFHPMAGCEGCNCSRRGTIEAAMPECDRDSGQCRCKPRITGRQCDR |
| | CASGFYRFPECVPCNCNRDGTEPGVCDPGTGACLCKENVEGTECNVC |
| | REGSFHLDPANLKGCTSCFCFGVNNQCHSSHKRRTKFVDMLGWHLET |
| | ADRVDIPVSFNPGSNSMVADLQELPATIHSASWVAPTSYLGDKVSSYG |
| | GYLTYQAKSFGLPGDMVLLEKKPDVQLTGQHMSIIYEETNTPRPDRLH |
| | HGRVHVVEGNFRHASSRAPVSREELMTVLSRLADVRIQGLYFTETQRL |
| | TLSEVGLEEASDTGSGRIALAVEICACPPAYAGDSCQGCSPGYYRDHK |
| | GLYTGRCVPCNCNGHSNQCQDGSGICVNCQHNTAGEHCERCQEGYY |
| | GNAVHGSCRACPCPHTNSFATGCVVNGGDVRCSCKAGYTGTQCERC |
| | APGYFGNPQKFGGSCQPCSCNSNGQLGSCHPLTGDCINQEPKDSSPAE |
| | ECDDCDSCVMTLLNDLATMGEQLRLVKSQLQGLSASAGLLEQMRHM |
| | ETQAKDLRNQLLNYRSAISNHGSKIEGLERELTDLNQEFETLQEKAQV |
| | NSRKAQTLNNNVNRATQSAKELDVKIKNVIRNVHMLNRIRTWQKTHQ |
| | GENNGLANSIRDSLNEYEAKLSDLRARLQEAAAQAKQANGLNQENER |
| | ALGAIQRQVKEINSLQSDFTKYLTTADSSLLQTNIALQLMEKSQKEYEK |
| | LAASLNEARQELSDKVRELSRSAGKTSLVEEAEKHARSLQELAKQLEE |
| | IKRNASGDELVRCAVDAATAYENILNAIKAAEDAANRAASASESALQT |
| | VIKEDLPRKAKTLSSNSDKLLNEAKMTQKKLKQEVSPALNNLQQTLNI |
| | VTVQKEVIDTNLTTLRDGLHGIQRGDIDAMISSAKSMVRKANDITDEV |
| | LDGLNPIQTDVERIKDTYGRTQNEDFKKALTDADNSVNKLTNKLPDL |
| | WRKIESINQQLLPLGNISDNMDRIRELIQQARDAASKVAVPMRENGKS |
| | GVEVRLPNDLEDLKGYTSLSLFLQRPNSRENGGTENMFVMYLGNKDA |
| | SRDYIGMAVVDGQLTCVYNLGDREAELQVDQILTKSETKEAVMDRV |
| | KFQRIYQFARLNYTKGATSSKPETPGVYDMDGRNSNTLLNLDPENVVF |
| | YVGGYPPDFKLPSRLSFPPYKGCIELDDLNENVLSLYNFKKTFNLNTTE |
| | VEPCRRRKEESDKNYFEGTGYARVPTQPHAPIPTFGQTIQTTVDRGLLF |
| | FAENGDRFISLNIEDGKLMVRYKLNSELPKERGVGDAINNGRDHSIQIK |
| | IGKLQKRMWINVDVQNTIIDGEVEDFSTYYLGGIPIAIRERFNISTPAFR |
| | GCMKNLKKTSGVVRLNDTVGVTKKCSEDWKLVRSASFSRGGQLSFT |
| | DLGLPPTDHLQASFGFQTFQPSGILLDHQTWTRNLQVTLEDGYIELSTS |
| | DSGGPIFKSPQTYMDGLLHYVSVISDNSGLRLLIDDQLLRNSKRLKHIS |
| | SSRQSLRLGGSNFEGCISNVFVQRLSLSPEVLDLTSNSLKRDVSLGGCS |
| | LNKPPFLMLLKGSTRFNKTKTFRINQLLQDTPVASPRSVKVWQDACSP |
| | LPKTQANHGALQFGDIPTSHLLFKLPQELLKPRSQFAVDMQTTSSRGL |
| | VFHTGTKNSFMALYLSKGRLVFALGTDGKKLRIKSKEKCNDGKWHTV |
| | VFGHDGEKGRLVVDGLRAREGSLPGNSTISIRAPVYLGSPPSGKPKSLP |
| | TNSFVGCLKNFQLDSKPLYTPSSSFGVSSCLGGPLEKGIYFSEEGGHVV |
| | LAHSVLLGPEFKLVFSIRPRSLTGILIHIGSQPGKHLCVYLEAGKVTASM |
| | DSGAGGTSTSVTPKQSLCDGQWHSVAVTIKQHILHLELDTDSSYTAGQ |
| | IPFPPASTQEPLHLGGAPANLTTLRIPVWKSFFGCLRNIHVNHIPVPVTE |
| | ALEVQGPVSLNGCPDQ |
| laminin | MPPAVRRSACSMGWLWIFGAALGQCLGYSSQQQRVPFLQPPGQSQLQ |
| subunit | ASYVEFRPSQGCSPGYYRDHKGLYTGRCVPCNCNGHSNQCQDGSGIC |
| alpha-3 | VNCQHNTAGEHCERCQEGYYGNAVHGSCRACPCPHTNSFATGCVVN |
| isoform 4 | GGDVRCSCKAGYTGTQCERCAPGYFGNPQKFGGSCQPCSCNSNGQLG |
| precursor | SCHPLTGDCINQEPKDSSPAEECDDCDSCVMTLLNDLATMGEQLRLVK |
| (LAMA3- | SQLQGLSASAGLLEQMRHMETQAKDLRNQLLNYRSAISNHGSKIEGLE |
| isoform 4); | RELTDLNQEFETLQEKAQVNSRKAQTLNNNVNRATQSAKELDVKIKN |
| SEQ ID | VIRNVHMLNRIRTWQKTHQGENNGLANSIRDSLNEYEAKLSDLRARL |
| NO: 31 | QEAAAQAKQANGLNQENERALGAIQRQVKEINSLQSDFTKYLTTADS |
| | SLLQTNIALQLMEKSQKEYEKLAASLNEARQELSDKVRELSRSAGKTS |
| | LVEEAEKHARSLQELAKQLEEIKRNASGDELVRCAVDAATAYENILNA |
| | IKAAEDAANRAASASESALQTVIKEDLPRKAKTLSSNSDKLLNEAKMT |
| | QKKLKQEVSPALNNLQQTLNIVTVQKEVIDTNLTTLRDGLHGIQRGDI |
| | DAMISSAKSMVRKANDITDEVLDGLNPIQTDVERIKDTYGRTQNEDFK |
| | KALTDADNSVNKLTNKLPDLWRKIESINQQLLPLGNISDNMDRIRELIQ |
| | QARDAASKVAVPMRFNGKSGVEVRLPNDLEDLKGYTSLSLFLQRPNS |
| | RENGGTENNIFVMYLGNKDASRDYIGMAVVDGQLTCVYNLGDREAEL |
| | QVDQILTKSETKEAVMDRVKFQRIYQFARLNYTKGATSSKPETPGVYD |
| | MDGRNSNTLLNLDPENVVFYVGGYPPDFKLPSRLSFPPYKGCIELDDL |
| | NENVLSLYNFKKTFNLNTTEVEPCRRRKEESDKNYFEGTGYARVPTQP |
| | HAPIPTFGQTIQTTVDRGLLFFAENGDRFISLNIEDGKLMVRYKLNSELP |
| | KERGVGDAINNGRDHSIQIKIGKLQKRMWINVDVQNTIIDGEVFDFSTY |
| | YLGGIPIAIRERFNISTPAFRGCMKNLKKTSGVVRLNDTVGVTKKCSED |
| | WKLVRSASFSRGGQLSFTDLGLPPTDHLQASFGFQTFQPSGILLDHQT |
| | WTRNLQVTLEDGYIELSTSDSGGPIFKSPQTYMDGLLHYVSVISDNSGL |
| | RLLIDDQLLRNSKRLKHISSSRQSLRLGGSNFEGCISNVFVQRLSLSPEV |
| | LDLTSNSLKRDVSLGGCSLNKPPFLMLLKGSTRFNKTKTFRINQLLQDT |
| | PVASPRSVKVWQDACSPLPKTQANHGALQFGDIPTSHLLFKLPQELLK |
| | PRSQFAVDMQTTSSRGLVFHTGTKNSFMALYLSKGRLVFALGTDGKK |
| | LRIKSKEKCNDGKWHTVVFGHDGEKGRLVVDGLRAREGSLPGNSTISI |
| | RAPVYLGSPPSGKPKSLPTNSFVGCLKNFQLDSKPLYTPSSSFGVSSCL |
| | GGPLEKGIYFSEEGGHVVLAHSVLLGPEFKLVFSIRPRSLTGILIHIGSQP |
| | GKHLCVYLEAGKVTASMDSGAGGTSTSVTPKQSLCDGQWHSVAVTIK |
| | QHILHLELDTDSSYTAGQIPFPPASTQEPLHLGGAPANLTTLRIPVWKSF |
| | FGCLRNIFIVNHIPVPVTEALEVQGPVSLNGCPDQ |
| laminin | MAAAARPRGRALGPVLPPTPLLLLVLRVLPACGATARDPGAAAGLSL |
| subunit | HPTYFNLAEAARIWATATCGERGPGEGRPQPELYCKLVGGPTAPGSGH |
| alpha-3 | TIQGQFCDYCNSEDPRKAHPVTNAIDGSERWWQSPPLSSGTQYNRVNL |
| isoform 5 | TLDLGQLFHVAYILIKFANSPRPDLWVLERSVDFGSTYSPWQYFAHSK |
| precursor | VDCLKEFGREANMAVTRDDDVLCVTEYSRIVPLENGEVVVSLINGRPG |
| (LAMA3- | AKNFTFSHTLREFTKATNIRLRFLRTNTLLGHLISKAQRDPTVTRRYYY |
| isoform 5); | SIKDISIGGQCVCNGHAEVCNINNPEKLFRCECQHHTCGETCDRCCTGY |
| SEQ ID | NQRRWRPAAWEQSHECEACNCHGHASNCYYDPDVERQQASLNTQGI |
| NO: 32 | YAGGGVCINCQHNTAGVNCEQCAKGYYRPYGVPVDAPDGCIRKFHF |
| | KLVYLSLCVLPQRSHQANFGSVNNFLHALSLQSISCARYVTSVTYTVS |
| | LNFGFIACKWK |
| laminin | MALSSAWRSVLPLWLLWSAACSRAASGDDNAFPFDIEGSSAVGRQDP |
| subunit | PETSEPRVALGRLPPAAEKCNAGFFHTLSGECVPCDCNGNSNECLDGS |
| alpha-4 | GYCVHCQRNTTGEHCEKCLDGYIGDSIRGAPQFCQPCPCPLPHLANFA |
| isoform 1 | ESCYRKNGAVRCICNENYAGPNCERCAPGYYGNPLLIGSTCKKCDCSG |
| precursor | NSDPNLIFEDCDEVTGQCRNCLRNTTGFKCERCAPGYYGDARIAKNCA |
| (LAMA4- | VCNCGGGPCDSVTGECLEEGFEPPTGMDCPTISCDKCVWDLTDDLRL |
| isoform 1); | AALSIEEGKSGVLSVSSGAAAHRHVNEINATIYLLKTKLSERENQYALR |
| SEQ ID | KIQINNAENTMKSLLSDVEELVEKENQASRKGQLVQKESMDTINHASQ |
| NO: 33 | LVEQAHDMRDKIQEINNKMLYYGEEHELSPKEISEKLVLAQKMLEEIR |
| | SRQPFFTQRELVDEEADEAYELLSQAESWQRLHNETRTLFPVVLEQLD |
| | DYNAKLSDLQEALDQALNYVRDAEDMNRATAARQRDHEKQQERVR |
| | EQMEVVNMSLSTSADSLTTPRLTLSELDDIIKNASGIYAEIDGAKSELQ |
| | VKLSNLSNLSHDLVQEAIDHAQDLQQEANELSRKLHSSDMNGLVQKA |
| | LDASNVYENIVNYVSEANETAEFALNTTDRIYDAVSGIDTQIIYHKDES |
| | ENLLNQARELQAKAESSSDEAVADTSRRVGGALARKSALKTRLSDAV |
| | KQLQAAERGDAQQRLGQSRLITEEANRTTMEVQQATAPMANNLTNW |
| | SQNLQHFDSSAYNTAVNSARDAVRNLTEVVPQLLDQLRTVEQKRPAS |
| | NVSASIQRIRELIAQTRSVASKIQVSMMFDGQSAVEVHSRTSMDDLKA |
| | FTSLSLYMKPPVKRPELTETADQFILYLGSKNAKKEYMGLAIKNDNLV |
| | YVYNLGTKDVEIPLDSKPVSSWPAYFSIVKIERVGKHGKVFLTVPSLSS |
| | TAEEKFIKKGEFSGDDSLLDLDPEDTVFYVGGVPSNFKLPTSLNLPGFV |
| | GCLELATLNNDVISLYNFKHIYNMDPSTSVPCARDKLAFTQSRAASYF |
| | FDGSGYAVVRDITRRGKFGQVTRFDIEVRTPADNGLILLMVNGSMFFR |
| | LEMRNGYLHVFYDFGFSGGPVHLEDTLKKAQINDAKYHEISIIYHNDK |
| | KMILVVDRRHVKSMDNEKMKIPFTDIYIGGAPPEILQSRALRAHLPLDI |
| | NFRGCMKGFQFQKKDFNLLEQTETLGVGYGCPEDSLISRRAYFNGQSF |
| | IASIQKISFFDGFEGGFNFRTLQPNGLLFYYASGSDVFSISLDNGTVIMD |
| | VKGIKVQSVDKQYNDGLSHFVISSVSPTRYELIVDKSRVGSKNPTKGKI |
| | EQTQASEKKFYFGGSPISAQYANFTGCISNAYFTRVDRDVEVEDFQRY |
| | TEKVHTSLYECPIESSPLFLLHKKGKNLSKPKASQNKKGGKSKDAPSW |
| | DPVALKLPERNTPRNSHCHLSNSPRAIEHAYQYGGTANSRQEFEHLKG |
| | DFGAKSQFSIRLRTRSSHGMIFYVSDQEENDFMTLFLAHGRLVYMFNV |
| | GHKKLKIRSQEKYNDGLWHDVIFIRERSSGRLVIDGLRVLEESLPPTEA |
| | TWKIKGPIYLGGVAPGKAVKNVQINSIYSFSGCLSNLQLNGASITSASQ |
| | TFSVTPCFEGPMETGTYFSTEGGYVVLDESFNIGLKFEIAFEVRPRSSSG |
| | TLVHGHSVNGEYLNVHMKNGQVIVKVNNGIRDFSTSVTPKQSLCDGR |
| | WHRITVIRDSNVVQLDVDSEVNHVVGPLNPKPIDHREPVFVGGVPESL |
| | LTPRLAPSKPFTGCIRHFVIDGHPVSFSKAALVSGAVSINSCPAA |
| laminin | MALSSAWRSVLPLWLLWSAACSRAASGDDNAFPFDIEGSSAVGRQDP |
| subunit | PETSEPRVALGRLPPAAEKCNAGFFHTLSGECVPCDCNGNSNECLDGS |
| alpha-4 | GYCVHCQRNTTGEHCEKCLDGYIGDSIRGAPQFCQPCPCPLPHLANFA |
| isoform 2 | ESCYRKNGAVRCICNENYAGPNCERCAPGYYGNPLLIGSTCKKCDCSG |
| precursor | NSDPNLIFEDCDEVTGQCRNCLRNTTGFKCERCAPGYYGDARIAKNCA |
| (LAMA4- | VCNCGGGPCDSVTGECLEEGFEPPTGCDKCVWDLTDDLRLAALSIEEG |
| isoform 2); | KSGVLSVSSGAAAHRHVNEINATIYLLKTKLSERENQYALRKIQINNAE |
| SEQ ID | NTMKSLLSDVEELVEKENQASRKGQLVQKESMDTINHASQLVEQAHD |
| NO: 34 | MRDKIQEINNKMLYYGEEHELSPKEISEKLVLAQKMLEEIRSRQPFFTQ |
| | RELVDEEADEAYELLSQAESWQRLHNETRTLFPVVLEQLDDYNAKLS |
| | DLQEALDQALNYVRDAEDMNRATAARQRDHEKQQERVREQMEVVN |
| | MSLSTSADSLTTPRLTLSELDDIIKNASGIYAEIDGAKSELQVKLSNLSN |
| | LSHDLVQEAIDHAQDLQQEANELSRKLHSSDMNGLVQKALDASNVYE |
| | NIVNYVSEANETAEFALNTTDRIYDAVSGIDTQIIYHKDESENLLNQAR |
| | ELQAKAESSSDEAVADTSRRVGGALARKSALKTRLSDAVKQLQAAER |
| | GDAQQRLGQSRLITEEANRTTMEVQQATAPMANNLTNWSQNLQHFD |
| | SSAYNTAVNSARDAVRNLTEVVPQLLDQLRTVEQKRPASNVSASIQRI |
| | RELIAQTRSVASKIQVSMMFDGQSAVEVHSRTSMDDLKAFTSLSLYM |
| | KPPVKRPELTETADQFILYLGSKNAKKEYMGLAIKNDNLVYVYNLGT |
| | KDVEIPLDSKPVSSWPAYFSIVKIERVGKHGKVFLTVPSLSSTAEEKFIK |
| | KGEFSGDDSLLDLDPEDTVFYVGGVPSNFKLPTSLNLPGFVGCLELATL |
| | NNDVISLYNFKHIYNMDPSTSVPCARDKLAFTQSRAASYFFDGSGYAV |
| | VRDITRRGKFGQVTRFDIEVRTPADNGLILLMVNGSMFFRLEMRNGYL |
| | HVFYDFGFSGGPVHLEDTLKKAQINDAKYHEISIIYHNDKKMILVVDR |
| | RHVKSMDNEKMKIPFTDIYIGGAPPEILQSRALRAHLPLDINFRGCMKG |
| | FQFQKKDFNLLEQTETLGVGYGCPEDSLISRRAYFNGQSFIASIQKISFF |
| | DGFEGGFNFRTLQPNGLLFYYASGSDVFSISLDNGTVIMDVKGIKVQS |
| | VDKQYNDGLSHFVISSVSPTRYELIVDKSRVGSKNPTKGKIEQTQASEK |
| | KFYFGGSPISAQYANFTGCISNAYFTRVDRDVEVEDFQRYTEKVHTSL |
| | YECPIESSPLFLLHKKGKNLSKPKASQNKKGGKSKDAPSWDPVALKLP |
| | ERNTPRNSHCHLSNSPRAIEHAYQYGGTANSRQEFEHLKGDFGAKSQF |
| | SIRLRTRSSHGMIFYVSDQEENDFMTLFLAHGRLVYMFNVGHKKLKIR |
| | SQEKYNDGLWHDVIFIRERSSGRLVIDGLRVLEESLPPTEATWKIKGPI |
| | YLGGVAPGKAVKNVQINSIYSFSGCLSNLQLNGASITSASQTFSVTPCF |
| | EGPMETGTYFSTEGGYVVLDESFNIGLKFEIAFEVRPRSSSGTLVHGHS |
| | VNGEYLNVHMKNGQVIVKVNNGIRDFSTSVTPKQSLCDGRWHRITVI |
| | RDSNVVQLDVDSEVNHVVGPLNPKPIDHREPVFVGGVPESLLTPRLAP |
| | SKPFTGCIRHFVIDGHPVSFSKAALVSGAVSINSCPAA |
| laminin | MALSSAWRSVLPLWLLWSAACSRAASGDDNAFPFDIEGSSAVGRQDP |
| subunit | PETSEPRVALGRLPPAAEVQCPCHCHPAGAPAPPRAVPHSSFSLSPPLSS |
| alpha-4 | PQCLESFTWARSVRKLEIKSFPL |
| isoform 3 | |
| precursor | |
| (LAMA4- | |
| isoform 3); | |
| SEQ ID | |
| NO: 35 | |
| laminin | MAKRLCAGSALCVRGPRGPAPLLLVGLALLGAARAREEAGGGFSLHP |
| subunit | PYFNLAEGARIAASATCGEEAPARGSPRPTEDLYCKLVGGPVAGGDPN |
| alpha-5 | QTIRGQYCDICTAANSNKAHPASNAIDGTERWWQSPPLSRGLEYNEVN |
| precursor | VTLDLGQVFHVAYVLIKFANSPRPDLWVLERSMDFGRTYQPWQFFAS |
| (LAMA5); | SKRDCLERFGPQTLERITRDDAAICTTEYSRIVPLENGEIVVSLVNGRPG |
| SEQ ID | AMNFSYSPLLREFTKATNVRLRFLRTNTLLGHLMGKALRDPTVTRRY |
| NO: 36 | YYSIKDISIGGRCVCHGHADACDAKDPTDPFRLQCTCQHNTCGGTCDR |
| | CCPGFNQQPWKPATANSANECQSCNCYGHATDCYYDPEVDRRRASQ |
| | SLDGTYQGGGVCIDCQHHTTGVNCERCLPGFYRSPNHPLDSPHVCRRC |
| | NCESDFTDGTCEDLTGRCYCRPNFSGERCDVCAEGFTGFPSCYPTPSSS |
| | NDTREQVLPAGQIVNCDCSAAGTQGNACRKDPRVGRCLCKPNFQGTH |
| | CELCAPGFYGPGCQPCQCSSPGVADDRCDPDTGQCRCRVGFEGATCD |
| | RCAPGYFHFPLCQLCGCSPAGTLPEGCDEAGRCLCQPEFAGPHCDRCR |
| | PGYHGFPNCQACTCDPRGALDQLCGAGGLCRCRPGYTGTACQECSPG |
| | FHGFPSCVPCHCSAEGSLHAACDPRSGQCSCRPRVTGLRCDTCVPGAY |
| | NFPYCEAGSCHPAGLAPVDPALPEAQVPCMCRAHVEGPSCDRCKPGF |
| | WGLSPSNPEGCTRCSCDLRGTLGGVAECQPGTGQCFCKPHVCGQACA |
| | SCKDGEEGLDQADYFGCRSCRCDIGGALGQSCEPRTGVCRCRPNTQGP |
| | TCSEPARDHYLPDLHHLRLELEEAATPEGHAVRFGFNPLEFENFSWRG |
| | YAQMAPVQPRIVARLNLTSPDLFWLVFRYVNRGAMSVSGRVSVREEG |
| | RSATCANCTAQSQPVAFPPSTEPAFITVPQRGEGEPEVLNPGTWALRVE |
| | AEGVLLDYVVLLPSAYYEAALLQLRVTEACTYRPSAQQSGDNCLLYT |
| | HLPLDGFPSAAGLEALCRQDNSLPRPCPTEQLSPSHPPLITCTGSDVDV |
| | QLQVAVPQPGRYALVVEYANEDARQEVGVAVHTPQRAPQQGLLSLH |
| | PCLYSTLCRGTARDTQDHLAVFHLDSEASVRLTAEQARFFLHGVTLVP |
| | IEEESPEFVEPRVSCISSHGAFGPNSAACLPSRFPKPPQPIILRDCQVIPLP |
| | PGLPLTHAQDLTPAMSPAGPRPRPPTAVDPDAEPTLLREPQATVVFTTH |
| | VPTLGRYAELLHGYQPAEIPTEPVEVLINAGRVWQGHANASFCPHGYG |
| | CRTLVVCEGQALLDVTHSELTVTVRVPKGRWLWLDYVLVVPENVYS |
| | FGYLREEPLDKSYDFISHCAAQGYHISPSSSSLFCRNAAASLSLFYNNG |
| | ARPCGCHEVGATGPTCEPFGGQCPCHAHVIGRDCSRCATGYWGFPNC |
| | RPCDCGARLCDELTGQCICPPRTIPPDCLLCQPQTFGCHPLVGCEECNC |
| | SGPGIQELTDPTCDTDSGQCKCRPNVTGRRCDTCSPGFHGYPRCRPCD |
| | CHEAGTAPGVCDPLTGQCYCKENVQGPKCDQCSLGTFSLDAANPKGC |
| | TRCFCFGATERCRSSSYTRQEFVDMEGWVLLSTDRQVVPHERQPGTE |
| | MLRADLRHVPEAVPEAFPELYWQAPPSYLGDRVSSYGGTLRYELHSE |
| | TQRGDVFVPMESRPDVVLQGNQMSITFLEPAYPTPGHVHRGQLQLVE |
| | GNFRHTETRNTVSREELMMVLASLEQLQIRALFSQISSAVFLRRVALEV |
| | ASPAGQGALASNVELCLCPASYRGDSCQECAPGFYRDVKGLFLGRCV |
| | PCQCHGHSDRCLPGSGVCVDCQHNTEGAHCERCQAGFVSSRDDPSAP |
| | CVSCPCPLSVPSNNFAEGCVLRGGRTQCLCKPGYAGASCERCAPGFFG |
| | NPLVLGSSCQPCDCSGNGDPNLLFSDCDPLTGACRGCLRHTTGPRCEIC |
| | APGFYGNALLPGNCTRCDCTPCGTEACDPHSGHCLCKAGVTGRRCDR |
| | CQEGHFGEDGCGGCRPCACGPAAEGSECHPQSGQCHCRPGTMGPQCR |
| | ECAPGYWGLPEQGCRRCQCPGGRCDPHTGRCNCPPGLSGERCDTCSQ |
| | QHQVPVPGGPVGHSIHCEVCDHCVVLLLDDLERAGALLPAIHEQLRGI |
| | NASSMAWARLHRLNASIADLQSQLRSPLGPRHETAQQLEVLEQQSTSL |
| | GQDARRLGGQAVGTRDQASQLLAGTEATLGHAKTLLAAIRAVDRTLS |
| | ELMSQTGHLGLANASAPSGEQLLRTLAEVERLLWEMRARDLGAPQAA |
| | AEAELAAAQRLLARVQEQLSSLWEENQALATQTRDRLAQHEAGLMD |
| | LREALNRAVDATREAQELNSRNQERLEEALQRKQELSRDNATLQATL |
| | HAARDTLASVFRLLHSLDQAKEELERLAASLDGARTPLLQRMQTFSPA |
| | GSKLRLVEAAEAHAQQLGQLALNLSSIILDVNQDRLTQRAIEASNAYS |
| | RILQAVQAAEDAAGQALQQADHTWATVVRQGLVDRAQQLLANSTAL |
| | EEAMLQEQQRLGLVWAALQGARTQLRDVRAKKDQLEAHIQAAQAM |
| | LAMDTDETSKKIAHAKAVAAEAQDTATRVQSQLQAMQENVERWQG |
| | QYEGLRGQDLGQAVLDAGHSVSTLEKTLPQLLAKLSILENRGVHNASL |
| | ALSASIGRVRELIAQARGAASKVKVPMKFNGRSGVQLRTPRDLADLA |
| | AYTALKFYLQGPEPEPGQGTEDRFVMYMGSRQATGDYMGVSLRDKK |
| | VHWVYQLGEAGPAVLSIDEDIGEQFAAVSLDRTLQFGHMSVTVERQM |
| | IQETKGDTVAPGAEGLLNLRPDDFVFYVGGYPSTFTPPPLLRFPGYRGC |
| | IEMDTLNEEVVSLYNEERTEQLDTAVDRPCARSKSTGDPWLTDGSYLD |
| | GTGEARISEDSQISTTKREEQELRLVSYSGVLEELKQQSQFLCLAVQEGS |
| | LVLLYDFGAGLKKAVPLQPPPPLTSASKAIQVFLLGGSRKRVLVRVER |
| | ATVYSVEQDNDLELADAYYLGGVPPDQLPPSLRRLFPTGGSVRGCVK |
| | GIKALGKYVDLKRLNTTGVSAGCTADLLVGRAMTFHGHGFLRLALSN |
| | VAPLTGNVYSGFGFHSAQDSALLYYRASPDGLCQVSLQQGRVSLQLL |
| | RTEVKTQAGFADGAPHYVAFYSNATGVWLYVDDQLQQMKPHRGPPP |
| | ELQPQPEGPPRLLLGGLPESGTIYNFSGCISNVFVQRLLGPQRVFDLQQ |
| | NLGSVNVSTGCAPALQAQTPGLGPRGLQATARKASRRSRQPARHPAC |
| | MLPPHLRTTRDSYQFGGSLSSHLEFVGILARHRNWPSLSMHVLPRSSR |
| | GLLLFTARLRPGSPSLALFLSNGHFVAQMEGLGTRLRAQSRQRSRPGR |
| | WHKVSVRWEKNRILLVTDGARAWSQEGPHRQHQGAEHPQPHTLFVG |
| | GLPASSHSSKLPVTVGFSGCVKRLRLHGRPLGAPTRMAGVTPCILGPLE |
| | AGLFFPGSGGVITLDLPGATLPDVGLELEVRPLAVTGLIFHLGQARTPP |
| | YLQLQVTEKQVLLRADDGAGEFSTSVTRPSVLCDGQWHRLAVMKSG |
| | NVLRLEVDAQSNHTVGPLLAAAAGAPAPLYLGGLPEPMAVQPWPPAY |
| | CGCMRRLAVNRSPVAMTRSVEVHGAVGASGCPAA |
| laminin | MGLLQLLAFSFLALCRARVRAQEPEFSYGCAEGSCYPATGDLLIGRAQ |
| subunit beta- | KLSVTSTCGLHKPEPYCIVSHLQEDKKCFICNSQDPYHETLNPDSHLIE |
| 1 precursor | NVVTTFAPNRLKIWWQSENGVENVTIQLDLEAEFHFTHLIMTFKTFRP |
| (LAMB1); | AAMLIERSSDFGKTWGVYRYFAYDCEASFPGISTGPMKKVDDIICDSR |
| SEQ ID | YSDIEPSTEGEVIFRALDPAFKIEDPYSPRIQNLLKITNLRIKFVKLHTLG |
| NO: 37 | DNLLDSRMEIREKYYYAVYDMVVRGNCFCYGHASECAPVDGFNEEV |
| | EGMVHGHCMCRHNTKGLNCELCMDFYHDLPWRPAEGRNSNACKKC |
| | NCNEHSISCHFDMAVYLATGNVSGGVCDDCQHNTMGRNCEQCKPFY |
| | YQHPERDIRDPNFCERCTCDPAGSQNEGICDSYTDFSTGLIAGQCRCKL |
| | NVEGEHCDVCKEGFYDLSSEDPFGCKSCACNPLGTIPGGNPCDSETGH |
| | CYCKRLVTGQHCDQCLPEHWGLSNDLDGCRPCDCDLGGALNNSCFA |
| | ESGQCSCRPHMIGRQCNEVEPGYYFATLDHYLYEAEEANLGPGVSIVE |
| | RQYIQDRIPSWTGAGFVRVPEGAYLEFFIDNIPYSMEYDILIRYEPQLPD |
| | HWEKAVITVQRPGRIPTSSRCGNTIPDDDNQVVSLSPGSRYVVLPRPVC |
| | FEKGTNYTVRLELPQYTSSDSDVESPYTLIDSLVLMPYCKSLDIFTVGG |
| | SGDGVVTNSAWETFQRYRCLENSRSVVKTPMTDVCRNIIFSISALLHQT |
| | GLACECDPQGSLSSVCDPNGGQCQCRPNVVGRTCNRCAPGTFGFGPS |
| | GCKPCECHLQGSVNAFCNPVTGQCHCFQGVYARQCDRCLPGHWGFPS |
| | CQPCQCNGHADDCDPVTGECLNCQDYTMGHNCERCLAGYYGDPIIGS |
| | GDHCRPCPCPDGPDSGRQFARSCYQDPVTLQLACVCDPGYIGSRCDDC |
| | ASGYFGNPSEVGGSCQPCQCHNNIDTTDPEACDKETGRCLKCLYHTEG |
| | EHCQFCRFGYYGDALQQDCRKCVCNYLGTVQEHCNGSDCQCDKATG |
| | QCLCLPNVIGQNCDRCAPNTWQLASGTGCDPCNCNAAHSFGPSCNEF |
| | TGQCQCMPGFGGRTCSECQELFWGDPDVECRACDCDPRGIETPQCDQ |
| | STGQCVCVEGVEGPRCDKCTRGYSGVFPDCTPCHQCFALWDVIIAELT |
| | NRTHRFLEKAKALKISGVIGPYRETVDSVERKVSEIKDILAQSPAAEPL |
| | KNIGNLFEEAEKLIKDVTEMMAQVEVKLSDTTSQSNSTAKELDSLQTE |
| | AESLDNTVKELAEQLEFIKNSDIRGALDSITKYFQMSLEAEERVNASTT |
| | EPNSTVEQSALMRDRVEDVMMERESQFKEKQEEQARLLDELAGKLQS |
| | LDLSAAAEMTCGTPPGASCSETECGGPNCRTDEGERKCGGPGCGGLV |
| | TVAHNAWQKAMDLDQDVLSALAEVEQLSKMVSEAKLRADEAKQSA |
| | EDILLKTNATKEKMDKSNEELRNLIKQIRNFLTQDSADLDSIEAVANEV |
| | LKMEMPSTPQQLQNLTEDIRERVESLSQVEVILQHSAADIARAEMLLEE |
| | AKRASKSATDVKVTADMVKEALEEAEKAQVAAEKAIKQADEDIQGT |
| | QNLLTSHESETAASEETLFNASQRISELERNVEELKRKAAQNSGEAEYIE |
| | KVVYTVKQSAEDVKKTLDGELDEKYKKVENLIAKKTEESADARRKAE |
| | MLQNEAKTLLAQANSKLQLLKDLERKYEDNQRYLEDKAQELARLEG |
| | EVRSLLKDISQKVAVYSTCL |
| laminin | MELTSRERGRGQPLPWELRLGLLLSVLAATLAQAPAPDVPGCSRGSCY |
| subunit beta- | PATGDLLVGRADRLTASSTCGLNGPQPYCIVSHLQDEKKCFLCDSRRP |
| 2 precursor | FSARDNPHSHRIQNVVTSFAPQRRAAWWQSENGIPAVTIQLDLEAEFH |
| (LAMB2); | FTHLIMTFKTFRPAAMLVERSADFGRTWHVYRYFSYDCGADFPGVPL |
| SEQ ID | APPRHWDDVVCESRYSEIEPSTEGEVIYRVLDPAIPIPDPYSSRIQNLLKI |
| NO: 38 | TNLRVNLTRLHTLGDNLLDPRREIREKYYYALYELVVRGNCFCYGHA |
| | SECAPAPGAPAHAEGMVHGACICKHNTRGLNCEQCQDFYRDLPWRPA |
| | EDGHSHACRKCECHGHTHSCHFDMAVYLASGNVSGGVCDGCQHNTA |
| | GRHCELCRPFFYRDPTKDLRDPAVCRSCDCDPMGSQDGGRCDSHDDP |
| | ALGLVSGQCRCKEHVVGTRCQQCRDGFFGLSISDRLGCRRCQCNARG |
| | TVPGSTPCDPNSGSCYCKRLVTGRGCDRCLPGHWGLSHDLLGCRPCD |
| | CDVGGALDPQCDEGTGQCHCRQHMVGRRCEQVQPGYFRPFLDHLIW |
| | EAEDTRGQVLDVVERLVTPGETPSWTGSGFVRLQEGQTLEFLVASVPK |
| | AMDYDLLLRLEPQVPEQWAELELIVQRPGPVPAHSLCGHLVPKDDRIQ |
| | GTLQPHARYLIFPNPVCLEPGISYKLHLKLVRTGGSAQPETPYSGPGLLI |
| | DSLVLLPRVLVLEMFSGGDAAALERQATFERYQCHEEGLVPSKTSPSE |
| | ACAPLLISLSTLIYNGALPCQCNPQGSLSSECNPHGGQCLCKPGVVGRR |
| | CDLCAPGYYGFGPTGCQACQCSHEGALSSLCEKTSGQCLCRTGAFGLR |
| | CDRCQRGQWGFPSCRPCVCNGHADECNTHTGACLGCRDHTGGEHCE |
| | RCIAGFHGDPRLPYGGQCRPCPCPEGPGSQRHFATSCHQDEYSQQIVC |
| | HCRAGYTGLRCEACAPGHFGDPSRPGGRCQLCECSGNIDPMDPDACD |
| | PHTGQCLRCLHHTEGPHCAHCKPGFHGQAARQSCHRCTCNLLGTNPQ |
| | QCPSPDQCHCDPSSGQCPCLPNVQGPSCDRCAPNFWNLTSGHGCQPCA |
| | CHPSRARGPTCNEFTGQCHCRAGFGGRTCSECQELHWGDPGLQCHAC |
| | DCDSRGIDTPQCHRFTGHCSCRPGVSGVRCDQCARGFSGIFPACHPCH |
| | ACFGDWDRVVQDLAARTQRLEQRAQELQQTGVLGAFESSFWHMQEK |
| | LGIVQGIVGARNTSAASTAQLVEATEELRREIGEATEHLTQLEADLTDV |
| | QDENFNANHALSGLERDRLALNLTLRQLDQHLDLLKHSNFLGAYDSIR |
| | HAHSQSAEAERRANTSALAVPSPVSNSASARHRTEALMDAQKEDFNS |
| | KHMANQRALGKLSAHTHTLSLTDINELVCGAPGDAPCATSPCGGAGC |
| | RDEDGQPRCGGLSCNGAAATADLALGRARHTQAELQRALAEGGSILS |
| | RVAETRRQASEAQQRAQAALDKANASRGQVEQANQELQELIQSVKDF |
| | LNQEGADPDSIEMVATRVLELSIPASAEQIQHLAGAIAERVRSLADVDA |
| | ILARTVGDVRRAEQLLQDARRARSWAEDEKQKAETVQAALEEAQRA |
| | QGIAQGAIRGAVADTRDTEQTLYQVQERMAGAERALSSAGERARQLD |
| | ALLEALKLKRAGNSLAASTAEETAGSAQGRAQEAEQLLRGPLGDQYQ |
| | TVKALAERKAQGVLAAQARAEQLRDEARDLLQAAQDKLQRLQELEG |
| | TYEENERALESKAAQLDGLEARMRSVLQAINLQVQIYNTCQ |
| laminin | MRPFFLLCFALPGLLHAQQACSRGACYPPVGDLLVGRTRFLRASSTCG |
| subunit beta- | LTKPETYCTQYGEWQMKCCKCDSRQPHNYYSHRVENVASSSGPMRW |
| 3 precursor | WQSQNDVNPVSLQLDLDRRFQLQEVMMEFQGPMPAGMLIERSSDFG |
| (LAMB3); | KTWRVYQYLAADCTSTFPRVRQGRPQSWQDVRCQSLPQRPNARLNG |
| SEQ ID | GKVQLNLMDLVSGIPATQSQKIQEVGEITNLRVNFTRLAPVPQRGYHP |
| NO: 39 | PSAYYAVSQLRLQGSCFCHGHADRCAPKPGASAGPSTAVQVHDVCVC |
| | QHNTAGPNCERCAPFYNNRPWRPAEGQDAHECQRCDCNGHSETCHF |
| | DPAVFAASQGAYGGVCDNCRDHTEGKNCERCQLHYFRNRRPGASIQE |
| | TCISCECDPDGAVPGAPCDPVTGQCVCKEHVQGERCDLCKPGFTGLTY |
| | ANPQGCHRCDCNILGSRRDMPCDEESGRCLCLPNVVGPKCDQCAPYH |
| | WKLASGQGCEPCACDPHNSLSPQCNQFTGQCPCREGFGGLMCSAAAI |
| | RQCPDRTYGDVATGCRACDCDFRGTEGPGCDKASGRCLCRPGLTGPR |
| | CDQCQRGYCNRYPVCVACHPCFQTYDADLREQALRFGRLRNATASL |
| | WSGPGLEDRGLASRILDAKSKIEQIRAVLSSPAVTEQEVAQVASAILSL |
| | RRTLQGLQLDLPLEEETLSLPRDLESLDRSFNGLLTMYQRKREQFEKIS |
| | SADPSGAFRMLSTAYEQSAQAAQQVSDSSRLLDQLRDSRREAERLVR |
| | QAGGGGGTGSPKLVALRLEMSSLPDLTPTFNKLCGNSRQMACTPISCP |
| | GELCPQDNGTACGSRCRGVLPRAGGAFLMAGQVAEQLRGFNAQLQR |
| | TRQMIRAAEESASQIQSSAQRLETQVSASRSQMEEDVRRTRLLIQQVR |
| | DFLTDPDTDAATIQEVSEAVLALWLPTDSATVLQKMNEIQAIAARLPN |
| | VDLVLSQTKQDIARARRLQAEAEEARSRAHAVEGQVEDVVGNLRQGT |
| | VALQEAQDTMQGTSRSLRLIQDRVAEVQQVLRPAEKLVTSMTKQLGD |
| | FWTRMEELRHQARQQGAEAVQAQQLAEGASEQALSAQEGFERIKQK |
| | YAELKDRLGQSSMLGEQGARIQSVKTEAEELFGETMEMMDRMKDME |
| | LELLRGSQAIMLRSADLTGLEKRVEQIRDHINGRVLYYATCK |
| laminin | MQFQLTLFLHLGWLSYSKAQDDCNRGACHPTTGDLLVGRNTQLMAS |
| subunit beta- | STCGLSRAQKYCILSYLEGEQKCFICDSRFPYDPYDQPNSHTIENVIVSF |
| 4 isoform 1 | EPDREKKWWQSENGLDHVSIRLDLEALFRFSHLILTFKTFRPAAMLVE |
| precursor | RSTDYGHNWKVFKYFAKDCATSFPNITSGQAQGVGDIVCDSKYSDIEP |
| (LAMB4- | STGGEVVLKVLDPSFEIENPYSPYIQDLVTLTNLRINFTKLHTLGDALLG |
| isoform 1); | RRQNDSLDKYYYALYEMIVRGSCFCNGHASECRPMQKMRGDVFSPPG |
| SEQ ID | MVHGQCVCQHNTDGPNCERCKDFFQDAPWRPAADLQDNACRSCSCN |
| NO: 40 | SHSSRCHFDMTTYLASGGLSGGVCEDCQHNTEGQHCDRCRPLFYRDP |
| | LKTISDPYACIPCECDPDGTISGGICVSHSDPALGSVAGQCLCKENVEG |
| | AKCDQCKPNHYGLSATDPLGCQPCDCNPLGSLPFLTCDVDTGQCLCLS |
| | YVTGAHCEECTVGYWGLGNHLHGCSPCDCDIGGAYSNVCSPKNGQC |
| | ECRPHVTGRSCSEPAPGYFFAPLNFYLYEAEEATTLQGLAPLGSETFGQ |
| | SPAVHVVLGEPVPGNPVTWTGPGFARVLPGAGLRFAVNNIPFPVDFTI |
| | AIHYETQSAADWTVQIVVNPPGGSEHCIPKTLQSKPQSFALPAATRIML |
| | LPTPICLEPDVQYSIDVYFSQPLQGESHAHSHVLVDSLGLIPQINSLENF |
| | CSKQDLDEYQLHNCVEIASAMGPQVLPGACERLIISMSAKLHDGAVAC |
| | KCHPQGSVGSSCSRLGGQCQCKPLVVGRCCDRCSTGSYDLGHHGCHP |
| | CHCHPQGSKDTVCDQVTGQCPCHGEVSGRRCDRCLAGYFGFPSCHPC |
| | PCNRFAELCDPETGSCFNCGGFTTGRNCERCIDGYYGNPSSGQPCRPCL |
| | CPDDPSSNQYFAHSCYQNLWSSDVICNCLQGYTGTQCGECSTGFYGNP |
| | RISGAPCQPCACNNNIDVTDPESCSRVTGECLRCLHNTQGANCQLCKP |
| | GHYGSALNQTCRRCSCHASGVSPMECPPGGGACLCDPVTGACPCLPN |
| | VTGLACDRCADGYWNLVPGRGCQSCDCDPRTSQSSHCDQLTGQCPC |
| | KLGYGGKRCSECQENYYGDPPGRCIPCDCNRAGTQKPICDPDTGMCR |
| | CREGVSGQRCDRCARGHSQEFPTCLQCHLCFDQWDHTISSLSKAVQG |
| | LMRLAANMEDKRETLPVCEADFKDLRGNVSEIERILKHPVFPSGKFLK |
| | VKDYHDSVRRQIMQLNEQLKAVYEFQDLKDTIERAKNEADLLLEDLQ |
| | EEIDLQSSVLNASIADSSENIKKYYHISSSAEKKINETSSTINTSANTRND |
| | LLTILDTLTSKGNLSLERLKQIKIPDIQILNEKVCGDPGNVPCVPLPCGG |
| | ALCTGRKGHRKCRGPGCHGSLTLSTNALQKAQEAKSIIRNLDKQVRGL |
| | KNQIESISEQAEVSKNNALQLREKLGNIRNQSDSEEENINLFIKKVKNFL |
| | LEENVPPEDIEKVANGVLDIHLPIPSQNLTDELVKIQKHMQLCEDYRTD |
| | ENRLNEEADGAQKLLVKAKAAEKAANILLNLDKTLNQLQQAQITQGR |
| | ANSTITQLTANITKIKKNVLQAENQTREMKSELELAKQRSGLEDGLSLL |
| | QTKLQRHQDHAVNAKVQAESAQHQAGSLEKEFVELKKQYAILQRKTS |
| | TTGLTKETLGKVKQLKDAAEKLAGDTEAKIRRITDLERKIQDLNLSRQ |
| | AKADQLRILEDQVVAIKNEIVEQEKKYARCYS |
| laminin | MQFQLTLFLHLGWLSYSKAQDDCNRGACHPTTGDLLVGRNTQLMAS |
| subunit beta- | STCGLSRAQKYCILSYLEGEQKCFICDSRFPYDPYDQPNSHTIENVIVSF |
| 4 isoform 2 | EPDREKKWWQSENGLDHVSIRLDLEALFRFSHLILTFKTFRPAAMLVE |
| precursor | RSTDYGHNWKVFKYFAKDCATSFPNITSGQAQGVGDIVCDSKYSDIEP |
| (LAMB4- | STGGEVVLKVLDPSFEIENPYSPYIQDLVTLTNLRINFTKLHTLGDALLG |
| isoform 2); | RRQNDSLDKYYYALYEMIVRGSCFCNGHASECRPMQKMRGDVFSPPG |
| SEQ ID | MVHGQCVCQHNTDGPNCERCKDFFQDAPWRPAADLQDNACRSCSCN |
| NO: 41 | SHSSRCHFDMTTYLASGGLSGGVCEDCQHNTEGQHCDRCRPLFYRDP |
| | LKTISDPYACIPCECDPDGTISGGICVSHSDPALGSVAGQCLCKENVEG |
| | AKCDQCKPNHYGLSATDPLGCQPCDCNPLGSLPFLTCDVDTGQCLCLS |
| | YVTGAHCEECTVGYWGLGNHLHGCSPCDCDIGGAYSNVCSPKNGQC |
| | ECRPHVTGRSCSEPAPGYFFAPLNFYLYEAEEATTLQGLAPLGSETFGQ |
| | SPAVHVVLGEPVPGNPVTWTGPGFARVLPGAGLRFAVNNIPFPVDFTI |
| | AIHYETQSAADWTVQIVVNPPGGSEHCIPKTLQSKPQSFALPAATRIML |
| | LPTPICLEPDVQYSIDVYFSQPLQGESHAHSHVLVDSLGLIPQINSLENF |
| | CSKQDLDEYQLHNCVEIASAMGPQVLPGACERLIISMSAKLHDGAVAC |
| | KCHPQGSVGSSCSRLGGQCQCKPLVVGRCCDRCSTGSYDLGHHGCHP |
| | CHCHPQGSKDTVCDQVTGQCPCHGEVSGRRCDRCLAGYFGFPSCHPC |
| | PCNRFAELCDPETGSCFNCGGFTTGRNCERCIDGYYGNPSSGQPCRPCL |
| | CPDDPSSNQYFAHSCYQNLWSSDVICNCLQGYTGTQCGECSTGFYGNP |
| | RISGAPCQPCACNNNIDVTDPESCSRVTGECLRCLHNTQGANCQLCKP |
| | GHYGSALNQTCRRCSCHASGVSPMECPPGGGACLCDPVTGACPCLPN |
| | VTGLACDRCADGYWNLVPGRGCQSCDCDPRTSQSSHCDQARYFKAY |
| laminin | MQFQLTLFLHLGWLSYSKAQDDCNRGACHPTTGDLLVGRNTQLMAS |
| subunit beta- | STCGLSRAQKYCILSYLEGEQKCFICDSRFPYDPYDQPNSHTIENVIVSF |
| 4 isoform 3 | EPDREKKWWQSENGLDHVSIRLDLEALFRFSHLILTFKTFRPAAMLVE |
| precursor | RSTDYGHNWKVFKYFAKDCATSFPNITSGQAQGVGDIVCDSKYSDIEP |
| (LAMB4- | STGGEVVLKVLDPSFEIENPYSPYIQDLVTLTNLRINFTKLHTLGDALLG |
| isoform 3); | RRQNDSLDKYYYALYEMIVRGSCFCNGHASECRPMQKMRGDVFSPPG |
| SEQ ID | MVHGQCVCQHNTDGPNCERCKDFFQDAPWRPAADLQDNACRSCSCN |
| NO: 42 | SHSSRCHFDMTTYLASGGLSGGVCEDCQHNTEGQHCDRCRPLFYRDP |
| | LKTISDPYACIPCECDPDGTISGGICVSHSDPALGSVAGQCLCKENVEG |
| | AKCDQCKPNHYGLSATDPLGCQPCDCNPLGSLPFLTCDVDTGQCLCLS |
| | YVTGAHCEECTVGYWGLGNHLHGCSPCDCDIGGAYSNVCSPKNGQC |
| | ECRPHVTGRSCSEPAPGYFFAPLNFYLYEAEEATTLQGLAPLGSETFGQ |
| | SPAVHVVLGEPVPGNPVTWTGPGFARVLPGAGLRFAVNNIPFPVDFTI |
| | AIHYETQSAADWTVQIVVNPPGGSEHCIPKTLQSKPQSFALPAATRIML |
| | LPTPICLEPDVQYSIDVYFSQPLQGESHAHSHVLVDSAAVQWHNLGSL |
| | QPPPPECKQFSCFSFPSSWDYREIPPPHLANFCIFSRDGVSPHWPGWSQT |
| | PDLR |
| laminin | MRGSHRAAPALRPRGRLWPVLAVLAAAAAAGCAQAAMDECTDEGG |
| subunit | RPQRCMPEFVNAAFNVTVVATNTCGTPPEEYCVQTGVTGVTKSCHLC |
| gamma-1 | DAGQPHLQHGAAFLTDYNNQADTTWWQSQTMLAGVQYPSSINLTLH |
| precursor | LGKAFDITYVRLKFHTSRPESFAIYKRTREDGPWIPYQYYSGSCENTYS |
| (LAMC1); | KANRGFIRTGGDEQQALCTDEFSDISPLTGGNVAFSTLEGRPSAYNFDN |
| SEQ ID | SPVLQEWVTATDIRVTLNRLNTFGDEVFNDPKVLKSYYYAISDFAVGG |
| NO: 43 | RCKCNGHASECMKNEFDKLVCNCKHNTYGVDCEKCLPFFNDRPWRR |
| | ATAESASECLPCDCNGRSQECYFDPELYRSTGHGGHCTNCQDNTDGA |
| | HCERCRENFFRLGNNEACSSCHCSPVGSLSTQCDSYGRCSCKPGVMGD |
| | KCDRCQPGFHSLTEAGCRPCSCDPSGSIDECNIETGRCVCKDNVEGFNC |
| | ERCKPGFFNLESSNPRGCTPCFCFGHSSVCTNAVGYSVYSISSTFQIDED |
| | GWRAEQRDGSEASLEWSSERQDIAVISDSYFPRYFIAPAKFLGKQVLSY |
| | GQNLSFSFRVDRRDTRLSAEDLVLEGAGLRVSVPLIAQGNSYPSETTV |
| | KYVFRLHEATDYPWRPALTPFEFQKLLNNLTSIKIRGTYSERSAGYLDD |
| | VTLASARPGPGVPATWVESCTCPVGYGGQFCEMCLSGYRRETPNLGP |
| | YSPCVLCACNGHSETCDPETGVCNCRDNTAGPHCEKCSDGYYGDSTA |
| | GTSSDCQPCPCPGGSSCAVVPKTKEVVCTNCPTGTTGKRCELCDDGYF |
| | GDPLGRNGPVRLCRLCQCSDNIDPNAVGNCNRLTGECLKCIYNTAGFY |
| | CDRCKDGFFGNPLAPNPADKCKACNCNLYGTMKQQSSCNPVTGQCE |
| | CLPHVTGQDCGACDPGFYNLQSGQGCERCDCHALGSTNGQCDIRTGQ |
| | CECQPGITGQHCERCEVNHFGFGPEGCKPCDCHPEGSLSLQCKDDGRC |
| | ECREGFVGNRCDQCEENYFYNRSWPGCQECPACYRLVKDKVADHRV |
| | KLQELESLIANLGTGDEMVTDQAFEDRLKEAEREVMDLLREAQDVKD |
| | VDQNLMDRLQRVNNTLSSQISRLQNIRNTIEETGNLAEQARAHVENTE |
| | RLIEIASRELEKAKVAAANVSVTQPESTGDPNNMTLLAEEARKLAERH |
| | KQEADDIVRVAKTANDTSTEAYNLLLRTLAGENQTAFEIEELNRKYEQ |
| | AKNISQDLEKQAARVHEEAKRAGDKAVEIYASVAQLSPLDSETLENEA |
| | NNIKMEAENLEQLIDQKLKDYEDLREDMRGKELEVKNLLEKGKTEQQ |
| | TADQLLARADAAKALAEEAAKKGRDTLQEANDILNNLKDFDRRVND |
| | NKTAAEEALRKIPAINQTITEANEKTREAQQALGSAAADATEAKNKAH |
| | EAERIASAVQKNATSTKAEAERTFAEVTDLDNEVNNMLKQLQEAEKE |
| | LKRKQDDADQDMMMAGMASQAAQEAEINARKAKNSVTSLLSIINDL |
| | LEQLGQLDTVDLNKLNEIEGTLNKAKDEMKVSDLDRKVSDLENEAKK |
| | QEAAIMDYNRDIEEIMKDIRNLEDIRKTLPSGCFNTPSIEKP |
| laminin | MPALWLGCCLCFSLLLPAARATSRREVCDCNGKSRQCIFDRELHRQTG |
| subunit | NGFRCLNCNDNTDGIHCEKCKNGFYRHRERDRCLPCNCNSKGSLSAR |
| gamma-2 | CDNSGRCSCKPGVTGARCDRCLPGFHMLTDAGCTQDQRLLDSKCDCD |
| isoform a | PAGIAGPCDAGRCVCKPAVTGERCDRCRSGYYNLDGGNPEGCTQCFC |
| precursor | YGHSASCRSSAEYSVHKITSTFHQDVDGWKAVQRNGSPAKLQWSQRH |
| (LAMC2- | QDVFSSAQRLDPVYFVAPAKFLGNQQVSYGQSLSFDYRVDRGGRHPS |
| isoform a); | AHDVILEGAGLRITAPLMPLGKTLPCGLTKTYTFRLNEHPSNNWSPQLS |
| SEQ ID | YFEYRRLLRNLTALRIRATYGEYSTGYIDNVTLISARPVSGAPAPWVEQ |
| NO: 44 | CICPVGYKGQFCQDCASGYKRDSARLGPFGTCIPCNCQGGGACDPDTG |
| | DCYSGDENPDIECADCP1GFYNDPHDPRSCKPCPCHNGFSCSVMPETEE |
| | VVCNNCPPGVTGARCELCADGYFGDPFGEHGPVRPCQPCQCNNNVDP |
| | SASGNCDRLTGRCLKCIHNTAGIYCDQCKAGYFGDPLAPNPADKCRA |
| | CNCNPMGSEPVGCRSDGTCVCKPGFGGPNCEHGAFSCPACYNQVKIQ |
| | MDQFMQQLQRMEALISKAQGGDGVVPDTELEGRMQQAEQALQDILR |
| | DAQISEGASRSLGLQLAKVRSQENSYQSRLDDLKMTVERVRALGSQY |
| | QNRVRDTHRLITQMQLSLAESEASLGNTNIPASDHYVGPNGFKSLAQE |
| | ATRLAESHVESASNMEQLTRETEDYSKQALSLVRKALHEGVGSGSGSP |
| | DGAVVQGLVEKLEKTKSLAQQLTREATQAEIEADRSYQHSLRLLDSVS |
| | RLQGVSDQSFQVEEAKRIKQKADSLSSLVTRHMDEFKRTQKNLGNWK |
| | EEAQQLLQNGKSGREKSDQLLSRANLAKSRAQEALSMGNATFYEVESI |
| | LKNLREFDLQVDNRKAEAEEAMKRLSYISQKVSDASDKTQQAERALG |
| | SAAADAQRAKNGAGEALEISSEIEQEIGSLNLEANVTADGALAMEKGL |
| | ASLKSEMREVEGELERKELEFDTNMDAVQMVITEAQKVDTRAKNAG |
| | VTIQDTLNTLDGLLHLMDQPLSVDEEGLVLLEQKLSRAKTQINSQLRP |
| | MMSELEERARQQRGHLHLLETSIDGILADVKNLENIRDNLPPGCYNTQ |
| | ALEQQ |
| laminin | MPALWLGCCLCFSLLLPAARATSRREVCDCNGKSRQCIFDRELHRQTG |
| subunit | NGFRCLNCNDNTDGIHCEKCKNGFYRHRERDRCLPCNCNSKGSLSAR |
| gamma-2 | CDNSGRCSCKPGVTGARCDRCLPGFHMLTDAGCTQDQRLLDSKCDCD |
| isoform b | PAGIAGPCDAGRCVCKPAVTGERCDRCRSGYYNLDGGNPEGCTQCFC |
| precursor | YGHSASCRSSAEYSVHKITSTFHQDVDGWKAVQRNGSPAKLQWSQRH |
| (LAMC2- | QDVFSSAQRLDPVYFVAPAKFLGNQQVSYGQSLSFDYRVDRGGRHPS |
| isoform b); | AHDVILEGAGLRITAPLMPLGKTLPCGLTKTYTFRLNEHPSNNWSPQLS |
| SEQ ID | YFEYRRLLRNLTALRIRATYGEYSTGYIDNVTLISARPVSGAPAPWVEQ |
| NO: 45 | CICPVGYKGQFCQDCASGYKRDSARLGPFGTCIPCNCQGGGACDPDTG |
| | DCYSGDENPDIECADCP1GFYNDPHDPRSCKPCPCHNGFSCSVMPETEE |
| | VVCNNCPPGVTGARCELCADGYFGDPFGEHGPVRPCQPCQCNNNVDP |
| | SASGNCDRLTGRCLKCIHNTAGIYCDQCKAGYFGDPLAPNPADKCRA |
| | CNCNPMGSEPVGCRSDGTCVCKPGFGGPNCEHGAFSCPACYNQVKIQ |
| | MDQFMQQLQRMEALISKAQGGDGVVPDTELEGRMQQAEQALQDILR |
| | DAQISEGASRSLGLQLAKVRSQENSYQSRLDDLKMTVERVRALGSQY |
| | QNRVRDTHRLITQMQLSLAESEASLGNTNIPASDHYVGPNGFKSLAQE |
| | ATRLAESHVESASNMEQLTRETEDYSKQALSLVRKALHEGVGSGSGSP |
| | DGAVVQGLVEKLEKTKSLAQQLTREATQAEIEADRSYQHSLRLLDSVS |
| | RLQGVSDQSFQVEEAKRIKQKADSLSSLVTRHMDEFKRTQKNLGNWK |
| | EEAQQLLQNGKSGREKSDQLLSRANLAKSRAQEALSMGNATFYEVESI |
| | LKNLREFDLQVDNRKAEAEEAMKRLSYISQKVSDASDKTQQAERALG |
| | SAAADAQRAKNGAGEALEISSEIEQEIGSLNLEANVTADGALAMEKGL |
| | ASLKSEMREVEGELERKELEFDTNMDAVQMVITEAQKVDTRAKNAG |
| | VTIQDTLNTLDGLLHLMGM |
| laminin | MAAAALLLGLALLAPRAAGAGMGACYDGAGRPQRCLPVFENAAFGR |
| subunit | LAQASHTCGSPPEDFCPHVGAAGAGAHCQRCDAADPQRHEINASYLT |
| gamma-3 | DFHSQDESTWWQSPSMAFGVQYPTSVNITLRLGKAYEITYVRLKFHTS |
| precursor | RPESFAIYKRSRADGPWEPYQFYSASCQKTYGRPEGQYLRPGEDERVA |
| (LAMC3); | FCTSEFSDISPLSGGNVAFSTLEGRPSAYNFEESPGLQEWVTSTELLISL |
| SEQ ID | DRLNTFGDDIFKDPKVLQSYYYAVSDFSVGGRCKCNGHASECGPDVA |
| NO: 46 | GQLACRCQHNTTGTDCERCLPFFQDRPWARGTAEAAHECLPCNCSGR |
| | SEECTFDRELFRSTGHGGRCHHCRDHTAGPHCERCQENFYHWDPRMP |
| | CQPCDCQSAGSLHLQCDDTGTCACKPTVTGWKCDRCLPGFHSLSEGG |
| | CRPCTCNPAGSLDTCDPRSGRCPCKENVEGNLCDRCRPGTFNLQPHNP |
| | AGCSSCFCYGHSKVCASTAQFQVHHILSDFHQGAEGWWARSVGGSEH |
| | PPQWSPNGVLLSPEDEEELTAPEKFLGDQRFSYGQPLILTFRVPPGDSPL |
| | PVQLRLEGTGLALSLRHSSLSGPQDAGHPREVELRFHLQETSEDVAPPL |
| | PPFHFQRLLANLTSLRLRVSPGPSPAGPVFLTEVRLTSARPGLSPPASW |
| | VEICSCPTGYTGQFCESCAPGYKREMPQGGPYASCVPCTCNQHGTCDP |
| | NTGICVCSHHTEGPSCERCLPGFYGNPFAGQADDCQPCPCPGQSACTTI |
| | PESREVVCTHCPPGQRGRRCEVCDDGFFGDPLGLFGHPQPCHQCQCSG |
| | NVDPNAVGNCDPLSGHCLRCLHNTTGDHCEHCQEGFYGSALAPRPAD |
| | KCMPCSCHPQGSVSEQMPCDPVTGQCSCLPHVTARDCSRCYPGFFDL |
| | QPGRGCRSCKCHPLGSQEDQCHPKTGQCTCRPGVTGQACDRCQLGFF |
| | GFSIKGCRACRCSPLGAASAQCHENGTCVCRPGFEGYKCDRCHDNFFL |
| | TADGTHCQQCPSCYALVKEEAAKLKARLTLTEGWLQGSDCGSPWGPL |
| | DILLGEAPRGDVYQGHEILLPGAREAFLEQMMSLEGAVKAAREQLQRL |
| | NKGARCAQAGSQKTCTQLADLEAVLESSEEEILHAAAILASLEIPQEGP |
| | SQPTKWSHLATEARALARSHRDTATKIAATAWRALLASNTSYALLWN |
| | LLEGRVALETQRDLEDRYQEVQAAQKALRTAVAEVLPEAESVLATVQ |
| | QVGADTAPYLALLASPGALPQKSRAEDLGLKAKALEKTVASWQHMA |
| | TEAARTLQTAAQATLRQTEPLTKLHQEARAALTQASSSVQAATVTVM |
| | GARTLLADLEGMKLQFPRPKDQAALQRKADSVSDRLLADTRKKTKQ |
| | AERMLGNAAPLSSSAKKKGREAEVLAKDSAKLAKALLRERKQAHRR |
| | ASRLTSQTQATLQQASQQVLASEARRQELEEAERVGAGLSEMEQQIRE |
| | SRISLEKDIETLSELLARLGSLDTHQAPAQALNETQWALERLRLQLGSP |
| | GSLQRKLSLLEQESQQQELQIQGFESDLAEIRADKQNLEAILHSLPENC |
| | ASWQ |

Further exemplary peptides useful in the methods and compositions of the disclosure include:

| SEQ | Name | |
|---|---|---|
| ID | (location) | Peptide |
| NO: | length | sequence |
| 1 | α3₃₀₄₃₋₃₀₆₇ | RLVFALGTDGKKLRIKSKEKCNDGK |
| | (LG4) | |
| | 25 aa. | |
| 9 | α3₃₀₃₁₋₃₀₄₃ | KNSFMALYLSKGR |
| | (LG4) | |
| | 13 aa. | |
| 2 | α3₂₉₃₂₋₂₉₅₁ | PPFLMLLKGSTRFNKTKTFR |
| | (Linker) | |
| | 20 aa. | |
| 3 | α4₁₅₂₁₋₁₅₄₃ | TLFLAHGRLVYMFNVGHKKLKIR |
| | (LG4) | |
| | 23 aa. | |
| 4 | α4₁₄₀₈₋₁₄₃₄ | PLFLLHKKGKNLSKPKASQNKKGGKSK |
| | (Linker) | |
| | 27 aa. | |
| 5 | α5₃₅₃₉₋₃₅₅₀ | TLPDVGLELEVR |
| | (LG5) | |
| | 12 aa. | |
| 6 | α5₃₄₁₇₋₃₄₃₆ | RQRSRPGRWHKVSVRWEKNR |
| | (LG4) | |
| | 20 aa. | |
| 10 | α5₃₃₁₂₋₃₃₂₅ | ARKASRRSRQPARH |
| | (Linker) | |
| | 14 aa. | |
| 7 | α5₃₃₀₀₋₃₃₃₀ | TPGLGPRGLQATARKASRRSRQPARHPACML |
| | (Linker) | |
| | 31 aa. | |
| 8 | α2PI₁₋₈- | NQEQVSPLRLVFALGTDGKKLRIKSKEKCNDGK |
| | α3₃₀₄₃₋₃₀₆₇ | |
| | 33 aa. | |
| 11 | α2PI₁₋₈- | NQEQVSPLARKASRRSRQPARH |
| | α5₃₃₁₂₋₃₃₂₅ | |
| | 22 aa. | |
| 12 | α2PI₁₋₈ | NQEQVSPL |
| 49 | vWF A1 | YIGLKDRKRPSELRRIASQVKYAC |

In some embodiments, the compositions and methods comprise a peptide from a LG4 domain or fragment thereof. Exemplary LG4 domains are shown below:

| SEQ | | |
|---|---|---|
| ID | | |
| NO: | Name | Sequence |
| 13 | LAMA3_Human, | ALQFGDIPTSHLLFKLPQELLKPRSQFAV |
| | LG4 domain | DMQTTSSRGLVFHTGTKNSFMALYLSKGR |
| | aa2986- | LVFALGTDGKKLRIKSKEKCNDGKWHTVV |
| | aa3150 | FGHDGEKGRLVVDGLRAREGSLPGNSTIS |
| | (UniprotKB | IRAPVYLGSPPSGKPKSLPTNSFVGCLKN |
| | database | FQLDSKPLYTPSSSFGVSSC |
| | Q16787) | |
| 14 | LAMA4_Human, | AYQYGGTANSRQEFEHLKGDFGAKSQFSI |
| | LG4 domain | RLRTRSSHGMIFYVSDQEENDFMTLFLAH |
| | aa1469- | GRLVYMFNVGHKKLKIRSQEKYNDGLWHD |
| | aa1640 | VIFIRERSSGRLVIDGLRVLEESLPPTEA |
| | (UniprotKB | TWKIKGPIYLGGVAPGKAVKNVQINSIYS |
| | database | FSGCLSNLQLNGASITSASQTFSVTPC |
| | Q16363) | |
| 15 | LAMA5_Human, | SYQFGGSLSSHLEFVGILARHRNWPSLSM |
| | LG4 domain | HVLPRSSRGLLLFTARLRPGSPSLALFLS |
| | aa3340- | NGHFVAQMEGLGTRLRAQSRQRSRPGRWH |
| | aa3513 | KVSVRWEKNRILLVTDGARAWSQEGPHRQ |
| | (UniprotKB | HQGAEHPQPHTLFVGGLPASSHSSKLPVT |
| | database | VGFSGCVKRLRLHGRPLGAPTRMAGVTPC |
| | O15230) | |

In some embodiments, the compositions and methods include an engineered Laminin peptide comprising a factor XIIIa transglutaminase substrate domain from the α₂-plasmin inhibitor. Such exemplary peptides are described below:

| SEQ | | |
|---|---|---|
| ID | | |
| NO. | Name | Sequence |
| 16 | Human α2PI₁₋₈- | NQEQVSPLGGSGALQFGDIPTSHLLFKLP |
| | LAMA3_LG4₂₉₈₆₋₃₁₅₀ | QELLKPRSQFAVDMQTTSSRGLVFHTGTK |
| | | NSFMALYLSKGRLVFALGTDGKKLRIKSK |
| | | EKCNDGKWHTVVFGHDGEKGRLVVDGLRA |
| | | REGSLPGNSTISIRAPVYLGSPPSGKPKS |
| | | LPTNSFVGCLKNFQLDSKPLYTPSSSFGV |
| | | SSC |
| 17 | Human α2PI₁₋₈- | NQEQVSPLGGSGAYQYGGTANSRQEFEHL |
| | LAMA4_LG4₁₄₆₉₋₁₆₄₀ | KGDFGAKSQFSIRLRTRSSHGMIFYVSDQ |
| | | EENDFMTLFLAHGRLVYMFNVGHKKLKIR |
| | | SQEKYNDGLWHDVIFIRERSSGRLVIDGL |
| | | RVLEESLPPTEATWKIKGPIYLGGVAPGK |
| | | AVKNVQINSIYSFSGCLSNLQLNGASITS |
| | | ASQTFSVTPC |
| 18 | Human α2PI₁₋₈- | NQEQVSPLGGSGSYQFGGSLSSHLEFVGI |
| | LAMA5_LG4₃₃₄₀₋₃₅₁₃ | LARHRNWPSLSMHVLPRSSRGLLLFTARL |
| | | RPGSPSLALFLSNGHFVAQMEGLGTRLRA |
| | | QSRQRSRPGRWHKVSVRWEKNRILLVTDG |
| | | ARAWSQEGPHRQHQGAEHPQPHTLFVGGL |
| | | PASSHSSKLPVTVGFSGCVKRLRLHGRPL |
| | | GAPTRMAGVTPC |

In some embodiments, the compositions and methods comprise peptides comprising a collagen binding peptide. Exemplary collagen binding peptides are shown below.

| SEQ | | |
|---|---|---|
| ID | | |
| NO. | Name | Sequence |
| 47 | vWF A3 | CSQPLDVILLLDGSSSFPASYFDEMKSFAKAFISKA |
| | domain | NIGPRLTQVSVLQYGSITTIDVPWNVVPEKAHLLS |
| | | LVDVMQREGGPSQIGDALGFAVRYLTSEMHGAR |
| | | PGASKAVVILVTDVSVDSVDAAADAARSNRVTV |
| | | FPIGIGDRYDAAQLRILAGPAGDSNVVKLQRIEDL |
| | | PTMVTLGNSFLHKLCSGFVRICTG |
| 48 | Decorin | CGPFQQRGLFDFMLEDEASGIGPEVPDDRDFEPSL |
| | | GPVCPFRCQCHLRVVQCSDLGLDKVPKDLPPDTT |
| | | LLDLQNNKITEIKDGDFKNLKNLHALILVNNKISK |
| | | VSPGAFTPLVKLERLYLSKNQLKELPEKMPKTLQ |
| | | ELRAHENEITKVRKVTFNGLNQMIVIELGTNPLKS |
| | | SGIENGAFQGMKKLSYIRIADTNITSIPQGLPPSL |
| | | TELHLDGNKISRVDAASLKGLNNLAKLGLSFNSIS |
| | | AVDNGSLANTPHLRELHLDNNKLTRVPGGLAEHKY |
| | | IQVVYLHNNNISVVGSSDFCPPGHNTKKASYSGVS |
| | | LFSNPVQYWEIQPSTFRCVYVRSAIQLGNYK |

The growth factor-binding peptide may be a peptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a peptide of the disclosure, such as peptides, proteins, or polypeptides defined by any one of SEQ ID NOS:1-50. The peptide or polypeptide may have one or more conservative or non-conservative substitutions. Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Embodiments of the disclosure include a peptide/polypeptide that is at least, at most, or exactly 75, 76, 77, 78, 79, 80, 81, 82, 83, 84, 85, 86, 87, 88, 89, 90, 91, 92, 93, 94, 95, 96, 97, 98, 99, or 100% identical (or any derivable range therein) to a peptide or polypeptide/polypeptide that has at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein to a peptide/polypeptide that starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, or 450 of any one of SEQ ID NOS:1-50.

The polypeptides or peptides described herein may include 1, 2, 3, 4, 5, 6, 7, 8, 9, 10, 11, 12, 13, 14, 15, or more (or any derivable range therein) variant amino acids within at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of a peptide or polypeptide of the disclosure, such as peptides, proteins, or polypeptides defined by any one of SEQ ID NOS:1-50.

A polypeptide segment as described herein may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein of a peptide or polypeptide of the disclosure, such as peptides, proteins, or polypeptides defined by any one of SEQ ID NOS:1-50.

The polypeptides or peptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein) or a peptide or polypeptide of the disclosure, such as peptides, proteins, or polypeptides defined by any one of SEQ ID NOS:1-50.

A linker sequence may be included in the peptide construction. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate a peptide of the disclosure, such as peptides, proteins, or polypeptides defined by any one of SEQ ID NOS:1-50, to an attached moiety, such as a transglutaminase-reactive peptide, a collagen binding peptide, cell adhesion moiety, tag, or functional moiety. In some embodiments, the linker comprises a glycine serine linker. In some embodiments, the linker comprises (GSGG)ₓ (SEQ ID NO:60), wherein x=1-6. In some embodiments, x=2. In some embodiments, x=1, 2, 3, 4, 5, or 6 (or any derivable range therein) In some embodiments, the linker comprises GSGGGSGG (SEQ ID NO:61).

### B. Exemplary Attachments to the Growth Factor Binding Peptides/Polypeptides

Embodiments include a growth factor binding peptide attached to moieties such as a functional moiety. In some embodiments, the functional moiety may be a therapeutic agent, marker, cell adhesion molecule, antigen, protein, protein drug, or cytokine. In some embodiments, the growth factor binding peptide is attached to a second growth factor binding peptide. In some embodiments, the growth factor binding peptide is attached to a chemical moiety, such as a marker or fluorescent marker. The fusion comprises the peptides conjugated directly or indirectly to each other. The peptides may be directly conjugated to each other or indirectly through a linker. The linker may be a peptide, a polymer, an aptamer, a nucleic acid, or a particle. The particle may be, e.g., a microparticle, a nanoparticle, a polymersome, a liposome, or a micelle. The polymer may be, e.g., natural, synthetic, linear, or branched. A fusion protein that comprises the first peptide and the second peptide is an example of a molecular fusion of the peptides, with the fusion protein comprising the peptides directly joined to each other or with intervening linker sequences and/or further sequences at one or both ends. The conjugation to the linker may be through covalent bonds. Methods include preparing a molecular fusion or a composition comprising the molecular fusion, including such a composition in a pharmaceutically acceptable form.

Embodiments include a molecular fusion of a polypeptide that comprises a growth factor binding peptide and a transglutaminase (TG)-reactive peptide. An embodiment of a TG-reactive peptide is a peptide that comprises residues 1-8 of alpha 2-plasmin inhibitor (NQEQVSPL) (SEQ ID NO:12). In some embodiments, the TG-reactive peptide is at the amino terminus of the growth factor binding peptide. In some embodiments, the TG-reactive peptide is at the carboxy terminus of the growth factor binding peptide. Embodiments include such a polypeptide being a recombinant fusion polypeptide. The molecular fusion may be further comprising a cell adhesion moiety having a specific binding affinity for a cell adhesion molecule. Various cell adhesion moieties are known, for instance, wherein the cell adhesion moiety comprises a ligand for a glycoprotein or a cell surface receptor. Or the cell adhesion moiety may comprise a ligand with specific binding to the cell adhesion molecule and the cell adhesion molecule is a cell surface receptor chosen from the group consisting of an integrin, and a cadherin. Or the cell adhesion moiety may comprise an integrin-binding peptide such as Tenascin III3, an RGD sequence.

In some aspects, the peptide or polypeptide of the disclosure is attached to a tag. The tag may be a purification tag, a signaling sequence, a detectable marker, a post-translational modifier, or a targeting moiety. In some embodiments, the peptide or polypeptide is attached to a functional moiety such as an enzyme, a fluorescent compound, or a therapeutic agent. Detectable markers include, for example, a radioactive atom, a chromophore, a fluorophore, or the like. Other examples of tags or functional moieties include enzymes, radioisotopes, fluorochromes, chemiluminescent compounds, dyes, and proteins. Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases. Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.). In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent. Attachment of a tag or functional moiety may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

In some embodiments, the functional moiety comprises an imaging agent. Exemplary imaging agents include gadolinium, iodine, barium, or a radio pharmaceutical such as calcium-47, carbon-11, carbon-14, chromium-51, cobalt-57, cobalt-58, erbium-169, fluorine-18, gallium-67, gallium-68, hydrogen-3, indium-111, iodine-123, iodine-125, iodine-131, iron-59, krypton-81m, nitrogen-13, oxygen-15, phosphorus-32, radium-223, rubidium-82, samarium-153, selenium-75, sodium-22, sodium-24, strontium-89, technetium-99m, thallium-201, xenon-133, and yttrium-90.

The term molecular fusion, or the term conjugated, refers to direct or indirect association by chemical bonds, including covalent, electrostatic ionic, or charge-charge. In some embodiments, the conjugation is through a peptide bond. The conjugation creates a unit that is sustained by chemical bonding. Direct conjugation refers to chemical bonding to the agent, with or without intermediate linkers or chemical groups. Indirect conjugation refers to chemical linkage to a carrier. The carrier may largely encapsulate the agent, e.g., a polymersome, a liposome or micelle or some types of nanoparticles, or have the agent on its surface, e.g., a metallic nanoparticle or bead, or both, e.g., a particle that includes some of the agent in its interior as well as on its exterior. The carrier may also encapsulate an antigen for immunotolerance. For instance a polymersome, liposome, or a particle may be made that encapsulates the antigen. The term encapsulate means to cover entirely, effectively without any portion being exposed, for instance, a polymersome may be made that encapsulates an antigen or an agent.

Conjugation may be accomplished by covalent bonding of the peptide to another molecule, with or without use of a linker. The formation of such conjugates is within the skill of artisans and various techniques are known for accomplishing the conjugation, with the choice of the particular technique being guided by the materials to be conjugated. The addition of amino acids to the polypeptide (C- or N-terminal) which contain ionizable side chains, i.e. aspartic acid, glutamic acid, lysine, arginine, cysteine, histidine, or tyrosine, and are not contained in the active portion of the polypeptide sequence, serve in their unprotonated state as a potent nucleophile to engage in various bioconjugation reactions with reactive groups attached to polymers, i.e. homo- or hetero-bi-functional PEG (e.g., Lutolf and Hubbell, Biomacromolecules 2003; 4:713-22, Hermanson, Bioconjugate Techniques, London. Academic Press Ltd; 1996). In some embodiments, a soluble polymer linker is used, and may be administered to a patient in a pharmaceutically acceptable form. Or a drug may be encapsulated in polymerosomes or vesicles or covalently attached to the peptide ligand.

The molecular fusion may comprise a particle. The growth factor binding peptide may be attached to the particle. An antigen, agent, or other substance may be in or on the particle. Examples of nanoparticles, micelles, and other particles are found at, e.g., US 2008/0031899, US 2010/0055189, US 2010/0003338, which applications are hereby incorporated by reference herein for all purposes, including combining the same with a ligand as set forth herein; in the case of conflict, however, the instant specification controls.

Nanoparticles may be prepared as collections of particles having an average diameter of between about 10 nm and about 200 nm, including all ranges and values between the explicitly articulated bounds, e.g., from about 20 to about 200, and from about 20 to about 40, to about 70, or to about 100 nm, depending on the polydispersity which is yielded by the preparative method. Various nanoparticle systems can be utilized, such as those formed from copolymers of poly(ethylene glycol) and poly(lactic acid), those formed from copolymers of poly(ethylene oxide) and poly(beta-amino ester), and those formed from proteins such as serum albumin. Other nanoparticle systems are known to those skilled in these arts. See also Devalapally et al., Cancer Chemother Pharmacol., Jul. 25, 2006; Langer et al., International Journal of Pharmaceutics, 257:169-180 (2003); and Tobio et al., Pharmaceutical Research, 15(2):270-275 (1998).

Larger particles of more than about 200 nm average diameter incorporating the growth factor binding peptides may also be prepared, with these particles being termed microparticles herein since they begin to approach the micron scale and fall approximately within the limit of optical resolution. For instance, certain techniques for making microparticles are set forth in U.S. Pat. Nos. 5,227,165, 6,022,564, 6,090,925, and 6,224,794.

Functionalization of nanoparticles to employ targeting capability requires association of the targeting polypeptide with the particle, e.g., by covalent binding using a bioconjugation technique, with choice of a particular technique being guided by the particle or nanoparticle, or other construct, that the polypeptide is to be joined to. In general, many bioconjugation techniques for attaching peptides to other materials are well known and the most suitable technique may be chosen for a particular material. For instance, additional amino acids may be attached to the polypeptide sequences, such as a cysteine in the case of attaching the polypeptide to thiol-reactive molecules.

The molecular fusion may comprise a polymer. The polymer may be branched or linear.

The molecular fusion may comprise a dendrimer. In general, soluble hydrophilic biocompatible polymers may be used so that the conjugate is soluble and is bioavailable after introduction into the patient. Examples of soluble polymers are polyvinyl alcohols, polyethylene imines, and polyethylene glycols (a term including polyethylene oxides) having a molecular weight of at least 100, 400, or between 100 and 400,000 (with all ranges and values between these explicit values being contemplated). Solubility in this context refers to a solubility in water or physiological saline of at least 1 gram per liter. Domains of biodegradable polymers may also be used, e.g., polylactic acid, polyglycolic acid, copolymers of polylactic and polyglycolic acid, polycaprolactones, polyhydroxy butyric acid, polyorthoesters, polyacetals, polydihydropyrans, and polycyanoacylates.

### II. NUCLEIC ACIDS

In certain embodiments, the current disclosure concerns recombinant polynucleotides encoding the proteins, polypeptides, and peptides of the disclosure.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular embodiments, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other embodiments, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. In some embodiments, the vector comprises pSeqTag-A or pcDNA3.1.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Various elements/promoters may be employed in the context of the present invention to regulate the expression of a gene. Examples of such inducible elements, which are regions of a nucleic acid sequence that can be activated in response to a specific stimulus, include but are not limited to Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990), Immunoglobulin Light Chain (Queen et al., 1983; Picard et al., 1984), T Cell Receptor (Luria et al., 1987; Winoto et al., 1989; Redondo et al.; 1990), HLA DQ α and/or DQ β (Sullivan et al., 1987), β Interferon (Goodbourn et al., 1986; Fujita et al., 1987; Goodbourn et al., 1988), Interleukin-2 (Greene et al., 1989), Interleukin-2 Receptor (Greene et al., 1989; Lin et al., 1990), MHC Class II 5 (Koch et al., 1989), MHC Class II HLA-DRα (Sherman et al., 1989), β-Actin (Kawamoto et al., 1988; Ng et al.; 1989), Muscle Creatine Kinase (MCK) (Jaynes et al., 1988; Horlick et al., 1989; Johnson et al., 1989), Prealbumin (Transthyretin) (Costa et al., 1988), Elastase I (Ornitz et al., 1987), Metallothionein (MTII) (Karin et al., 1987; Culotta et al., 1989), Collagenase (Pinkert et al., 1987; Angel et al., 1987), Albumin (Pinkert et al., 1987; Tronche et al., 1989, 1990), α-Fetoprotein (Godbout et al., 1988; Campere et al., 1989), γ-Globin (Bodine et al., 1987; Perez-Stable et al., 1990), β-Globin (Trudel et al., 1987), c-fos (Cohen et al., 1987), c-Ha-Ras (Triesman, 1986; Deschamps et al., 1985), Insulin (Edlund et al., 1985), Neural Cell Adhesion Molecule (NCAM) (Hirsh et al., 1990), α1-Antitrypain (Latimer et al., 1990), H2B (TH2B) Histone (Hwang et al., 1990), Mouse and/or Type I Collagen (Ripe et al., 1989), Glucose-Regulated Proteins (GRP94 and GRP78) (Chang et al., 1989), Rat Growth Hormone (Larsen et al., 1986), Human Serum Amyloid A (SAA) (Edbrooke et al., 1989), Troponin I (TN I) (Yutzey et al., 1989), Platelet-Derived Growth Factor (PDGF) (Pech et al., 1989), Duchenne Muscular Dystrophy (Klamut et al., 1990), SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988), Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell et al., 1988), Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989), Papilloma Virus (Campo et al., 1983; Lusky et al., 1983; Spandidos and Wilkie, 1983; Spalholz et al., 1985; Lusky et al., 1986; Cripe et al., 1987; Gloss et al., 1987; Hirochika et al., 1987; Stephens et al., 1987), Hepatitis B Virus (Bulla et al., 1986; Jameel et al., 1986; Shaul et al., 1987; Spandau et al., 1988; Vannice et al., 1988), Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989), Cytomegalovirus (CMV) IE (Weber et al., 1984; Boshart et al., 1985; Foecking et al., 1986), Gibbon Ape Leukemia Virus (Holbrook et al., 1987; Quinn et al., 1989).

Inducible elements include, but are not limited to MT II—Phorbol Ester (TFA)/Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)—Glucocorticoids (Huang et al., 1981; Lee et al., 1981; Majors et al., 1983; Chandler et al., 1983; Lee et al., 1984; Ponta et al., 1985; Sakai et al., 1988); β-Interferon—poly(rI)x/poly(rc) (Tavernier et al., 1983); Adenovirus 5 E2—ElA (Imperiale et al., 1984); Collagenase—Phorbol Ester (TPA) (Angel et al., 1987a); Stromelysin Phorbol Ester (TPA) (Angel et al., 1987b); SV40—Phorbol Ester (TPA) (Angel et al., 1987b); Murine MX Gene—Interferon, Newcastle Disease Virus (Hug et al., 1988); GRP78 Gene—A23187 (Resendez et al., 1988); α-2-Macroglobulin—IL-6 (Kunz et al., 1989); Vimentin—Serum (Rittling et al., 1989); MHC Class I Gene H-2κb—Interferon (Blanar et al., 1989); HSP70—ElA/SV40 Large T Antigen (Taylor et al., 1989, 1990a, 1990b); Proliferin—Phorbol Ester/TPA (Mordacq et al., 1989); Tumor Necrosis Factor—PMA (Hensel et al., 1989); and Thyroid Stimulating Hormone a Gene—Thyroid Hormone (Chatterjee et al., 1989).

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain embodiments of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. As an alternative, 2A peptides could be used to introduce ribosomal skips to enable expression of multiple polypeptidic or protein sequences.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### III. COMPOSITIONS

In some embodiments, pharmaceutical compositions are administered to a subject. Different aspects involve administering an effective amount of a composition to a subject. In some embodiments, a composition comprising a peptide of the disclosure may be administered to the subject or patient to treat wounds or facilitate wound, tissue, or bone repair. Additionally, such compositions can be administered in combination with an additional therapy.

### A. Carriers and Excipients

Pharmaceutically acceptable carriers or excipients may be used to deliver embodiments as described herein. Excipient refers to an inert substance used as a diluent or vehicle for a therapeutic agent. Pharmaceutically acceptable carriers are used, in general, with a compound (eg. peptide of the disclosure) so as to make the compound useful for a therapy or as a product. In general, for any substance, a carrier is a material that is combined with the substance for delivery to an animal. Conventional pharmaceutical carriers, aqueous, powder or oily bases, thickeners and the like may be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable salts of the compounds described herein may be synthesized according to methods known to those skilled in the arts. Thus a pharmaceutically acceptable compositions are highly purified to be free of contaminants, are sterile, biocompatible and not toxic, and further may include a carrier, salt, or excipient suited to administration to a patient. In the case of water as the carrier, the water is highly purified and processed to be free of contaminants, e.g., endotoxins.

The compounds described herein may be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. Thus the deliverable compound may be made in a form suitable for oral, rectal, topical, intravenous injection, intra-articular injection, intradermal, intramuscular, and/or parenteral administration. Carriers include solids or liquids, and the type of carrier is chosen based on the type of administration being used. Suitable binders, lubricants, disintegrating agents, coloring agents, flavoring agents, flow-inducing agents, and melting agents may be included as carriers, e.g., for pills. For instance, an active component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. The compounds can be administered orally in solid dosage forms, such as capsules, tablets, and powders, or in liquid dosage forms, such as elixirs, syrups, and suspensions. The active compounds can also be administered parentally, in sterile liquid dosage forms. Buffers for achieving a physiological pH or osmolarity may also be used.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

### B. Dosage

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein of an agent of the disclosure (e.g. growth factor, cytokine, peptide, polypeptide, functional moiety, etc. . . . ).

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### C. Growth Factors and Cytokines

Certain embodiments of the disclosure relate to compositions, molecular complexes, biomaterials, and implants comprising growth factors and cytokines. Exemplary non-limiting growth factors and cytokines include mammalian proteins such as ANG-1, ANG-2, EGF, EPO, NGF, FGF-2, FGF-4, FGF-6, FGF-7, FGF-10, FGF-17, FGF-18, TGF-α, TGF-β, TGF-β1, TGF-β2, TGF-β3, NGF, NT-3, BDNF, PlGF-1, PlGF-2, PlGF-3, BMP-2, BMP-7, BMP-9 PDGF-AA, PDGF-AB, PDGF-BB, PDGF-DD, VEGF-A165, VEGF-A121, VEGF-B, VEGF-C, VEGF-D, IGF-1, IGF-BP3, IGF-BP5, HGF, EGF, HB-EGF, CXCL12, or CXCL11. In some embodiments, the growth factor or cytokine is a mammalian growth factor or cytokine. In some embodiments, the growth factor or cytokine is a human, mouse, pig, monkey, horse, goat, rabbit, sheep or rat growth factor or cytokine. In some embodiments, one or more of ANG-1, ANG-2, EGF, EPO, NGF, FGF-2, FGF-4, FGF-6, FGF-7, FGF-10, FGF-17, FGF-18, TGF-α, TGF-β, TGF-β1, TGF-β2, NT-3, BDNF, PlGF-1, PlGF-2, PlGF-3, BMP-2, BMP-7, PDGF-AA, PDGF-AB, PDGF-BB, PDGF-DD, VEGF-A165, VEGF-A121, VEGF-B, VEGF-C, IGF-1, IGF-BP3, IGF-BP5, or HGF are specifically excluded from the compositions, molecular complexes, scaffolds, implants, or matrices described herein. In some embodiments, at least, at most, or exactly, 1, 2, 3, 4, 5, 6, 7, 8, 9, 10, 15, 20, 25, or 30 (or any derivable range therein) of ANG-1, ANG-2, EGF, EPO, NGF, FGF-2, FGF-4, FGF-6, FGF-7, FGF-10, FGF-17, FGF-18, TGF-α, TGF-β, TGF-β1, TGF-β2, NT-3, BDNF, PlGF-1, PlGF-2, PlGF-3, BMP-2, BMP-7, PDGF-AA, PDGF-AB, PDGF-BB, PDGF-DD, VEGF-A165, VEGF-A121, VEGF-B, VEGF-C, IGF-1, IGF-BP3, IGF-BP5, or HGF is included in the embodiments of the disclosure

### IV. BIOMATERIAL SCAFFOLD AND IMPLANTS

Certain embodiments of the disclosure relate to biomaterial scaffolds or matrix comprising the peptide or polypeptides of the disclosure. The term matrix refers to a three-dimensional structure, including a block, gel, sheet, or film; it is a term used in contrast to a soluble or fluid material. The scaffolds have to withstand mechanical loads, contain suitable degradation kinetics, and present bioactive molecules. Scaffolds function as a fusion of cell carrier and drug delivery device for the purpose of tissue engineering. To mimic the natural microenvironment for cells in order to induce tissue repair and regeneration, synthetic materials can be modified with ECM fragments. ECM fragments described herein may be designed to form a molecular fusion with a transglutaminase (TG) peptide at the N or C terminus. In some embodiments, the TG-reactive peptide consists of residues 1-8 of the protein alpha2 plasmin inhibitor (α₂PI₁₋₈, NQEQVSPL (SEQ ID NO:12)). Factor XIIIa can therefore be used as a transglutaminase to catalyze the reaction between the glutamines of this sequence (NQEQVSPL) and the lysines of different biomaterials. The coagulation enzyme, factor XIIIa, will covalently bind the free amine group of the lysines (Lys) to the gamma-carboxamide group of glutamine (Gln), resulting in bonds that exhibit high resistance to proteolytic degradation. For example, natural fibrin hydrogels are cross-linked by this mechanism and TG-TNC III1-5 can therefore be cross-linked inside the gel (Schense and Hubbell, 1999).

Modification of synthetic hydrogels is possible by engineering counter-substrates for transglutaminases, such as lysines inside poly ethylene glycol (PEG-Lys) hydrogels. PEG is modified with lysines by chemically cross-linking a lysine containing peptide that includes a cysteine to form a disulfide-bridged polymer conjugate with PEG-vinyl sulfone (PEG-VS). The SH group of the cysteine functions as nucleophile (Mikael donor) in a Mikael type addition, with VS functioning as Michael acceptor (Lutolf, Lauer-Fields, et al., 2003). This technology has been used to make TG-PEG gels, which are cross-linked by two multi-arm PEG-peptide conjugates, PEG-Lys and PEG-Gln, in the presence of factor XIII, which allows for incorporation of other proteins containing a TG substrate (Ehrbar, Rizzi, et al., 2007). Alternatively, chemical crosslinking through cysteine residues may be used to attach proteins, peptides, and polypeptides to polymeric compositions and gels.

The peptide, polypeptides, compositions, and molecular complexes of the disclosure can be further immobilized into biomaterial matrices, forming additional embodiments. The peptides and polypeptides can be fused to a transglutaminase substrate that can covalently bind to natural protein biomaterials such as fibrin or to synthetic biomaterials engineered to comprise counter-substrates for transglutaminases.

Biomaterial scaffolds useful in the embodiments of the disclosure may comprise ceramics, synthetic polymers, and/or natural polymers. Ceramic scaffolds include, for example, hydroxyapatite (HA) and tri-calcium phosphate (TCP). Ceramic scaffolds are typically characterized by high mechanical stiffness (Young's modulus), very low elasticity, and a hard brittle surface. Examples of synthetic polymers include polystyrene, poly-l-lactic acid (PLLA), polyglycolic acid (PGA) and poly-dl-lactic-co-glycolic acid (PLGA). Exemplary natural polymers include collagen, proteoglycans, alginate-based substrates, and chitosan. Natural polymers are biologically active and typically promote excellent cell adhesion and growth. Furthermore, they are also biodegradable and so allow host cells, over time, to produce their own extracellular matrix and replace the degraded scaffold. In some embodiments, the biomaterial scaffold may comprise different components such as ceramics and natural or synthetic polymers.

According to a further aspect of the present invention, the biomaterial scaffold or implant comprises synthetic cartilage, bone, ligament, tendon, meniscus, periodontal tissue, dentine, enamel, intervertebral disc, annulus fibrosus, or nucleus pulposus implant, graft, substitute, scaffold, filler, coating or cement.

The biomaterial or implants may further comprise cells. The cells may be stem or progenitor cells, differentiated cells, terminally differentiated cells, or combinations thereof. The cells may be totipotent, pluripotent or unipotent stem cells, or induced pluripotent stem cells. The cells may be human embryonic stem cells, derived via a technology which does not necessitate the destruction of the human embryo, for example via an established cell line. Mesenchymal stem cells (also referred to as marrow stromal cells, multipotent stromal cells, or MSCs) are pluripotent stem cells which can differentiate into a variety of cell types including osteoblasts, tenocytes, chondrocytes, myocytes, adipocytes. These cell types have the ability to generate bone, tendon, ligament, cartilage, muscle, and fat. The cells may be MSCs or any cell within the MSC lineage. Progenitor cells can go through several rounds of cell division before terminally differentiating into a mature cells, and the cells may be these intermediary cells. The cells may be selected from the group consisting of: MSCs (marrow stromal cells, mesenchymal stem cells, multipotent stromal cells), chondrocytes, fibrochondrocytes, osteocytes, osteoblasts, osteoclasts, synoviocytes, adipocytes, bone marrow cells, mesenchymal cells, stromal cells, genetically transformed cells, or combinations thereof. The cells may be autologous or heterologous.

In some embodiments, the biomaterial scaffold comprises fibrin. Other materials may also be engineered to include peptides of the disclosure. Such materials are described in U.S. Pat. Nos. 7,241,730, 6,331,422, 6,607,740, 6,723,344, US Pub 2007/0202178, US Pub 2007/0264227, which are hereby incorporated herein by reference for all purposes.

In some embodiments, the biomaterial scaffold comprises collagen. Collagen scaffolds are described in, for example, US Publications: 2017/0182212, 20170173216, 20160199538, and 20150367030, which are hereby incorporated herein by reference for all purposes.

### V. THERAPEUTIC METHODS

After damage, tissue repair or regeneration is the result of a spatio-temporal coordination of cell fate processes that are controlled by a multitude of cell-signaling events coming from the extracellular microenvironment and recruited cells at the site of injury (Gurtner, Werner, et al., 2008). To site few, tissue healing processes such as angiogenesis (Herbert and Stainier, 2011), stem cells homing (Karp and Leng Teo, 2009), or inflammation (Eming, Hammerschmidt, et al., 2009) are all tightly coordinated and controlled by a cascade of cell-signaling events. Angiogenesis, the formation of new blood vessels, is crucial to provide oxygen and nutrients to the regenerating tissue. Various approaches have been made with a goal of providing amenable and tissue-specific matrices to control cell processes, such as adhesion, migration, proliferation, differentiation (Lutolf and Hubbell, 2005; Atala, 2008; Huebsch and Mooney, 2009). A goal is to provide matrices to contain signals that directly act on tissue-damaged cells, attract regeneration-competent cells, block regeneration-suppressing signals, and guide cell fate. Powerful molecules to control these processes are secreted cell-signaling molecules such as morphogens (Affolter and Basler, 2007), cytokines (Vilcek and Feldmann, 2004), and growth factors (Cross and Dexter, 1991).

The embodiments of the disclosure may facilitate these processes and can be used to assist in the healing of normal wounds, including those resulting from accidents, surgery or failure of healing of a surgical wound (e.g., a dehiscent wound). Certain aspects of the disclosure will accelerate wound healing, reduce scarring and ultimately promote repair, regeneration and restoration of structure and function in all tissues.

The embodiments of the disclosure can be used to treat external wounds caused by, but not limited to scrapes, cuts, lacerated wounds, bite wounds, bullet wounds, stab wounds, burn wounds, sun burns, chemical burns, surgical wounds, bed sores, radiation injuries, all kinds of acute and chronic wounds, wounds or lesions created by cosmetic skin procedures and also ameliorate the effects of skin aging. The embodiments of the disclosure may accelerate wound healing in all kinds of external wounds and improve the cosmetic appearance of wounded areas, and skin subject to aging and disease. In certain embodiments, the composition, peptide, polypeptide, implant, molecular complex, scaffold, or matrix of the disclosure may be provided directly, as a pre-treatment, as a pre-conditioning, coincident with injury, pre-injury, or post-injury. The composition be used to treat internal injury caused by, but not limited to, disease, surgery, gunshots, stabbing, accidents, infarcts, ischemic injuries, to organs and tissues including but not limited to heart, bone, brain, spinal cord, retina, peripheral nerves and other tissues and organs commonly subject to acute and chronic injury, disease, congenital and developmental malformation and aging processes. Injury to internal organs causes a fibrotic response, which leads to loss of structure and function in organ systems.

In certain aspects, regenerative processes aided by the compositions peptides, polypeptides, implants, molecular complexes scaffolds, or matrices of the disclosure may include, but are not limited to internal and external injury, regeneration of tissues, organs, or other body parts, healing and restoration of function following vascular occlusion and ischemia, brain stroke, myocardial infarction, spinal cord damage, brain damage, peripheral nerve damage, ocular damage (e.g., to corneal tissue), bone damage and other insults to tissues causing destruction, damage or otherwise resulting from, but not limited to, injury, surgery, cancer, congenital and developmental malformation, and diseases causing progressive loss of tissue structure and function, including but not limited to diabetes, bacterial, viral and prion-associated diseases, Alzheimer's disease, Parkinson's disease, AIDs and other genetically determined, environmentally determined or idiopathic disease processes causing loss of tissue/organ/body part structure and function. In addition, the compositions described herein can be administered with drugs or other compounds promoting tissue and cellular regeneration including, but not limited to, trophic factors in processes including, but not limited to, brain, retina, spinal cord and peripheral nervous system regeneration (e.g., NGFs, FGFs, Neurtrophins, Neuregulins, Endothelins, GDNFs, BDNF. BMPs, TGFs, Wnts), as well as pre-conditioning factors or stimuli e.g., hypoxia, norepinephrine, bradykinin, anesthetics, nitrate, ethanol, Alda-1, ALDH2 antagonists, PKC-epsilon agonists, exogenous ligands that activate opioid receptors (DPDPE, deltorphin II, methadone, SNC-80, BW373U86, DPI-287, DPI-3290) delivered in a prospective pre-treatment prior to a surgery of other procedure disrupting tissue in a subject.

Embodiments of the disclosure further include the use of the peptides, compositions, polypeptides, implants, molecular complexes, scaffolds, or matrices of the disclosure to aid in the healing of pathological wounds, such as through use of a contractile toroid for assisting the closure of slow healing wounds e.g., diabetic wounds. Diabetic wounds are examples of difficult to heal wound can include, for example, a wound that is often characterized by slower than normal re-epithelialization/closure inflammatory phase and delayed formation and remodeling of extracellular matrix.

The present disclosure can also assist in the healing of chronic wounds or wounds that do not heal. Wounds that have not healed within three months, for example, are said to be chronic. Chronic wounds include, diabetic, diabetic foot, ischemic, venous, venous stasis, arterial, pressure, vasculitic, infectious, decubitis, burn, trauma-induced, gangrenous and mixed ulcers. Chronic wounds include, wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed embodiments in would healing and tissue regeneration would include in humans and agricultural, sports and pet animals.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Laminin Heparin-Binding Peptides Bind to Several Growth Factors and Enhance Diabetic Wound Healing

1. Results

a. Multiple GFs Bind to Multiple Isoforms of Laminin

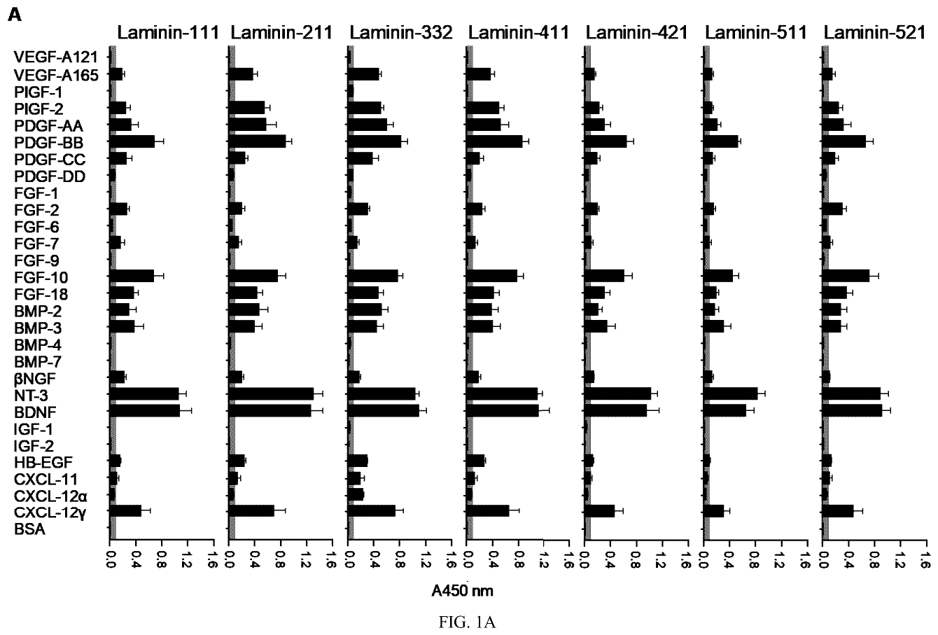

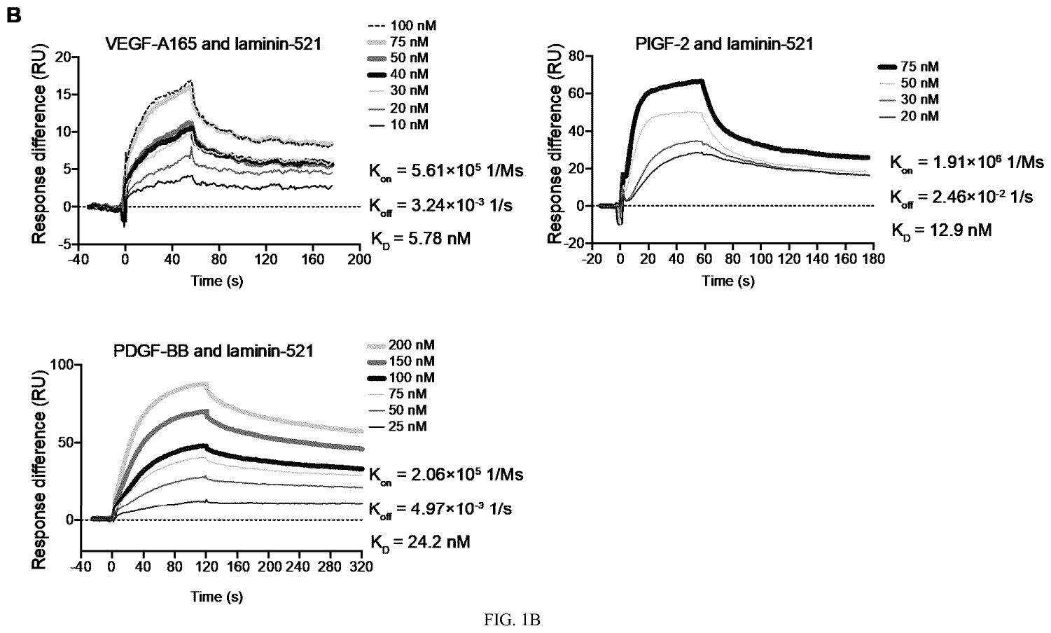

The inventors first examined the capacity of a variety of full-length laminin isoforms (−111, −211, −332, −411, −421, −511, and −521) to bind GFs from the VEGF/PDGF, FGF, BMP, NT, IGF, EGF and CXCL chemokine families, for which the inventors have previously observed binding to other ECM proteins, including fibronectin, vitronectin, tenascin-C, osteopontin, and fibrinogen, as well as that reportedly modulate wound-healing. Binding of laminin to absorbed GFs was detected using an antibody against laminin, and signals greater than 0.1 were considered to be indicative of a binding event. Overall, it was found that multiple GFs strongly bound to all tested laminin isoforms (FIG. 1A). Specifically, from the VEGF/PDGF family, VEGF-A165, PlGF-2, PDGF-AA, PDGF-BB, and PDGF-CC bound to all isoforms of laminin, in contrast to VEGF-A121, PlGF-1, and PDGF-DD which did not show binding. From the FGF family, the inventors observed that FGF-2, FGF-7, FGF-10, and FGF-18 bound to all laminin isoforms, whereas FGF-1, FGF-6, and FGF-9 did not. Among the BMPs, BMP-2 and BMP-3 showed binding to laminins, but not BMP-4 and BMP-7. NT-3 and BDNF showed strong binding towards all tested laminin isoforms, while βNGF bound only weakly. Neither IGF-1 nor IGF-2 displayed significant binding to laminins. In addition, HB-EGF weakly bound to laminins. As to the tested chemokines, CXCL-12γ bound to all laminin isoforms, whereas CXCL-11 and CXCL-12a bound weakly to laminin-332 but not to the other isoforms.

Next, the inventors measured the affinities between laminin-521, as an example, and VEGF-A165, PlGF-2, and PDGF-BB using surface plasmon resonance (SPR). SPR chips were functionalized with laminin-521, and growth factors were flowed over the surface. The obtained binding curves were fitted with Langmuir binding kinetics to calculate specific dissociation constants (K_{D}) (FIG. 1B). K_{D} values were 5.8 nM for VEGF-A165, 12.9 nM for PlGF-2, and 24.2 nM for PDGF-BB. The nM range of K_{D} values demonstrated the strong binding affinities of laminin-521 to the selected GFs.

b. GFs Bind to the HBDs of Laminin

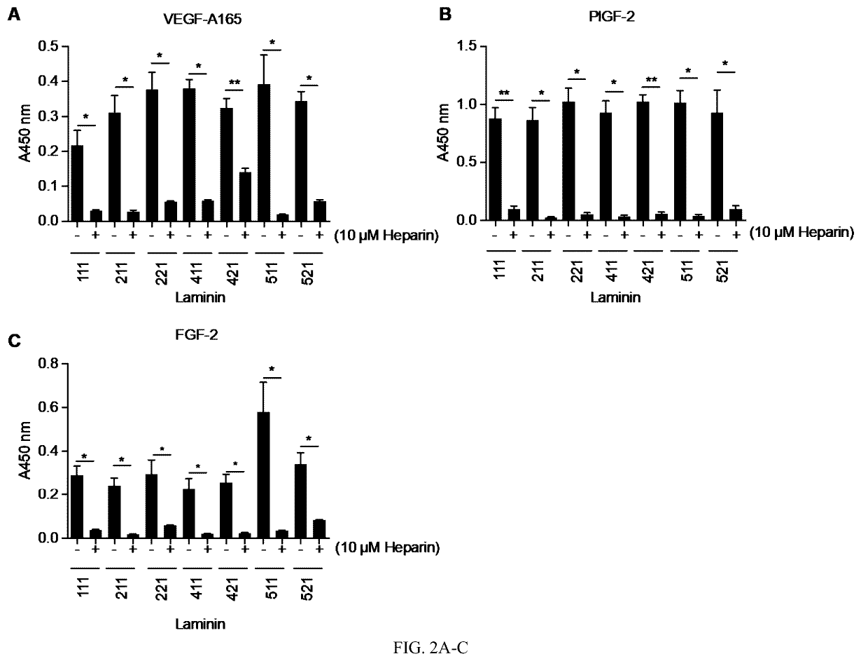

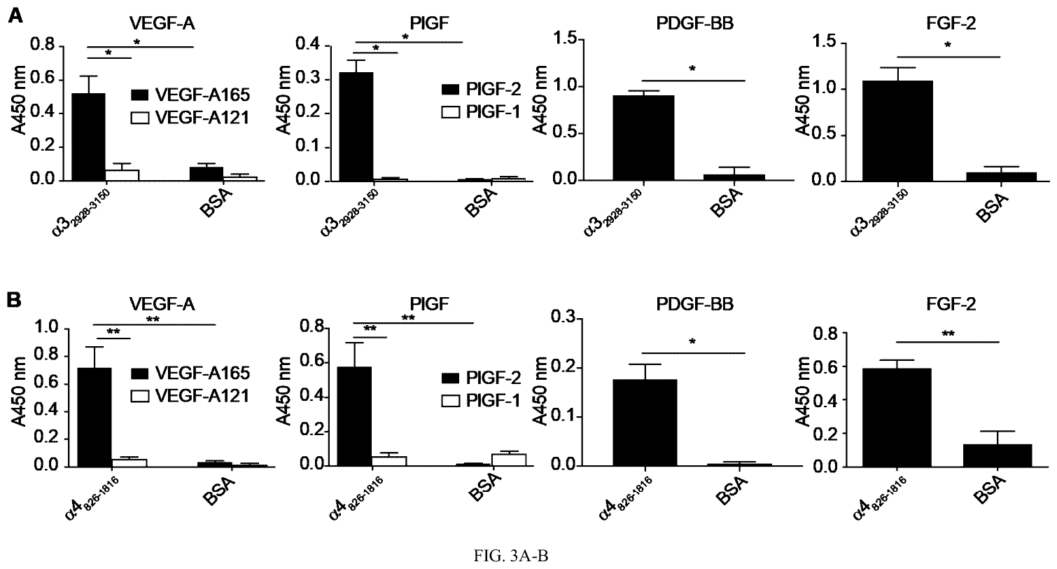

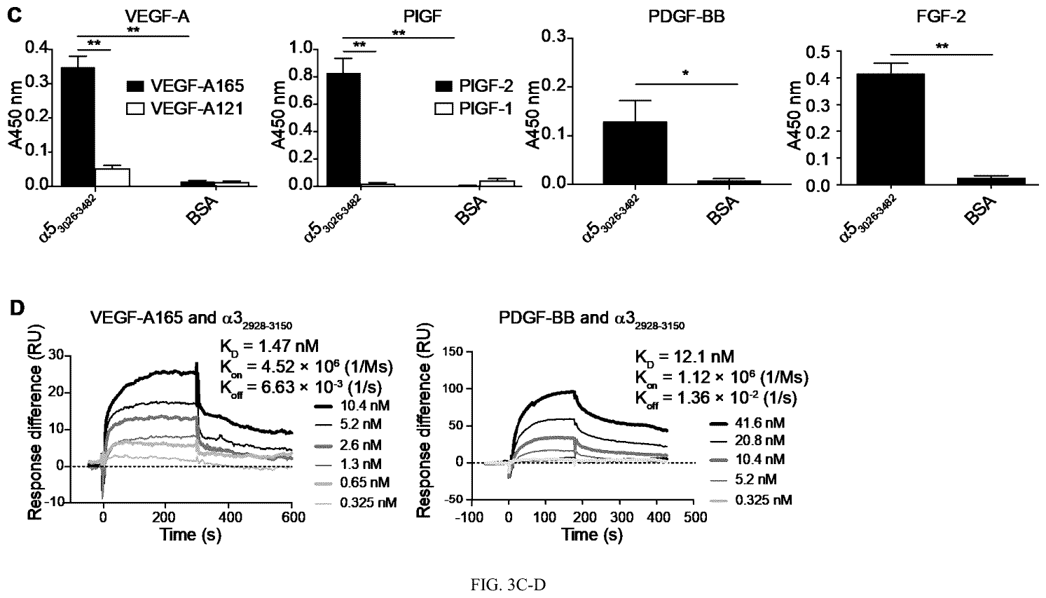

Because the GFs that bound to laminins have also been previously reported to bind to other ECM glycoproteins through HBDs, it was hypothesized that HBDs of laminins might be responsible for the interactions between GFs and laminin. To address this hypothesis, ELISA assays were repeated for VEGF-A165, PlGF-2 or FGF-2 in the presence of heparin added in excess (10 μM). As a result, the inventors observed that excess heparin inhibited GF binding to laminin (FIG. 2A-C), supporting that laminin HBDs mediated interactions with GFs. To further confirm this, the inventors tested direct GF binding to the LG domains from human laminin α3, α4 and α5, within which HBDs of laminin were localized. It was found that VEGF-A165, PlGF-2, PDGF-BB, and FGF-2 bound to laminin LG domains α3₂₉₂₈₋₃₁₅₀, α4₈₂₆₋₁₈₁₆ and α5₃₀₂₆₋₃₄₈₂, in contrast to VEGF-A121 and PlGF-1 which did not show any binding (FIG. 3A-C), as tested by ELISA. The binding affinities between α3₂₉₂₈₋₃₁₅₀ and VEGF-A165 or PDGF-BB were then measured by SPR, and K_{D} values were 1.2 nM for VEGF-A165, and 10.2 nM for PDGF-BB (FIG. 3D). These data again demonstrated the strong affinities of the laminin LG domain to the tested GFs.

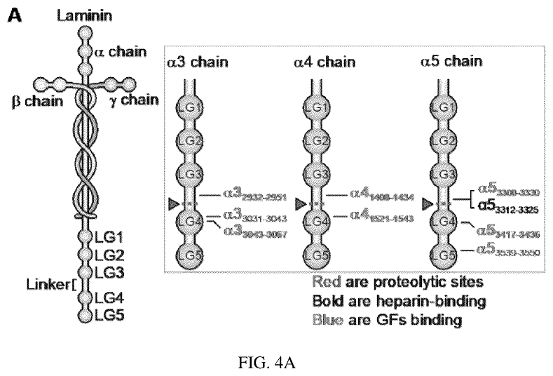

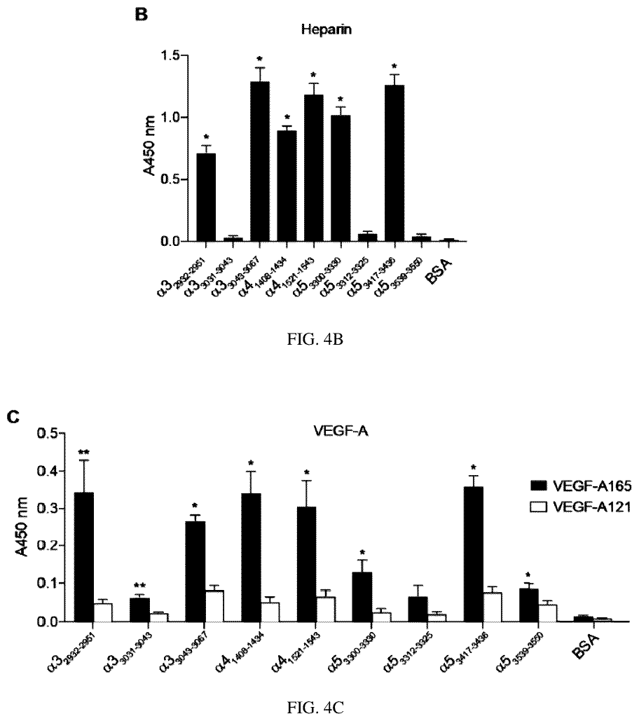

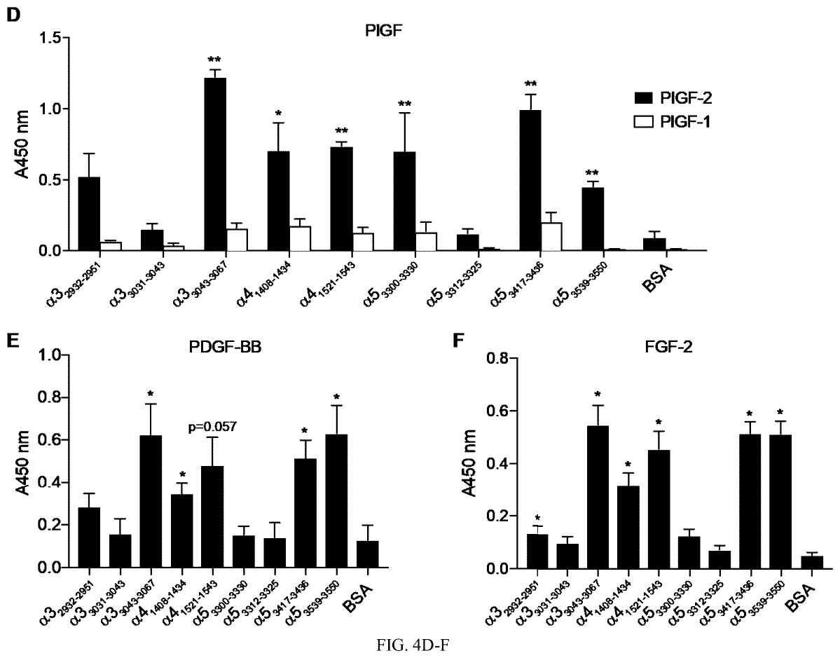

The inventors next examined the binding of GFs to chemically synthesized laminin LG domain peptides, the sequences of which are all derived from human laminin sequences (Table 1, FIG. 4A). These peptides are putative HBDs; they were determined based on previous reports with mouse or human HBD sequences, or are positively charged sequences located within the linker domain between the LG3 and LG4 domains in laminin α3, α4 and α5 chains. Of 9 tested peptides, 6 bound to heparin (i.e. HBDs), namely α3₂₉₃₂₋₂₉₅₁, α3₃₀₄₃₋₃₀₆₇, α4₁₄₀₈₋₁₄₃₄, α4₁₅₂₁₋₁₅₄₃, α5₃₃₀₀₋₃₃₃₀, and α5₃₄₁₇₋₃₄₃₆ among which α3₂₉₃₂₋₂₉₅₁, α4₁₄₀₈₋₁₄₃₄, and α5₃₃₀₀₋₃₃₃₀ are derived from the LG3-LG4 linker. Interestingly, α5₃₃₁₂₋₃₃₂₅, which is a subdomain of α5₃₃₀₀₋₃₃₃₀, did not bind to heparin.

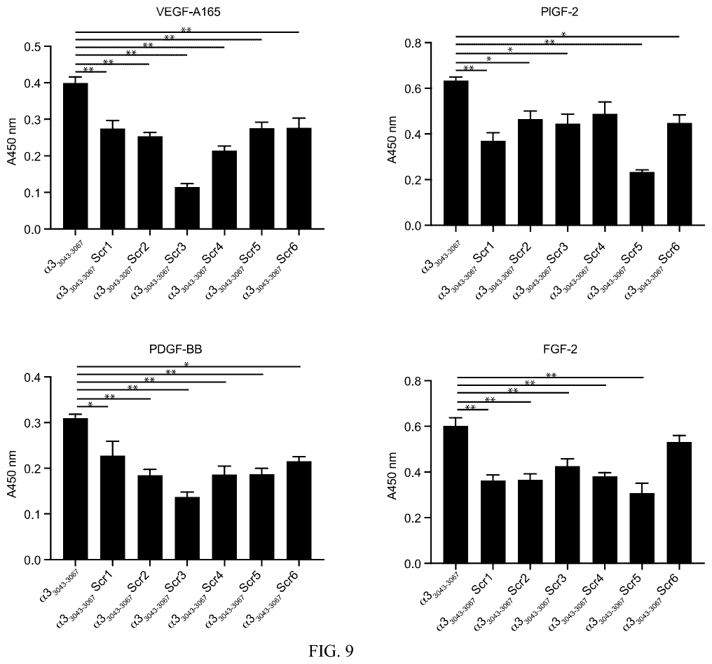

Finally, the affinities of VEGF-A, PlGF, PDGF-BB, and FGF-2 to these peptides were examined (FIG. 4B-F). The inventors observed that all heparin-binding peptides showed significant binding to some GFs. Indeed, α3₃₀₄₃₋₃₀₆₇, α4₁₄₀₈₋₁₄₃₄, and α5₃₄₁₇₋₃₄₃₆ bound to VEGF-A165, PlGF-2, PDGF-BB, and FGF-2. α4₁₅₂₁₋₁₅₄₃ showed similar results except for the binding to PDGF-BB, which was not statistically significant. α3₂₉₃₂₋₂₉₅₁ and α5₃₃₀₀₋₃₃₃₀ preferentially bound to VEGF-A165 and FGF-2, and VEGF-A165 and PlGF-2 respectively. As to the non-heparin-binding peptides, α5₃₃₁₂₋₃₃₂₅ did not show particular binding to any tested GF. Interestingly, α5₃₅₃₉₋₃₅₅₀, which did not show binding to heparin, significantly bound to all tested GFs, and α3₃₀₃₁₋₃₀₄₃ bound to VEGF-A165. None of the tested laminin-derived peptides bound to VEGF-A121 nor to PlGF-1, consistent with the results obtained in FIG. 1 and FIG. 3. To examine sequence specificity of this binding to GFs, the inventors produced a scrambled sequence α3₃₀₄₃₋₃₀₆₇ peptide (FIG. 9); scrambling the sequence of α3₃₀₄₃₋₃₀₆₇ decreased the binding signals between α3₃₀₄₃₋₃₀₆₇ and VEGF-A165, PlGF-2, PDGF-BB, and FGF-2, compared to its native form. Taken together, these data suggest that GFs bind to the HBDs of laminin, located in the LG3-LG4 linker or in LG4-LG5 domains.

c. Laminin HBD Peptides Promote Adhesion of Multiple Types of Cells

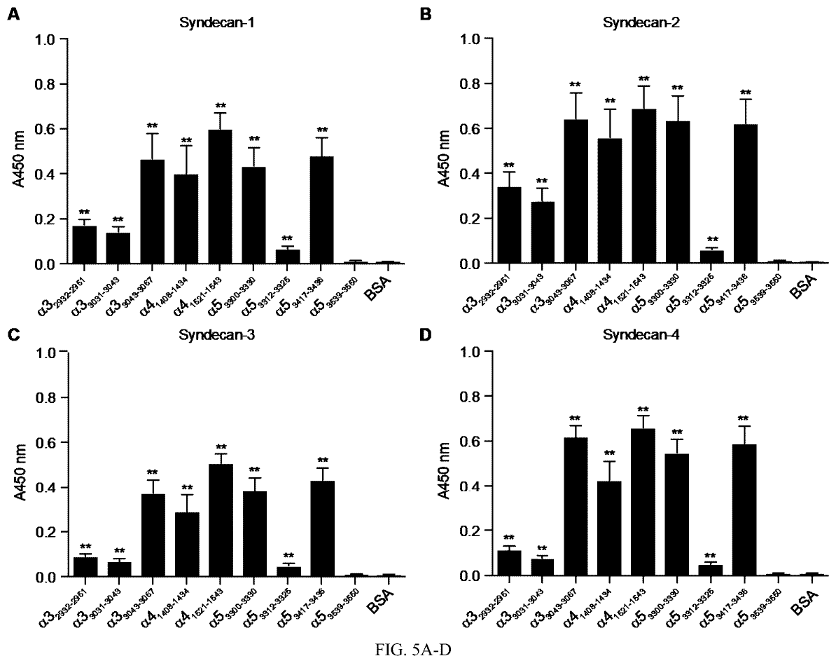

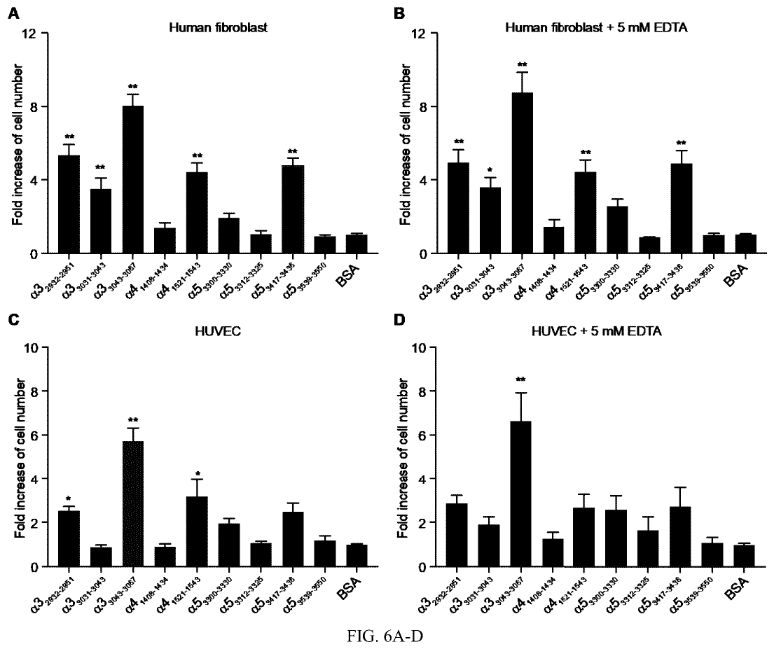

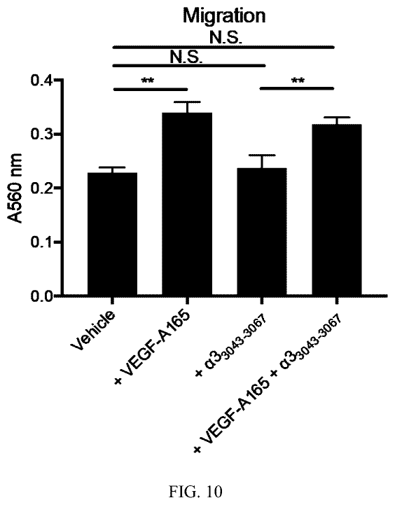

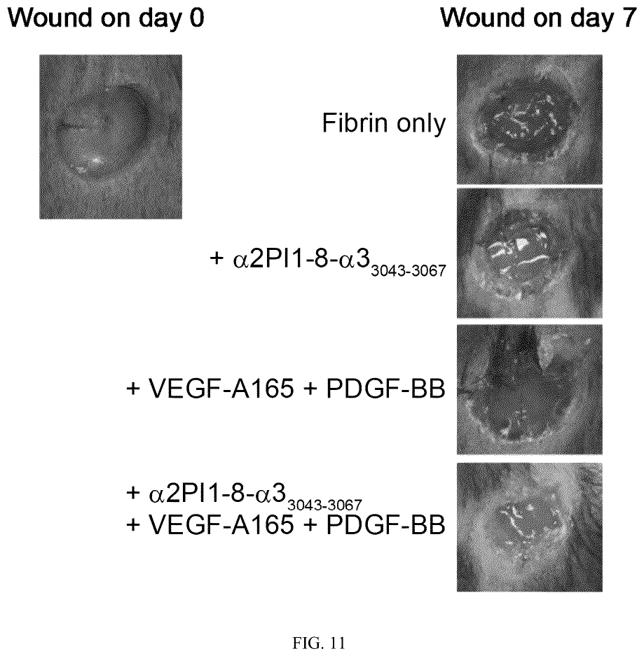

Because the laminin HBDs have been reported to bind to syndecan, a key cell surface adhesion molecule, the inventors tested syndecan binding to the synthesized laminin-derived peptides (FIG. 5A-D). α3₃₀₄₃₋₃₀₆₇, α4₁₅₂₁₋₁₅₄₃, α4₁₄₀₈₋₁₄₃₄, α5₃₄₁₇₋₃₄₃₆, and α5₃₃₀₀₋₃₃₃₀ showed significant binding to all isoforms of recombinant syndecans, i.e. syndecan 1-4. α3₂₉₃₂₋₂₉₅₁, α3₃₀₃₁₋₃₀₄₃, and α5₃₃₁₂₋₃₃₂₅ showed weak binding to the tested syndecans, while α5₃₅₃₉₋₃₅₅₀ did not show binding to any syndecan isoform. Because laminin-derived peptides that interact with syndecans may further promote cell adhesion by providing binding substrates, the inventors tested fibroblasts and HUVEC adhesion to plates coated with these peptides. The inventors observed enhancement of fibroblast attachment on α3₂₉₃₂₋₂₉₅₁, α3₃₀₃₁₋₃₀₄₃, α3₃₀₄₃₋₃₀₆₇, α4₁₅₂₁₋₁₅₄₃ and α5₃₄₁₇₋₃₄₃₆-coated surfaces (FIG. 6A). Fibroblast binding was observed even in the presence of EDTA, consistent with syndecan function (FIG. 6B). Of these peptides, α3₂₉₃₂₋₂₉₅₁, α3₃₀₄₃₋₃₀₆₇, and α4₁₅₂₁₋₁₅₄₃ also promoted HUVEC attachment (FIG. 6C), even in the presence of EDTA in the case of α3₃₀₄₃₋₃₀₆₇ (FIG. 6D). Interestingly, peptides that promoted both fibroblast and HUVEC adhesion in vitro through syndecan binding were those that the inventors previously found to be laminin HBDs (FIG. 4A). VEGF-A165 increases the degree of migration of HUVEC cells in vitro (FIG. 10). However, both in the presence and absence of VEGF-A165, α3₃₀₄₃₋₃₀₆₇ did not increase the degree of cell migration.

d. Retention of VEGF-A165 and PDGF-BB in Fibrin Matrix is Increased by the Incorporation of Laminin HBD Peptides

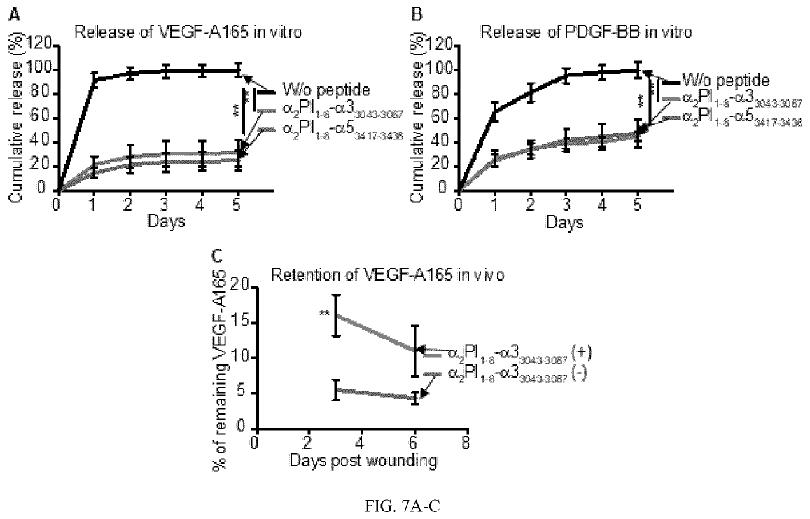

The inventors then sought to determine whether laminin HBD peptides, which showed binding to GFs, were able to improve the retention of VEGF-A165 and PDGF-BB within fibrin matrix. VEGF-A165 and PDGF-BB are both crucial factors for angiogenesis. These GFs are known to be quickly released from fibrin matrices upon delivery, which limits their wound healing efficacy in vivo. For this purpose, the inventors selected α3₃₀₄₃₋₃₀₆₇ and α5₃₄₁₇₋₃₄₃₆ laminin HBD peptides, and fused them to a transglutaminase-reactive sequence from the α₂-plasmin inhibitor to allow their covalent incorporation by factor XIIIa into fibrin matrices during polymerization. GF release from fibrin matrices containing α₂PI₁₋₈-α3₃₀₄₃₋₃₀₆₇, α₂PI₁₋₈-α5₃₄₁₇₋₃₄₃₆ or no laminin-derived peptide were then monitored daily and quantified by ELISA (FIG. 7A, B). As expected, the inventors observed that VEGF-A165 and PDGF-BB were quickly released from the fibrin matrix (>85% released after 24 h). However, incorporation of either α₂PI₁₋₈-α3₃₀₄₃₋₃₀₆₇ or α₂PI₁₋₈-α5₃₄₁₇₋₃₄₃₆ allowed significant retention of VEGF-A165 and PDGF-BB into matrices, which were respectively released after 5 days, for VEGF-A165 (α₂PI₁₋₈-α3₃₀₄₃₋₃₀₆₇: 25%, α₂PI₁₋₈-α5₃₄₁₇₋₃₄₃₆: 31%) and for PDGF-BB (α₂PI₁₋₈-α3₃₀₄₃₋₃₀₆₇: 45%, α₂PI₁₋₈-α5₃₄₁₇₋₃₄₃₆: 47%). This data highlights the key biological role of laminin in sequestering GFs into ECM, and demonstrates the potential of laminin HBD peptides to control GF delivery from fibrin biomaterials (FIG. 7A, B). The inventors next evaluated the effect of α₂PI₁₋₈-α3₃₀₄₃₋₃₀₆₇ on GF retention in diabetic wounds in the type 2 diabetic db/db mouse in vivo (FIG. 7C). Incorporation of α₂PI₁₋₈-α3₃₀₄₃₋₃₀₆₇ into fibrin matrices significantly enhanced the amount of VEGF-A165 remaining in the wounds 3 days after treatment, showing that incorporation of α₂PI₁₋₈-α3₃₀₄₃₋₃₀₆₇ prolongs retention of GFs in vivo.

e. Laminin HBD-Functionalized Fibrin Matrices Potentiate GFs and Promote Wound Healing In Vivo

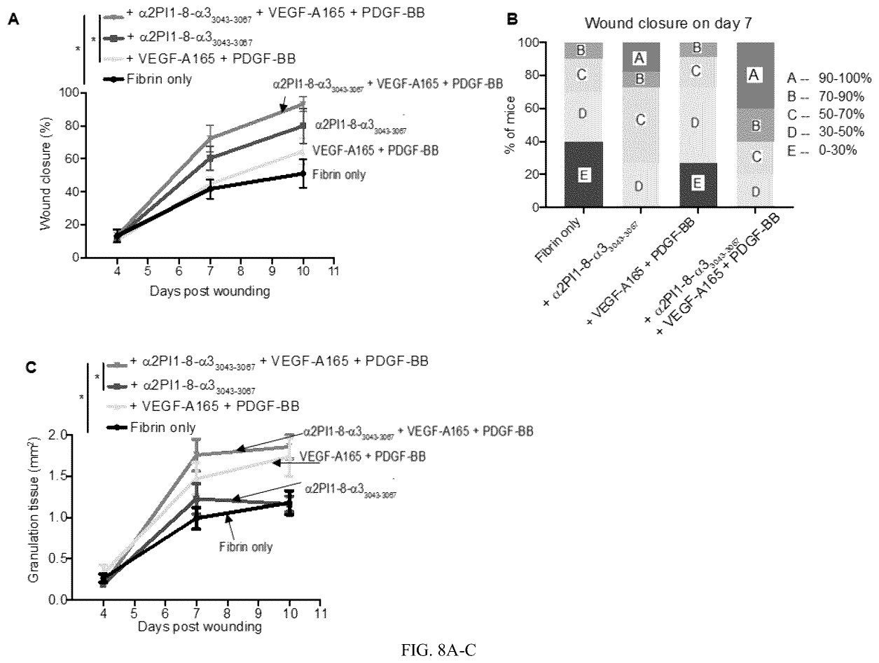

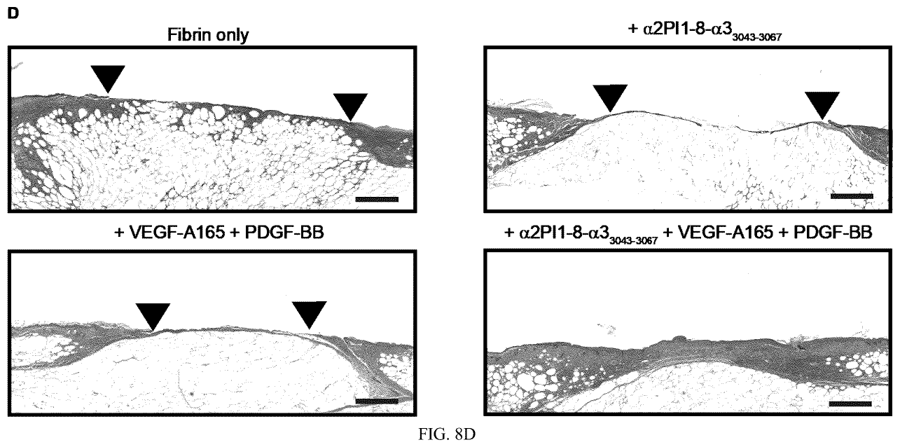

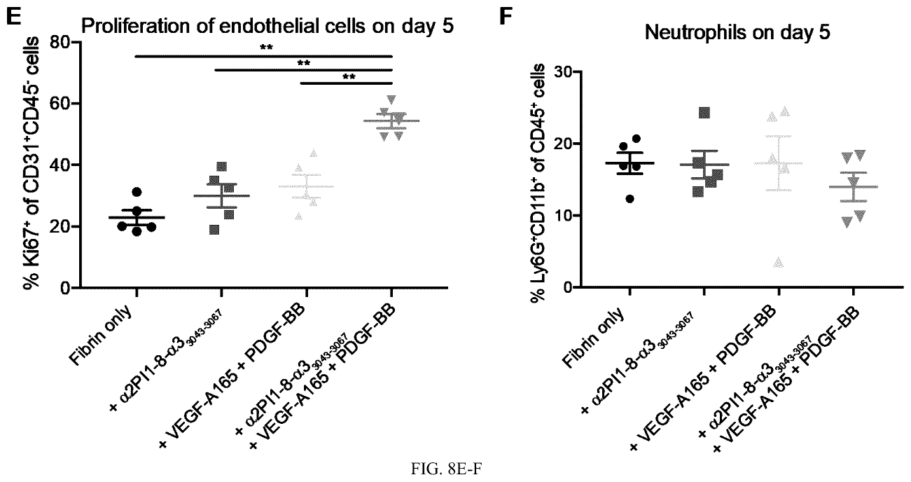

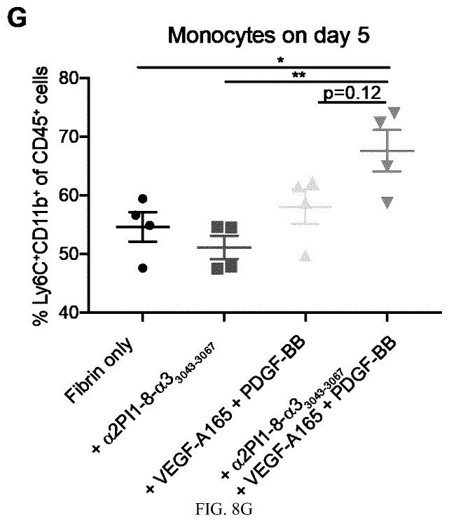

Although the etiology of non-healing wounds is multi-faceted in diabetes, the progression to a non-healing phenotype is related to poor blood vessel formation. Thus, induction of mature blood vessels is a crucial step for diabetic wound-healing. Previous studies have reported a synergistic effect between angiogenesis inducers VEGF-A 165 and PDGF-BB in wound healing, more precisely topical application of VEGF-A165 improves wound closure and PDGF-BB promotes the amount of granulation tissue in the type 2 diabetic db/db mouse. The inventors further evaluated whether fibrin matrices engineered with laminin-HBD peptides could enhance skin repair in a model of delayed wound healing, by controlling the release of VEGF-A165 and PDGF-BB in vivo. VEGF-A165 (100 ng/wound) and PDGF-BB (50 ng/wound) were co-delivered from fibrin matrix onto full-thickness back-skin wounds in db/db mice, which provides a well-established and clinically-relevant model of impaired wound healing. Here, the inventors particularly functionalized fibrin with the laminin peptide α3₃₀₄₃₋₃₀₆₇, since it bound to GFs and syndecans, and promoted fibroblast and endothelial cells adhesion in vitro (FIG. 4-6). Four groups were tested: fibrin only, fibrin functionalized with α₂PI₁₋₈-α3₃₀₄₃₋₃₀₆₇, fibrin containing GFs, and fibrin functionalized with α₂PI₁₋₈-α3₃₀₄₃₋₃₀₆₇ and containing GFs. Wound histology was analyzed after 4, 7 and 10 days, considering that wounds are normally fully closed after 15 days when treated with fibrin matrix. As a result, wounds that received fibrin matrices containing GFs or α₂PI₁₋₈-α3₃₀₄₃₋₃₀₆₇ peptide only did not differ from wounds treated with fibrin alone on day 7, neither in amount of granulation tissue nor in extent of wound closure (FIG. 8A-C). In contrast, the co-delivery of VEGF-A165 and PDGF-BB in fibrin functionalized with α₂PI₁₋₈-α3₃₀₄₃₋₃₀₆₇ led to a significantly faster wound closure after 7 days, as well as a significant increase in granulation tissue formation (FIG. 8A-C). GFs alone improved the amount of granulation tissue but not wound closure on day 10, suggesting that α₂PI₁₋₈-α3₃₀₄₃₋₃₀₆₇ peptide speeds the wound healing process by these GFs. Representative wound morphology for all four treatments is presented in FIG. 8D. Clear differences in granulation tissue thickness and extent of re-epithelialization can be visualized when GFs were delivered within the α₂PI₁₋₈-α3₃₀₄₃₋₃₀₆₇ peptide-functionalized fibrin matrix compared to the other conditions.

Angiogenesis is a crucial step of wound-healing in diabetic wounds, and both VEGF-A165 and PDGF-BB are angiogenesis inducers. The inventors next examined endothelial cell proliferation (FIG. 8E). Co-delivery of VEGF-A165 and PDGF-BB in fibrin functionalized with α₂PI₁₋₈-α3₃₀₄₃₋₃₀₆₇ led to a significantly increased frequency of Ki67⁺, a proliferation marker, within CD31⁺CD45⁻ endothelial cells compared to other treatment groups on day 5. This is consistent with the increase in granulation tissue observed on day 7 as a result of delivery of GFs in fibrin functionalized with α₂PI₁₋₈-α3₃₀₄₃₋₃₀₆₇ (FIG. 8C).

Immune cells play crucial role in wound-healing regulation. The inventors next examined the immune cell population in the wound in each treatment group. Delivery of GFs in fibrin functionalized with α₂PI₁₋₈-α3₃₀₄₃₋₃₀₆₇ slightly decreased the frequency of neutrophils within CD45⁺ cells compared to other treatment groups. On the other hand, delivery of GFs in fibrin functionalized with α₂PI₁₋₈-α3₃₀₄₃₋₃₀₆₇ increased the frequency of monocytes within CD45⁺ cells compared to other treatment groups. Among immune cells, neutrophils migrate first into wounds and then monocytes appear^{43,44}. Therefore, this set of data suggests that delivery of GFs in fibrin functionalized with α₂PI₁₋₈-α3₃₀₄₃₋₃₀₆₇ promotes wound healing immunologically as well. Inclusion of α₂PI₁₋₈-α3₃₀₄₃₋₃₀₆₇ improved the GF delivery capacity of fibrin in vivo, resulting in an accelerated wound healing.

2. Discussion

It was unexpectedly found that GF binding to laminin does not seem to be limited to HBDs, as a few non-heparin binding peptides also bound to some GFs, notably α3₃₀₃₁₋₃₀₄₃ and α5₃₅₃₉₋₃₅₅₀. These peptides are human alignments of reported mouse HBD peptides, called A3G75 and A5G94 respectively. Thus, the mechanism of GF-binding to laminin still remains incompletely clarified and may be resolved by further crystallography studies of GF-laminin complex.

Physiologically, proteolytic cleavage of LG4 and LG5 domains is crucial for the deposition of laminin in the native ECM. Upon tissue injury, laminin is overexpressed, and LG4-LG5 domains accumulate in wounds, wherein they promote tissue healing mechanisms. In this study, the inventors characterized laminin-derived peptides that are located just before the proteolytic cleavage site, in the linker between the LG3 and LG4 domains, or within the LG4-LG5 domains (Table. 2, FIG. 4A). On one side, the inventors discovered 3 novel heparin-, GF- and syndecan-binding peptides within the LG3-LG4 linker regions of α3, α4, and α5 chains, namely α3₂₉₃₂₋₂₉₅₁, α4₁₄₀₈₋₁₄₃₄, and α5₃₃₀₀₋₃₃₃₀, identifiable through their highly cationic sequences (FIG. 4). Since α3, α4 and α5 chains are known to be predominantly present in their processed form (i.e. lacking LG4-LG5) in mature, unwounded skin, it is likely that these peptides are exposed in vivo under homeostatic conditions, thus providing both GF ligands and cell adhesion sites in basement membranes. Interestingly, laminin α1 chain, which is not proteolytically processed, and α2 chain do not contain such cationic sequences in the LG3-LG4 linker region, which might reflect functional differences between α chain isoforms. On the other side, the inventors identified 5 peptides in the LG4 and LG5 domains of α3, α4 and α5 chains that displayed specific binding to GFs, in particular to VEGF-A165. Among them, α3₃₀₄₃₋₃₀₆₇, α5₃₅₃₉₋₃₅₅₀, and α5₃₄₁₇₋₃₄₃₆ additionally bound to PDGF-BB, FGF-2 and PlGF-2 with high affinities (FIG. 4). These growth factors are well-known as key regulators of the wound healing cascade, and are particularly involved in wound angiogenesis. Therefore, it is proposed that the reported positive effects of LG4-LG5 domains during wound healing might be related to promiscuous interactions with GFs, in addition to binding to syndecans and release of laminin-derived pro-angiogenic peptides.

In this study, the inventors identified 5 laminin HBDs that are able to bind to both GFs and syndecan cell-surface receptors (FIGS. 4 and 5), among which α3₃₀₄₃₋₃₀₆₇, α4₁₅₂₁₋₁₅₄₃ and α5₃₄₁₇₋₃₄₄₆ further promoted cell attachment (FIG. 6). Although syndecans are not known to directly activate major signaling pathways, they support cell adhesion and integrin signaling. Moreover, direct binding of laminin peptides from LG domains to integrins has also been reported; for example, the integrin α3β1 binds to α3₂₉₃₂₋₂₉₄₃. Nevertheless, in the assays, EDTA did not abolish cell adhesion, suggesting that initial cell attachment was mediated by syndecans rather than integrins (the binding of which is Ca²⁺-dependent). Consequently, and considering the short length of the laminin HBD peptides, it is unlikely that laminin HBD peptides can enhance GF signaling via synergy with integrins. It is believed that GF binding properties, more than cell adhesion properties, of laminin HBDs in fibrin matrices substantially contribute to the promotion of wound healing.

Although GFs are promising drugs for tissue regeneration, their uncontrolled delivery upon application on wounded tissue has limited their clinical efficacy and safety to date. For example, recombinant human VEGF-A has not been approved for clinical use by the U.S. Food and Drug Administration (FDA) due to a negative result in phase II clinical trials. PDGF-BB (Regranex in the clinic) has shown clinical efficacy, but safety issues such as cancer risk have been flagged, potentially due to high dosing. Because 20 μg per wound of VEGF-A165 applied topically for five consecutive days were known to promote wound healing in the db/db mouse and 10 μg per wound of PDGF-BB did not significantly enhance wound healing, the inventors treated full-thickness back-skin wounds with a roughly 40- to 250-fold lower dose of GFs (combination of 100 ng VEGF-A165 and 50 ng of PDGF-BB) delivered once in a fibrin matrix. Thus, controlling GF delivery to improve efficacy and dose reduction seems essential in future GF-based therapies and could be achieved by use of biomaterials matrices.

Here, the inventors showed that covalent incorporation of an engineered GF-binding domain derived from laminin, α₂PI₁₋₈-α3₃₀₄₃₋₃₀₆₇, into fibrin matrix significantly enhanced the effect of VEGF-A165 and PDGF-BB on skin wound healing, by highly increasing GF retention into fibrin both in vitro and in vivo (FIG. 8). In contrast, wounds treated with fibrin matrix containing GFs only, in which PDGF-BB and VEGF-A165 were not specifically retained in the fibrin matrices, had no detectable effect on wound healing at the tested dose (FIG. 8). Wounds treated with fibrin matrix containing α₂PI₁₋₈-α3₃₀₄₃₋₃₀₆₇ only promoted wound-closure slightly. This might be the result of trapping endogenous GFs. Considering the importance of angiogenesis in diabetic wounds and the inventors' observation of increased Ki67⁺ within CD31⁺CD45⁻ endothelial cells, the healing process induced by fibrin matrix containing α₂PI₁₋₈-α3₃₀₄₃₋₃₀₆₇ and GFs was driven by enhanced angiogenesis in the wounds. Improved angiogenesis, which sustains the newly formed granulation tissue, resulted from effective sequestration of VEGF-A165 and PDGF-BB (FIG. 7). Granulation tissue morphogenesis translated to improved morphogenesis at the level of the dermal epithelium, as reflected by faster wound closure.

One advantage of using the laminin HBD peptide for wound healing, is production simplicity: the laminin HBD peptide is short enough to be chemically synthesized in large scale, rather than requiring recombinant expression. Furthermore, the inventors showed that a laminin HBD can functionalize fibrin matrix in both aspects as a GF reservoir and an adhesion-promoting cell scaffold (FIGS. 6 and 7).

In conclusion, the inventors found that multiple isoforms of laminin promiscuously bind GFs from the VEGF/PDGF, FGF, BMP, and NT families, in addition to HB-EGF and CXCL12γ, through their HBDs. By engineering a fibrin matrix displaying the α3₃₀₄₃₋₃₀₆₇ laminin HBD, as a demonstrative example, the inventors have shown that the laminin HBD peptide promotes skin wound closure in the db/db mouse, as a model of delayed wound healing, when associated with VEGF-A165 and PDGF-BB. In addition to highlighting a GF-modulating function for laminin, an important tissue homeostasis and repair protein, the inventors show that both GF- and cell-binding characters of a laminin HBD can promote tissue repair when incorporated within fibrin matrix, which may be clinically useful.

3. Tables

TABLE 1
The sequences of laminin-derived peptides.

| Name | |
|---|---|
| (location) | |
| length | Peptide sequence |
| α3₂₉₃₂₋₂₉₅₁ | PPFLMLLKGSTRFNKTKTFR |
| (Linker) | (SEQ ID NO: 2) |
| 20 aa. | |
| α 3₃₀₃₁₋₃₀₄₃ | KNSFMALYLSKGR |
| (LG4) 13 aa. | (SEQ ID NO: 9) |
| α 3₃₀₄₃₋₃₀₆₇ | RLVFALGTDGKKLRIKSKEKCNDGK |
| (LG4) 25 aa. | (SEQ ID NO: 1) |
| α 4₁₄₀₈₋₁₄₃₄ | PLFLLHKKGKNLSKPKASQNKKGGKSK |
| (Linker) | (SEQ ID NO: 4) |
| 27 aa. | |
| α 4₁₅₂₁₋₁₅₄₃ | TLFLAHGRLVYMFNVGHKKLKIR |
| (LG4) 23 aa. | (SEQ ID NO: 3) |
| α 5₃₃₀₀₋₃₃₃₀ | TPGLGPRGLQATARKASRRSRQPARHPACML |
| (Linker) | (SEQ ID NO: 7) |
| 31 aa. | |
| α 5₃₃₁₂₋₃₃₂₅ | ARKASRRSRQPARH |
| (Linker) | (SEQ ID NO: 10) |
| 14 aa. | |
| α 5₃₄₁₇₋₃₄₃₆ | RQRSRPGRWHKVSVRWEKNR |
| (LG4) 20 aa. | (SEQ ID NO: 6) |
| α 5₃₅₃₉₋₃₅₅₀ | TLPDVGLELEVR |
| (LG5) 12 aa. | (SEQ ID NO: 5) |
| α 3₃₀₄₃₋₃₀₆₇ | RLVKALKTDKFLGRIGSEKCNDKGK |
| Scr1 25 aa. | (SEQ ID NO: 74) |
| α 3₃₀₄₃₋₃₀₆₇ | RKTDALELVFLKKGGIGSKKCNDKR |
| Scr2 25 aa. | (SEQ ID NO: 75) |
| α 3₃₀₄₃₋₃₀₆₇ | CRKKKRKKKALLLGIGDFNSEVTDG |
| Scr3 25 aa. | (SEQ ID NO: 76) |
| α 3₃₀₄₃₋₃₀₆₇ | KKRKLVALTDFLGICGSENDGRKKK |
| Scr4 25 aa. | (SEQ ID NO: 77) |
| α 3₃₀₄₃₋₃₀₆₇ | LVRAKLTDKFLGKRIGSKECNKDKG |
| Scr5 25 aa. | (SEQ ID NO: 78) |
| α 3₃₀₄₃₋₃₀₆₇ | ALLLGIGRDFNKKKRKKKSEVTDGC |
| Scr6 25 aa. | (SEQ ID NO: 79) |
| α ₂PI₁₋₈- | NQEQVSPLRLVFALGTDGKKLRIKSKEKCNDGK |
| α 3₃₀₄₃₋₃₀₆₇ | (SEQ ID NO: 8) |
| 33 aa. | |
| α ₂PI₁₋₈- | NQEQVSPLARKASRRSRQPARH |
| α 5₃₃₁₂₋₃₃₂₅ | (SEQ ID NO: 11) |
| 22 aa. | |

TABLE 2
Summary of laminin-derived peptide interactions.

| Laminin-derived | Interaction with | | | Cell adhesion | |
|---|---|---|---|---|---|
| peptides | Heparin | GFs | Syndecans | Fibroblasts | HUVECs |
| α 3₂₉₃₂₋₂₉₅₁ | ++ | + | + | + | + |
| α 3₃₀₃₁₋₃₀₄₃ | | + | + | + | |
| [symbol] | [symbol] | [symbol] | [symbol] | [symbol] | [symbol] |
| α 4₁₄₀₈₋₁₄₃₄ | ++ | ++ | ++ | | |
| α 4₁₅₂₁₋₁₅₄₃ | ++ | + | ++ | + | + |
| α 5₃₃₀₀₋₃₃₃₀ | ++ | + | ++ | | |
| α 5₃₃₁₂₋₃₃₂₅ | | | + | | |
| α 5₃₄₁₇₋₃₄₃₆ | ++ | ++ | ++ | + | |
| α 5₃₅₃₉₋₃₅₅₀ | | + | | | |

++ indicates high affinities, + indicates medium/low affinities. The laminin-derived peptide tested in vivo is highlighted in bold.

4. Materials and Methods

a. Growth Factors and Chemokines

All growth factors (GFs) and chemokines were purchased in their mature forms, highly pure (>95% pure), carrier-free, and lyophilized¹. Vascular endothelial growth factor (VEGF)-A121, VEGF-A165, placental growth factor (PlGF)-1, PlGF-2, platelet-derived growth factor (PDGF)-AA, PDGF-BB, PDGF-CC, PDGF-DD, fibroblast growth factor (FGF)-1, FGF-2, FGF-6, FGF-7, FGF-9, FGF-10, FGF-18, bone morphogenetic protein (BMP)-2, BMP-3, BMP-4, BMP-7, β-nerve growth factor (NGF), neurotrophin (NT)-3, brain-derived neurotrophic factor (BDNF), insulin-like growth factor (IGF)-1, IGF-2, heparin-binding epidermal growth factor (HB-EGF), C—X—C motif ligand (CXCL)-11, and CXCL-12a were purchased from PeproTech. CXCL-12γ was purchased from R&D systems. Except for PDGF-DD and BMP-7, which were produced in eukaryotic cells, all GFs were produced in Escherichia coli and thus were not glycosylated. All GFs were reconstituted and stored according to the provider's instructions to regain full activity and prevent loss of protein.

b. Detection of Laminin Binding to Recombinant GFs

ELISA tests were performed as previously reported. In brief, ELISA plates (med-binding, Greiner Bio-One) were coated with 50 nM GFs at 37° C. for more than 2 hrs. After blocking with 2% BSA solution containing PBS and 0.05% Tween 20 (PBS-T), 10 nM recombinant human laminin isoforms (−111, −211, −332, −411, −421, −511, and −521) (>95% purity tested by SDS-PAGE, BioLamina) were added. Bound laminin was detected with rabbit anti-human laminin γ1 chain antibody (1:1000 dilution, Assay biotech) or rabbit anti-human laminin α3 chain antibody (1:1000 dilution, Assay biotech). After incubation with biotinylated anti-rabbit antibody for 60 min at room temperature (RT), HRP conjugated streptavidin (Jackson ImmunoResearch) was added. After 60 min of incubation at RT, 50 μL TMB substrate (Sigma-Aldrich) was added. The reactions were stopped by adding 25 μL of 2 N H₂SO₄. Subsequently, the absorbance at 450 nm was measured with a reference of 570 nm.

c. Production and Purification of Recombinant Laminin α3₂₉₂₈₋₃₁₅₀ Protein

Protein production and purification were performed as described previously¹. The sequence encoding for human laminin alpha 3 LG domain Ser2928-Cys3150 (linker domain and LG4 domain) was synthesized and subcloned into the mammalian expression vector pcDNA3.1(+) by Genscript. A sequence encoding for 6 His was added at the N-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×10⁶ cells/mL. 1 μg/mL plasmid DNA, 2 μg/mL linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO₂. 6 days after transfection, the cell culture medium was collected by centrifugation and filtered through a 0.22 μm filter. Culture media was loaded into a HisTrap HP 5 mL column (GE Healthcare), using an ÄKTA pure 25 (GE Healthcare). After washing of the column with wash buffer (20 mM imidazole, 20 mM NaH₂PO₄, 0.5 M NaCl, pH 7.4), protein was eluted with a gradient of 500 mM imidazole (in 20 mM NaH₂PO₄, 0.5 M NaCl, pH 7.4). The elusion solution was further purified with size exclusion chromatography using a HiLoad Superdex 200PG column (GE healthcare). All purification steps were carried out at 4° C. The expression of laminin LG domain was determined by western blotting using anti-His tag antibody (BioLegend) and the proteins were verified as >90% pure by SDS-PAGE.

d. Surface Plasmon Resonance (SPR)

SPR analysis was performed as described previously². In brief, measurements were made with a Biacore 3000 SPR system (GE Healthcare). Laminin-521 or laminin α3₂₉₂₈₋₃₁₅₀ was immobilized via amine coupling on a C1 chip (GE Healthcare) for ˜2000 or ˜1000 resonance units (RU), respectively, according to the manufacturer's instructions. VEGF-A165, PDGF-BB, or PlGF-2 was flowed at increasing concentrations in the running buffer at 20 μL/min. The sensor chip was regenerated with 50 mM NaOH for every cycle. Specific bindings of GFs to laminin were calculated by comparison to a non-functionalized channel used as a reference. Experimental results were fitted with Langmuir binding kinetics using BIAevaluation software (GE Healthcare).

e. Inhibition of Laminin-GF Binding by Heparin

ELISA plates (med-binding) were coated with 10 μg/mL laminin isoforms (−111, −211, −221, −411, −421, −511, and −521) in PBS for 2 hrs at 37° C. Then, wells were blocked with 2% BSA-containing PBS-T and further incubated with 1 μg/mL each of VEGF-A165, PlGF-2, or FGF-2 for 60 min at RT with 10 μM heparin. Next, the wells were incubated with biotinylated anti-VEGF, anti-PlGF, or anti-FGF-2 antibodies (R&D Systems). The antibodies were detected by streptavidin-HRP (R&D Systems). Signals were revealed and measured as described above.

f. Detection of GF Binding to Recombinant Laminin LG Domain Protein and the Synthesized Laminin HBD Peptides

ELISA tests were performed as described above. In brief, ELISA plates were coated with 1 μg/mL of laminin alpha 3 LG domain recombinant protein, laminin alpha 4 LG domain recombinant protein (R&D systems), laminin alpha 5 LG domain recombinant protein (LD BioPharma), or laminin peptide (sequences are described in Table 1, chemically synthesized by Genscript) in PBS for 2 hrs at 37° C. 1 μg/mL of BSA served as non-binding protein control. After blocking with 2% BSA PBS-0.05% Tween 20 (PBS-T) solution, 1 μg/mL of the recombinant human proteins (VEGF-A121, VEGF-A165, PlGF-1, PlGF-2, PDGF-BB or FGF-2) or 10 μg/mL of biotinylated heparin (Sigma-Aldrich) were added. Bound GF was detected with biotinylated antibodies for human VEGF, PlGF, PDGF-BB, or FGF-2 (R&D Systems). The antibodies were detected by streptavidin-HRP (R&D Systems). Signals were revealed and measured as described above.

g. Detection of Recombinant Syndecan Binding to the Synthesized Laminin HBD Peptides

ELISA tests were performed as described above. In brief, ELISA plates were coated with 1 μg/mL laminin peptide (sequences are described in Table 1, chemically synthesized by Genscript) in PBS for 2 hrs at 37° C. 1 μg/mL of BSA served as non-binding protein control. After blocking with 2% BSA PBS-T solution, 1 μg/mL of the recombinant human syndecan-1, syndecan-2, syndecan-3, syndecan-4 (all syndecan proteins are histidine-tagged; SinoBiological) were added. Bound GF was detected with anti-histidine tag antibody (1:1000 dilution, BioLegend). Signals were revealed and measured as described above.

h. Cell Adhesion Assay

96-well plates (non-tissue culture treated, Greiner Bio-one) were pre-coated with 1 μg/mL with laminin HBD peptides in PBS for 2 hrs at 37° C., followed by blocking with 2% BSA PBS for 1 h at RT. Cell adhesion assays were performed using human lung fibroblasts (Lonza) in FGM-2 medium (Lonza) or human umbilical vein endothelial cells (HUVEC; Lonza) in EGM-2 medium (Lonza) supplemented with 1% fetal bovine serum (FBS) and 100 μg/mL VEGF-A165, with or without 5 mM EDTA (Sigma-Aldrich). Cells were plated at 3000 cells/well on laminin peptide pre-coated plates and incubated for 30 min at 37° C., 5% CO₂. Then, the medium was removed, and wells were quickly washed three times with PBS. Cell numbers were quantified using a CyQUANT assay, according to the manufacturer's instructions (Invitrogen). All cell lines were checked for mycoplasma contamination and used in passages from 5 to 8.

i. Migration Assay

A migration assay was performed as described previously³. A QCM 24-Well Colorimetric Cell Migration Assay kit was used to perform migration assay. Both sides of inserts were coated with 0.1 μM of bovine collagen I (C4243, Sigma-Aldrich) for 1 hr at 37° C. Then, the inserts were washed with water, dried in a laminar flow cabinet and disposed on 24-well cell culture plate covers. Solutions containing 30 ng/mL of VEGF-A165 preincubated with or without 0.1 μM of α3₃₀₄₃₋₃₀₆₇ peptide in medium (MCDB-131, 0.05% BSA) were added to the bottom side of the transwell (500 μL/well). Directly thereafter, HUVEC cells in medium containing 0.05% BSA (300 μL/transwell, 4×10⁴ cells/transwell) were added to the transwell upper parts. After 6 hr, migrated cells were stained and absorbance at 560 nm was measured according to the manufacturer's instructions.

j. Release of GF from Fibrin Matrix

Fibrin matrices were generated with human fibrinogen (VWF and fibronectin depleted, Enzyme Research Laboratories) as described previously¹. In brief, fibrin matrices were generated with 8 mg/mL fibrinogen, 2 U/mL human thrombin (Sigma-Aldrich), 4 U/mL factor XIIIa (Fibrogammin; Behring), 5 mM calcium chloride (Sigma-Aldrich), 2 μM α₂PI₁₋₈-laminin peptide (sequences are described in Table 1, chemically synthesized by Genscript), and 500 ng/mL recombinant human VEGF-A165 or PDGF-BB. Thus, the peptides were incorporated into the 3D fibrin matrix through enzymatic coupling, via the coagulation transglutaminase factor XIIIa, of the α₂PI₁₋₈ peptide sequence (NQEQVSPL) fused to the laminin peptide. Fibrin matrix was polymerized at 37° C. for 1 hr and transferred into 24-well Ultra Low Cluster plates (Corning) containing 500 μL of buffer (20 mM Tris-HCl, 150 mM NaCl, and 0.1% BSA; pH 7.4). A control well that served as a 100% released control contained only the GF in 500 μL of buffer. Every 24 hrs, buffers were removed, stored at −20° C., and replaced with fresh buffer. For the 100% released control well, 20 μL of buffer was removed each day and stored at −20° C. After 5 days, the cumulative release of GF was quantified by ELISA (DuoSet; R&D Systems), using the 100% released control as a reference.

k. Retention of VEGF-A165 at the Wound Site

Retention assays were performed as previously reported¹. Briefly, C57BLKS/J-m/Lepr db (db/db) mice ages 10 to 11 wks were used. Their backs were shaved and four full-thickness punch-biopsy wounds (6 mm in diameter) were created in each mouse. Directly after, fibrin matrices [80 μL total, fibrinogen (10 mg/mL), 2 U/mL human thrombin, 4 U/mL factor XIII, 5 mM calcium chloride, 2 μM α₂PI₁₋₈-α3₃₀₄₃₋₃₀₆₇, 200 ng of recombinant human VEGF-A165] were polymerized on the wounds. To avoid drying of the matrices, the wounds were covered with non-adhering dressing (Adaptic, Johnson&Johnson), and then with adhesive film dressing (Hydrofilm, Hartmann). After 3 or 6 days, mice were sacrificed. The wounds were punched again, in order to recover the fibrinous matrices. Moreover, the tissue surrounding the wounds (2 mm beyond the wound margin) was removed. The tissue was transferred in 0.9 mL of tissue T-PER Tissue Protein Extraction Reagent (Thermo Scientific) containing 1 mg/mL of collagenase IV (Sigma-Aldrich), and homogenized with a tissue homogenizer. The tissue lysate was incubated 1 hr at 37° C. and 100 μL of a 5 M NaCl solution containing protease inhibitors (1 tablet of protease inhibitor cocktail for 10 mL) was added to the lysate. The samples were centrifuged at 10000×g for 5 min, and the supernatants were stored at −80° C. Recombinant human VEGF-A165 remaining in the fibrinous matrix and in the tissue surrounding the wound were quantified by ELISA (DuoSet, R&D Systems), using 200 ng of recombinant human VEGF-A165 as 100%.

l. Mouse Skin Chronic Wound Healing Model

Skin wound healing assays were performed as previously reported¹. Briefly, C57BLKS/J-m/Lepr db (db/db) male mice were 10 to 12 wks old at the start of the experiments. Their backs were shaved and four full-thickness punch biopsy wounds (6 mm in diameter) were created in each mouse. Directly after, fibrin matrices [80 μL total, fibrinogen (10 mg/mL), 2 U/mL human thrombin, 4 U/mL factor XIII, 5 mM calcium chloride, 2 μM α₂PI₁₋₈-α3₃₀₄₃₋₃₀₆₇, 100 ng of VEGF-A165, and 50 ng of PDGF-BB] were polymerized on the wounds. The wounds were covered with adhesive film dressing. Mice were single-caged after the wound surgery. After 4, 7, 10 days, mice were euthanized and the skin wounds were carefully harvested for histological analysis.

m. Histomorphometric Analysis of Wound Tissue Sections

Histomorphometric analyses were performed as previously reported¹. Briefly, an area of 8 mm in diameter, which includes the complete epithelial margins, was excised. Wounds were cut in the center into two and embedded into paraffin. Histological analysis was performed on 5 μm serial sections. Images were captured with an EVOS FL Auto microscope (Life Technologies). The extent of re-epithelialization and granulation tissue formation was measured by histomorphometric analysis of tissue sections (H&E stain) using ImageJ software (NIH). For analysis of re-epithelialization, the distance that the epithelium had traveled across the wound was measured; the muscle edges of the panniculus carnosus were used as indicator for the initial wound edges; and re-epithelialization was calculated as the percentage of the distance of edges of the panniculus carnosus muscle. For granulation tissue quantification, the area covered by a highly cellular tissue was determined.

n. Flow Cytometric Analysis of the Wounds

Skin wounds were treated with fibrin matrices as described above. After 5 days, the wounded skins were removed as described above, cut into small pieces (<0.5 mm²) and transferred to 1 mL of an enzyme solution (collagenase D (1 mg/mL)) and agitated for 1 hr at 37° C. Then, the cells from digested wounds were re-suspended in PBS, passed through a cell strainer and centrifuged. Then, cells were stained for 15 min in 100 μL of FACS buffer containing antibodies: anti-CD31 (MEC13.3, BD Biosciences), anti-Ki67 (B56, BD Biosciences), anti-CD45 (30-F11), anti-Ly6G (1A8), anti-Ly6C (HK1.4), and anti-CD11b (M1/70). All antibodies were purchased from BioLegend if not otherwise described. Fixable live/dead cell discrimination was performed using Fixable Viability Dye eFluor 455 (eBioscience) according to the manufacturer's instructions. Intracellular staining was performed using the Intracellular Staining Permeabilization Wash Buffer according to manufacturer's instructions (BioLegend). Cells were analyzed using a Fortessa (BD Biosciences) flow cytometer and analyzed using FlowJo software (FlowJo, LLC.).

o. Statistical Analysis

Statistical methods were not used to predetermine necessary sample size, but sample sizes were chosen based on estimates from pilot experiments and previously published results such that appropriate statistical tests could yield significant results. Statistically significant differences between experimental groups were determined by one-way ANOVA followed by Tukey's HSD post hoc test with Prism software (v7, GraphPad). Variance between groups was found to be similar by the Brown-Forsythe test. For non-parametric data, the Kruskal-Wallis test followed by Dunn's multiple comparison test was used. For ELISA data, the two-tailed Mann-Whitney U test was used. For the animal studies, experiments were not performed in a blinded fashion. Mice were randomized into treatment groups within a cage immediately before the wound surgery and treated in the same way. All animal experiments were performed with approval from the Veterinary Authority of the Institutional Animal Care and Use Committee of the University of Chicago. GF-laminin binding ELISA assays were repeated 4 times. Wound healing assays were repeated 3 times. The P values less than 0.05 are considered to be significantly different. The P values less than 0.05 and 0.01 indicate symbols * and **, respectively.

### Example 2—Use of Recombinant Laminin A-Chain LG4 Domain for Controlled Delivery of Growth Factor/Chemokines from Biomaterials

Controlling the release kinetic of therapeutic proteins, such as growth factors (GFs) and chemokines, is essential to fully exploit their biological effects. In regenerative medicine, for example, GFs that are rapidly release from an injured site showed very modest clinical efficacy, thus implying their use at supra-physiological doses. As a consequence of such high non-physiological dosing, several GF-based therapies received safety warnings due to serious side effects directly related to the GF activity (e.g. ectopic tissue growth, tumor development). In this context, it has been demonstrated that engineering the slow-release of therapeutic proteins from biomaterials significantly increase their biological effects at reduced doses.

The inventors showed that LG4 domains located in the α-chain of the different laminin isoforms strongly bind to multiple GFs and chemokines. In this example, the use of these high affinity and promiscuous interactions between the laminin α-chain LG4 domains and GFs/chemokines to control GFs/chemokines delivery from biomaterials is described. Indeed, the incorporation of the LG4 domains in biomaterials can substantially increase retention of GF/chemokines, by providing high-affinity binding substrates.

Experimental design: Here, the incorporation of recombinant laminin LG4 domains into biomaterials through enzymatic cross-linking within the biomaterial is exemplified. More precisely, the LG4 domain of α3, α4 or α5-chain isoforms of laminin can be incorporated into fibrin-containing biomaterials through enzymatic crosslinking by the factor XIIIa during fibrin polymerization.

Other incorporation methods may include direct chemical conjugation of recombinant laminin LG4 to the biomaterial, or fusion of LG4 domains to protein sequences displaying strong but non-covalent binding to the biomaterial.

Methods: In this approach, the DNA sequence encoding for the transglutaminase substrate domain of the α₂-plasmin inhibitor, named α₂PI₁₋₈ (amino acid sequence: NQEQVSPL), followed by the DNA sequence of a short GGSG linker, can be fused to the 5′-end of the DNA sequence encoding for a LG4 domain of laminin α3, α4 or α5-chains; so that the end construct will be α₂PI₁₋₈-GGSG-LG4 (see sequences below).

Modified recombinant LG4 domains sequences can be then inserted into a DNA plasmid suitable for protein production. For production in mammalian cells, plasmids generally contain a Kozak sequence, a start codon and a signal sequence for protein secretion (e.g. IgGκ signal sequence), downstream of a strong ubiquitous promotor (e.g. CMV). The termination of the protein is achieved by a stop codon added at the C-terminus of the DNA sequence. An additional tag, such as a 6× histidine-tag, can be added at the N-terminus of the recombinant protein (i.e. after the signal sequence) or at its C-terminus (i.e. before the stop codon), to further facilitate protein purification. Following this design, recombinant LG4 domains will be produced by transient transfection of HEK293F cells over 7 days, and directly purified from the cell supernatant by affinity chromatography (e.g. to the histidine tag, to heparin) and/or physicochemical-based chromatography (e.g. size exclusion or ion-exchange chromatography). Final purity and identity of the recombinant laminin LG4 domain will be confirmed by SDS-PAGE and western blot analyses.

Results: Recombinant LG4 domains fused to the α₂PI₁₋₈ domain can be first assessed for their ability to remain incorporated into fibrin matrix. This is commonly achieved by performing release assays; after incorporation, the amount of recombinant LG4 domain released from fibrin matrix can be daily quantified either by ELISA or by fluorescence measurements, considering that LG4 domains could be fluorescently-labeled prior to incorporation.

As soon as the functionality of the α₂PI₁₋₈ domain as a substrate for crosslinking into fibrin can be established, the retention of GF/chemokines into fibrin containing laminin LG4 domains (versus fibrin alone) can be evaluated by ELISA-based release assays. Upon confirmation of successful GF/chemokines retention into fibrin by the recombinant laminin LG4 domains, fibrin matrices containing LG4 domains can be further characterized as a GF/chemokines delivery system in vivo, similarly to what was done in Example 1 with the α₂PI₁₋₈-fused LG4-derived peptides.

Interpretation: This molecular engineering of LG4 domains of α3, α4 and α5-chains of human laminin illustrates the use of recombinant LG4 domain as an additive to biomaterials, to enhance pharmacokinetic properties of biomaterials in delivering of GF/chemokines. Particularly in this example, the fusion of LG4 domains with the transglutaminase substrate sequence from α₂-plasmin inhibitor could leverage the GF/chemokines delivery properties of fibrin. Fusion of recombinant LG4 domains to other peptidic domains able to be sequestered into natural or synthetic biomaterials could be similarly envisioned.

1. Native Human Sequences of Laminin α-Chain Isoforms

LAMA3 Human, LG4 domain aa2986-aa3150 (UniprotKB
database Q16787):
(SEQ ID NO: 13)
ALQFGDIPTSHLLFKLPQELLKPRSQFAVDMQTTSSRGLVFHTGTKNSFM
ALYLSKGRLVFALGTDGKKLRIKSKEKCNDGKWHTVVFGHDGEKGRLVVD
GLRAREGSLPGNSTISIRAPVYLGSPPSGKPKSLPTNSFVGCLKNFQLDS
KPLYTPSSSFGVSSC
NCBI-CCDS database (CCDS11880.1):
(SEQ ID NO: 19)
GCCCTCCAGTTTGGGGACATTCCCACCAGCCACTTGCTATTCAAGCTTCC
TCAGGAGCTGCTGAAACCCAGGTCACAGTTTGCTGTGGACATGCAGACAA
CATCCTCCAGAGGACTGGTGTTTCACACGGGCACTAAGAACTCCTTTATG
GCTCTTTATCTTTCAAAAGGACGTCTGGTCTTTGCACTGGGGACAGATGG
GAAAAAATTGAGGATCAAAAGCAAGGAGAAATGCAATGATGGGAAATGGC
ACACGGTGGTGTTTGGCCATGATGGGGAAAAGGGGCGCTTGGTTGTGGAT
GGACTGAGGGCCCGGGAGGGAAGTTTGCCTGGAAACTCCACCATCAGCAT
CAGAGCGCCAGTTTACCTGGGATCACCTCCATCAGGGAAACCAAAGAGCC
TCCCCACAAACAGCTTTGTGGGATGCCTGAAGAACTTTCAGCTGGATTCA
AAACCCTTGTATACCCCTTCTTCAAGCTTCGGGGTGTCTTCCTGC.
LAMA4_Human, LG4 domain aa1469-aa1640 (UniprotKB
database Q16363):
(SEQ ID NO: 14)
AYQYGGTANSRQEFEHLKGDFGAKSQFSIRLRTRSSHGMIFYVSDQEEND
FMTLFLAHGRLVYMFNVGHKKLKIRSQEKYNDGLWHDVIFIRERSSGRLV
IDGLRVLEESLPPTEATWKIKGPIYLGGVAPGKAVKNVQINSIYSFSGCL
SNLQLNGASITSASQTFSVTPC.
NCBI-CCDS database (CCDS34514.1):
(SEQ ID NO: 20)
GCCTATCAATATGGAGGAACAGCCAACAGCCGCCAAGAGTTTGAACACTT
AAAAGGAGATTTTGGTGCCAAATCTCAGTTTTCCATTCGTCTGAGAACTC
GTTCCTCCCATGGCATGATCTTCTATGTCTCAGATCAAGAAGAGAATGAC
TTCATGACTCTATTTTTGGCCCATGGCCGCTTGGTTTACATGTTTAATGT
TGGTCACAAAAAACTGAAGATTAGAAGCCAGGAGAAATACAATGATGGCC
TGTGGCATGATGTGATATTTATTCGAGAAAGGAGCAGTGGCCGACTGGTA
ATTGATGGTCTCCGAGTCCTAGAAGAAAGTCTTCCTCCTACTGAAGCTAC
CTGGAAAATCAAGGGTCCCATTTATTTGGGAGGTGTGGCTCCTGGAAAGG
CTGTGAAAAATGTTCAGATTAACTCCATCTACAGTTTTAGTGGCTGTCTC
AGCAATCTCCAGCTCAATGGGGCCTCCATCACCTCTGCTTCTCAGACATT
CAGTGTGACCCCTTGC
LAMA5_Human, LG4 domain aa3340-aa3513 (UniprotKB
database O15230):
(SEQ ID NO: 15)
SYQFGGSLSSHLEFVGILARHRNWPSLSMHVLPRSSRGLLLFTARLRPGS
PSLALFLSNGHFVAQMEGLGTRLRAQSRQRSRPGRWHKVSVRWEKNRILL
VTDGARAWSQEGPHRQHQGAEHPQPHTLFVGGLPASSHSSKLPVTVGFSG
CVKRLRLHGRPLGAPTRMAGVTPC
NCBI-CCDS database (CCDS33502.1):
(SEQ ID NO: 21)
TCCTACCAGTTTGGGGGTTCCCTGTCCAGTCACCTGGAGTTTGTGGGCAT
CCTGGCCCGACATAGGAACTGGCCCAGTCTCTCCATGCACGTCCTCCCGC
GAAGCTCCCGAGGCCTCCTCCTCTTCACTGCCCGTCTGAGGCCCGGCAGC
CCCTCCCTGGCGCTCTTCCTGAGCAATGGCCACTTCGTTGCACAGATGGA
AGGCCTCGGGACTCGGCTCCGCGCCCAGAGCCGCCAGCGCTCCCGGCCTG
GCCGCTGGCACAAGGTCTCCGTGCGCTGGGAGAAGAACCGGATCCTGCTG
GTGACGGACGGGGCCCGGGCCTGGAGCCAGGAGGGGCCGCACCGGCAGCA
CCAGGGGGCAGAGCACCCCCAGCCCCACACCCTCTTTGTGGGCGGCCTCC
CGGCCAGCAGCCACAGCTCCAAACTTCCGGTGACCGTCGGGTTCAGCGGC
TGTGTGAAGAGACTGAGGCTGCACGGGAGGCCCCTGGGGGCCCCCACACG
GATGGCAGGGGTCACACCCTGC

2. Engineered Human Sequences of Laminin α-Chain Isoforms

Sequence design: The factor XIIIa transglutaminase substrate domain from the α₂-plasmin inhibitor (NQEQVSPL—SEQ ID NO:12) was added at the N-terminus of laminin LG4 domains, and separated from the LG4 domain by a short linker GGSG. The α2-plasmin inhibitor domain (NQEQVSPL—SEQ ID NO:12) could have been alternatively added to the C-terminus of LG4 domains (sequences not shown).

Human α₂PI₁₋₈-LAMA3_LG4₂₉₈₆₋₃₁₅₀:
(SEQ ID NO: 16)
NQEQVSPLGGSGALQFGDIPTSHLLFKLPQELLKPRSQFAVDMQTTSSRG
LVFHTGTKNSFMALYLSKGRLVFALGTDGKKLRIKSKEKCNDGKWHTVVF
GHDGEKGRLVVDGLRAREGSLPGNSTISIRAPVYLGSPPSGKPKSLPTNS
FVGCLKNFQLDSKPLYTPSSSFGVSSC.
Possible DNA sequence of human
α₂PI₁₋₈-LAMA3_LG4₂₉₈₆₋₃₁₅₀:
(SEQ ID NO: 22)
AACCAGGAGCAGGTGTCCCCACTTGGTGGATCCGGCGCCCTCCAGTTTGG
GGACATTCCCACCAGCCACTTGCTATTCAAGCTTCCTCAGGAGCTGCTGA
AACCCAGGTCACAGTTTGCTGTGGACATGCAGACAACATCCTCCAGAGGA
CTGGTGTTTCACACGGGCACTAAGAACTCCTTTATGGCTCTTTATCTTTC
AAAAGGACGTCTGGTCTTTGCACTGGGGACAGATGGGAAAAAATTGAGGA
TCAAAAGCAAGGAGAAATGCAATGATGGGAAATGGCACACGGTGGTGTTT
GGCCATGATGGGGAAAAGGGGCGCTTGGTTGTGGATGGACTGAGGGCCCG
GGAGGGAAGTTTGCCTGGAAACTCCACCATCAGCATCAGAGCGCCAGTTT
ACCTGGGATCACCTCCATCAGGGAAACCAAAGAGCCTCCCCACAAACAGC
TTTGTGGGATGCCTGAAGAACTTTCAGCTGGATTCAAAACCCTTGTATAC
CCCTTCTTCAAGCTTCGGGGTGTCTTCCTGC.
Human α₂PI₁₋₈-LAMA4_LG4₁₄₆₉₋₁₆₄₀:
(SEQ ID NO: 17)
NQEQVSPLGGSGAYQYGGTANSRQEFEHLKGDFGAKSQFSIRLRTRSSHG
MIFYVSDQEENDFMTLFLAHGRLVYMFNVGHKKLKIRSQEKYNDGLWHDV
IFIRERSSGRLVIDGLRVLEESLPPTEATWKIKGPIYLGGVAPGKAVKNV
QINSIYSFSGCLSNLQLNGASITSASQTFSVTPC.
Possible DNA sequence of human
α₂PI₁₋₈-LAMA4_LG4₁₄₆₉₋₁₆₄₀:
(SEQ ID NO: 23)
AACCAGGAGCAGGTGTCCCCACTTGGTGGATCCGGCGCCTATCAATATGG
AGGAACAGCCAACAGCCGCCAAGAGTTTGAACACTTAAAAGGAGATTTTG
GTGCCAAATCTCAGTTTTCCATTCGTCTGAGAACTCGTTCCTCCCATGGC
ATGATCTTCTATGTCTCAGATCAAGAAGAGAATGACTTCATGACTCTATT
TTTGGCCCATGGCCGCTTGGTTTACATGTTTAATGTTGGTCACAAAAAAC
TGAAGATTAGAAGCCAGGAGAAATACAATGATGGCCTGTGGCATGATGTG
ATATTTATTCGAGAAAGGAGCAGTGGCCGACTGGTAATTGATGGTCTCCG
AGTCCTAGAAGAAAGTCTTCCTCCTACTGAAGCTACCTGGAAAATCAAGG
GTCCCATTTATTTGGGAGGTGTGGCTCCTGGAAAGGCTGTGAAAAATGTT
CAGATTAACTCCATCTACAGTTTTAGTGGCTGTCTCAGCAATCTCCAGCT
CAATGGGGCCTCCATCACCTCTGCTTCTCAGACATTCAGTGTGACCCCTT
GC
Human α₂PI₁₋₈-LAMA5_LG4₃₃₄₀₋₃₅₁₃:
(SEQ ID NO: 18)
NQEQVSPLGGSGSYQFGGSLSSHLEFVGILARHRNWPSLSMHVLPRSSRG
LLLFTARLRPGSPSLALFLSNGHFVAQMEGLGTRLRAQSRQRSRPGRWHK
VSVRWEKNRILLVTDGARAWSQEGPHRQHQGAEHPQPHTLFVGGLPASSH
SSKLPVTVGFSGCVKRLRLHGRPLGAPTRMAGVTPC.
Possible DNA sequence of human
α₂PI₁₋₈-LAMA5_LG4₃₃₄₀₋₃₅₁₃:
(SEQ ID NO: 24)
AACCAGGAGCAGGTGTCCCCACTTGGTGGATCCGGCTCCTACCAGTTTGG
GGGTTCCCTGTCCAGTCACCTGGAGTTTGTGGGCATCCTGGCCCGACATA
GGAACTGGCCCAGTCTCTCCATGCACGTCCTCCCGCGAAGCTCCCGAGGC
CTCCTCCTCTTCACTGCCCGTCTGAGGCCCGGCAGCCCCTCCCTGGCGCT
CTTCCTGAGCAATGGCCACTTCGTTGCACAGATGGAAGGCCTCGGGACTC
GGCTCCGCGCCCAGAGCCGCCAGCGCTCCCGGCCTGGCCGCTGGCACAAG
GTCTCCGTGCGCTGGGAGAAGAACCGGATCCTGCTGGTGACGGACGGGGC
CCGGGCCTGGAGCCAGGAGGGGCCGCACCGGCAGCACCAGGGGGCAGAGC
ACCCCCAGCCCCACACCCTCTTTGTGGGCGGCCTCCCGGCCAGCAGCCAC
AGCTCCAAACTTCCGGTGACCGTCGGGTTCAGCGGCTGTGTGAAGAGACT
GAGGCTGCACGGGAGGCCCCTGGGGGCCCCCACACGGATGGCAGGGGTCA
CACCCTGC.

### Example 3—Use of Recombinant Laminin A-Chain LG4 Domain (or Other ECM Protein-Derived Growth Factor-Binding Domain) for Controlled Release of the Bone Morphogenetic Protein from Collagen Biomaterials

Collagen biomaterials are widely used in regenerative medicine, serving as a biocompatible supporting scaffold to promote cell activities during tissue regeneration, and to modulate the release of drugs (e.g. growth factors) upon implantation. As an example, the clinical product InFUSE® Bone Graft (Medtronic) is composed of a bovine Type I collagen sponge laden with the bone morphogenetic protein-2 (BMP-2), a well-known growth factor promoting bone regeneration. In the clinic, delivery of supraphysiological doses of BMP-2 (order of milligrams) into patients raised serious side effects, including ectopic bone formation, nerve injuries and increased cancer risk. Consequently, engineering delivery systems to control the release of BMP-2, as well as other growth factors, from collagen biomaterials constitutes a strong matter of interest for therapeutic use of growth factors. Here, the inventors exemplified the use of the laminin α-chain LG4 domain (LamLG4) and the fibrinogen β-chain heparin-binding domain (FgHBD) (Martino et al., PNAS, 2012), as growth factor-binding domains, to control the retention of BMP-2 into collagen biomaterials, and subsequently slow down their release.

1. Protein Designs

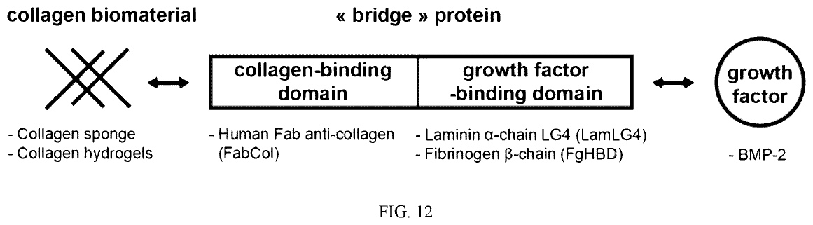

The inventors have engineered bipartite «bridge» proteins composed of a growth factor-binding domain linked to a collagen I-binding domain, which are able to retain BMP-2 into collagen biomaterials via non-covalent interactions (FIG. 12). The growth factors binding domains, namely LamLG4 or FgHBD, display strong affinity to BMP-2, and the collagen I-binding domain display strong affinity to collagen biomaterials, more particularly to bovine type I collagen hydrogels and sponges. In this example, the collagen-binding domain is made of a human antigen-binding fragment Fab from an anti-collagen I antibody (here named FabCol) patented elsewhere (WO 2016016269 A1).

2. Materials and Methods

a. DNA Sequences Preparation

The sequences of the variable regions of FabCol were taken from the patent WO 2016016269A1 (clone C11) and synthesized by Genscript (USA), before being incorporated into a plasmid containing human Fab constant regions. Both recombinant light chain and heavy chain were placed under the control of CMV promoters. LamLG4 and p(FgHBD) sequences were synthesized by Genscript. To prepare the FabCol-LamLG4 recombinant fusion protein DNA sequence, LamLG4 domain was placed at the C-terminus of the FabCol heavy chain, and separated from it by an 8 amino acids glycine-serine linker. As to the FabCol-p(FgHBD) fusion protein, 3 copies of the FgHBD domain were inserted at the C-termini of both the light and the heavy chains of FabCol, each copy linked to another by a 8 amino acids glycine-serine linker.

b. Protein Production of FabCol, FabCol-LamLG4, FabCol-p(FgHBD)

DNA plasmids of FabCol, FabCol-LamLG4 and FabCol-p(FgHBD) were prepared using NucleoBond Xtra maxiprep kits (Macherey-Nagel, USA). Plasmids were then transfected into human embryonic kidney cells (HEK293-F) using polyethyleneimine-mediated transfection and 1.5 mg plasmid per L of culture. The cells were cultured in suspension for 7 days in Freestyle 293 medium (ThermoFisher Scientific, USA). The culture supernatant was then collected and purified using HiTrap MabSelect column and an Akta PureM25 fast protein liquid chromatography FPLC systems (GE Healthcare Life Sciences, USA) according to the manufacturer instructions. FabCol-LamLG4 and FabCol-p(FgHBD) recombinant fusion proteins were further purified using HiTrap Heparin HP columns (GE Healthcare). Proteins were then dialyzed in phosphate saline buffer (PBS; pH 7.4), sterile-filtered and stored at −80° C.

c. Chemical Conjugation of FgHBD to FabCol

FgHBD peptide (>95% pure) was synthesized by Genscript (USA). FgHBD was chemically conjugated to FabCol using sulfo-SMCC crosslinker (ThermoFisher Scientific). One mg of FabCol was incubated with 30-fold molar excess of the sulfo-SMCC in PBS at room temperature for 1 h, after what the excess crosslinker was removed using Zeba Spin desalting columns, 7K MWCO (ThermoFisher Scientific). FgHBD peptide was then added to the FabCol at 30-fold molar excess, and the mixture was incubated for 1 h at room temperature. Unconjugated peptides were then removed using an Amicon 30 kDa centrifugal filters by diluting FabCol-p(FgHBD) conjugates into PBS and re-concentrating them, in repeated cycles. The removal of unconjugated FgHBD was assessed by SDS-PAGE gel chromatography. The conjugates were kept at 4° C. for maximum 2 weeks prior to experimentation.

d. SDS-PAGE Analyses

SDS-PAGE was used to assess size of the different FabCol variants. Protein samples were diluted in Laemmli buffer and loaded on MiniProtean TGX precast gels (gradient 4-20%; BioRad, Hercules CA, USA). Electrophoresis was run in Tris-Glycine-SDS buffer at 130 V for 1 h. Proteins were visualized using SimplyBlue SafeStain staining (ThermoFisher scientific).

e. Binding Assay to Bovine Type I Collagen

ELISA plates (NUNC MaxiSorp, ThermoFisher Scientific) were coated overnight with 10 μg/mL of bovine type I collagen (PureCol, Advanced BioMatrix, San Diego CA, USA) at room temperature. The plate was further blocked using 2% bovine serum albumine (BSA) for 2 h at room temperature. Then, appropriate amount of the FabCol-FgHBD conjugates, FabCol-LamLG4 or FabCol-p(FgHBD) recombinant proteins were diluted in PBS-0.05% Tween (PBST)+0.1% BSA to reach concentrations ranging from 0.01 nM to 30 nM, and were incubated for 1 h at room temperature. The plate was washed thrice in PBST, and an horseradish peroxidase-conjugated anti-human Fab antibody (Jackson ImmunoResearch,) was used to detect bound FabCol variants. The plate was revealed using TMB substrate solution, and stop with 1 M H2SO4. Absorbance at 450 nm was read using a Jackson ImmunoResearch, and corrected using the absorbance at 570 nm. Curve fits and dissociation constant KD were computed using Prism (GraphPad Software Inc., USA).

f. Binding Assay to rhBMP-2

ELISA plates (NUNC MaxiSorp) were coated with 50 nM of recombinant human BMP-2 (CHO produced, R&D Systems, Minneapolis MN, USA) overnight at room temperature. The plate was then blocked using 2% BSA for 2 h at room temperature, after which the plate was washed in PBST and incubated with 50 nM of the FabCol-FgHBD conjugates, FabCol-LamLG4 or FabCol-p(FgHBD) recombinant proteins diluted in PBS-0.05% Tween (PBST)+0.1% BSA. Bound FabCol variants were detected and revealed as described above.

g. Binding Assay to Engineered Super-Affinity Growth Factors

Engineered super-affinity growth factors and mouse wild-type VEGF-C were produced as described in Martino et al., Science, 2014. Other wild-type recombinant human growth factors were purchased from R&D Systems or Peprotech (Rocky Hill NJ, USA). Growth factors were coated on medium-binding plates (Greiner) at a concentration of 100 nM for 1 h at 37° C. Plates were then blocked with 2% BSA in PBS for 2 h at room temperature. Then, the FabCol variants (100 nM) were diluted in 1% BSA and incubated in the wells for 1 h at room temperature. The plate was washed four times in PBST and an HRP-anti-human Fab antibody was used to detect bound FabCol variants. Plate absorbance was read as described above.

h. Release from Collagen Matrix

Collagen hydrogels of 150 μL were prepared using PureCol bovine type I collagen (Advanced BioMatrix). FabCol variants (120 nM) and rhBMP-2 (500 ng/mL) were mixed with collagen (2.4 mg/mL) and 1× Minimum Essential Medium (MEM), used as a pH indicator. Under agitation, the pH was neutralized by adding 1 M NaOH, after what the mixture was directly plated into a 48-well plate, previously blocked overnight with 2% BSA in PBS. Gels were then polymerized for 1 h at 37° C. Release buffer (1 mL; Tris 20 mM, NaCl 150 mM, 0.1% BSA, 1% Penicillin-Streptomycin) was then added to the wells, and the gels were gently detached from the plate. The release buffer was collected and refreshed daily, and stored at −20° C. until analysis. A well that contained only BMP-2 served as a 100% released control. The amount of released rhBMP-2 was quantified using human BMP-2 DuoSet ELISA kit (R&D Systems), according to the manufacturer's instructions.

i. Immunohistochemistry Assessment of rhBMP-2 Retention into Collagen Sponge

Recombinant human BMP-2 (0.1 mg/mL in PBS) mixed with the FabCol variants at a 1:1 molar ratio was dripped onto collagen sponges (7 μL; Integra LifeSciences, Plainsboro Township NJ, USA), and further incubated 15 min at room temperature. Sponges were washed twice for 2.5 h in 10 mL of PBS containing 2% Fetal Bovine Serum (FBS). Sponges were then fixed in 2% paraformaldehyde (PFA) for 30 min. Sponges were again washed in PBS-2% FBS, and stained using a biotinylated anti-hBMP-2 (R&D Systems) and a streptavidin-AF594 using standard staining procedures. Sponges were imaged using a Leica DMi8 microscope (Leica, Wetzlar, Germany) and analysed using Fiji software (ImageJ, National Institute of Health, USA).

3. Results:

a. Conjugation of a Collagen-Binding Domain FabCol to a Growth Factor-Binding Domain FgHBD

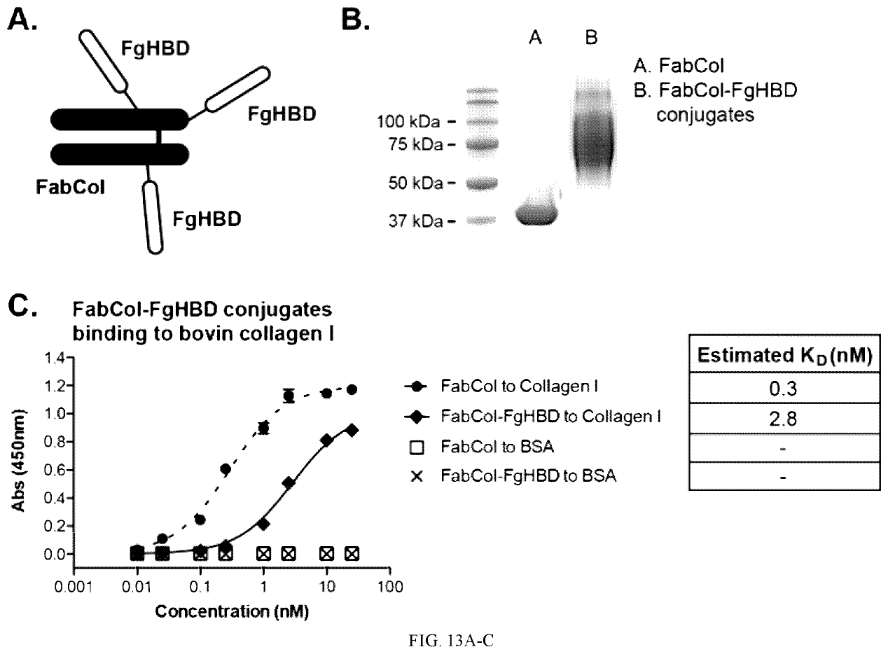

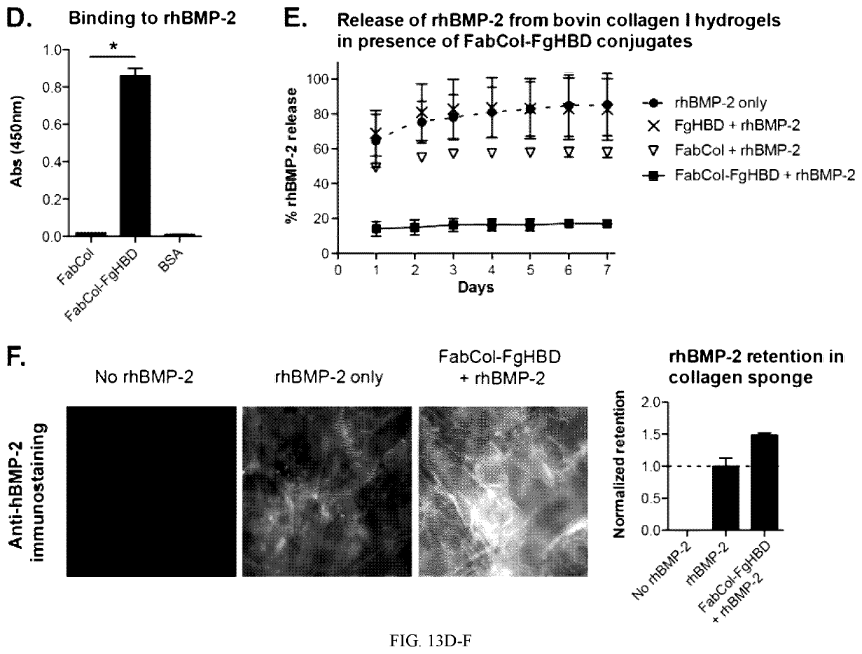

In this example, fibrinogen-derived domain FgHBD is used as the growth factor binding domain. The laminin-derived growth factor binding domains, such as LamLG4 may also be used. To engineer a bridge protein able to link growth factors into collagen biomaterials, FgHBD was chemically conjugated to FabCol using a sulfo-SMCC linker (FIG. 13A). Conjugation was confirmed by SDS-PAGE analysis, which revealed a shift of about 35 kDa in size between the non-conjugated FabCol and the FabCol-FgHBD conjugates. Such a size difference suggests that multiple copies of the FgHBD peptides were conjugated to the FabCol (FIG. 13B). After conjugation, the binding of FabCol-FgHBD conjugates to bovine type I collagen was preserved, although the affinity was reduced compared to non-conjugated FabCol. The dissociation constant K_{D} of FabCol-FgHBD conjugates to collagen I was determined by ELISA to be of high affinity, around 2.8 nM (FIG. 13C). In addition, FabCol-FgHBD conjugates strongly bound to rhBMP-2, whereas FabCol only did not (FIG. 13D).

b. FabCol-FgHBD Conjugates Increased Retention of rhBMP-2 into Collagen Biomaterials

When incorporated into collagen hydrogels, FabCol-FgHBD strikingly increased the retention of rhBMP-2 (FIG. 13E); indeed, only 20% of rhBMP-2 was released after 7 days, in contrast to 80% for the gels containing rhBMP-2 only or in presence of FgHBD peptides, and 50% for the gels containing FabCol. In collagen sponges, increased sequestration in presence of FabCol-FgHBD, added at a 1:1 molar ratio with rhBMP-2, was visualized by immunohistochemistry (FIG. 13F). Under the tested experimental conditions, rhBMP-2 showed some retention into collagen sponge, yet the presence of FabCol-FgHBD conjugates substantially increased this retention.

c. Engineering Recombinant Fusion Protein Linking a Collagen-Binding Domain FabCol to LamLG4 or FgHBD Growth Factor-Binding Domains to Sequester rhBMP-2 into Collagen Biomaterials

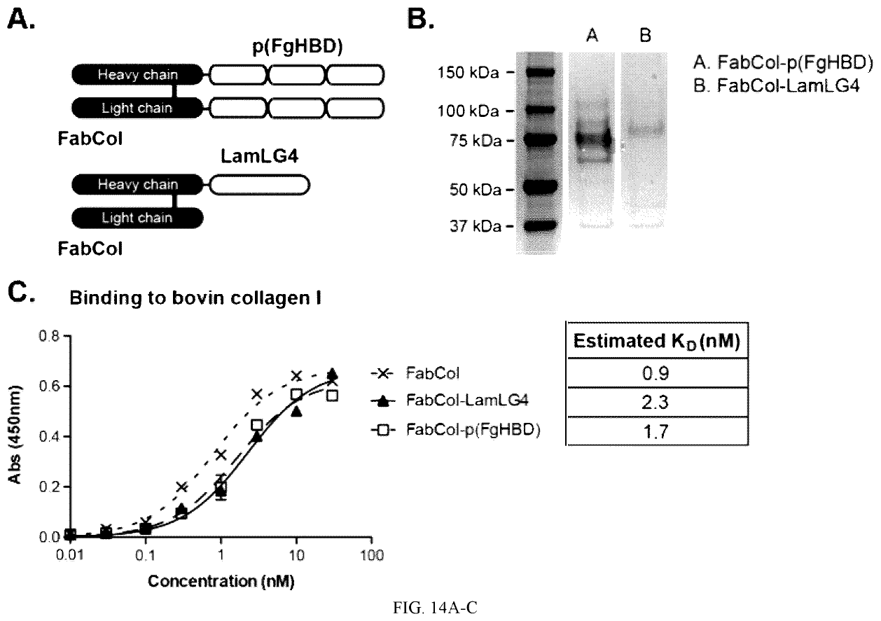

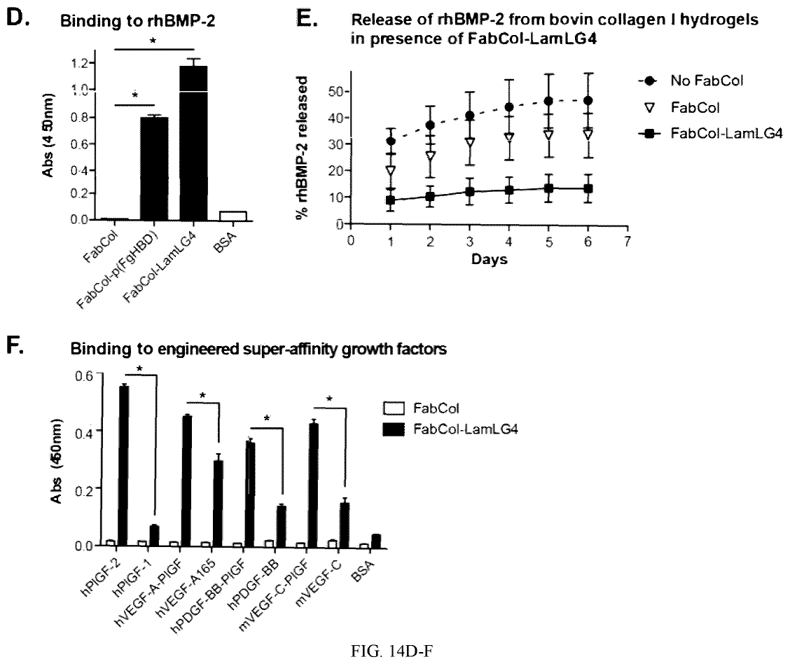

Two recombinant fusion proteins were made to bridge growth factors, particularly rhBMP-2, to collagen biomaterials (FIG. 14A). In a first design, 3 sequential repeats of FgHBD domain separated by glycine-serine linkers were fused to both C-termini of the FabCol light and heavy chains. In a second design, the LamLG4 domain was fused to the C-terminus of the FabCol heavy chain. Both fusion proteins were successfully produced in HEK293 cells and purified using protein A and heparin affinity, confirming the presence of FabCol and the growth factor-binding domains on the fusion proteins. Indeed, both FgHBD and LamLG4 were shown to bind to heparin (Ishihara et al., Nature Communications 2018; Martino et al., PNAS 2013). Purified proteins were analysed by SDS-PAGE, which revealed the presence of multiple bands around 75 kDa for the FabCol-p(FgHBD) variant, which theoretical size is 80 kDa. In contrast, FabCol-LamLG4 variant appeared as a single band around 80 kDa while its theoretical size is 71 kDa (FIG. 14B). Importantly, strong affinity of FabCol-p(FgHBD) and FabCol-LamLG4 to bovine type I collagen was observed by ELISA, with K_{D}s around 1.7 nM and 2.3 nM respectively (FIG. 14C). Similarly, both variants strongly bound to rhBMP-2, with FabCol-LamLG4 being superior to FabCol-p(FgHBD) (FIG. 14D). Finally, release tests showed that rhBMP-2 sequestration into type I collagen is substantially increased in presence of FabCol-LamLG4 (FIG. 14E).

d. Combining FabCol-LamLG4 Bridge Protein Technology with the Engineering of Super-Affinity ECM-Binding Growth Factors to Further Enhance Growth Factors Delivery

Interestingly, the inventors further assessed the affinity of FabCol-LamLG4 to other growth factors and growth factors engineered for super-affinity to the ECM (Martino et al. Science, 2014, WO2014006082A1). Super-affinity growth factors were engineered as fusion of wild-type growth factors with an ECM-binding domain derived from the placental growth factor-2, which allow their strong retention within physiological ECMs, mostly through interactions to glycoproteins (e.g. fibronectin, vitronectin, tenascin) and glycosaminoglycans (e.g. heparan-sulfates GAGs). Because LamLG4 is derived from laminin, a well-known ECM protein of the basement matrix, PlGF-2 engineered growth factors are expected to exhibit higher affinities to FabCol-LamLG4 than the wild-type growth factors. Indeed, one can appreciate in FIG. 14F that the binding of FabCol-LamLG4 to PlGF-2-engineered growth factors was significantly higher than the one to non-engineered wild-type growth factors. This results would suggests that retention of growth factors into collagen biomaterials in presence of FabCol-LamLG4 might be further increased by the engineering of the growth factor using the PlGF-2-derived ECM-binding domain, and so that these two technologies could rationally be used in combination.

FabCol light chain with the human Fab constant region:
(SEQ ID NO: 62)
EIVLTQSPGTLSLSPGERATLSCRASQSVSSSYLAWYQQKPGQAPRLLIY
GASSRATGIPDRFSGSGSGTDFTLTISRLEPEDFAVYYCQQAIGFPQTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
Anti-Collagen light chain variable region:
(SEQ ID NO: 63)
EIVLTQSPGTLSLSPGERATLSCRASQSVSSSYLAWYQQKPGQAPRLLIY
GASSRATGIPDRFSGSGSGTDFTLTISRLEPEDFAVYYCQQAIGFPQTFG
QGTKVEIK
FabCol heavy chain with the human Fab constant
region:
(SEQ ID NO: 64)
EVQLLESGGGLVQPGGSLRLSCAASGFTFSSYAMSWVRQAPGKGLEQVSA
ISGSGGSTYYADSVKGRFTISRDNSKNTLYLQMNSLRAEDTAVYYCAKTL
AAFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCGS
Anti-Collagen heavy chain variable region:
(SEQ ID NO: 65)
EVQLLESGGGLVQPGGSLRLSCAASGFTFSSYAMSWVRQAPGKGLEQVSA
ISGSGGSTYYADSVKGRFTISRDNSKNTLYLQMNSLRAEDTAVYYCAKTL
AAFDYWGQGTLVTV
FgHBD (used for conjugation):
(SEQ ID NO: 66)
GCGGSLRPAPPPISGGGYRARPAKAAATQKKVERKAPDA
In some embodiments, the FgHBD comprises:
(SEQ ID NO: 67)
SLRPAPPPISGGGYRARPAKAAATQKKVERKAPDA
FabCol-LamLG4 heavy chain with the human Fab constant
region (LamLG4 is displayed in italic):
(SEQ ID NO: 71)
EVQLLESGGGLVQPGGSLRLSCAASGFTFSSYAMSWVRQAPGKGLEQVSA
ISGSGGSTYYADSVKGRFTISRDNSKNTLYLQMNSLRAEDTAVYYCAKTL
AAFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCGSGGGSGG

[symbol]

[symbol]

[symbol]

[symbol]

[symbol]

[symbol]

[symbol]

CLKNFQLDSKPLYTPSSSFGVSSCTG.
LamLG4:
(SEQ ID NO: 68)
SLNKPPFLMLLKGSTRFNKTKTFRINQLLQDTPVASPRSVKVWQDACSPL
PKTQANHGALQFGDIPTSHLLFKLPQELLKPRSQFAVDMQTTSSRGLVFH
TGTKNSFMALYLSKGRLVFALGTDGKKLRIKSKEKCNDGKWHTVVFGHDG
EKGRLVVDGLRAREGSLPGNSTISIRAPVYLGSPPSGKPKSLPTNSFVGC
LKNFQLDSKPLYTPSSSFGVSSCTG.
FabCol-p(FgHBD) light chain with the human Fab
constant region (the 3 repeats of p(FgHBD) are
displayed in italic):
(SEQ ID NO: 72)
EIVLTQSPGTLSLSPGERATLSCRASQSVSSSYLAWYQQKPGQAPRLLIY
GASSRATGIPDRFSGSGSGTDFTLTISRLEPEDFAVYYCQQAIGFPQTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGECGAGGGSGG

[symbol]

[symbol]

[symbol]

[symbol]

[symbol]

[symbol]

[symbol]

.
Three repeats of p(FgHBD):
(SEQ ID NO: 69)
GHRPLDKKREEAPSLRPAPPPISGGGYRARPAKAAATQKKVERKAPDAGG
GSGGGSGGGHRPLDKKREEAPSLRPAPPPISGGGYRARPAKAAATQKKVE
RKAPDAGGGSGGGSGGGHRPLDKKREEAPSLRPAPPPISGGGYRARPAKA
AATQKKVERKAPDAGGGT
or
(SEQ ID NO: 70)
GHRPLDKKREEAPSLRPAPPPISGGGYRARPAKAAATQKKVERKAPDAGG
GSGGGSGGGHRPLDKKREEAPSLRPAPPPISGGGYRARPAKAAATQKKVE
RKAPDAGGGSGGGSGGGHRPLDKKREEAPSLRPAPPPISGGGYRARPAKA
AATQKKVERKAPDAGGTG
FabCol-p(FgHBD) heavy chain with the human Fab
constant region (the 3 repeats of p(FgHBD) are
displayed in italic):
(SEQ ID NO: 73)
EVQLLESGGGLVQPGGSLRLSCAASGFTFSSYAMSWVRQAPGKGLEQVSA
ISGSGGSTYYADSVKGRFTISRDNSKNTLYLQMNSLRAEDTAVYYCAKTL
AAFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCGSGGGSGG

[symbol]

[symbol]

[symbol]

[symbol]

[symbol]

[symbol]

.

### Example 4: A Peptide from Von Willebrand Factor that Functions as a Growth Factor Reservoir to Promote Wound Healing

During wound healing, the distribution, availability and signaling of growth factors (GFs) are orchestrated by their binding to extracellular components in the wound microenvironment and provisional matrix. The hemostatic protein von Willebrand factor (vWF) regulates angiogenesis; its deficiency or dysfunction is associated with vascular malformations. This example shows that vWF deficiency delays wound healing accompanied by decreased angiogenesis and decreased amounts of vascular endothelial growth factor-A (VEGF-A) in the wound. In vitro, vWF binds to several GFs and vWF binds to GFs in human serum. Serum from a type 2B von Willebrand disease (vWD) patient carrying the R1341Q point mutation within the vWF peptide showed reduced vWF-GF associations. Incorporation of the vWF peptide into fibrin matrices enabled sequestration and slow release of incorporated GFs. Treatment of chronic skin wounds with VEGF-A165 and platelet derived growth factor (PDGF)-BB incorporated within vWF peptide-functionalized fibrin matrices accelerated wound healing, with increased angiogenesis and smooth muscle cell proliferation. Therefore, the vWF peptide can function as a GFs reservoir, leading to effective angiogenesis and tissue regeneration.

1. Materials and Methods

a. Wound-Healing of vWF-Deficient Mice

Mouse surgical preparation, wounding, splinting, and bandaging was performed as previously described. Briefly, vWF deficient and littermate control mice ages 20 to 24 wk were used. Their backs were shaved and two full-thickness punch biopsy wounds (6 mm in diameter) were created in each mouse. Donut-like silicone disc was used as a splint. The splint was placed on the wound and anchor the splint with 6-0 nylon sutures to ensure positioning. Then, wounds were covered with a adhesive film dressing (Hydrofilm, Hartmann). After 5 d, wounds were collected and used for further analysis. All animal experiments were performed with approval from the Veterinary Authority of the Institutional Animal Care and Use Committee of the University of Chicago and Imperial College London in accordance with the UK Animals (Scientific Procedures) act of 1986.

b. Histomorphometric Analysis of Wound Tissue Sections

[symbol]

Histomorphometric analyses were performed as previously reported. Briefly, an area of 8 mm in diameter, which includes the complete epithelial margins, was excised. Wounds were fixed with 2% PFA and cut in the center into two and embedded into paraffin. Histological analysis was performed on 5 μm serial sections. Images were captured with an EVOS FL Auto microscope (Life Technologies). The extent of re-epithelialization and granulation tissue formation were measured by histomorphometric analysis of tissue sections (H&E staining) using ImageJ software. For analysis of re-epithelialization, the distance that the epithelium had traveled across the wound was measured; the muscle edges of the panniculus carnosus were used as an indicator for the initial wound edges, and re-epithelialization was calculated as the percentage of the distance of edges of the panniculus carnosus muscle. For granulation tissue quantification, the area covered by a highly cellular tissue was determined.

c. Flow Cytometric Analysis of the Wounds

The wounded skins regions were removed, cut into small pieces (<0.5 mm²) and transferred to 1 mL of an enzyme solution (collagenase D (1 mg/mL)) and agitated for 1 hr at 37° C. Then, the cells from digested wounds were re-suspended in PBS, passed through a cell strainer, and centrifuged. Then, cells were stained for 15 min in 100 μL of FACS buffer containing antibodies: anti-CD31 (MEC13.3, BD Biosciences), anti-Ki67 (B56, BD Biosciences), anti-CD45 (30-F11), anti-α-smooth muscle actin (SMA) (R & D systems). Fixable live/dead cell discrimination was performed using Fixable Viability Dye eFluor 455 (eBioscience) according to the manufacturer's instructions. Intracellular staining was performed using the Intracellular Staining Permeabilization Wash Buffer according to manufacturer's instructions (BioLegend). Cells were analyzed using a Fortessa (BD Biosciences) flow cytometer and data was analyzed using FlowJo software (FlowJo, LLC).

d. Quantification of VEGF-A in the Wounds

Wounds were harvested using an 8 mm diameter biopsy punch. The tissue was transferred in 0.9 mL of tissue T-PER Tissue Protein Extraction Reagent (Thermo Scientific) containing 1 mg/mL of collagenase IV (Sigma-Aldrich), and homogenized with a tissue homogenizer. The tissue lysate was incubated 1 hr at 37° C. and 100 μL of a 5 M NaCl solution containing protease inhibitors (1 tablet of protease inhibitor cocktail for 10 mL) added to the lysate. The samples were centrifuged at 10000×g for 5 min, and the supernatants were stored at −80° C. Recombinant human VEGF-A165 in the wound tissue was quantified by ELISA (DuoSet, R&D Systems).

e. Mouse Diabetic Skin Wound Healing Model

Diabetic skin wound healing assays were performed in the mouse as previously reported. Briefly, C57BLKS/J-m/Lepr db (db/db) male mice were 10 to 12 wk old at the start of the experiments. Their backs were shaved and four full-thickness punch biopsy wounds (6 mm in diameter) were created in each mouse. Directly after, fibrin matrices [80 mL total, fibrinogen (10 mg/mL), 2 μM α₂PI₁₋₈-vWF peptide, 100 ng of VEGF-A165, and 50 ng of PDGF-BB] were polymerized on the wounds; the N-terminal α2 plasmin inhibitor peptide (α₂PI₁₋₈) is a substrate for factor XIIIa and provides covalent incorporation of the vWF peptide into fibrin during coagulation, as previously reported for other biomolecules. To avoid drying of the matrices, the wounds were covered with adhesive film dressing (Hydrofilm, Hartmann). Mice were single-caged after the wound surgery. After 7 d, mice were sacrificed and the skin wounds were carefully harvested for histological analysis.

f. Statistical Analysis

Statistically significant differences between experimental groups were determined by one-way ANOVA followed by Tukey's HSD post hoc test with Prism software (v7, GraphPad). For single comparisons, a two-tailed Student's t-test was used. The symbols * and ** indicate p values less than 0.05 and 0.01, respectively; N.S., not significant.

2. Results

a. vWF Deficiency Results in Delayed Wound Healing by Decreased Angiogenesis

The inventors first tested whether endogenous vWF plays a role in dermal wound healing. Full-thickness back-skin wounds were made on vWF-deficient mice and littermate wild-type (WT) controls. After 5 d, wounds were analyzed (FIG. 15). As a result, vWF deficiency significantly delayed wound closure, which was associated with poor granulation tissue formation (FIG. 15A-B). vWF deficiency decreased the proliferation of CD31⁺ endothelial cells and smooth muscle cells (SMCs), in the wounds, suggesting impaired angiogenesis (FIG. 8C-D). The inventors next tested the amount of VEGF-A, a strong angiogenesis inducer, per wound. ELISA after homogenization of wound tissue samples revealed that vWF deficiency decreased the amount of the VEGF-A in the wounds (FIG. 15E). These results suggest that vWF contributes to skin tissue repair through angiogenesis and GF involvement.

b. vWF Binds to Multiple GFs

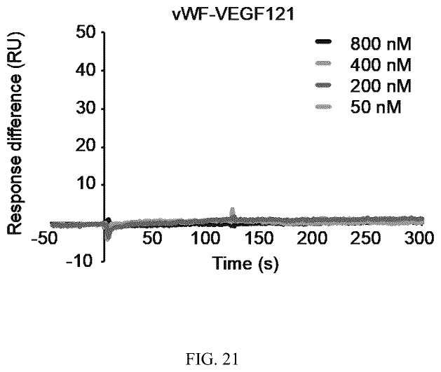

The inventors then tested the hypothesis that vWF promiscuously binds to GFs. A panel of GFs from the PDGF/VEGF, FGF, TGFβ/bone morphogenetic protein (BMP), neurotrophin, and chemokine families were selected. VEGF-A121, which did not show significant binding to vWF by surface plasmon resonance (SPR) (FIG. 21), was used as non-binding reference. The results of the binding screening are shown (FIG. 16A-B). As a result, vWF bound to VEGF-A165, placenta growth factor (PlGF)-2, PDGF-AA, PDGF-BB, PDGF-CC, and PDGF-DD, but not to VEGF-A121 or PlGF-1, neither of which bind heparin. From the FGF family, vWF bound to FGF-2, FGF-7, and FGF-18, but not to FGF-1 or FGF-6. Among the transforming growth factor (TGF)β/bone morphogenetic protein (BMP) family, vWF showed strong binding to TGF-β1 and BMP-2, but not to TGF-03 or BMP-7. Regarding the neurotrophins, both nerve growth factor (β-NGF) and neurotrophin-3 (NT-3) showed relevant binding. Neither insulin-like growth factor-I (IGF-I) nor IGF-II bound to vWF. In addition, epidermal growth factor (EGF) did not show binding to vWF. From the chemokine family, CXCL-11 bound to vWF and CXCL-12α did not, whereas its isoform CXCL-12γ which has an additional HBD in its C-terminus, showed strong binding signal to vWF. These data indicate that vWF binds to multiple heparin binding GFs.

The binding affinity of vWF to VEGF-A165 and PDGF-BB was determined by SPR (FIG. 16C-D). The curves obtained for the specific binding signals were fitted with Langmuir binding kinetics. The binding affinity between VEGF-A165 and vWF was described by a single dissociation constant (K_{D} value) of 27 nM. PDGF-BB had two estimated binding sites, with the lowest K_{D} value of 24 nM. The nM range of K_{D} values demonstrate strong binding affinities of vWF to the tested heparin-binding GFs.

c. vWF Binds to VEGF-A in Human Serum

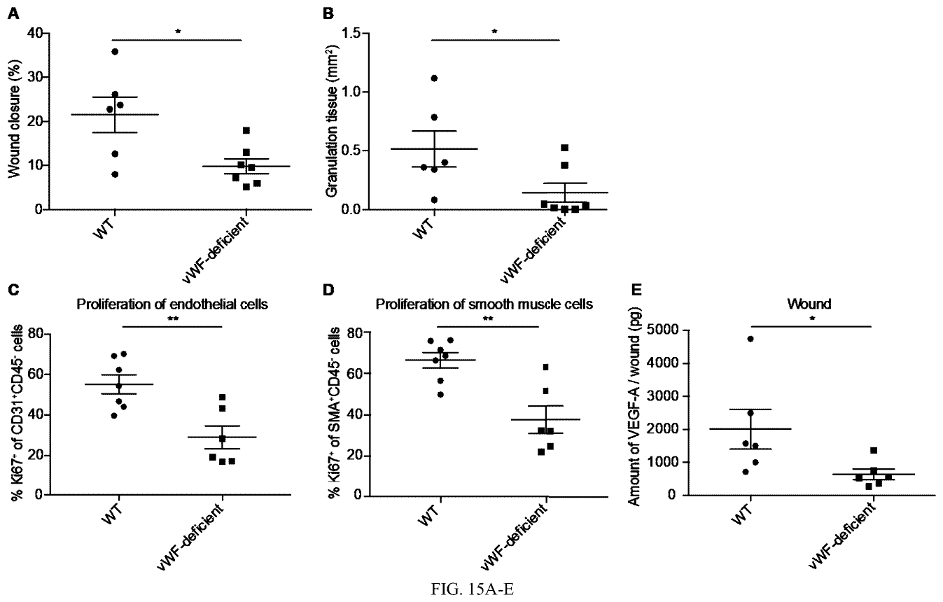

The inventors next tested the presence of the GF-vWF complex in pooled serum from healthy donors. Both sandwich ELISA and immunoprecipitation followed by Western blotting showed that vWF binds to VEGF-A in two different lots of pooled human serum (FIG. 17A-B). These data suggest that VEGF-A-vWF complexes are present in the circulation.

d. The HBD of vWF A1 Domain Binds to Multiple GFs

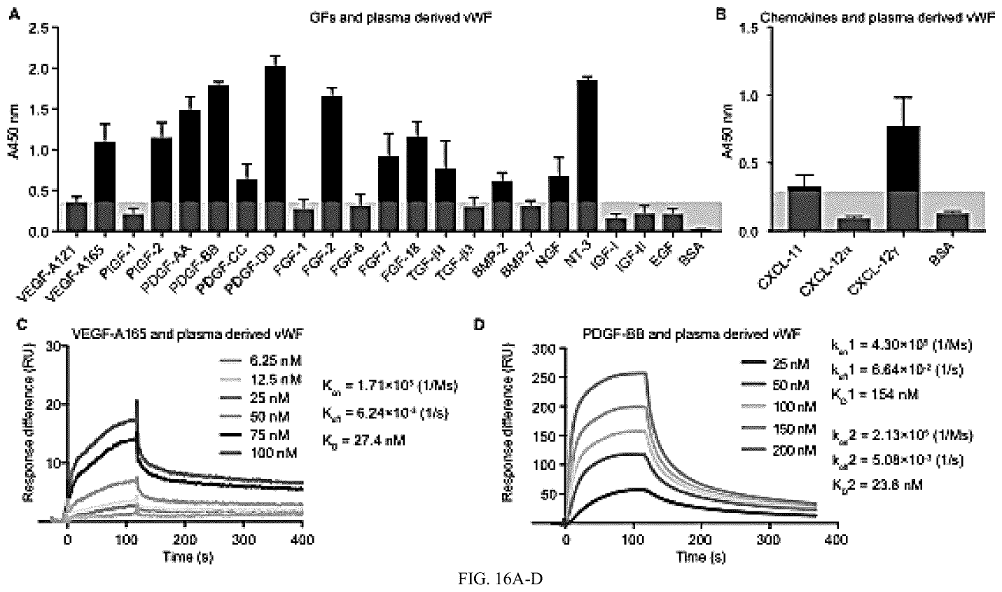

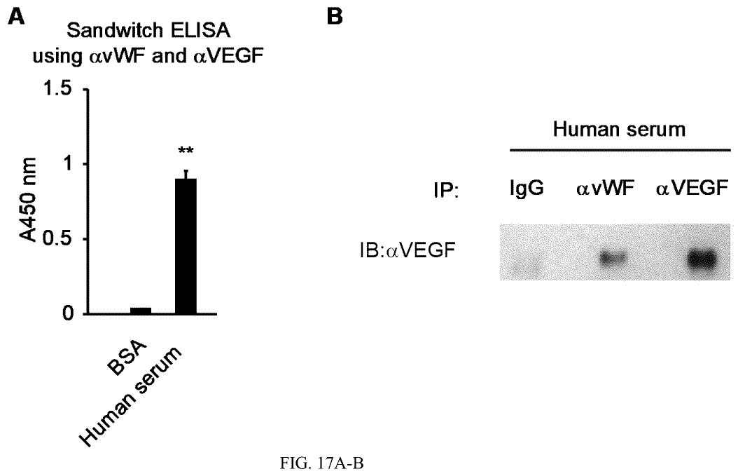

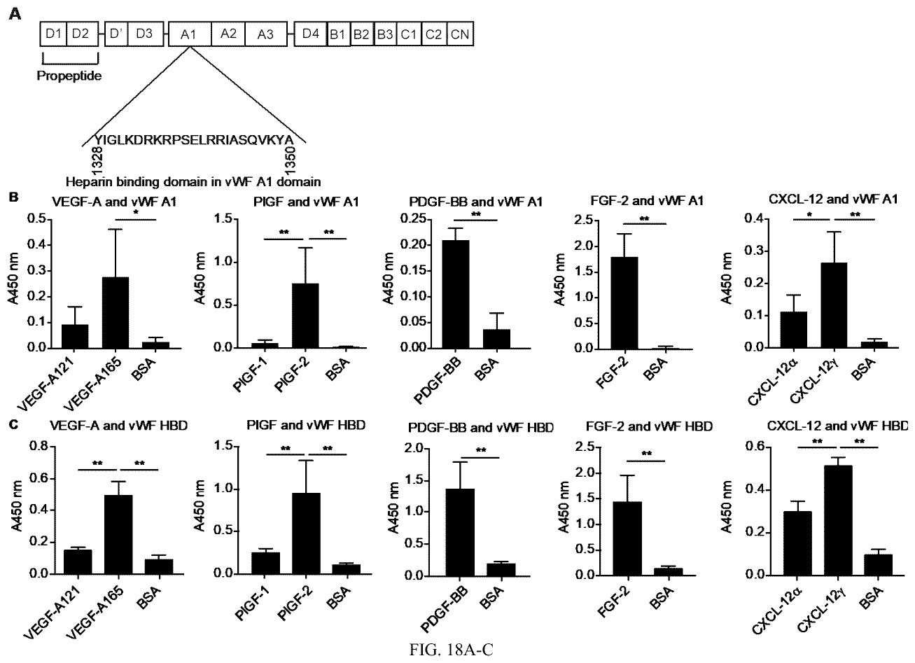

The inventors next investigated the domain within vWF responsible for association with GFs. ELISA assays for vWF binding to VEGF-A165, PlGF-2 or FGF-2, were carried out in the presence of excess (10 μM) heparin. Excess heparin inhibited vWF binding to the GFs (FIG. 22), indicating involvement of HBDs. The HBD of vWF is located in the A1 domain (FIG. 18A); thus the inventors evaluated GF binding to the recombinant A1 domain. VEGF-A165, PlGF-2, PDGF-BB, FGF-2 and CXCL-12γ showed strong binding to recombinant A1 domain, as measured by ELISA (FIG. 18B). The inventors next used a chemically synthesized vWF HBD (24-amino acid peptide, Table 1). In these studies, VEGF-A165, PlGF-2, PDGF-BB, FGF-2 and CXCL-12γ showed binding to the vWF HBD, whereas neither VEGF-A121 nor PlGF-1 were able to bind to the vWF HBD, consistent with the results in FIG. 16 (FIG. 18C). These data show that the vWF A1 peptide binds to GFs.

e. vWF Binds to Heparin-Binding VEGF-A Via the HBD within the A1 Domain

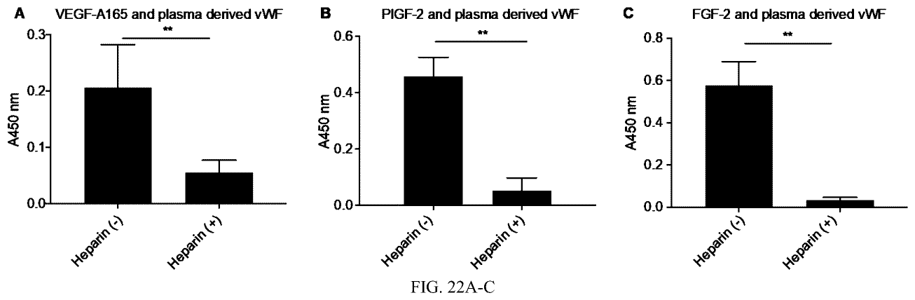

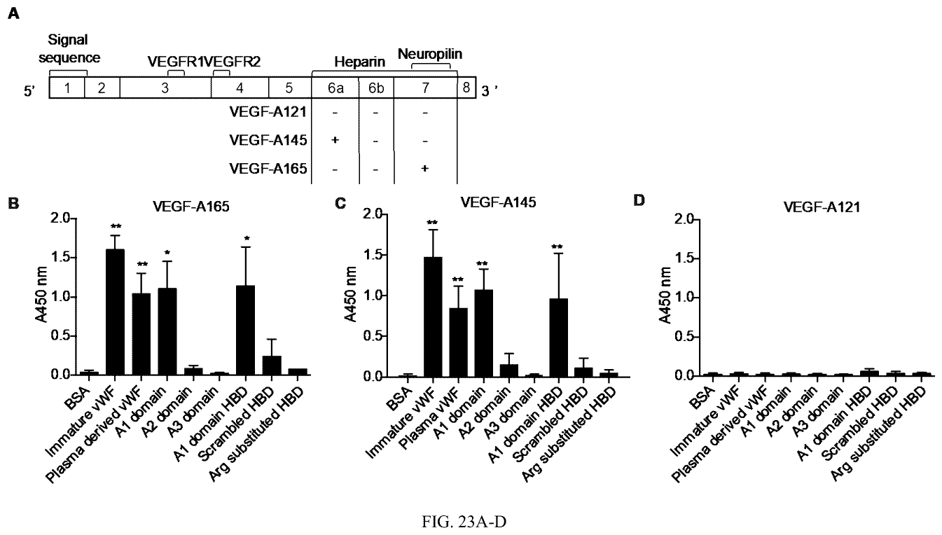

The inventors examined the association between multiple recombinant isoforms of VEGF-A and vWF domains (FIG. 23A). VEGF-A165 was found to bind plasma-derived purified vWF as well as immature, pro-peptide-containing recombinant vWF (FIG. 23B). Similarly, VEGF-A145, which also contains VEGF's HBD, bound to vWF (FIG. 23C), whilst VEGF-A121, which lacks a HBD, did not (FIG. 23D). The vWF A1 domain bound to VEGF-A165 and VEGF-A145. However, no binding of the vWF A2 or A3 domains to VEGF-A165 or VEGF-A145 was detected (FIG. 23B-C). The vWF A1 HBD peptide was also able to bind to VEGF-A165 and VEGF-A145, with a similar magnitude. Scrambling of the amino acid sequence of the vWF A1 HBD abolished the binding (FIG. 23B-C), suggesting that the sequence, not just the total charge, is crucial for the association with VEGF-A165 and VEGF-A145. In addition, substitutions of Arg with Ser in the vWF A1 HBD sequence impaired the binding (FIG. 23B-C), indicating that the positively charged residues are essential. These data demonstrate that the HBDs in vWF A1 domain and in VEGF-A are responsible for binding between the two proteins.

f. Type 2B vWD R1341 Mutation Impairs vWF Binding to GF In Vitro and in Human Serum

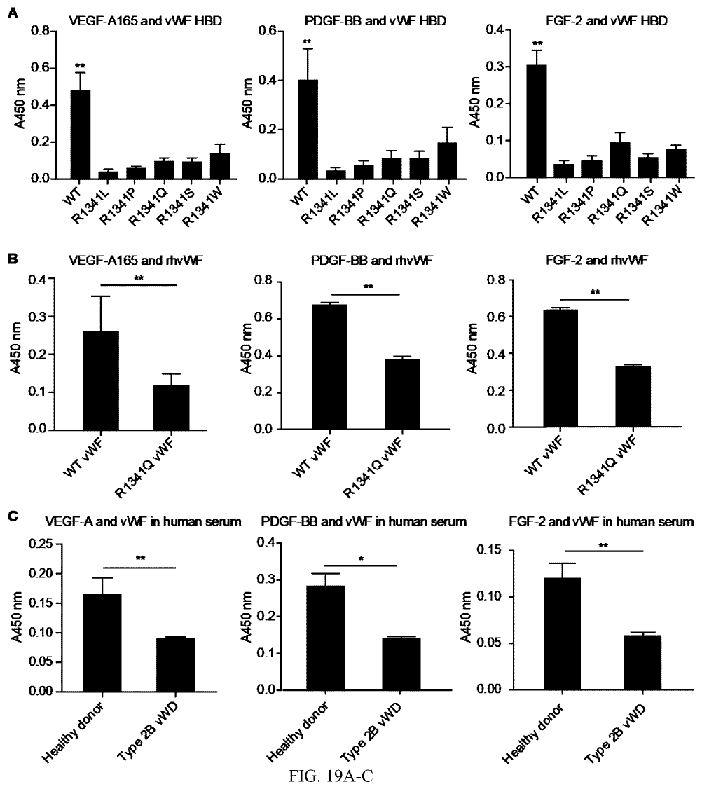

Missense point mutations within the A1 domain of vWF have been reported in patients with type 2B vWD, a subtype where the increased affinity of vWF for GPIbα results in spontaneous platelet aggregation, loss of the most active high molecular weight vWF multimers, thrombocytopenia and bleeding. Type 2B mutations are clustered in exon 28 of the vWF gene, encoding the vWF A1 domain, and some map within the HBD. One such mutation, affecting R1341 within the HBD, has been reported in several patients with type 2B vWD (vWF Variant Database found on the world wide web at vWF.group.shef.ac.uk/), with substitutions to either Leu, Pro, Gln, or Trp. Because Arg in HBDs seems to be crucial for the GF binding (FIG. 23B-C), The inventors next investigated whether this mutation could affect vWF-GF binding. Mutation of R1341 to any of these residues, or Ser, abolished binding between the vWF A1 HBD and GFs (considering VEGF-A165, PDGF-BB, and FGF-2) (FIG. 19A). These data indicate that the R1341 residue is indispensable for binding between vWF A1 HBD and GFs. Crucially, the R1341Q mutation also decreased binding to GFs (i.e. VEGF-A165, PDGF-BB, and FGF-2) to full-length recombinant human vWF, compared to its WT form (FIG. 19B). Moreover, serum from a patient with type 2B vWD carrying the R1341Q mutation displayed decreased vWF binding to GFs (i.e. VEGF-A165, PDGF-BB, and FGF-2), compared to serum from healthy donors (FIG. 19C).

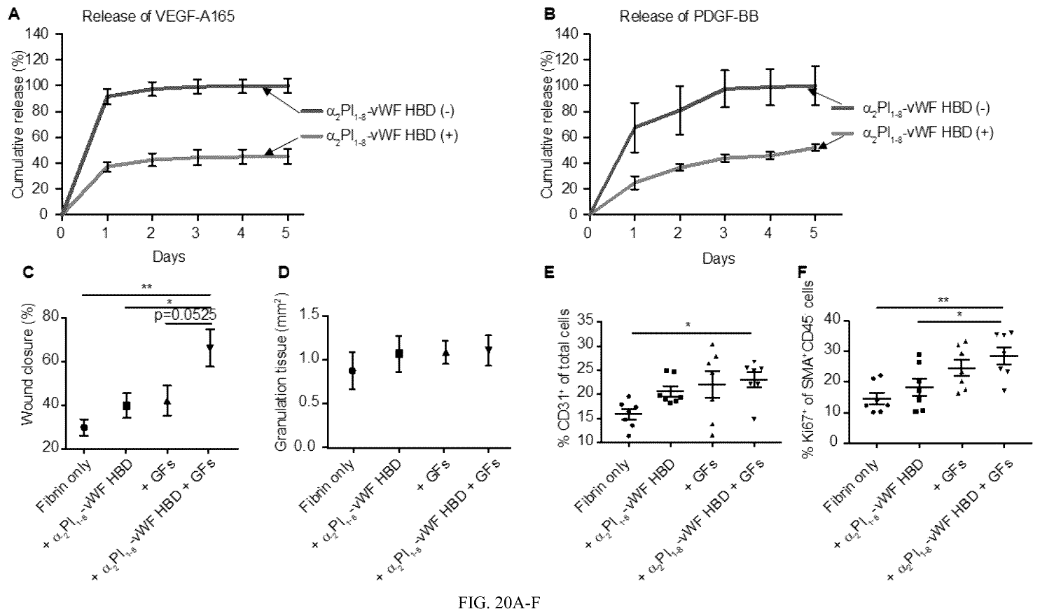

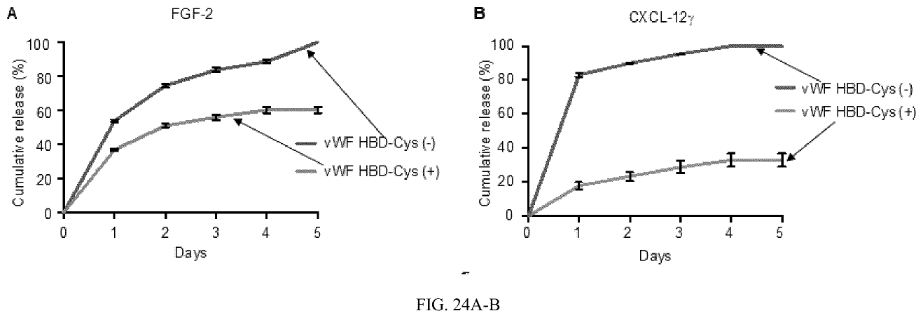

Next, the inventors examined whether vWF HBD peptide is able to improve GF retention within a fibrin matrix, using VEGF-A165 and PDGF-BB, which have been observed to be quickly released from fibrin. Fibrinogen solutions containing GFs and the vWF HBD with integrated factor XIIIa transglutaminase reactive substrate sequence, i.e. α₂PI₁₋₈-vWF HBD, were polymerized to form a fibrin matrix using thrombin and factor XIII. GF release from the matrix was determined by ELISA (FIG. 20A-B). As previously shown, VEGF-A165 and PDGF-BB were quickly released from the unmodified fibrin matrix (>85% released after 1 d). However, by incorporating the α₂PI₁₋₈-vWF HBD peptide, VEGF-A165 and PDGF-BB were retained within the fibrin matrices (45% and 52% retention on day 5, respectively). These results demonstrate that the vWF HBD enhances the function of a fibrin matrix as a GF reservoir. The inventors also observed the effect of vWF HBD on slow-release of other GFs (i.e. CXCL-12γ and FGF-2) from a poly ethylene glycol (PEG)-based synthetic matrix, which has no intrinsic affinity for GFs (FIG. 24). These data show that vWF HBD serves as a GFs reservoir in multiple contexts and for multiple factors.

g. α₂PI₁₋₈-vWF HBD Peptide Functionalized Fibrin Matrix Promotes Chronic Wound Healing In Vivo.

The inventors hypothesized that fibrin matrices functionalized with the vWF HBD peptide could potentiate the effect of GFs due to GF sequestration and resulting slow release from matrices, resulted in enhancing skin wound healing in a delayed wound healing model. A genetic mouse model of type 2 diabetes provides a well-established and clinically relevant experimental system of delayed wound healing, and induction of angiogenesis reportedly promotes wound healing in this model. VEGF-A165 and PDGF-BB, which are crucial angiogenesis inducers and exhibited binding to the vWF HBD, were incorporated within a fibrin matrix. As above, the inventors used the Factor XIIIa-induced coupling of the α₂PI₁₋₈ sequence to fibrin with the α₂PI₁₋₈-vWF HBD to functionalize the matrix. Four groups of treatment were established: fibrin only, fibrin functionalized with α₂PI₁₋₈-vWF HBD, fibrin containing the GFs, and fibrin functionalized with α₂PI₁₋₈-vWF HBD containing the GFs. After 7 d, histology of wounded skin was analyzed. The wounds that received fibrin matrices containing only GFs or vWF HBD did not differ from wounds treated with fibrin alone, in either amount of granulation tissue or degree of wound closure (FIG. 20C). In contrast, the combined delivery of VEGF-A165 and PDGF-BB by fibrin functionalized with α₂PI₁₋₈-vWF HBD led to significantly faster wound closure due to re-epithelialization. The development of granulation tissue was maintained (FIG. 20D). The inventors next examined endothelial cells in the wounds (FIG. 20E). Co-delivery of VEGF-A165 and PDGF-BB in fibrin functionalized with α₂PI₁₋₈-vWF HBD led to a significantly increased frequency of CD31⁺CD45⁻ endothelial cells compared to fibrin only group after 5 d of wounding. Co-delivery of VEGF-A165 and PDGF-BB in α₂PI₁₋₈-vWF HBD functionalized fibrin significantly increased frequency of Ki67±, a proliferation marker, within SMCs compared to fibrin only and α₂PI₁₋₈-vWF HBD functionalized fibrin only treatment groups on day 5 (FIG. 20F). These data show that treatment with α₂PI₁₋₈-vWF HBD and GFs incorporated within a fibrin matrix promoted wound healing via angiogenesis by sequestration and slow release of VEGF-A165 and PDGF-BB.

h. vWF HBD does not Affect Endothelial or Fibroblast Proliferation In Vitro.

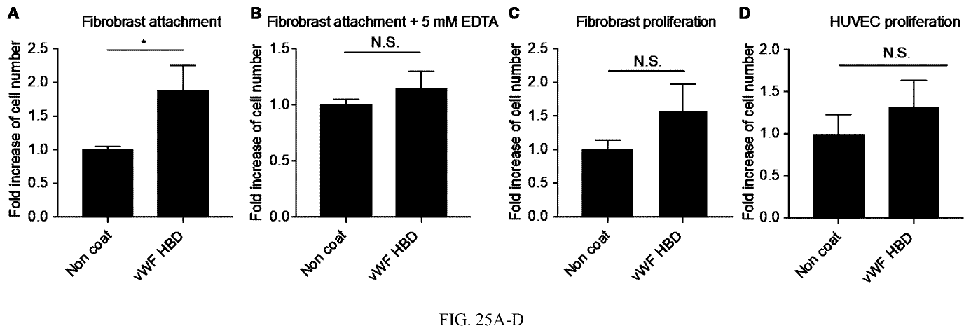

The inventors next tested functions of the vWF HBD on fibroblast and endothelial cell attachment and proliferation. vWF HBD peptide coating significantly enhanced fibroblast attachment (FIG. 25A); this effect was inhibited by adding 5 mM ethylenediaminetetraacetic acid (EDTA) to the in vitro culture, suggesting that vWF HBD peptide may bind to cation-dependent cell adhesion receptors such as integrins (FIG. 25B). Coating of the vWF HBD peptide on cell culture plates did not significantly affect fibroblast proliferation in the presence of FGF-2, suggesting that the vWF HBD may slightly enhance cell adhesion, but did not induce cell proliferation in concert with at least this GF in vitro (FIG. 25C). Similarly, vWF HBD did not affect endothelial proliferation in vitro (FIG. 25D). These data support that, in the context of wound healing and tissue repair, the vWF HBD acts as a GF reservoir rather than a cell scaffold, promoting effective wound healing and angiogenesis through its binding to the growth factors.

i. Growth Factors and Chemokines

All GFs and chemokines were purchased in their mature forms, highly pure (>95% pure), carrier-free, and lyophilized, as previously reported1. VEGF-A121, VEGF-A165, PlGF-1, PlGF-2, PDGF-AA, PDGF-BB, PDGF-CC, PDGF-DD, FGF-1, FGF-2, FGF-6, FGF-7, FGF-18, TGF-β1, TGF-β3, BMP-2, BMP-7, NGF, NT-3, IGF-I, IGF-II, EGF, CXCL-11, and CXCL-12α were purchased from PeproTech. CXCL-12γ was purchased from R & D Systems. Except for PDGF-DD, TGF-β1, TGF-β3, and BMP-7, which were produced in eukaryotic cells, all GFs were produced in Escherichia coli and thus were not glycosylated. All GFs were reconstituted and stored according to the provider's instructions to regain full activity and prevent loss of protein.

j. Detection of vWF Binding to Recombinant GFs

ELISA tests were performed as previously reported1. In brief, ELISA plates (med-binding, Greiner Bio-One) were coated with 50 nM GFs at 37° C. for 2 hrs. After blocking with 2% BSA solution containing PBS-T, 1 μg/mL of plasma-derived vWF (EMD Millipore) was added. Bound vWF was detected with 1 μg/mL of rabbit anti-human vWF antibody (Sino Biological). Then, HRP conjugated goat anti-rabbit antibody (Jackson ImmunoResearch) was added. After 60 min of incubation, 50 μL TMB substrate (Sigma-Aldrich) was added. The reaction was stopped by adding 25 μL of 2N H2SO4. Subsequently, the absorbance at 450 nm was measured and subtracted the absorbance at 570 nm.

k. Surface Plasmon Resonance (SPR)

SPR assays were performed as described previously2. In brief, measurements were made with a Biacore X100 SPR system or Biacore 3000 SPR system (GE Healthcare). Plasma-derived vWF was immobilized via amine coupling on a C1 chip (GE Healthcare) for ˜2000 resonance units (RU) according to the manufacturer's instructions. Recombinant human VEGF-A165, VEGF-A121, or PDGF-BB was flowed at increasing concentrations in the running buffer at 30 μL/min. The sensor chip was regenerated with glycine at pH 2 for every cycle. Specific binding of GFs to vWF was calculated automatically using the response to a non-functionalized channel as a reference. Binding curves were fitted using BIAevalution software (GE Healthcare). vWF-VEGF-A165 binding results were fitted with Langmuir binding kinetics (1:1 binding with drifting baseline Rmax local). vWF-PDGF-BB binding results were fitted with heterogeneous ligand-parallel reaction.

l. Inhibition of vWF-GF Binding by Heparin

ELISA plates (med-binding) were coated with 10 μg/mL vWF. Then, wells were blocked with 2% BSA-containing PBS-T and further incubated with 1 μg/mL each of VEGF-A, PlGF-2, or FGF-2 for 60 min at room temperature (RT) with 10 μM heparin. Next, the wells were incubated with biotinylated anti-VEGF-A, anti-PlGF, or anti-FGF-2 antibodies (R & D Systems). The antibodies were detected by streptavidin-HRP (R & D Systems). Color development and the absorbance measurement were done as described above.

m. Detection of vWF Binding to VEGF-A by Western Blotting

One mL of human serum was immunoprecipitated with 10 μg of monoclonal rabbit anti-human vWF antibody (SinoBiological) or control rabbit IgG (EMD Millipore) and 50 μL of protein G-agarose (Thermo Fisher Scientific) overnight at 4° C. The resulting pellet was dissolved in Laemmli buffer and subjected to Western blot analysis. Western blot analysis was performed after SDS-PAGE (4-20% gradient gel, Bio-Rad) and transfer onto MS nitrocellulose membranes (Membrane Solutions). GFs were detected using 1 μg/mL biotinylated antibodies for human VEGF-A (R & D Systems), followed by incubation with HRP conjugated streptavidin (R & D Systems) at 1:200 dilutions. The proteins were detected and visualized with the ECL Plus Western Blotting Detection System (GE Healthcare).

n. Detection of vWF Binding to GFs in Human Serum by ELISA

The study was approved by the ethics committees of the Hammersmith, Queen Charlotte's, and Royal Marsden hospitals; informed consent was obtained from all individuals in accordance with the Declaration of Helsinki. ELISA plates (med-binding) were coated with 10 μg/mL rabbit monoclonal anti-human vWF antibody (clone: 111, SinoBiological). Then, wells were blocked with 2% BSA-containing PBS-T and further incubated with human serum derived from healthy donor (Sigma-Aldrich) or type 2B vWD patient for 60 min at RT. Next, the wells were incubated with biotinylated antibodies for human VEGF-A, PDGF-BB or FGF-2 (R & D Systems). The antibodies were detected by streptavidin-HRP (R & D Systems). Color development and the absorbance measurement were done as described above.

o. Expression of Recombinant vWF

The expression vector pcDNA-full length(FL)-vWF has been previously described3. R1341 residue was mutated to Glutamine (Q) using the QuikChange® XL site-directed mutagenesis kit (Stratagene). The sequences were verified and fragments containing mutations were subcloned into a vector containing full length vWF. Briefly, the 5′ XhoI to KpnI fragment was digested from pGEM (XhoI-KpnI) while the 5′KpnI to AgeI fragment from pcDNA3.1-A2-CK vector, those were then cloned into pcDNA 3.1 FL-vWF-KpnI that had been digested with the same enzymes. Recombinant WT and R1341Q vWF were expressed in HEK293T cells as previously described using 10 mM polyethylenimine (PEI) as transfection reagent3. The conditioned medium was collected after 3 days, filtered and if required, concentrated or purified for further analysis. Recombinant vWF was purified using a combination of ion-exchange and heparinSepharose affinity chromatography as previously described3,4. Briefly, filtered vWF expression medium was applied to an SK-16 chromatography column (Amersham Pharmacia, UK) previously packed with Fractogel-EMD-TMAE+ (Merck) according to manufacturers instructions. The VWF was then eluted using 20 mM Tris, 500 mM NaCl, pH 7.4 and dialysed into 20 mM Tris, 150 mM NaCl, pH 7.4 and further purified using a HeparinSepharose 6 fast flow column (Amersham Pharmacia, UK). The purity of vWF was assessed by SDS-PAGE gel electrophoresis and concentration determined by vWF-ELISA.

p. Detection of Recombinant GF Binding to the vWF Recombinant Protein and A1 HBD Peptide.

ELISA tests were performed as described above. In brief, ELISA plates were coated with 1 μg/mL of FL-vWF (WT or R1341Q), 1 μg/mL of vWF A1 recombinant protein (U-Protein Express) or 1 μg/mL of vWF A1 HBD peptide (sequence YIGLKDRKRPSELRRIASQVKYA, (SEQ ID NO:50) chemically synthesized by Genscript) at 37° C. overnight. After blocking with 2% BSA solution containing PBS-T, 1 μg/mL of the recombinant human proteins VEGF-A121, VEGF-A165, PlGF-1, PlGF-2, PDGF-BB, FGF-2, CXCL-12α and CXCL-12γ were added. 1 μg/mL of BSA served as non-binding protein control. Bound GF or chemokine was detected with biotinylated antibodies for human VEGF-A, PlGF, PDGF-BB, FGF-2, or CXCL-12 (R & D Systems). The antibodies were detected by streptavidin-HRP (R & D Systems). Color development and the absorbance measurement were done as described above.

q. Detection of vWF Binding to Recombinant VEGF-A Isoforms

ELISA was performed as previously reported1. In brief, ELISA plates (med binding: Greiner Bio-One) were coated with 50 nM BSA (GE Healthcare), pro-peptide containing recombinant vWF (Sino Biological), plasma-derived vWF (EMD Millipore), recombinant human vWF A1 domain (U-Protein Express), recombinant human vWF A2 domain (R & D systems), recombinant human vWF A3 domain (U-Protein Express), vWF A1 HBD peptide or scrambled/mutated HBD peptide (all peptides were synthesized by Genscript). After blocking with 2% BSA solution containing PBS-T, 1 μg/mL of recombinant human VEGF-A121 (PeproTech), recombinant human VEGF-A145 (R & D Systems), or recombinant human VEGF-A165 (PeproTech) was added. Bound VEGF-A was detected with 1 μg/mL of mouse anti-human VEGF-A antibody (clone: 26503, R & D systems). After 60 min of incubation, horseradish peroxidase (HRP) conjugated goat anti-mouse antibody (1:2000 dilution, Dako) was added and incubated for another 60 min. Color development and the absorbance measurement were done as described above.

r. Release of GF from Fibrin Matrix

Fibrin matrices were generated with human fibrinogen as described previously1,5. In brief, fibrin matrices were generated with 8 mg/mL fibrinogen, 2 U/mL human thrombin (Sigma-Aldrich), 4 U/mL factor XIIIa (Fibrogammin; Behring), 5 mM calcium chloride, 2 μM □2PI1-8-vWF HBD peptide (NQEQVSPLYIGLKDRKRPSELRRIASQVKYA (SEQ ID NO:51), chemically synthesized by Genscript), and 500 ng/mL recombinant human VEGF-A165 or PDGF-BB. Fibrin gels were polymerized at 37° C. for 1 hr and transferred into 24-well Ultra Low Cluster plates (Corning) containing 500 μL of buffer (20 mM Tris-HCl, 150 mM NaCl, and 0.1% BSA; pH 7.4). A control well that served as a 100% released control contained only the GF in 500 μL of buffer. Every 24 hr, buffers were removed, stored at −20° C., and replaced with fresh buffer. For the 100% released control well, 20 μL of buffer was removed each day. After 5 d, the cumulative release of GF was quantified by ELISA (DuoSet; R&D Systems), using the 100% released control as a reference.

s. Release of GFs from Fibrin-Mimetic Matrix

Fibrin-mimetic matrices were formed from reactive PEG precursors as previously described6. Matrices (50 μL) were generated in 50 mM Tris buffer (pH 7.6) to obtain 1.75% (wt/vol) PEG, 10 μM vWF HBD-Cys (YIGLKDRKRPSELRRIASQVKYAC (SEQ ID NO:49), chemically synthesized by Genscript), 10 U/mL factor XIIIa, 50 mM CaCl2, 1 μg/mL FGF-2 and 1 μg/mL CXCL-12γ. Fibrin-mimetic gels were polymerized at 37° C. for 1 hr and then transferred into 24-well Ultra Low Cluster plates (Corning) containing 1 mL of buffer (20 mM Tris-HCl, 150 mM NaCl, and 0.1% BSA; pH 7.4). A control well that served as 100% released control contained only the GFs in 1 mL of buffer. Every 24 hr, buffers were removed, stored at −20° C., and replaced with fresh buffer. For the 100% released control well, 20 μL of buffer was removed each day and stored at −20° C. Cumulative release of GF was quantified by ELISA (DuoSet; R&D Systems), using the 100% released control as a reference.

t. Cell Adhesion Assay

Cell adhesion assays were performed using starved human lung fibroblasts (Lonza) in FGM-2 medium (Lonza) with or without 5 mM EDTA. Cells were plated at 3000 cells/well on 1 μg/mL vWF HBD pre-coated 96-well plates (non-tissue culture treated, Greiner Bio-one) and incubated for 30 min at 37° C. Then, the medium was removed, and wells were further washed three times with new FGM-2 medium. Cell numbers were quantified using a CyQUANT assay (Invitrogen).

u. Cell Proliferation Assay with vWF HBD

Cell proliferation assays were performed as previously reported1. Briefly, human lung fibroblasts (Lonza) were cultured using FGM-2 medium (Lonza) (1000 cells/well) or human umbilical vein endothelial cells (HUVEC, Lonza) were cultured using EGM-2 medium (Lonza) (1000 cells/well) on 1 μg/mL vWF HBD pre-coated 96-well plates (Tissue culture treated, Falcon). Cell numbers were quantified after 72 hrs using a CyQUANT assay (Invitrogen).

TABLE 1
THE SEQUENCES OF VWF A1 HBD PEPTIDES.

| SEQ | | |
|---|---|---|
| ID | | |
| NO | Name | Peptide sequence |
| 49 | vWF A1 HBD | YIGLKDRKRPSELRRIASQVKYAC |
| 52 | Scrambled HBD | LYCEIARGYSLKRKVPDQIRSRKA |
| 53 | Arg substituted | YIGLKDSKSPSELSSIASQVKYAC |
| | HBD | |
| 54 | Naïve | YIGLKDRKRPSELRRIASQVKYA |
| 55 | R1341L | YIGLKDRKRPSELLRIASQVKYA |
| 56 | R1341P | YIGLKDRKRPSELPRIASQVKYA |
| 57 | R1341Q | YIGLKDRKRPSELQRIASQVKYA |
| 58 | R1341W | YIGLKDRKRPSELWRIASQVKYA |
| 59 | R1341S | YIGLKDRKRPSELSRIASQVKYA |

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references, to the extent that they provide exemplary procedural or other details supplementary to those set forth herein, are specifically incorporated herein by reference.
1. De Cristofaro, R, Peyvandi, F, Baronciani, L et al. Molecular mapping of the chloride-binding site in von Willebrand factor (VWF): energetics and conformational effects on the VWF/ADAMTS-13 interaction. J Biol Chem. 2006; 281(41):30400-30411.
2. Emsley, J, Cruz, M, Handin, R, Liddington, R. Crystal structure of the von Willebrand Factor A1 domain and implications for the binding of platelet glycoprotein Ib. J Biol Chem. 1998; 273(17):10396-10401.
3. Randi, A M, Laffan, M A. Von Willebrand factor and angiogenesis: basic and applied issues. J Thromb Haemost. 2017; 15(1):13-20.
4. Selvam, S N, Casey, L J, Bowman, M L et al. Abnormal angiogenesis in blood outgrowth endothelial cells derived from von Willebrand disease patients. Blood Coagul Fibrinolysis. 2017; 28(7):521-533.
5. Barg, K, Wiewiorski, M, Anderson, A E et al. Total ankle replacement in patients with von Willebrand disease: mid-term results of 18 procedures. Haemophilia. 2015; 21(5):e389-401.
6. Denis, C V, Andre, P, Saffaripour, S, Wagner, D D. Defect in regulated secretion of P-selectin affects leukocyte recruitment in von Willebrand factor-deficient mice. Proc Natl Acad Sci USA. 2001; 98(7):4072-4077.
7. Jenkins, P V, Pasi, K J, Perkins, S J. Molecular modeling of ligand and mutation sites of the type A domains of human von Willebrand factor and their relevance to von Willebrand's disease. Blood. 1998; 91(6):2032-2044.
8. Morales, L D, Martin, C, Cruz, M A. The interaction of von Willebrand factor-A1 domain with collagen: mutation G1324S (type 2M von Willebrand disease) impairs the conformational change in A1 domain induced by collagen. J Thromb Haemost. 2006; 4(2):417-425.
9. Sobel, M, McNeill, P M, Carlson, P L et al. Heparin inhibition of von Willebrand factor-dependent platelet function in vitro and in vivo. J Clin Invest. 1991; 87(5):1787-1793.
10. Martino, M M, Briquez, P S, Guc, E et al. Growth factors engineered for super-affinity to the extracellular matrix enhance tissue healing. Science. 2014; 343(6173):885-888.
11. Martino, M M, Briquez, P S, Ranga, A, Lutolf, M P, Hubbell, J A. Heparin-binding domain of fibrin (ogen) binds growth factors and promotes tissue repair when incorporated within a synthetic matrix. Proceedings of the National Academy of Sciences. 2013; 110(12):4563-4568.
12. Martino, M M,Hubbell, J A. The 12th-14th type III repeats of fibronectin function as a highly promiscuous growth factor-binding domain. The FASEB Journal. 2010; 24(12):4711-4721.
13. De Laporte, L, Rice, J J, Tortelli, F, Hubbell, J A. Tenascin C promiscuously binds growth factors via its fifth fibronectin type III-like domain. PLoS One. 2013; 8(4):e62076.
14. O'Regan, A, Berman, J S. Osteopontin: a key cytokine in cell-mediated and granulomatous inflammation. Int J Exp Pathol. 2000; 81(6):373-390.
15. Dalton, B A, McFarland, C D, Underwood, P A, Steele, J G. Role of the heparin binding domain of fibronectin in attachment and spreading of human bone-derived cells. J Cell Sci. 1995; 108 (Pt 5)(5):2083-2092.
16. Briquez, P S, Hubbell, J A, Martino, M M. Extracellular Matrix-Inspired Growth Factor Delivery Systems for Skin Wound Healing. Adv Wound Care (New Rochelle). 2015; 4(8):479-489.
17. Mitchell, AC, Briquez, P S, Hubbell, J A, Cochran, J R. Engineering growth factors for regenerative medicine applications. Acta Biomater. 2016; 30:1-12.
18. Martino, M M, Tortelli, F, Mochizuki, M et al. Engineering the growth factor microenvironment with fibronectin domains to promote wound and bone tissue healing. Sci Transl Med. 2011; 3(100):100ra189.
19. Chen, T T, Luque, A, Lee, S, Anderson, S M, Segura, T, Iruela-Arispe, M L. Anchorage of VEGF to the extracellular matrix conveys differential signaling responses to endothelial cells. J Cell Biol. 2010; 188(4):595-609.
20. Lutolf, M P, Weber, F E, Schmoekel, H G et al. Repair of bone defects using synthetic mimetic s of collagenous extracellular matrices. Nature biotechnology. 2003; 21(5):513-518.
21. Dunn, L, Prosser, H C, Tan, J T, Vanags, L Z, Ng, M K, Bursill, C A. Murine model of wound healing. J Vis Exp. 2013(75):e50265.
22. Starke, R D, Ferraro, F, Paschalaki, K E et al. Endothelial von Willebrand factor regulates angiogenesis. Blood. 2011; 117(3):1071-1080.
23. Schense, J C, Hubbell, J A. Cross-linking exogenous bifunctional peptides into fibrin gels with factor XIIIa. Bioconjugate chemistry. 1999; 10(1):75-81.
24. Vempati, P, Popel, A S, Mac Gabhann, F. Extracellular regulation of VEGF: isoforms, proteolysis, and vascular patterning. Cytokine Growth Factor Rev. 2014; 25(1):1-19.
25. Rueda, P, Balabanian, K, Lagane, B, Staropoli, I, Chow, K, Gold, J A. The CXCL12c Chemokine Displays Unprecedented Structural and Functional. 2008.
26. Janowski, M. Functional diversity of SDF-1 splicing variants. Cell Adh Migr. 2009; 3(3):243-249.
27. Fujimura, Y, Titani, K, Holland, L Z et al. A heparin-binding domain of human von Willebrand factor. Characterization and localization to a tryptic fragment extending from amino acid residue Val-449 to Lys-728. J Biol Chem. 1987; 262(4):1734-1739.
28. Nurden, P, Debili, N, Vainchenker, W et al. Impaired megakaryocytopoiesis in type 2B von Willebrand disease with severe thrombocytopenia. Blood. 2006; 108(8):2587-2595.
29. Tischer, A, Madde, P, Moon-Tasson, L, Auton, M. Misfolding of vWF to pathologically disordered conformations impacts the severity of von Willebrand disease. Biophys J. 2014; 107(5):1185-1195.
30. Wood, N, Standen, G R, Murray, E W et al. Rapid genotype analysis in type 2B von Willebrand's disease using a universal heteroduplex generator. Br J Haematol. 1995; 89(1):152-156.
31. Sullivan, S R, Underwood, R A, Gibran, N S et al. Validation of a model for the study of multiple wounds in the diabetic mouse (db/db). Plast Reconstr Surg. 2004; 113(3):953-960.
32. Krilleke, D, Ng, Y S, Shima, D T. The heparin-binding domain confers diverse functions of VEGF-A in development and disease: a structure-function study. Biochem Soc Trans. 2009; 37(Pt 6):1201-1206.
33. Stalmans, I, Ng, Y S, Rohan, R et al. Arteriolar and venular patterning in retinas of mice selectively expressing VEGF isoforms. J Clin Invest. 2002; 109(3):327-336.
34. Gerhardt, H, Golding, M, Fruttiger, M et al. VEGF guides angiogenic sprouting utilizing endothelial tip cell filopodia. J Cell Biol. 2003; 161(6):1163-1177.
35. Lee, R J, Springer, M L, Blanco-Bose, W E, Shaw, R, Ursell, P C, Blau, H M. VEGF gene delivery to myocardium: deleterious effects of unregulated expression. Circulation. 2000; 102(8):898-901.
36. Sundberg, C, Nagy, J A, Brown, L F et al. Glomeruloid microvascular proliferation follows adenoviral vascular permeability factor/vascular endothelial growth factor-164 gene delivery. Am J Pathol. 2001; 158(3):1145-1160.
37. Randi, A M, Laffan, M A, Starke, R D. Von Willebrand factor, angiodysplasia and angiogenesis. Mediterr J Hematol Infect Dis. 2013; 5(1):e2013060.
38. Zanetta, L, Marcus, S G, Vasile, J et al. Expression of Von Willebrand factor, an endothelial cell marker, is up-regulated by angiogenesis factors: a potential method for objective assessment of tumor angiogenesis. Int J Cancer. 2000; 85(2):281-288.
39. Fonder, M A, Lazarus, G S, Cowan, D A, Aronson-Cook, B, Kohli, A R, Mamelak, A J. Treating the chronic wound: A practical approach to the care of nonhealing wounds and wound care dressings. J Am Acad Dermatol. 2008; 58(2):185-206.
40. Falanga, V. Wound healing and its impairment in the diabetic foot. Lancet. 2005; 366(9498):1736-1743.
41. Marti-Carvajal, A J, Gluud, C, Nicola, S et al. Growth factors for treating diabetic foot ulcers. Cochrane Database Syst Rev. 2015(10):CD008548.
42. Galiano, R D, Tepper, O M, Pelo, C R et al. Topical Vascular Endothelial Growth Factor Accelerates Diabetic Wound Healing through Increased Angiogenesis and by Mobilizing and Recruiting Bone Marrow-Derived Cells. The American Journal of Pathology. 2004; 164(6):1935-1947.
43. Chan, R K, Liu, P H, Pietramaggiori, G, Ibrahim, S I, Hechtman, H B, Orgill, D P. Effect of recombinant platelet-derived growth factor (Regranex) on wound closure in genetically diabetic mice. J Burn Care Res. 2006; 27(2):202-205.
44. Martino, M M, Briquez, P S, Guc, E et al. Growth factors engineered for super-affinity to the extracellular matrix enhance tissue healing. Science. 2014; 343(6173):885-888.
45. Martino, M M, Briquez, P S, Ranga, A, Lutolf, M P, Hubbell, J A. Heparin-binding domain of fibrin (ogen) binds growth factors and promotes tissue repair when incorporated within a synthetic matrix. Proceedings of the National Academy of Sciences. 2013; 110(12):4563-4568.
46. Nowak, A A, Canis, K, Riddell, A, Laffan, M A, McKinnon, T A. O-linked glycosylation of von Willebrand factor modulates the interaction with platelet receptor glycoprotein Ib under static and shear stress conditions. Blood. 2012; 120(1):214-222.
47. McKinnon, T A, Chion, A C, Millington, A J, Lane, D A, Laffan, M A. N-linked glycosylation of VWF modulates its interaction with ADAMTS13. Blood. 2008; 111(6):3042-3049.
48. Martino, M M, Tortelli, F, Mochizuki, M et al. Engineering the growth factor microenvironment with fibronectin domains to promote wound and bone tissue healing. Sci Transl Med. 2011; 3(100):100ra189.
49. Lutolf, M P, Weber, F E, Schmoekel, H G et al. Repair of bone defects using synthetic mimetics of collagenous extracellular matrices. Nature biotechnology. 2003; 21(5):513-518.

1. A peptide comprising a growth factor binding domain having an amino acid sequence that is at least 80% identical to one of SEQ ID NOS:1-7, 13-15, 49-50, or 66-70, or a fragment thereof; wherein the peptide is less than 300 amino acids in length.

2. The peptide of claim 1, wherein the peptide is attached to a transglutaminase-reactive peptide.

3. (canceled)

4. The peptide of claim 2, wherein the transglutaminase-reactive peptide is from the α2-plasmin inhibitor.

5-11. (canceled)

12. The peptide of claim 1, wherein the peptide is linked to one or more additional peptides, wherein each additional peptide has an amino acid sequence that is at least 80% identical to one of SEQ ID NOS:1-7, 13-15, 49-50, or 66-70, or a fragment thereof.

13. The peptide of claim 12, wherein the peptides are separated by one or more linkers.

14-15. (canceled)

16. The peptide of claim 1, wherein the peptide is attached to a collagen binding peptide.

17-24. (canceled)

25. The peptide of claim 1, wherein the collagen binding peptide comprises a collagen-binding fragment from an anti-collagen antibody or a collagen-binding fragment derived from an anti-collagen antibody.

26-32. (canceled)

33. The peptide of claim 1, wherein the peptide comprises a methionine immediately adjacent to the first amino acid of one of SEQ ID NOS:1-7 or 13-15.

34. The peptide of claim 1, wherein the peptide is attached to a cell adhesion moiety.

35-36. (canceled)

37. The peptide of claim 1, wherein the peptide is attached to a tag or a functional moiety.

38-42. (canceled)

43. The peptide of claim 1, wherein the peptide comprises two or more growth factor binding domains, wherein each growth factor binding domain has an amino acid sequence that is at least 80% identical to one of SEQ ID NOS:1-7, 13-15, 49-50, or 66-70.

44-46. (canceled)

47. A molecular complex comprising the peptide of claim 1.

48-53. (canceled)

54. A composition comprising the peptide of claim 1.

55-56. (canceled)

57. A biomaterial scaffold comprising the peptide of claim 1.

58-66. (canceled)

67. An implant comprising the peptide of claim 1.

68. (canceled)

69. A method for regenerating tissue in a subject, the method comprising administering the biomaterial of claim 57.

70. A method for facilitating wound or tissue healing in a subject, the method comprising administering the biomaterial of claim 57.

71-81. (canceled)

82. A method for treating von Willebrand Disease, angiodysplasia, and/or mucosal/cutaneous bleeding in a subject, the method comprising administering the biomaterial scaffold of claim 57 to the subject.

83-87. (canceled)

88. A biomaterial scaffold comprising the peptide of SEQ ID NO:49, wherein the peptide is covalently linked to fibrin, and wherein the biomaterial scaffold further comprises exogenously added VEGF and PDGF.

89. A method for facilitating wound or tissue healing in a subject, the method comprising administering the biomaterial scaffold of claim 88 to the subject.


SEQUENCE LISTING (81 sequences)

SEQ ID NO: 1 - protein, 75 aa, synthetic construct
ARGLEUVALPHEALALEUGLYTHRASPGLYLYSLYSLEUARGILELYSSERLYSGLULYSCYSASNASPGLYLYS

SEQ ID NO: 2 - protein, 20 aa, synthetic construct
PPFLMLLKGSTRFNKTKTFR

SEQ ID NO: 3 - protein, 23 aa, synthetic construct
TLFLAHGRLVYMFNVGHKKLKIR

SEQ ID NO: 4 - protein, 27 aa, synthetic construct
PLFLLHKKGKNLSKPKASQNKKGGKSK

SEQ ID NO: 5 - protein, 12 aa, synthetic construct
TLPDVGLELEVR

SEQ ID NO: 6 - protein, 20 aa, synthetic construct
RQRSRPGRWHKVSVRWEKNR

SEQ ID NO: 7 - protein, 31 aa, synthetic construct
TPGLGPRGLQATARKASRRSRQPARHPACML

SEQ ID NO: 8 - protein, 33 aa, synthetic construct
NQEQVSPLRLVFALGTDGKKLRIKSKEKCNDGK

SEQ ID NO: 9 - protein, 13 aa, synthetic construct
KNSFMALYLSKGR

SEQ ID NO: 10 - protein, 14 aa, synthetic construct
ARKASRRSRQPARH

SEQ ID NO: 11 - protein, 22 aa, synthetic construct
NQEQVSPLARKASRRSRQPARH

SEQ ID NO: 12 - protein, 8 aa, synthetic construct
NQEQVSPL

SEQ ID NO: 13 - protein, 165 aa, Homo sapiens
ALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSC

SEQ ID NO: 14 - protein, 172 aa, Homo sapiens
AYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPC

SEQ ID NO: 15 - protein, 174 aa, Homo sapiens
SYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPC

SEQ ID NO: 16 - protein, 177 aa, synthetic construct
NQEQVSPLGGSGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSC

SEQ ID NO: 17 - protein, 184 aa, synthetic construct
NQEQVSPLGGSGAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPC

SEQ ID NO: 18 - protein, 186 aa, synthetic construct
NQEQVSPLGGSGSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPC

SEQ ID NO: 19 - DNA, 495 bp, Homo sapiens
  source 1..495: mol_type = unassigned DNA
gccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgc

SEQ ID NO: 20 - DNA, 516 bp, Homo sapiens
  source 1..516: mol_type = unassigned DNA
gcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgc

SEQ ID NO: 21 - DNA, 522 bp, Homo sapiens
  source 1..522: mol_type = unassigned DNA
tcctaccagtttgggggttccctgtccagtcacctggagtttgtgggcatcctggcccgacataggaactggcccagtctctccatgcacgtcctcccgcgaagctcccgaggcctcctcctcttcactgcccgtctgaggcccggcagcccctccctggcgctcttcctgagcaatggccacttcgttgcacagatggaaggcctcgggactcggctccgcgcccagagccgccagcgctcccggcctggccgctggcacaaggtctccgtgcgctgggagaagaaccggatcctgctggtgacggacggggcccgggcctggagccaggaggggccgcaccggcagcaccagggggcagagcacccccagccccacaccctctttgtgggcggcctcccggccagcagccacagctccaaacttccggtgaccgtcgggttcagcggctgtgtgaagagactgaggctgcacgggaggcccctgggggcccccacacggatggcaggggtcacaccctgc

SEQ ID NO: 22 - DNA, 531 bp, synthetic construct
  source 1..531: mol_type = other DNA
aaccaggagcaggtgtccccacttggtggatccggcgccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgc

SEQ ID NO: 23 - DNA, 552 bp, synthetic construct
  source 1..552: mol_type = other DNA
aaccaggagcaggtgtccccacttggtggatccggcgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgc

SEQ ID NO: 24 - DNA, 558 bp, synthetic construct
  source 1..558: mol_type = other DNA
aaccaggagcaggtgtccccacttggtggatccggctcctaccagtttgggggttccctgtccagtcacctggagtttgtgggcatcctggcccgacataggaactggcccagtctctccatgcacgtcctcccgcgaagctcccgaggcctcctcctcttcactgcccgtctgaggcccggcagcccctccctggcgctcttcctgagcaatggccacttcgttgcacagatggaaggcctcgggactcggctccgcgcccagagccgccagcgctcccggcctggccgctggcacaaggtctccgtgcgctgggagaagaaccggatcctgctggtgacggacggggcccgggcctggagccaggaggggccgcaccggcagcaccagggggcagagcacccccagccccacaccctctttgtgggcggcctcccggccagcagccacagctccaaacttccggtgaccgtcgggttcagcggctgtgtgaagagactgaggctgcacgggaggcccctgggggcccccacacggatggcaggggtcacaccctgc

SEQ ID NO: 25 - protein, 3075 aa, Homo sapiens
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 26 - protein, 3122 aa, Homo sapiens
MPGAAGVLLLLLLSGGLGGVQAQRPQQQRQSQAHQQRGLFPAVLNLASNALITTNATCGEKGPEMYCKLVEHVPGQPVRNPQCRICNQNSSNPNQRHPITNAIDGKNTWWQSPSIKNGIEYHYVTITLDLQQVFQIAYVIVKAANSPRPGNWILERSLDDVEYKPWQYHAVTDTECLTLYNIYPRTGPPSYAKDDEVICTSFYSKIHPLENGEIHISLINGRPSADDPSPELLEFTSARYIRLRFQRIRTLNADLMMFAHKDPREIDPIVTRRYYYSVKDISVGGMCICYGHARACPLDPATNKSRCECEHNTCGDSCDQCCPGFHQKPWRAGTFLTKTECEACNCHGKAEECYYDENVARRNLSLNIRGKYIGGGVCINCTQNTAGINCETCTDGFFRPKGVSPNYPRPCQPCHCDPIGSLNEVCVKDEKHARRGLAPGSCHCKTGFGGVSCDRCARGYTGYPDCKACNCSGLGSKNEDPCFGPCICKENVEGGDCSRCKSGFFNLQEDNWKGCDECFCSGVSNRCQSSYWTYGKIQDMSGWYLTDLPGRIRVAPQQDDLDSPQQISISNAEARQALPHSYYWSAPAPYLGNKLPAVGGQLTFTISYDLEEEEEDTERVLQLMIILEGNDLSISTAQDEVYLHPSEEHTNVLLLKEESFTIHGTHFPVRRKEFMTVLANLKRVLLQITYSFGMDAIFRLSSVNLESAVSYPTDGSIAAAVEVCQCPPGYTGSSCESCWPRHRRVNGTIFGGICEPCQCFGHAESCDDVTGECLNCKDHTGGPYCDKCLPGFYGEPTKGTSEDCQPCACPLNIPSNNFSPTCHLDRSLGLICDGCPVGYTGPRCERCAEGYFGQPSVPGGSCQPCQCNDNLDFSIPGSCDSLSGSCLICKPGTTGRYCELCADGYFGDAVDAKNCQPCRCNAGGSFSEVCHSQTGQCECRANVQGQRCDKCKAGTFGLQSARGCVPCNCNSFGSKSFDCEESGQCWCQPGVTGKKCDRCAHGYFNFQEGGCTACECSHLGNNCDPKTGRCICPPNTIGEKCSKCAPNTWGHSITTGCKACNCSTVGSLDFQCNVNTGQCNCHPKFSGAKCTECSRGHWNYPRCNLCDCFLPGTDATTCDSETKKCSCSDQTGQCTCKVNVEGIHCDRCRPGKFGLDAKNPLGCSSCYCFGTTTQCSEAKGLIRTWVTLKAEQTILPLVDEALQHTTTKGIVFQHPEIVAHMDLMREDLHLEPFYWKLPEQFEGKKLMAYGGKLKYAIYFEAREETGFSTYNPQVIIRGGTPTHARIIVRHMAAPLIGQLTRHEIEMTEKEWKYYGDDPRVHRTVTREDFLDILYDIHYILIKATYGNFMRQSRISEISMEVAEQGRGTTMTPPADLIEKCDCPLGYSGLSCEACLPGFYRLRSQPGGRTPGPTLGTCVPCQCNGHSSLCDPETSICQNCQHHTAGDFCERCALGYYGIVKGLPNDCQQCACPLISSSNNFSPSCVAEGLDDYRCTACPRGYEGQYCERCAPGYTGSPGNPGGSCQECECDPYGSLPVPCDPVTGFCTCRPGATGRKCDGCKHWHAREGWECVFCGDECTGLLLGDLARLEQMVMSINLTGPLPAPYKMLYGLENMTQELKHLLSPQRAPERLIQLAEGNLNTLVTEMNELLTRATKVTADGEQTGQDAERTNTRAKSLGEFIKELARDAEAVNEKAIKLNETLGTRDEAFERNLEGLQKEIDQMIKELRRKNLETQKEIAEDELVAAEALLKKVKKLFGESRGENEEMEKDLREKLADYKNKVDDAWDLLREATDKIREANRLFAVNQKNMTALEKKKEAVESGKRQIENTLKEGNDILDEANRLADEINSIIDYVEDIQTKLPPMSEELNDKIDDLSQEIKDRKLAEKVSQAESHAAQLNDSSAVLDGILDEAKNISFNATAAFKAYSNIKDYIDEAEKVAKEAKDLAHEATKLATGPRGLLKEDAKGCLQKSFRILNEAKKLANDVKENEDHLNGLKTRIENADARNGDLLRTLNDTLGKLSAIPNDTAAKLQAVKDKARQANDTAKDVLAQITELHQNLDGLKKNYNKLADSVAKTNAVVKDPSKNKIIADADATVKNLEQEADRLIDKLKPIKELEDNLKKNISEIKELINQARKQANSIKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLSMKARPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTGKPLEVNFAKALELRGVQPVSCPAN

SEQ ID NO: 27 - protein, 3118 aa, Homo sapiens
MPGAAGVLLLLLLSGGLGGVQAQRPQQQRQSQAHQQRGLFPAVLNLASNALITTNATCGEKGPEMYCKLVEHVPGQPVRNPQCRICNQNSSNPNQRHPITNAIDGKNTWWQSPSIKNGIEYHYVTITLDLQQVFQIAYVIVKAANSPRPGNWILERSLDDVEYKPWQYHAVTDTECLTLYNIYPRTGPPSYAKDDEVICTSFYSKIHPLENGEIHISLINGRPSADDPSPELLEFTSARYIRLRFQRIRTLNADLMMFAHKDPREIDPIVTRRYYYSVKDISVGGMCICYGHARACPLDPATNKSRCECEHNTCGDSCDQCCPGFHQKPWRAGTFLTKTECEACNCHGKAEECYYDENVARRNLSLNIRGKYIGGGVCINCTQNTAGINCETCTDGFFRPKGVSPNYPRPCQPCHCDPIGSLNEVCVKDEKHARRGLAPGSCHCKTGFGGVSCDRCARGYTGYPDCKACNCSGLGSKNEDPCFGPCICKENVEGGDCSRCKSGFFNLQEDNWKGCDECFCSGVSNRCQSSYWTYGKIQDMSGWYLTDLPGRIRVAPQQDDLDSPQQISISNAEARQALPHSYYWSAPAPYLGNKLPAVGGQLTFTISYDLEEEEEDTERVLQLMIILEGNDLSISTAQDEVYLHPSEEHTNVLLLKEESFTIHGTHFPVRRKEFMTVLANLKRVLLQITYSFGMDAIFRLSSVNLESAVSYPTDGSIAAAVEVCQCPPGYTGSSCESCWPRHRRVNGTIFGGICEPCQCFGHAESCDDVTGECLNCKDHTGGPYCDKCLPGFYGEPTKGTSEDCQPCACPLNIPSNNFSPTCHLDRSLGLICDGCPVGYTGPRCERCAEGYFGQPSVPGGSCQPCQCNDNLDFSIPGSCDSLSGSCLICKPGTTGRYCELCADGYFGDAVDAKNCQPCRCNAGGSFSEVCHSQTGQCECRANVQGQRCDKCKAGTFGLQSARGCVPCNCNSFGSKSFDCEESGQCWCQPGVTGKKCDRCAHGYFNFQEGGCTACECSHLGNNCDPKTGRCICPPNTIGEKCSKCAPNTWGHSITTGCKACNCSTVGSLDFQCNVNTGQCNCHPKFSGAKCTECSRGHWNYPRCNLCDCFLPGTDATTCDSETKKCSCSDQTGQCTCKVNVEGIHCDRCRPGKFGLDAKNPLGCSSCYCFGTTTQCSEAKGLIRTWVTLKAEQTILPLVDEALQHTTTKGIVFQHPEIVAHMDLMREDLHLEPFYWKLPEQFEGKKLMAYGGKLKYAIYFEAREETGFSTYNPQVIIRGGTPTHARIIVRHMAAPLIGQLTRHEIEMTEKEWKYYGDDPRVHRTVTREDFLDILYDIHYILIKATYGNFMRQSRISEISMEVAEQGRGTTMTPPADLIEKCDCPLGYSGLSCEACLPGFYRLRSQPGGRTPGPTLGTCVPCQCNGHSSLCDPETSICQNCQHHTAGDFCERCALGYYGIVKGLPNDCQQCACPLISSSNNFSPSCVAEGLDDYRCTACPRGYEGQYCERCAPGYTGSPGNPGGSCQECECDPYGSLPVPCDPVTGFCTCRPGATGRKCDGCKHWHAREGWECVFCGDECTGLLLGDLARLEQMVMSINLTGPLPAPYKMLYGLENMTQELKHLLSPQRAPERLIQLAEGNLNTLVTEMNELLTRATKVTADGEQTGQDAERTNTRAKSLGEFIKELARDAEAVNEKAIKLNETLGTRDEAFERNLEGLQKEIDQMIKELRRKNLETQKEIAEDELVAAEALLKKVKKLFGESRGENEEMEKDLREKLADYKNKVDDAWDLLREATDKIREANRLFAVNQKNMTALEKKKEAVESGKRQIENTLKEGNDILDEANRLADEINSIIDYVEDIQTKLPPMSEELNDKIDDLSQEIKDRKLAEKVSQAESHAAQLNDSSAVLDGILDEAKNISFNATAAFKAYSNIKDYIDEAEKVAKEAKDLAHEATKLATGPRGLLKEDAKGCLQKSFRILNEAKKLANDVKENEDHLNGLKTRIENADARNGDLLRTLNDTLGKLSAIPNDTAAKLQAVKDKARQANDTAKDVLAQITELHQNLDGLKKNYNKLADSVAKTNAVVKDPSKNKIIADADATVKNLEQEADRLIDKLKPIKELEDNLKKNISEIKELINQARKQANSIKVSVSSGGDCIRTYKPEIKKGSYNNIVVNVKTAVADNLLFYLGSAKFIDFLAIEMRKGKVSFLWDVGSGVGRVEYPDLTIDDSYWYRIVASRTGRNGTISVRALDGPKASIVPSTHHSTSPPGYTILDVDANAMLFVGGLTGKLKKADAVRVITFTGCMGETYFDNKPIGLWNFREKEGDCKGCTVSPQVEDSEGTIQFDGEGYALVSRPIRWYPNISTVMFKFRTFSSSALLMYLATRDLRDFMSVELTDGHIKVSYDLGSGMASVVSNQNHNDGKWKSFTLSRIQKQANISIVDIDTNQEENIATSSSGNNFGLDLKADDKIYFGGLPTLRNLRPEVNLKKYSGCLKDIEISRTPYNILSSPDYVGVTKGCSLENVYTVSFPKPGFVELSPVPIDVGTEINLSFSTKNESGIILLGSGGTPAPPRRKRRQTGQAYYVILLNRGRLEVHLSTGARTMRKIVIRPEPNLFHDGREHSVHVERTRGIFTVQVDENRRYMQNLTVEQPIEVKKLFVGGAPPEFQPSPLRNIPPFEGCIWNLVINSVPMDFARPVSFKNADIGRCAHQKLREDEDGAAPAEIVIQPEPVPTPAFPTPTPVLTHGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTGKPLEVNFAKALELRGVQPVSCPAN

SEQ ID NO: 28 - protein, 3333 aa, Homo sapiens
MAAAARPRGRALGPVLPPTPLLLLVLRVLPACGATARDPGAAAGLSLHPTYFNLAEAARIWATATCGERGPGEGRPQPELYCKLVGGPTAPGSGHTIQGQFCDYCNSEDPRKAHPVTNAIDGSERWWQSPPLSSGTQYNRVNLTLDLGQLFHVAYILIKFANSPRPDLWVLERSVDFGSTYSPWQYFAHSKVDCLKEFGREANMAVTRDDDVLCVTEYSRIVPLENGEVVVSLINGRPGAKNFTFSHTLREFTKATNIRLRFLRTNTLLGHLISKAQRDPTVTRRYYYSIKDISIGGQCVCNGHAEVCNINNPEKLFRCECQHHTCGETCDRCCTGYNQRRWRPAAWEQSHECEACNCHGHASNCYYDPDVERQQASLNTQGIYAGGGVCINCQHNTAGVNCEQCAKGYYRPYGVPVDAPDGCIPCSCDPEHADGCEQGSGRCHCKPNFHGDNCEKCAIGYYNFPFCLRIPIFPVSTPSSEDPVAGDIKGCDCNLEGVLPEICDAHGRCLCRPGVEGPRCDTCRSGFYSFPICQACWCSALGSYQMPCSSVTGQCECRPGVTGQRCDRCLSGAYDFPHCQGSSSACDPAGTINSNLGYCQCKLHVEGPTCSRCKLLYWNLDKENPSGCSECKCHKAGTVSGTGECRQGDGDCHCKSHVGGDSCDTCEDGYFALEKSNYFGCQGCQCDIGGALSSMCSGPSGVCQCREHVVGKVCQRPENNYYFPDLHHMKYEIEDGSTPNGRDLRFGFDPLAFPEFSWRGYAQMTSVQNDVRITLNVGKSSGSLFRVILRYVNPGTEAVSGHITIYPSWGAAQSKEIIFLPSKEPAFVTVPGNGFADPFSITPGIWVACIKAEGVLLDYLVLLPRDYYEASVLQLPVTEPCAYAGPPQENCLLYQHLPVTRFPCTLACEARHFLLDGEPRPVAVRQPTPAHPVMVDLSGREVELHLRLRIPQVGHYVVVVEYSTEAAQLFVVDVNVKSSGSVLAGQVNIYSCNYSVLCRSAVIDHMSRIAMYELLADADIQLKGHMARFLLHQVCIIPIEEFSAEYVRPQVHCIASYGRFVNQSATCVSLAHETPPTALILDVLSGRPFPHLPQQSSPSVDVLPGVTLKAPQNQVTLRGRVPHLGRYVFVIHFYQAAHPTFPAQVSVDGGWPRAGSFHASFCPHVLGCRDQVIAEGQIEFDISEPEVAATVKVPEGKSLVLVRVLVVPAENYDYQILHKKSMDKSLEFITNCGKNSFYLDPQTASRFCKNSARSLVAFYHKGALPCECHPTGATGPHCSPEGGQCPCQPNVIGRQCTRCATGHYGFPRCKPCSCGRRLCEEMTGQCRCPPRTVRPQCEVCETHSFSFHPMAGCEGCNCSRRGTIEAAMPECDRDSGQCRCKPRITGRQCDRCASGFYRFPECVPCNCNRDGTEPGVCDPGTGACLCKENVEGTECNVCREGSFHLDPANLKGCTSCFCFGVNNQCHSSHKRRTKFVDMLGWHLETADRVDIPVSFNPGSNSMVADLQELPATIHSASWVAPTSYLGDKVSSYGGYLTYQAKSFGLPGDMVLLEKKPDVQLTGQHMSIIYEETNTPRPDRLHHGRVHVVEGNFRHASSRAPVSREELMTVLSRLADVRIQGLYFTETQRLTLSEVGLEEASDTGSGRIALAVEICACPPAYAGDSCQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 29 - protein, 1724 aa, Homo sapiens
MPPAVRRSACSMGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 30 - protein, 3277 aa, Homo sapiens
MAAAARPRGRALGPVLPPTPLLLLVLRVLPACGATARDPGAAAGLSLHPTYFNLAEAARIWATATCGERGPGEGRPQPELYCKLVGGPTAPGSGHTIQGQFCDYCNSEDPRKAHPVTNAIDGSERWWQSPPLSSGTQYNRVNLTLDLGQLFHVAYILIKFANSPRPDLWVLERSVDFGSTYSPWQYFAHSKVDCLKEFGREANMAVTRDDDVLCVTEYSRIVPLENGEVVVSLINGRPGAKNFTFSHTLREFTKATNIRLRFLRTNTLLGHLISKAQRDPTVTRRYYYSIKDISIGGQCVCNGHAEVCNINNPEKLFRCECQHHTCGETCDRCCTGYNQRRWRPAAWEQSHECEACNCHGHASNCYYDPDVERQQASLNTQGIYAGGGVCINCQHNTAGVNCEQCAKGYYRPYGVPVDAPDGCIPCSCDPEHADGCEQGSGRCHCKPNFHGDNCEKCAIGYYNFPFCLRIPIFPVSTPSSEDPVAGDIKGCDCNLEGVLPEICDAHGRCLCRPGVEGPRCDTCRSGFYSFPICQACWCSALGSYQMPCSSVTGQCECRPGVTGQRCDRCLSGAYDFPHCQGSSSACDPAGTINSNLGYCQCKLHVEGPTCSRCKLLYWNLDKENPSGCSECKCHKAGTVSGTGECRQGDGDCHCKSHVGGDSCDTCEDGYFALEKSNYFGCQGCQCDIGGALSSMCSGPSGVCQCREHVVGKVCQRPENNYYFPDLHHMKYEIEDGSTPNGRDLRFGFDPLAFPEFSWRGYAQMTSVQNDVRITLNVGKSSGSLFRVILRYVNPGTEAVSGHITIYPSWGAAQSKEIIFLPSKEPAFVTVPGNGFADPFSITPGIWVACIKAEGVLLDYLVLLPRDYYEASVLQLPVTEPCAYAGPPQENCLLYQHLPVTRFPCTLACEARHFLLDGEPRPVAVRQPTPAHPVMVDLSGREVELHLRLRIPQVGHYVVVVEYSTEAAQLFVVDVNVKSSGSVLAGQVNIYSCNYSVLCRSAVIDHMSRIAMYELLADADIQLKGHMARFLLHQVCIIPIEEFSAEYVRPQVHCIASYGRFVNQSATCVSLAHETPPTALILDVLSGRPFPHLPQQSSPSVDVLPGVTLKAPQNQVTLRGRVPHLGRYVFVIHFYQAAHPTFPAQVSVDGGWPRAGSFHASFCPHVLGCRDQVIAEGQIEFDISEPEVAATVKVPEGKSLVLVRVLVVPAENYDYQILHKKSMDKSLEFITNCGKNSFYLDPQTASRFCKNSARSLVAFYHKGALPCECHPTGATGPHCSPEGGQCPCQPNVIGRQCTRCATGHYGFPRCKPCSCGRRLCEEMTGQCRCPPRTVRPQCEVCETHSFSFHPMAGCEGCNCSRRGTIEAAMPECDRDSGQCRCKPRITGRQCDRCASGFYRFPECVPCNCNRDGTEPGVCDPGTGACLCKENVEGTECNVCREGSFHLDPANLKGCTSCFCFGVNNQCHSSHKRRTKFVDMLGWHLETADRVDIPVSFNPGSNSMVADLQELPATIHSASWVAPTSYLGDKVSSYGGYLTYQAKSFGLPGDMVLLEKKPDVQLTGQHMSIIYEETNTPRPDRLHHGRVHVVEGNFRHASSRAPVSREELMTVLSRLADVRIQGLYFTETQRLTLSEVGLEEASDTGSGRIALAVEICACPPAYAGDSCQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHMLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 31 - protein, 1668 aa, Homo sapiens
MPPAVRRSACSMGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHMLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGGPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 32 - protein, 488 aa, Homo sapiens
MAAAARPRGRALGPVLPPTPLLLLVLRVLPACGATARDPGAAAGLSLHPTYFNLAEAARIWATATCGERGPGEGRPQPELYCKLVGGPTAPGSGHTIQGQFCDYCNSEDPRKAHPVTNAIDGSERWWQSPPLSSGTQYNRVNLTLDLGQLFHVAYILIKFANSPRPDLWVLERSVDFGSTYSPWQYFAHSKVDCLKEFGREANMAVTRDDDVLCVTEYSRIVPLENGEVVVSLINGRPGAKNFTFSHTLREFTKATNIRLRFLRTNTLLGHLISKAQRDPTVTRRYYYSIKDISIGGQCVCNGHAEVCNINNPEKLFRCECQHHTCGETCDRCCTGYNQRRWRPAAWEQSHECEACNCHGHASNCYYDPDVERQQASLNTQGIYAGGGVCINCQHNTAGVNCEQCAKGYYRPYGVPVDAPDGCIRKFHFKLVYLSLCVLPQRSHQANFGSVNNFLHALSLQSISCARYVTSVTYTVSLNFGFIACKWK

SEQ ID NO: 33 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 34 - protein, 1816 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGCDKCVWDLTDDLRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 35 - protein, 120 aa, Homo sapiens
MALSSAWRSVLPLWLLWSAACSRAASGDDNAFPFDIEGSSAVGRQDPPETSEPRVALGRLPPAAEVQCPCHCHPAGAPAPPRAVPHSSFSLSPPLSSPQCLESFTWARSVRKLEIKSFPL

SEQ ID NO: 36 - protein, 3695 aa, Homo sapiens
MAKRLCAGSALCVRGPRGPAPLLLVGLALLGAARAREEAGGGFSLHPPYFNLAEGARIAASATCGEEAPARGSPRPTEDLYCKLVGGPVAGGDPNQTIRGQYCDICTAANSNKAHPASNAIDGTERWWQSPPLSRGLEYNEVNVTLDLGQVFHVAYVLIKFANSPRPDLWVLERSMDFGRTYQPWQFFASSKRDCLERFGPQTLERITRDDAAICTTEYSRIVPLENGEIVVSLVNGRPGAMNFSYSPLLREFTKATNVRLRFLRTNTLLGHLMGKALRDPTVTRRYYYSIKDISIGGRCVCHGHADACDAKDPTDPFRLQCTCQHNTCGGTCDRCCPGFNQQPWKPATANSANECQSCNCYGHATDCYYDPEVDRRRASQSLDGTYQGGGVCIDCQHHTTGVNCERCLPGFYRSPNHPLDSPHVCRRCNCESDFTDGTCEDLTGRCYCRPNFSGERCDVCAEGFTGFPSCYPTPSSSNDTREQVLPAGQIVNCDCSAAGTQGNACRKDPRVGRCLCKPNFQGTHCELCAPGFYGPGCQPCQCSSPGVADDRCDPDTGQCRCRVGFEGATCDRCAPGYFHFPLCQLCGCSPAGTLPEGCDEAGRCLCQPEFAGPHCDRCRPGYHGFPNCQACTCDPRGALDQLCGAGGLCRCRPGYTGTACQECSPGFHGFPSCVPCHCSAEGSLHAACDPRSGQCSCRPRVTGLRCDTCVPGAYNFPYCEAGSCHPAGLAPVDPALPEAQVPCMCRAHVEGPSCDRCKPGFWGLSPSNPEGCTRCSCDLRGTLGGVAECQPGTGQCFCKPHVCGQACASCKDGFFGLDQADYFGCRSCRCDIGGALGQSCEPRTGVCRCRPNTQGPTCSEPARDHYLPDLHHLRLELEEAATPEGHAVRFGFNPLEFENFSWRGYAQMAPVQPRIVARLNLTSPDLFWLVFRYVNRGAMSVSGRVSVREEGRSATCANCTAQSQPVAFPPSTEPAFITVPQRGFGEPFVLNPGTWALRVEAEGVLLDYVVLLPSAYYEAALLQLRVTEACTYRPSAQQSGDNCLLYTHLPLDGFPSAAGLEALCRQDNSLPRPCPTEQLSPSHPPLITCTGSDVDVQLQVAVPQPGRYALVVEYANEDARQEVGVAVHTPQRAPQQGLLSLHPCLYSTLCRGTARDTQDHLAVFHLDSEASVRLTAEQARFFLHGVTLVPIEEFSPEFVEPRVSCISSHGAFGPNSAACLPSRFPKPPQPIILRDCQVIPLPPGLPLTHAQDLTPAMSPAGPRPRPPTAVDPDAEPTLLREPQATVVFTTHVPTLGRYAFLLHGYQPAHPTFPVEVLINAGRVWQGHANASFCPHGYGCRTLVVCEGQALLDVTHSELTVTVRVPKGRWLWLDYVLVVPENVYSFGYLREEPLDKSYDFISHCAAQGYHISPSSSSLFCRNAAASLSLFYNNGARPCGCHEVGATGPTCEPFGGQCPCHAHVIGRDCSRCATGYWGFPNCRPCDCGARLCDELTGQCICPPRTIPPDCLLCQPQTFGCHPLVGCEECNCSGPGIQELTDPTCDTDSGQCKCRPNVTGRRCDTCSPGFHGYPRCRPCDCHEAGTAPGVCDPLTGQCYCKENVQGPKCDQCSLGTFSLDAANPKGCTRCFCFGATERCRSSSYTRQEFVDMEGWVLLSTDRQVVPHERQPGTEMLRADLRHVPEAVPEAFPELYWQAPPSYLGDRVSSYGGTLRYELHSETQRGDVFVPMESRPDVVLQGNQMSITFLEPAYPTPGHVHRGQLQLVEGNFRHTETRNTVSREELMMVLASLEQLQIRALFSQISSAVFLRRVALEVASPAGQGALASNVELCLCPASYRGDSCQECAPGFYRDVKGLFLGRCVPCQCHGHSDRCLPGSGVCVDCQHNTEGAHCERCQAGFVSSRDDPSAPCVSCPCPLSVPSNNFAEGCVLRGGRTQCLCKPGYAGASCERCAPGFFGNPLVLGSSCQPCDCSGNGDPNLLFSDCDPLTGACRGCLRHTTGPRCEICAPGFYGNALLPGNCTRCDCTPCGTEACDPHSGHCLCKAGVTGRRCDRCQEGHFGFDGCGGCRPCACGPAAEGSECHPQSGQCHCRPGTMGPQCRECAPGYWGLPEQGCRRCQCPGGRCDPHTGRCNCPPGLSGERCDTCSQQHQVPVPGGPVGHSIHCEVCDHCVVLLLDDLERAGALLPAIHEQLRGINASSMAWARLHRLNASIADLQSQLRSPLGPRHETAQQLEVLEQQSTSLGQDARRLGGQAVGTRDQASQLLAGTEATLGHAKTLLAAIRAVDRTLSELMSQTGHLGLANASAPSGEQLLRTLAEVERLLWEMRARDLGAPQAAAEAELAAAQRLLARVQEQLSSLWEENQALATQTRDRLAQHEAGLMDLREALNRAVDATREAQELNSRNQERLEEALQRKQELSRDNATLQATLHAARDTLASVFRLLHSLDQAKEELERLAASLDGARTPLLQRMQTFSPAGSKLRLVEAAEAHAQQLGQLALNLSSIILDVNQDRLTQRAIEASNAYSRILQAVQAAEDAAGQALQQADHTWATVVRQGLVDRAQQLLANSTALEEAMLQEQQRLGLVWAALQGARTQLRDVRAKKDQLEAHIQAAQAMLAMDTDETSKKIAHAKAVAAEAQDTATRVQSQLQAMQENVERWQGQYEGLRGQDLGQAVLDAGHSVSTLEKTLPQLLAKLSILENRGVHNASLALSASIGRVRELIAQARGAASKVKVPMKFNGRSGVQLRTPRDLADLAAYTALKFYLQGPEPEPGQGTEDRFVMYMGSRQATGDYMGVSLRDKKVHWVYQLGEAGPAVLSIDEDIGEQFAAVSLDRTLQFGHMSVTVERQMIQETKGDTVAPGAEGLLNLRPDDFVFYVGGYPSTFTPPPLLRFPGYRGCIEMDTLNEEVVSLYNFERTFQLDTAVDRPCARSKSTGDPWLTDGSYLDGTGFARISFDSQISTTKRFEQELRLVSYSGVLFFLKQQSQFLCLAVQEGSLVLLYDFGAGLKKAVPLQPPPPLTSASKAIQVFLLGGSRKRVLVRVERATVYSVEQDNDLELADAYYLGGVPPDQLPPSLRRLFPTGGSVRGCVKGIKALGKYVDLKRLNTTGVSAGCTADLLVGRAMTFHGHGFLRLALSNVAPLTGNVYSGFGFHSAQDSALLYYRASPDGLCQVSLQQGRVSLQLLRTEVKTQAGFADGAPHYVAFYSNATGVWLYVDDQLQQMKPHRGPPPELQPQPEGPPRLLLGGLPESGTIYNFSGCISNVFVQRLLGPQRVFDLQQNLGSVNVSTGCAPALQAQTPGLGPRGLQATARKASRRSRQPARHPACMLPPHLRTTRDSYQFGGSLSSHLEFVGILARHRNWPSLSMHVLPRSSRGLLLFTARLRPGSPSLALFLSNGHFVAQMEGLGTRLRAQSRQRSRPGRWHKVSVRWEKNRILLVTDGARAWSQEGPHRQHQGAEHPQPHTLFVGGLPASSHSSKLPVTVGFSGCVKRLRLHGRPLGAPTRMAGVTPCILGPLEAGLFFPGSGGVITLDLPGATLPDVGLELEVRPLAVTGLIFHLGQARTPPYLQLQVTEKQVLLRADDGAGEFSTSVTRPSVLCDGQWHRLAVMKSGNVLRLEVDAQSNHTVGPLLAAAAGAPAPLYLGGLPEPMAVQPWPPAYCGCMRRLAVNRSPVAMTRSVEVHGAVGASGCPAA

SEQ ID NO: 37 - protein, 1786 aa, Homo sapiens
MGLLQLLAFSFLALCRARVRAQEPEFSYGCAEGSCYPATGDLLIGRAQKLSVTSTCGLHKPEPYCIVSHLQEDKKCFICNSQDPYHETLNPDSHLIENVVTTFAPNRLKIWWQSENGVENVTIQLDLEAEFHFTHLIMTFKTFRPAAMLIERSSDFGKTWGVYRYFAYDCEASFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGNCFCYGHASECAPVDGFNEEVEGMVHGHCMCRHNTKGLNCELCMDFYHDLPWRPAEGRNSNACKKCNCNEHSISCHFDMAVYLATGNVSGGVCDDCQHNTMGRNCEQCKPFYYQHPERDIRDPNFCERCTCDPAGSQNEGICDSYTDFSTGLIAGQCRCKLNVEGEHCDVCKEGFYDLSSEDPFGCKSCACNPLGTIPGGNPCDSETGHCYCKRLVTGQHCDQCLPEHWGLSNDLDGCRPCDCDLGGALNNSCFAESGQCSCRPHMIGRQCNEVEPGYYFATLDHYLYEAEEANLGPGVSIVERQYIQDRIPSWTGAGFVRVPEGAYLEFFIDNIPYSMEYDILIRYEPQLPDHWEKAVITVQRPGRIPTSSRCGNTIPDDDNQVVSLSPGSRYVVLPRPVCFEKGTNYTVRLELPQYTSSDSDVESPYTLIDSLVLMPYCKSLDIFTVGGSGDGVVTNSAWETFQRYRCLENSRSVVKTPMTDVCRNIIFSISALLHQTGLACECDPQGSLSSVCDPNGGQCQCRPNVVGRTCNRCAPGTFGFGPSGCKPCECHLQGSVNAFCNPVTGQCHCFQGVYARQCDRCLPGHWGFPSCQPCQCNGHADDCDPVTGECLNCQDYTMGHNCERCLAGYYGDPIIGSGDHCRPCPCPDGPDSGRQFARSCYQDPVTLQLACVCDPGYIGSRCDDCASGYFGNPSEVGGSCQPCQCHNNIDTTDPEACDKETGRCLKCLYHTEGEHCQFCRFGYYGDALQQDCRKCVCNYLGTVQEHCNGSDCQCDKATGQCLCLPNVIGQNCDRCAPNTWQLASGTGCDPCNCNAAHSFGPSCNEFTGQCQCMPGFGGRTCSECQELFWGDPDVECRACDCDPRGIETPQCDQSTGQCVCVEGVEGPRCDKCTRGYSGVFPDCTPCHQCFALWDVIIAELTNRTHRFLEKAKALKISGVIGPYRETVDSVERKVSEIKDILAQSPAAEPLKNIGNLFEEAEKLIKDVTEMMAQVEVKLSDTTSQSNSTAKELDSLQTEAESLDNTVKELAEQLEFIKNSDIRGALDSITKYFQMSLEAEERVNASTTEPNSTVEQSALMRDRVEDVMMERESQFKEKQEEQARLLDELAGKLQSLDLSAAAEMTCGTPPGASCSETECGGPNCRTDEGERKCGGPGCGGLVTVAHNAWQKAMDLDQDVLSALAEVEQLSKMVSEAKLRADEAKQSAEDILLKTNATKEKMDKSNEELRNLIKQIRNFLTQDSADLDSIEAVANEVLKMEMPSTPQQLQNLTEDIRERVESLSQVEVILQHSAADIARAEMLLEEAKRASKSATDVKVTADMVKEALEEAEKAQVAAEKAIKQADEDIQGTQNLLTSIESETAASEETLFNASQRISELERNVEELKRKAAQNSGEAEYIEKVVYTVKQSAEDVKKTLDGELDEKYKKVENLIAKKTEESADARRKAEMLQNEAKTLLAQANSKLQLLKDLERKYEDNQRYLEDKAQELARLEGEVRSLLKDISQKVAVYSTCL

SEQ ID NO: 38 - protein, 1798 aa, Homo sapiens
MELTSRERGRGQPLPWELRLGLLLSVLAATLAQAPAPDVPGCSRGSCYPATGDLLVGRADRLTASSTCGLNGPQPYCIVSHLQDEKKCFLCDSRRPFSARDNPHSHRIQNVVTSFAPQRRAAWWQSENGIPAVTIQLDLEAEFHFTHLIMTFKTFRPAAMLVERSADFGRTWHVYRYFSYDCGADFPGVPLAPPRHWDDVVCESRYSEIEPSTEGEVIYRVLDPAIPIPDPYSSRIQNLLKITNLRVNLTRLHTLGDNLLDPRREIREKYYYALYELVVRGNCFCYGHASECAPAPGAPAHAEGMVHGACICKHNTRGLNCEQCQDFYRDLPWRPAEDGHSHACRKCECHGHTHSCHFDMAVYLASGNVSGGVCDGCQHNTAGRHCELCRPFFYRDPTKDLRDPAVCRSCDCDPMGSQDGGRCDSHDDPALGLVSGQCRCKEHVVGTRCQQCRDGFFGLSISDRLGCRRCQCNARGTVPGSTPCDPNSGSCYCKRLVTGRGCDRCLPGHWGLSHDLLGCRPCDCDVGGALDPQCDEGTGQCHCRQHMVGRRCEQVQPGYFRPFLDHLIWEAEDTRGQVLDVVERLVTPGETPSWTGSGFVRLQEGQTLEFLVASVPKAMDYDLLLRLEPQVPEQWAELELIVQRPGPVPAHSLCGHLVPKDDRIQGTLQPHARYLIFPNPVCLEPGISYKLHLKLVRTGGSAQPETPYSGPGLLIDSLVLLPRVLVLEMFSGGDAAALERQATFERYQCHEEGLVPSKTSPSEACAPLLISLSTLIYNGALPCQCNPQGSLSSECNPHGGQCLCKPGVVGRRCDLCAPGYYGFGPTGCQACQCSHEGALSSLCEKTSGQCLCRTGAFGLRCDRCQRGQWGFPSCRPCVCNGHADECNTHTGACLGCRDHTGGEHCERCIAGFHGDPRLPYGGQCRPCPCPEGPGSQRHFATSCHQDEYSQQIVCHCRAGYTGLRCEACAPGHFGDPSRPGGRCQLCECSGNIDPMDPDACDPHTGQCLRCLHHTEGPHCAHCKPGFHGQAARQSCHRCTCNLLGTNPQQCPSPDQCHCDPSSGQCPCLPNVQGPSCDRCAPNFWNLTSGHGCQPCACHPSRARGPTCNEFTGQCHCRAGFGGRTCSECQELHWGDPGLQCHACDCDSRGIDTPQCHRFTGHCSCRPGVSGVRCDQCARGFSGIFPACHPCHACFGDWDRVVQDLAARTQRLEQRAQELQQTGVLGAFESSFWHMQEKLGIVQGIVGARNTSAASTAQLVEATEELRREIGEATEHLTQLEADLTDVQDENFNANHALSGLERDRLALNLTLRQLDQHLDLLKHSNFLGAYDSIRHAHSQSAEAERRANTSALAVPSPVSNSASARHRTEALMDAQKEDFNSKHMANQRALGKLSAHTHTLSLTDINELVCGAPGDAPCATSPCGGAGCRDEDGQPRCGGLSCNGAAATADLALGRARHTQAELQRALAEGGSILSRVAETRRQASEAQQRAQAALDKANASRGQVEQANQELQELIQSVKDFLNQEGADPDSIEMVATRVLELSIPASAEQIQHLAGAIAERVRSLADVDAILARTVGDVRRAEQLLQDARRARSWAEDEKQKAETVQAALEEAQRAQGIAQGAIRGAVADTRDTEQTLYQVQERMAGAERALSSAGERARQLDALLEALKLKRAGNSLAASTAEETAGSAQGRAQEAEQLLRGPLGDQYQTVKALAERKAQGVLAAQARAEQLRDEARDLLQAAQDKLQRLQELEGTYEENERALESKAAQLDGLEARMRSVLQAINLQVQIYNTCQ

SEQ ID NO: 39 - protein, 1172 aa, Homo sapiens [sequence omitted]

SEQ ID NO: 40 - protein, 1761 aa, Homo sapiens
MQFQLTLFLHLGWLSYSKAQDDCNRGACHPTTGDLLVGRNTQLMASSTCGLSRAQKYCILSYLEGEQKCFICDSRFPYDPYDQPNSHTIENVIVSFEPDREKKWWQSENGLDHVSIRLDLEALFRFSHLILTFKTFRPAAMLVERSTDYGHNWKVFKYFAKDCATSFPNITSGQAQGVGDIVCDSKYSDIEPSTGGEVVLKVLDPSFEIENPYSPYIQDLVTLTNLRINFTKLHTLGDALLGRRQNDSLDKYYYALYEMIVRGSCFCNGHASECRPMQKMRGDVFSPPGMVHGQCVCQHNTDGPNCERCKDFFQDAPWRPAADLQDNACRSCSCNSHSSRCHFDMTTYLASGGLSGGVCEDCQHNTEGQHCDRCRPLFYRDPLKTISDPYACIPCECDPDGTISGGICVSHSDPALGSVAGQCLCKENVEGAKCDQCKPNHYGLSATDPLGCQPCDCNPLGSLPFLTCDVDTGQCLCLSYVTGAHCEECTVGYWGLGNHLHGCSPCDCDIGGAYSNVCSPKNGQCECRPHVTGRSCSEPAPGYFFAPLNFYLYEAEEATTLQGLAPLGSETFGQSPAVHVVLGEPVPGNPVTWTGPGFARVLPGAGLRFAVNNIPFPVDFTIAIHYETQSAADWTVQIVVNPPGGSEHCIPKTLQSKPQSFALPAATRIMLLPTPICLEPDVQYSIDVYFSQPLQGESHAHSHVLVDSLGLIPQINSLENFCSKQDLDEYQLHNCVEIASAMGPQVLPGACERLIISMSAKLHDGAVACKCHPQGSVGSSCSRLGGQCQCKPLVVGRCCDRCSTGSYDLGHHGCHPCHCHPQGSKDTVCDQVTGQCPCHGEVSGRRCDRCLAGYFGFPSCHPCPCNRFAELCDPETGSCFNCGGFTTGRNCERCIDGYYGNPSSGQPCRPCLCPDDPSSNQYFAHSCYQNLWSSDVICNCLQGYTGTQCGECSTGFYGNPRISGAPCQPCACNNNIDVTDPESCSRVTGECLRCLHNTQGANCQLCKPGHYGSALNQTCRRCSCHASGVSPMECPPGGGACLCDPVTGACPCLPNVTGLACDRCADGYWNLVPGRGCQSCDCDPRTSQSSHCDQLTGQCPCKLGYGGKRCSECQENYYGDPPGRCIPCDCNRAGTQKPICDPDTGMCRCREGVSGQRCDRCARGHSQEFPTCLQCHLCFDQWDHTISSLSKAVQGLMRLAANMEDKRETLPVCEADFKDLRGNVSEIERILKHPVFPSGKFLKVKDYHDSVRRQIMQLNEQLKAVYEFQDLKDTIERAKNEADLLLEDLQEEIDLQSSVLNASIADSSENIKKYYHISSSAEKKINETSSTINTSANTRNDLLTILDTLTSKGNLSLERLKQIKIPDIQILNEKVCGDPGNVPCVPLPCGGALCTGRKGHRKCRGPGCHGSLTLSTNALQKAQEAKSIIRNLDKQVRGLKNQIESISEQAEVSKNNALQLREKLGNIRNQSDSEEENINLFIKKVKNFLLEENVPPEDIEKVANGVLDIHLPIPSQNLTDELVKIQKHMQLCEDYRTDENRLNEEADGAQKLLVKAKAAEKAANILLNLDKTLNQLQQAQITQGRANSTITQLTANITKIKKNVLQAENQTREMKSELELAKQRSGLEDGLSLLQTKLQRHQDHAVNAKVQAESAQHQAGSLEKEFVELKKQYAILQRKTSTTGLTKETLGKVKQLKDAAEKLAGDTEAKIRRITDLERKIQDLNLSRQAKADQLRILEDQVVAIKNEIVEQEKKYARCYS

SEQ ID NO: 41 - protein, 1101 aa, Homo sapiens
MQFQLTLFLHLGWLSYSKAQDDCNRGACHPTTGDLLVGRNTQLMASSTCGLSRAQKYCILSYLEGEQKCFICDSRFPYDPYDQPNSHTIENVIVSFEPDREKKWWQSENGLDHVSIRLDLEALFRFSHLILTFKTFRPAAMLVERSTDYGHNWKVFKYFAKDCATSFPNITSGQAQGVGDIVCDSKYSDIEPSTGGEVVLKVLDPSFEIENPYSPYIQDLVTLTNLRINFTKLHTLGDALLGRRQNDSLDKYYYALYEMIVRGSCFCNGHASECRPMQKMRGDVFSPPGMVHGQCVCQHNTDGPNCERCKDFFQDAPWRPAADLQDNACRSCSCNSHSSRCHFDMTTYLASGGLSGGVCEDCQHNTEGQHCDRCRPLFYRDPLKTISDPYACIPCECDPDGTISGGICVSHSDPALGSVAGQCLCKENVEGAKCDQCKPNHYGLSATDPLGCQPCDCNPLGSLPFLTCDVDTGQCLCLSYVTGAHCEECTVGYWGLGNHLHGCSPCDCDIGGAYSNVCSPKNGQCECRPHVTGRSCSEPAPGYFFAPLNFYLYEAEEATTLQGLAPLGSETFGQSPAVHVVLGEPVPGNPVTWTGPGFARVLPGAGLRFAVNNIPFPVDFTIAIHYETQSAADWTVQIVVNPPGGSEHCIPKTLQSKPQSFALPAATRIMLLPTPICLEPDVQYSIDVYFSQPLQGESHAHSHVLVDSLGLIPQINSLENFCSKQDLDEYQLHNCVEIASAMGPQVLPGACERLIISMSAKLHDGAVACKCHPQGSVGSSCSRLGGQCQCKPLVVGRCCDRCSTGSYDLGHHGCHPCHCHPQGSKDTVCDQVTGQCPCHGEVSGRRCDRCLAGYFGFPSCHPCPCNRFAELCDPETGSCFNCGGFTTGRNCERCIDGYYGNPSSGQPCRPCLCPDDPSSNQYFAHSCYQNLWSSDVICNCLQGYTGTQCGECSTGFYGNPRISGAPCQPCACNNNIDVTDPESCSRVTGECLRCLHNTQGANCQLCKPGHYGSALNQTCRRCSCHASGVSPMECPPGGGACLCDPVTGACPCLPNVTGLACDRCADGYWNLVPGRGCQSCDCDPRTSQSSHCDQARYFKAY

SEQ ID NO: 42 - protein, 772 aa, Homo sapiens
MQFQLTLFLHLGWLSYSKAQDDCNRGACHPTTGDLLVGRNTQLMASSTCGLSRAQKYCILSYLEGEQKCFICDSRFPYDPYDQPNSHTIENVIVSFEPDREKKWWQSENGLDHVSIRLDLEALFRFSHLILTFKTFRPAAMLVERSTDYGHNWKVFKYFAKDCATSFPNITSGQAQGVGDIVCDSKYSDIEPSTGGEVVLKVLDPSFEIENPYSPYIQDLVTLTNLRINFTKLHTLGDALLGRRQNDSLDKYYYALYEMIVRGSCFCNGHASECRPMQKMRGDVFSPPGMVHGQCVCQHNTDGPNCERCKDFFQDAPWRPAADLQDNACRSCSCNSHSSRCHFDMTTYLASGGLSGGVCEDCQHNTEGQHCDRCRPLFYRDPLKTISDPYACIPCECDPDGTISGGICVSHSDPALGSVAGQCLCKENVEGAKCDQCKPNHYGLSATDPLGCQPCDCNPLGSLPFLTCDVDTGQCLCLSYVTGAHCEECTVGYWGLGNHLHGCSPCDCDIGGAYSNVCSPKNGQCECRPHVTGRSCSEPAPGYFFAPLNFYLYEAEEATTLQGLAPLGSETFGQSPAVHVVLGEPVPGNPVTWTGPGFARVLPGAGLRFAVNNIPFPVDFTIAIHYETQSAADWTVQIVVNPPGGSEHCIPKTLQSKPQSFALPAATRIMLLPTPICLEPDVQYSIDVYFSQPLQGESHAHSHVLVDSAAVQWHNLGSLQPPPPECKQFSCFSFPSSWDYRHPPPHLANFCIFSRDGVSPHWPGWSQTPDLR

SEQ ID NO: 43 - protein, 1609 aa, Homo sapiens
MRGSHRAAPALRPRGRLWPVLAVLAAAAAAGCAQAAMDECTDEGGRPQRCMPEFVNAAFNVTVVATNTCGTPPEEYCVQTGVTGVTKSCHLCDAGQPHLQHGAAFLTDYNNQADTTWWQSQTMLAGVQYPSSINLTLHLGKAFDITYVRLKFHTSRPESFAIYKRTREDGPWIPYQYYSGSCENTYSKANRGFIRTGGDEQQALCTDEFSDISPLTGGNVAFSTLEGRPSAYNFDNSPVLQEWVTATDIRVTLNRLNTFGDEVFNDPKVLKSYYYAISDFAVGGRCKCNGHASECMKNEFDKLVCNCKHNTYGVDCEKCLPFFNDRPWRRATAESASECLPCDCNGRSQECYFDPELYRSTGHGGHCTNCQDNTDGAHCERCRENFFRLGNNEACSSCHCSPVGSLSTQCDSYGRCSCKPGVMGDKCDRCQPGFHSLTEAGCRPCSCDPSGSIDECNIETGRCVCKDNVEGFNCERCKPGFFNLESSNPRGCTPCFCFGHSSVCTNAVGYSVYSISSTFQIDEDGWRAEQRDGSEASLEWSSERQDIAVISDSYFPRYFIAPAKFLGKQVLSYGQNLSFSFRVDRRDTRLSAEDLVLEGAGLRVSVPLIAQGNSYPSETTVKYVFRLHEATDYPWRPALTPFEFQKLLNNLTSIKIRGTYSERSAGYLDDVTLASARPGPGVPATWVESCTCPVGYGGQFCEMCLSGYRRETPNLGPYSPCVLCACNGHSETCDPETGVCNCRDNTAGPHCEKCSDGYYGDSTAGTSSDCQPCPCPGGSSCAVVPKTKEVVCTNCPTGTTGKRCELCDDGYFGDPLGRNGPVRLCRLCQCSDNIDPNAVGNCNRLTGECLKCIYNTAGFYCDRCKDGFFGNPLAPNPADKCKACNCNLYGTMKQQSSCNPVTGQCECLPHVTGQDCGACDPGFYNLQSGQGCERCDCHALGSTNGQCDIRTGQCECQPGITGQHCERCEVNHFGFGPEGCKPCDCHPEGSLSLQCKDDGRCECREGFVGNRCDQCEENYFYNRSWPGCQECPACYRLVKDKVADHRVKLQELESLIANLGTGDEMVTDQAFEDRLKEAEREVMDLLREAQDVKDVDQNLMDRLQRVNNTLSSQISRLQNIRNTIEETGNLAEQARAHVENTERLIEIASRELEKAKVAAANVSVTQPESTGDPNNMTLLAEEARKLAERHKQEADDIVRVAKTANDTSTEAYNLLLRTLAGENQTAFEIEELNRKYEQAKNISQDLEKQAARVHEEAKRAGDKAVEIYASVAQLSPLDSETLENEANNIKMEAENLEQLIDQKLKDYEDLREDMRGKELEVKNLLEKGKTEQQTADQLLARADAAKALAEEAAKKGRDTLQEANDILNNLKDFDRRVNDNKTAAEEALRKIPAINQTITEANEKTREAQQALGSAAADATEAKNKAHEAERIASAVQKNATSTKAEAERTFAEVTDLDNEVNNMLKQLQEAEKELKRKQDDADQDMMMAGMASQAAQEAEINARKAKNSVTSLLSIINDLLEQLGQLDTVDLNKLNEIEGTLNKAKDEMKVSDLDRKVSDLENEAKKQEAAIMDYNRDIEEIMKDIRNLEDIRKTLPSGCFNTPSIEKP

SEQ ID NO: 44 - protein, 1193 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPLGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMDQPLSVDEEGLVLLEQKLSRAKTQINSQLRPMMSELEERARQQRGHLHLLETSIDGILADVKNLENIRDNLPPGCYNTQALEQQ

SEQ ID NO: 45 - protein, 1111 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPLGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMGM

SEQ ID NO: 46 - protein, 1575 aa, Homo sapiens
MAAAALLLGLALLAPRAAGAGMGACYDGAGRPQRCLPVFENAAFGRLAQASHTCGSPPEDFCPHVGAAGAGAHCQRCDAADPQRHHNASYLTDFHSQDESTWWQSPSMAFGVQYPTSVNITLRLGKAYEITYVRLKFHTSRPESFAIYKRSRADGPWEPYQFYSASCQKTYGRPEGQYLRPGEDERVAFCTSEFSDISPLSGGNVAFSTLEGRPSAYNFEESPGLQEWVTSTELLISLDRLNTFGDDIFKDPKVLQSYYYAVSDFSVGGRCKCNGHASECGPDVAGQLACRCQHNTTGTDCERCLPFFQDRPWARGTAEAAHECLPCNCSGRSEECTFDRELFRSTGHGGRCHHCRDHTAGPHCERCQENFYHWDPRMPCQPCDCQSAGSLHLQCDDTGTCACKPTVTGWKCDRCLPGFHSLSEGGCRPCTCNPAGSLDTCDPRSGRCPCKENVEGNLCDRCRPGTFNLQPHNPAGCSSCFCYGHSKVCASTAQFQVHHILSDFHQGAEGWWARSVGGSEHPPQWSPNGVLLSPEDEEELTAPEKFLGDQRFSYGQPLILTFRVPPGDSPLPVQLRLEGTGLALSLRHSSLSGPQDAGHPREVELRFHLQETSEDVAPPLPPFHFQRLLANLTSLRLRVSPGPSPAGPVFLTEVRLTSARPGLSPPASWVEICSCPTGYTGQFCESCAPGYKREMPQGGPYASCVPCTCNQHGTCDPNTGICVCSHHTEGPSCERCLPGFYGNPFAGQADDCQPCPCPGQSACTTIPESREVVCTHCPPGQRGRRCEVCDDGFFGDPLGLFGHPQPCHQCQCSGNVDPNAVGNCDPLSGHCLRCLHNTTGDHCEHCQEGFYGSALAPRPADKCMPCSCHPQGSVSEQMPCDPVTGQCSCLPHVTARDCSRCYPGFFDLQPGRGCRSCKCHPLGSQEDQCHPKTGQCTCRPGVTGQACDRCQLGFFGFSIKGCRACRCSPLGAASAQCHENGTCVCRPGFEGYKCDRCHDNFFLTADGTHCQQCPSCYALVKEEAAKLKARLTLTEGWLQGSDCGSPWGPLDILLGEAPRGDVYQGHHLLPGAREAFLEQMMSLEGAVKAAREQLQRLNKGARCAQAGSQKTCTQLADLEAVLESSEEEILHAAAILASLEIPQEGPSQPTKWSHLATEARALARSHRDTATKIAATAWRALLASNTSYALLWNLLEGRVALETQRDLEDRYQEVQAAQKALRTAVAEVLPEAESVLATVQQVGADTAPYLALLASPGALPQKSRAEDLGLKAKALEKTVASWQHMATEAARTLQTAAQATLRQTEPLTKLHQEARAALTQASSSVQAATVTVMGARTLLADLEGMKLQFPRPKDQAALQRKADSVSDRLLADTRKKTKQAERMLGNAAPLSSSAKKKGREAEVLAKDSAKLAKALLRERKQAHRRASRLTSQTQATLQQASQQVLASEARRQELEEAERVGAGLSEMEQQIRESRISLEKDIETLSELLARLGSLDTHQAPAQALNETQWALERLRLQLGSPGSLQRKLSLLEQESQQQELQIQGFESDLAEIRADKQNLEAILHSLPENCASWQ

SEQ ID NO: 47 - protein, 196 aa, synthetic construct
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 48 - protein, 344 aa, synthetic construct
CGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 49 - protein, 24 aa, synthetic construct
YIGLKDRKRPSELRRIASQVKYAC

SEQ ID NO: 50 - protein, 23 aa, synthetic construct
YIGLKDRKRPSELRRIASQVKYA

SEQ ID NO: 51 - protein, 31 aa, synthetic construct
NQEQVSPLYIGLKDRKRPSELRRIASQVKYA

SEQ ID NO: 52 - protein, 24 aa, synthetic construct
LYCEIARGYSLKRKVPDQIRSRKA

SEQ ID NO: 53 - protein, 24 aa, synthetic construct
YIGLKDSKSPSELSSIASQVKYAC

SEQ ID NO: 54 - protein, 23 aa, synthetic construct
YIGLKDRKRPSELRRIASQVKYA

SEQ ID NO: 55 - protein, 23 aa, synthetic construct
YIGLKDRKRPSELLRIASQVKYA

SEQ ID NO: 56 - protein, 23 aa, synthetic construct
YIGLKDRKRPSELPRIASQVKYA

SEQ ID NO: 57 - protein, 23 aa, synthetic construct
YIGLKDRKRPSELQRIASQVKYA

SEQ ID NO: 58 - protein, 23 aa, synthetic construct
YIGLKDRKRPSELWRIASQVKYA

SEQ ID NO: 59 - protein, 23 aa, synthetic construct
YIGLKDRKRPSELSRIASQVKYA

SEQ ID NO: 60 - protein, 24 aa, synthetic construct
GSGGGSGGGSGGGSGGGSGGGSGG

SEQ ID NO: 61 - protein, 8 aa, synthetic construct
GSGGGSGG

SEQ ID NO: 62 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAIGFPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAIGFPQTFGQGTKVEIK

SEQ ID NO: 64 - protein, 221 aa, synthetic construct
EVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGS

SEQ ID NO: 65 - protein, 114 aa, synthetic construct
EVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTV

SEQ ID NO: 66 - protein, 39 aa, synthetic construct
GCGGSLRPAPPPISGGGYRARPAKAAATQKKVERKAPDA

SEQ ID NO: 67 - protein, 35 aa, synthetic construct
SLRPAPPPISGGGYRARPAKAAATQKKVERKAPDA

SEQ ID NO: 68 - protein, 225 aa, synthetic construct
SLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCTG

SEQ ID NO: 69 - protein, 168 aa, synthetic construct
GHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGGT

SEQ ID NO: 70 - protein, 168 aa, synthetic construct
GHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGTG

SEQ ID NO: 71 - protein, 452 aa, synthetic construct
EVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGSGGSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCTG

SEQ ID NO: 72 - protein, 391 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQAIGFPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGAGGGSGGGHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGGT

SEQ ID NO: 73 - protein, 395 aa, synthetic construct
EVQLLESGGGLVQPGGSLRLSCAASGFTFSSYAMSWVRQAPGKGLEQVSAISGSGGSTYYADSVKGRFTISRDNSKNTLYLQMNSLRAEDTAVYYCAKTLAAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGSGGGHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGGSGGGSGGGHRPLDKKREEAPSLRPAPPPISGGGYRARPAKAAATQKKVERKAPDAGGTG

SEQ ID NO: 74 - protein, 25 aa, synthetic construct
RLVKALKTDKFLGRIGSEKCNDKGK

SEQ ID NO: 75 - protein, 25 aa, synthetic construct
RKTDALELVFLKKGGIGSKKCNDKR

SEQ ID NO: 76 - protein, 25 aa, synthetic construct
CRKKKRKKKALLLGIGDFNSEVTDG

SEQ ID NO: 77 - protein, 25 aa, synthetic construct
KKRKLVALTDFLGICGSENDGRKKK

SEQ ID NO: 78 - protein, 25 aa, synthetic construct
LVRAKLTDKFLGKRIGSKECNKDKG

SEQ ID NO: 79 - protein, 25 aa, synthetic construct
ALLLGIGRDFNKKKRKKKSEVTDGC

SEQ ID NO: 80 - protein, 6 aa, synthetic construct
HHHHHH

SEQ ID NO: 81 - protein, 4 aa, synthetic construct
GGSG